(19) United States
(12) Patent Application Publication
(10) Pub. No.: US 2022/0235005 A1
(43) Pub. Date: Jul. 28, 2022

(54) FUNGICIDAL N-(PYRID-3-YL)CARBOXAMIDES

(71) Applicant: BASF SE, Ludwigshafen (DE)

(72) Inventors: Bernd Mueller, Ludwigshafen (DE); Michael Seet, Ludwigshafen (DE); Georg Christoph Rudolf, Ludwigshafen (DE); Wassilios Grammenos, Ludwigshafen (DE); Benjamin Juergen Merget, Ludwigshafen (DE); Andreas Koch, Limburgerhof (DE); Nadine Riediger, Limburgerhof (DE); Christine Wiebe, Ludwigshafen (DE); Thomas Grote, Ludwigshafen (DE); Jan Klaas Lohmann, Ludwigshafen (DE); Christian Harald Winter, Ludwigshafen (DE); Anja Weber, Duesseldorf (DE)

(21) Appl. No.: 17/608,497

(22) PCT Filed: May 26, 2020
(86) PCT No.: PCT/EP2020/064573

§ 371 Date: Nov. 3, 2021

(30) Foreign Application Priority Data
Jun. 6, 2019 (EP) 19178605.2

(51) Int. Cl. C07D 213/75; A01N 47/36; A01P 3/00

(52) U.S. Cl. CPC C07D 213/75; A01P 3/00; A01N 47/36

(57) ABSTRACT

The present invention relates to the use as fungicides of compounds of formula (I) wherein the variables are defined as given in the description and claims. The invention further relates to the compounds I and composition for compounds of formula I.

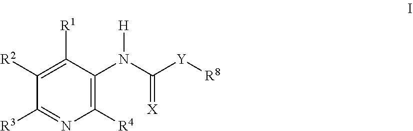

The present invention relates to the use of pyridine compounds and the N-oxides and the salts thereof as fungicides as well to new pyridine compounds. The invention also relates to the composition comprising at least one compound I, to the method for combating phytopathogenic fungi and to the seed coated with at least one compound of the formula I.

In many cases, in particular at low application rates, the fungicidal activity of known compounds is unsatisfactory. Based on this, it was an objective of the present invention to provide compounds having improved activity and/or a broader activity spectrum against phytopathogenic fungi. Another object of the present invention is to provide fungicides with improved toxicological properties or with improved environmental fate properties. These and further objects are achieved by the use of pyridine carboxamides of formula (I), as defined below, and by their agriculturally suitable salts as well by the new pyridine carboxamides of formula (I).

Accordingly, the present invention relates to use of the compounds of formula I

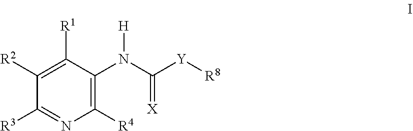

wherein
X O or S;
R¹ is in each case independently selected from hydrogen, halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl, C₃-C₆-cycloalkyl, wherein the acyclic and cyclic moieties of R¹ are unsubstituted or substituted by one to six groups R^{1a} which independently of one another are selected from:
   halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl; and wherein the groups R^{1a} are unsubstituted or substituted by one to six halogen or CN;
R² is in each case independently selected from halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl, C₃-C₆-cycloalkyl, wherein the acyclic and cyclic moieties of R² are unsubstituted or substituted by one to six groups R^{2a} which independently of one another are selected from:
   halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl; and wherein the groups R^{2a} are unsubstituted or substituted by one to six halogen or CN;
R³ is in each case independently selected from halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl, C₃-C₆-cycloalkyl, wherein the acyclic and cyclic moieties of R³ are unsubstituted or substituted by one to six groups R^{3a} which independently of one another are selected from:
   halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl; and wherein the groups R^{3a} are unsubstituted or substituted by one to six halogen or CN;
R⁴ is in each case independently selected from hydrogen, halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl, C₃-C₆-cycloalkyl, wherein the acyclic and cyclic moieties of R⁴ are unsubstituted or substituted by one to six groups R^{4a} which independently of one another are selected from:
   halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl; and wherein the groups R^{4a} are unsubstituted or substituted by one to six halogen or CN;
      R² and R³ together with the carbon atoms they are attached to can form a substituted ring of the formula II:

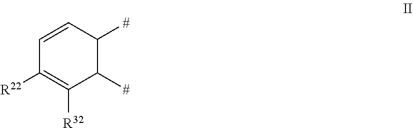

wherein
# means the connection to the pyridine ring of the formula I:
R²² is selected from the group consisting of H, halogen, C₁-C₆-alkyl, C₁-C₆-halogenalkyl.
R³² is selected from the group consisting of halogen, C₁-C₆-alkyl, C₁-C₆-halogenalkyl; and
Y is NR⁵, CR⁶R⁷;
R⁵ is in each case independently selected from CN, C₁-C₆-alkyl, C₂-C₆-alkenyl, C₂-C₆-alkynyl, O—C₁-C₆-alkyl, O—C₂-C₆-alkenyl, O—C₂-C₆-alkynyl, C₃-C₆-cycloalkyl, CH₂—C₃-C₆-cycloalkyl, O—C₃-C₆-cycloalkyl, C₃-C₆-cycloalkenyl, O—C₃-C₆-cycloalkenyl, C(R″)═N—OR′, phenyl, —CH₂-phenyl, a five- or six-membered heteroaryl, or CH₂-heteroaryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
   wherein the acyclic and cyclic moieties of R⁵ are unsubstituted or substituted by one to six groups R^{5a} which independently of one another are selected from:
   halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein the acyclic and cyclic moieties of R^{5a} are unsubstituted or substituted by one to six groups R^{5b} halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C(R″)═N—OR′;
   wherein the groups R^{5b} are unsubstituted or substituted by one to six halogen or CN;
R′ is in each case independently selected from C₁-C₆-alkyl, C₂-C₆-alkenyl, C₂-C₆-alkynyl, wherein the acyclic moieties of R″ are unsubstituted or substituted by one to six groups R^{R′} independently of one another are selected from:
   halogen, CN, O—C₁-C₆-alkyl, C₃-C₆-cycloalkyl, O—C₃-C₆-cycloalkyl, phenyl, phenoxy;
   wherein the acyclic and cyclic moieties of R^{R′} are unsubstituted or substituted by one to six groups selected from halogen, CN, C₁-C₆-alkyl, C₁-C₆-halogenalkyl, C₃-C₆-cycloalkyl, O—C₁-C₆-alkyl, O—C₁-C₆-halogenalkyl;
R″ is in each case independently selected from hydrogen, C₁-C₆-alkyl, C₂-C₆-alkenyl, C₂-C₆-alkynyl, C₃-C₆-cycloalkyl, C₃-C₆-cycloalkenyl, C(R″)═N—OR′, phenyl, a five- or six-membered heteroaryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
   the acyclic and cyclic moieties of R″ are unsubstituted or substituted by one to six groups halogen, CN, C₁-C₆-alkyl, C₁-C₆-halogenalkyl, C₃-C₆-cycloalkyl, O—C₁-C₆—O-alkyl, O—C₁-C₆-halogenalkyl;
R⁶ is in each case independently selected from halogen, CN, C₁-C₈-alkyl, C₂-C₈-alkenyl, C₂-C₈-alkynyl, C₁-C₈-halogenalkyl, C₂-C₈-halogenalkenyl, C₂-C₈-halogenalkynyl, O—C₁-C₈-alkyl, O—C₂-C₈-alkenyl, O—C₂-C₈-alkynyl, C₃-C₆-cycloalkyl, O—C₃-C₆-cycloalkyl, CH₂—C₃-C₆-cycloalkyl, C₃-C₆-cycloalkenyl, O—C₃-C₆-cycloalkenyl, CH₂—C₃-C₆-cycloalkenyl, C(R″)═N—OR′, phenyl, phenoxy, —CH₂-phenyl, a five- or six-membered heteroaryl, heteroaryloxy or CH₂-heteroaryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
   wherein the acyclic and cyclic moieties of R⁶ are unsubstituted or substituted by one to six groups R^{6a} which independently of one another are selected from:
   halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C₃-C₆-cycloalkenyl, O—C₃-C₆-cycloalkenyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
   the acyclic and cyclic moieties of R^{6a} are unsubstituted or substituted by one to six groups R^{6b} halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C(R″)═N—OR′;
   wherein the groups R^{6b} are unsubstituted or substituted by one to six halogen or CN;
R⁷ is in each case independently selected from hydrogen, OH, halogen, CN, C₁-C₈-alkyl, C₂-C₈-alkenyl, C₂-C₈-alkynyl, C₁-C₈-halogenalkyl, C₂-C₈-halogenalkenyl, C₂-C₈-halogenalkynyl, O—C₁-C₈-alkyl, O—C₂-C₈-alkenyl, O—C₂-C₈-alkynyl, C₃-C₆-cycloalkyl, O—C₃-C₆-cycloalkyl, CH₂—C₃-C₆-cycloalkyl, C₃-C₆-cycloalkenyl, O—C₃-C₆-cycloalkenyl, CH₂—C₃-C₆-cycloalkenyl, C(R″)═N—OR′, phenyl, phenoxy, —CH₂-phenyl, a five- or six-membered heteroaryl, heteroaryloxy or CH₂-heteroaryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
   wherein the acyclic and cyclic moieties of R⁷ are unsubstituted or substituted by one to six groups R^{7a} which independently of one another are selected from:
   halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C₃-C₆-cycloalkenyl, O—C₃-C₆-cycloalkenyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
   the acyclic and cyclic moieties of R^{7a} are unsubstituted or substituted by one to six groups R^{7b} halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C(R″)═N—OR′;

wherein the groups R^{7b} are unsubstituted or substituted by one to six halogen or CN;

or
R⁶ and R⁷ form together with the C atom to which they are bound a group ═N—OR′;
R⁸ is in each case independently selected from C₃-C₁₀-cycloalkyl, C₃-C₁₀-cycloalkenyl, phenyl or a five- or six-membered heteroaryl, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
   wherein the acyclic and cyclic moieties of R⁸ are unsubstituted or substituted by one to six groups R^{8a} which independently of one another are selected from:
   halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C₃-C₆-cycloalkenyl, O—C₃-C₆-cycloalkenyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
   the acyclic and cyclic moieties of R^{8a} are unsubstituted or substituted by one to six groups R^{8b} halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C₃-C₆-cycloalkenyl, O—C₃-C₆-cycloalkenyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein

the groups R^{8b} are unsubstituted or substituted by one to six halogen or CN;
with the proviso that:
if Y is CR⁶R⁷, R¹ and R⁴ are H and
R⁶ is unsubstituted C₁-C₄-alkyl and
R⁷ is H, OH, CH₃, C₂H₅,
R⁸ is not an unsubstituted phenyl;
if Y is CR⁶R⁷, R¹ and R⁴ are H and
R⁶ is F and
R⁷ is F,
R⁸ is not an unsubstituted phenyl;
if Y is CR⁶R⁷, R¹ and R⁴ are H and
R⁶ is CH₃ and
R⁷ is CH₃,
R⁸ is not cyclopropyl;
if Y is CR⁶R⁷, R¹ and R⁴ are H and
R⁶ is CH₂-phenyl and
R⁷ is H,
R⁸ is not cyclopropyl;
if Y is CR⁶R⁷, R¹ and R⁴ are H and
R⁶ is i-propyl and
R⁷ is H,
R⁸ is not 4-methoxyphenyl;
and following compounds are excluded

I.A-1

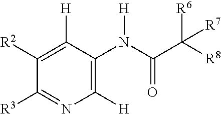

| R² | R³ | R⁶ | R⁷ | R⁸ |
|---|---|---|---|---|
| —CH₃ | —CH₃ | cyclopentyl | H | phenyl |
| —CH₃ | —CH₃ | CF₃ | —OCH₃ | phenyl |
| —CH₃ | —CH₃ | O—C(CH₃)₃ | H | phenyl |

and the N-oxides and the agriculturally acceptable salts thereof as fungicides.

The N-oxides may be prepared from the inventive compounds according to conventional oxidation methods, e. g. by treating compounds I with an organic peracid such as metachloroperbenzoic acid (cf. WO 03/64572 or J. Med. Chem. 38(11), 1892-903, 1995); or with inorganic oxidizing agents such as hydrogen peroxide (cf. J. Heterocyc. Chem. 18(7), 1305-8, 1981) or oxone (cf. J. Am. Chem. Soc. 123(25), 5962-5973, 2001). The oxidation may lead to pure mono-N-oxides or to a mixture of different N-oxides, which can be separated by conventional methods such as chromatography.

Agriculturally acceptable salts of the compounds of the formula I encompass especially the salts of those cations or the acid addition salts of those acids whose cations and anions, respectively, have no adverse effect on the fungicidal action of the compounds I. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may be substituted with one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of acceptable acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting a compound I with an acid of the corresponding anion, preferably of hydrochloric acid, hydrobromic acid, sulfuric acid, phosphoric acid or nitric acid.

Compounds of the formula I can exist as one or more stereoisomers. The various stereoisomers include enantiomers, diastereomers, atropisomers arising from restricted rotation about a single bond of asymmetric groups and geometric isomers. They also form part of the subject matter of the present invention. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, e.g. a racemate, individual stereoisomers, or as an optically active form.

Compounds of the formula I can be present in different crystal modifications whose biological activity may differ. They also form part of the subject matter of the present invention.

In respect of the variables, the embodiments of the intermediates obtained during preparation of compounds I correspond to the embodiments of the compounds of formula I. The term “compounds I” refers to compounds of the formula I.

In the following, the intermediate compounds are further described. A skilled person will readily understand that the preferences for the substituents, also in particular the ones given in the tables below for the respective substituents, given herein in connection with compounds I apply for the intermediates accordingly. Thereby, the substituents in each case have independently of each other or more preferably in combination the meanings as defined herein.

If the synthesis yields mixtures of isomers, a separation is generally not necessarily required since in some cases the individual isomers can be interconverted during work-up for use or during application (e. g. under the action of light, acids or bases). Such conversions may also take place after use, e. g. in the treatment of plants in the treated plant, or in the harmful fungus to be controlled.

In the definitions of the variables given above, collective terms are used which are generally representative for the substituents in question. The term “Cₙ-Cₘ” indicates the number of carbon atoms possible in each case in the substituent or substituent moiety in question.

The term “halogen” refers to fluorine, chlorine, bromine and iodine.

The term “C₁-C₆-alkyl” refers to a straight-chained or branched saturated hydrocarbon group having 1 to 6 carbon atoms, e.g. methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Likewise, the term “C₂-C₄-alkyl” refers to a straight-chained or branched alkyl group having 2 to 4 carbon atoms, such as ethyl, propyl (n-propyl), 1-methylethyl (iso-propoyl), butyl, 1-methylpropyl (sec.-butyl), 2-methylpropyl (iso-butyl), 1,1-dimethylethyl (tert.-butyl).

The term “C₁-C₆-halogenalkyl” refers to an alkyl group having 1 or 6 carbon atoms as defined above, wherein some or all of the hydrogen atoms in these groups may be replaced by halogen atoms as mentioned above. Examples are “C₁-C₂-halogenalkyl” groups such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl or pentafluoroethyl.

The term “C₁-C₆-alkoxy” refers to a straight-chain or branched alkyl group having 1 to 6 carbon atoms which is bonded via an oxygen, at any position in the alkyl group. Examples are “C₁-C₄-alkoxy” groups, such as methoxy, ethoxy, n-propoxy, 1-methylethoxy, butoxy, 1-methyl¬propoxy, 2-methylpropoxy or 1,1-dimethylethoxy.

The term “C₁-C₆-halogenalkoxy” refers to a C₁-C₆-alkoxy radical as defined above, wherein some or all of the hydrogen atoms in these groups may be replaced by halogen atoms as mentioned above. Examples are “C₁-C₄-halogenalkoxy” groups, such as OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chlorothoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoro¬propoxy, 2 chloropropoxy, 3-chloropropoxy, 2,3-dichloropropoxy, 2-bromopropoxy, 3 bromopropoxy, 3,3,3-trifluoropropoxy, 3,3,3-trichloropropoxy, OCH₂—C₂F₅, OCF₂—C₂F₅, 1-fluoromethyl-2-fluoroethoxy, 1-chloromethyl-2-chloroethoxy, 1-bromomethyl-2-bromoethoxy, 4-fluorobutoxy, 4-chlorobutoxy, 4-bromobutoxy or nonafluorobutoxy.

The term “C₂-C₆-alkenyl” refers to a straight-chain or branched unsaturated hydrocarbon radical having 2 to 6 carbon atoms and a double bond in any position. Examples are “C₂-C₄-alkenyl” groups, such as ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₂-C₆-halogenalkenyl” refers to an alkyl group having 2 or 6 carbon atoms as defined above, wherein some or all of the hydrogen atoms in these groups may be replaced by halogen atoms as mentioned above.

The term “C₂-C₆-alkenyloxy” refers to a straight-chain or branched alkenyl group having 2 to 6 carbon atoms which is bonded via an oxygen, at any position in the alkenyl group. Examples are “C₂-C₄-alkenyloxy” groups.

The term “C₂-C₆-alkynyl” refers to a straight-chain or branched unsaturated hydrocarbon radical having 2 to 6 carbon atoms and containing at least one triple bond. Examples are “C₂-C₄-alkynyl” groups, such as ethynyl, prop-1-ynyl, prop-2-ynyl (propargyl), but-1-ynyl, but-2-ynyl, but-3-ynyl, 1-methyl-prop-2-ynyl.

The term “C₂-C₆-halogenalkynyl” refers to an alkyl group having 2 or 6 carbon atoms as defined above, wherein some or all of the hydrogen atoms in these groups may be replaced by halogen atoms as mentioned above.

The term “C₂-C₆-alkynyloxy” refers to a straight-chain or branched alkynyl group having 2 to 6 carbon atoms which is bonded via an oxygen, at any position in the alkynyl group. Examples are “C₂-C₄-alkynyloxy” groups.

The term “C₃-C₆-cycloalkyl” refers to monocyclic saturated hydrocarbon radicals having 3 to 6 carbon ring members, such as cyclopropyl, cyclobutyl, cyclopentyl, cyclohexyl. Accordingly, a saturated three-, four-, five-, six-, seven-, eight-, nine or ten-membered carbocyclyl or carbocycle is a “C₃-C₁₀-cycloalkyl”.

The term “C₃-C₆-cycloalkenyl” refers to a monocyclic partially unsaturated 3-, 4-5- or 6-membered carbocycle having 3 to 6 carbon ring members and at least one double bond, such as cyclopentenyl, cyclopentadienyl, cyclohexadienyl. Accordingly, a partially unsaturated three-, four-, five-, six-, seven-, eight-, nine or ten-membered carbocyclyl or carbocycle is a “C₃-C₁₀-cycloalkenyl”.

The term “C₃-C₈-cycloalkyl-C₁-C₄-alkyl” refers to alkyl having 1 to 4 carbon atoms (as defined above), where According to one hydrogen atom of the alkyl radical is replaced by a cycloalkyl radical having 3 to 8 carbon atoms (as defined above).

The term “saturated or partially unsaturated three-, four-, five-, six-, seven-, eight-, nine or ten-membered heterocyclyl or heterocycle, wherein the heterocyclyl or heterocycle contains 1, 2, 3 or 4 heteroatoms selected from N, O and S” is to be understood as meaning both saturated and partially unsaturated heterocycles, wherein the ring member atoms of the heterocycle include besides carbon atoms 1, 2, 3 or 4 heteroatoms independently selected from the group of O, N and S. For example:

a 3- or 4-membered saturated heterocycle which contains 1 or 2 heteroatoms from the group consisting of O, N and S as ring members such as oxirane, aziridine, thiirane, oxetane, azetidine, thiethane, [1,2]dioxetane, [1,2]dithietane, [1,2]diazetidine; and
a 5- or 6-membered saturated or partially unsaturated heterocycle which contains 1, 2 or 3 heteroatoms from the group consisting of O, N and S as ring members such as 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl and also the corresponding -ylidene radicals; and
a 7-membered saturated or partially unsaturated heterocycle such as tetra- and hexahydroazepinyl, such as 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, hexahydroazepin-1-, -2-, -3- or -4-yl, tetra- and hexahydrooxepinyl such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, hexahydroazepin-1-, -2-, -3- or -4-yl, tetra- and hexahydro-1,3-diazepinyl, tetra- and hexahydro-1,4-diazepinyl, tetra- and hexahydro-1,3-oxazepinyl, tetra- and hexahydro-1,4-oxazepinyl, tetra- and hexahydro-1,3-dioxepinyl, tetra- and hexahydro-1,4-dioxepinyl and the corresponding -ylidene radicals.

The term “substituted” refers to substituted with 1, 2, 3 or up to the maximum possible number of substituents.

The term “5- or 6-membered heteroaryl” or “5- or 6-membered heteroaromatic” refers to aromatic ring systems including besides carbon atoms, 1, 2, 3 or 4 heteroatoms independently selected from the group consisting of N, O and S, for example,

a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol-4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl; or
a 6-membered heteroaryl, such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl.

In the following, particular embodiments of the inventive compounds are described. Therein, specific meanings of the respective substituents are further detailed, wherein the meanings are in each case on their own but also in any combination with one another, particular embodiments of the present invention.

Furthermore, in respect of the variables, generally, the embodiments of the compounds I also apply to the intermediates.

According to one embodiment of formula I, R¹ is H, halogen, C₁-C₆-alkyl or C₁-C₆-halogenalkyl, in particular H, F, Cl, CH₃, C₂H₅, CF₃ more specifically H, CH₃, F or Cl most preferred H, F or Cl, especially R¹ is hydrogen.

Particularly preferred embodiments of R¹ according to the invention are in Table P1 below, wherein each line of lines P1-1 to P1-13 corresponds to one particular embodiment of the invention. Thereby, for every R¹ that is present in the inventive compounds, these specific embodiments and preferences apply independently of the meaning of any other R¹ that may be present in the ring:

TABLE P1

| No. | R¹ |
|---|---|
| P1-1 | H |
| P1-2 | Cl |
| P1-3 | F |
| P1-4 | Br |
| P1-5 | CN |
| P1-6 | CH₃ |
| P1-7 | CH₂OH₃ |
| P1-8 | CF₃ |
| P1-9 | CHF₂ |
| P1-10 | OCH₃ |
| P1-11 | OCH₂OH₃ |
| P1-12 | OCF₃ |
| P1-13 | OCHF₂ |

According to one embodiment of formula I, R² is selected from the group consisting of halogen, C₁-C₆-alkyl, C₁-C₆-halogenalkyl, —O—C₁-C₆-alkyl, O—C₁-C₆-halogenalkyl, in particular F, Cl, CH₃, C₂H₅, CF₃, CH₂F, CHF₂, OCH₃, OC₂H₅, OCHF₂ more specifically CH₃, CH₂F, CF₂H, CF₃, OCH₃, more preferred CH₃, CHF₂, OCH₃, most preferred CH₃, OCH₃.

According to one embodiment of formula I, R² is selected from the group consisting of halogen, C₁-C₆-alkyl, C₁-C₆-halogenalkyl, —O—C₁-C₆-alkyl, in particular F, Cl, CH₃, C₂H₅, CF₃, CH₂F, CHF₂, OCH₃, OC₂H₅, more specifically CH₃, CH₂F, CF₂H, CF₃, most preferred CH₃, CHF₂.

According to still another embodiment of formula I, R² is halogen, in particular F, Cl, Br or I, more specifically F, Cl or Br, in particular F or Cl.

According to still another embodiment of formula I, R² is F.

According to still another embodiment of formula I, R² is C.

According to still another embodiment of formula I, R² is Br.

According to still another embodiment of formula I, R² is C₁-C₆-alkyl, in particular C₁-C₄-alkyl, such as CH₃. or C₂H₅, in particular CH₃ or CH₂CH₃.

According to still another embodiment of formula I, R² is C₁-C₆-halogenalkyl, in particular C₁-C₄-halogenalkyl, such as CF₃, CCl₃, FCH₂, ClCH₂, F₂CH, Cl₂CH, CF₃CH₂, CCl₃CH₂ or CF₂CHF₂.

According to still a further embodiment of formula I, R² is C₂-C₆-alkenyl, in particular C₂-C₄-alkenyl, such as CH═CH₂, C(CH₃)═CH₂, CH₂CH═CH₂.

According to a further specific embodiment of formula I, R² is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CH₂CH═CHF, CH₂CH═CHCl, CH₂CH═CF₂, CH₂CH═CCl₂, CF₂CH═CF₂, CCl₂CH═CCl₂, CF₂CF═CF₂, CCl₂CCl═CCl₂.

According to still a further embodiment of formula I, R² is C₂-C₆-alkynyl or C₂-C₆-halogenalkynyl, in particular C₂-C₄-alkynyl or C₂-C₄-halogenalkynyl, such as C≡CH, CH₂C≡CH, C≡CCl, CH₂C≡CCl, or CCl₂C≡CCl.

According to a further specific embodiment of formula I, R² is O—C₁-C₆-alkyl, in particular C₁-C₄-alkyl, more specifically C₁-C₂-alkoxy. R² is such as OCH₃ or OCH₂CH₃.

According to a further specific embodiment of formula I, R² is O—C₂-C₆-alkenyl in particular C₂-C₄-alkenyl, more specifically C₂-C₃-alkenyl. R² is such as OCH═CH₂, OCH₂CH═CH₂.

According to a further specific embodiment of formula I, R² is O—C₂-C₆-alkynyl, in particular C₂-C₆-alkynyl, in particular C₂-C₄-alkynyl, more specifically C₂-C₃-alkynyl. R² is such as O—CH₂—C≡CH.

According to a further specific embodiment of formula I, R² is O—C₁-C₆-halogenalkyl, in particular OCF₃, OCCl₃, OFCH₂, OClCH₂, OF₂CH, OCl₂CH, OCF₃CH₂, OCCl₃H₂ or OCF₂CHF₂, more specifically OCF₃, OF₂CH, OFCH₂.

According to still another embodiment of formula I, R² is C₃-C₆-cycloalkyl, in particular cyclopropyl.

According to still another embodiment of formula I, R² is C₃-C₆-halogencycloalkyl. In a special embodiment R^{2a} is fully or partially halogenated cyclopropyl, such as 1-F-cyclopropyl, 1-Cl-cyclopropyl, 2,2-F₂-cyclopropyl, 2,2-C₂-cyclopropyl.

Particularly preferred embodiments of R² according to the invention are in Table P2 below, wherein each line of lines P2- to P2-27 corresponds to one particular embodiment of the invention, wherein P2- to P2-27 are also in any combination with one another a preferred embodiment of the present invention. The connection point to the carbon atom, to which R² is bound is marked with “#” in the drawings.

TABLE P2

| No. | R² |
|---|---|
| P2-1 | CH₃ |
| P2-2 | CH₂F |
| P2-3 | CHF₂ |
| P2-4 | CF₃ |
| P2-5 | C₂H₅ |
| P2-6 | CH(CH₃)₂ |
| P2-7 | CH₂CH₂CH₃ |
| P2-8 | CH₂CH₂CH₂CH₃ |
| P2-9 | CH₂CH(CH₃)₂ |
| P2-10 | C(CH₃)₃ |
| P2-11 | CH₂CH₂CH₂CH₂CH₃ |
| P2-12 | CH═CH₂ |
| P2-13 | CH₂CH═CH₂ |
| P2-14 | C≡CH |
| P2-15 | CH₂C≡CH |
| P2-16 | OCF₃ |
| P2-17 | OCH₃ |
| P2-18 | OCHF₂ |
| P2-19 | OC₂H₅ |
| P2-20 | CN |
| P2-21 | F |
| P2-22 | Cl |
| P2-23 | Br |
| P2-24 | 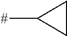 |
| P2-25 | 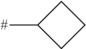 |
| P2-26 | 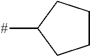 |
| P2-27 | 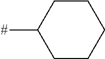 |

According to one embodiment of formula I, R³ is selected from the group consisting of halogen, C₁-C₆-alkyl, C₁-C₆-halogenalkyl, —O—C₁-C₆-alkyl, O—C₁-C₆-halogenalkyl, in particular F, Cl, CH₃, C₂H₅, CF₃, CH₂F, CHF₂, OCH₃, OC₂H₅, OCHF₂ more specifically CH₃, CH₂F, CF₂H, CF₃, most preferred CH₃, CHF₂.

According to one embodiment of formula I, R³ is selected from the group consisting of halogen, C₁-C₆-alkyl, C₁-C₆-halogenalkyl, —O—C₁-C₆-alkyl, in particular F, Cl, CH₃, C₂H₅, CF₃, CH₂F, CHF₂, OCH₃, OC₂H₅, more specifically CH₃, CH₂F, CF₂H, CF₃, most preferred CH₃, CHF₂.

According to still another embodiment of formula I, R³ is halogen, in particular F, Cl, Br or I, more specifically F, Cl or Br, in particular F or Cl.

According to still another embodiment of formula I, R³ is F.

According to still another embodiment of formula I, R³ is Cl.

According to still another embodiment of formula I, R³ is Br.

According to still another embodiment of formula I, R³ is C₁-C₆-alkyl, in particular C₁-C₄-alkyl, such as CH₃ or C₂H₅, in particular CH₃ or CH₂CH₃.

According to still another embodiment of formula I, R³ is C₁-C₆-halogenalkyl, in particular C₁-C₄-halogenalkyl, such as CF₃, CCl₃, FCH₂, ClCH₂, F₂CH, Cl₂CH, CF₃CH₂, CCl₃CH₂ or CF₂CHF₂.

According to still a further embodiment of formula I, R³ is C₂-C₆-alkenyl, in particular C₂-C₄-alkenyl, such as CH═CH₂, C(CH₃)═CH₂, CH₂CH═CH₂.

According to a further specific embodiment of formula I, R³ is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CH₂CH═CHF, CH₂CH═CHCl, CH₂CH═CF₂, CH₂CH═CCl₂, CF₂CH═CF₂, CCl₂CH═CCl₂, CF₂CF═CF₂, CCl₂CCl═CCl₂.

According to still a further embodiment of formula I, R³ is C₂-C₆-alkynyl or C₂-C₆-halogenalkynyl, in particular C₂-C₄-alkynyl or C₂-C₄-halogenalkynyl, such as C≡CH, CH₂C≡CH, C≡CCl, CH₂C≡CCl, or CCl₂C≡CCl.

According to a further specific embodiment of formula I, R³ is O—C₁-C₆-alkyl, in particular C₁-C₄-alkyl, more specifically C₁-C₂-alkoxy. R³ is such as OCH₃ or OCH₂CH₃.

According to a further specific embodiment of formula I, R³ is O—C₂-C₆-alkenyl in particular C₂-C₄-alkenyl, more specifically C₂-C₃-alkenyl. R³ is such as OCH═CH₂, OCH₂CH═CH₂.

According to a further specific embodiment of formula I, R³ is O—C₂-C₆-alkynyl, in particular C₂-C₆-alkynyl, in particular C₂-C₄-alkynyl, more specifically C₂-C₃-alkynyl. R³ is such as O—CH₂—C≡CH.

According to a further specific embodiment of formula I, R² is O—C₁-C₆-halogenalkyl, in particular OCF₃, OCCl₃, OFCH₂, OClCH₂, CF₂CH, OCl₂CH, OCF₃CH₂, OCCl₃H₂ or OCF₂CHF₂, more specifically OCF₃, OF₂CH, OFCH₂.

According to still another embodiment of formula I, R³ is C₃-C₆-cycloalkyl, in particular cyclopropyl.

According to still another embodiment of formula I, R³ is C₃-C₆-halogencycloalkyl. In a special embodiment R^{3a} is fully or partially halogenated cyclopropyl, such as 1-F-cyclopropyl, 1-Cl-cyclopropyl, 2,2-F₂-cyclopropyl, 2,2-Cl₂-cyclopropyl.

Particularly preferred embodiments of R³ according to the invention are in Table P3 below, wherein each line of lines P3-1 to P3-27 corresponds to one particular embodiment of the invention, wherein P3-1 to P3-27 are also in any combination with one another a preferred embodiment of the present invention. The connection point to the carbon atom, to which R² is bound is marked with “#” in the drawings.

TABLE P3

| No. | R³ |
|---|---|
| P3-1 | CH₃ |
| P3-2 | CH₂F |
| P3-3 | CHF₂ |
| P3-4 | CF₃ |
| P3-5 | C₂H₅ |
| P3-6 | CH(CH₃)₂ |
| P3-7 | CH₂CH₂CH₃ |
| P3-8 | CH₂CH₂CH₂CH₃ |
| P3-9 | CH₂CH(CH₃)₂ |
| P3-10 | C(CH₃)₃ |
| P3-11 | CH₂CH₂CH₂CH₂CH₃ |
| P3-12 | CH═CH₂ |
| P3-13 | CH₂CH═CH₂ |
| P3-14 | C≡CH |
| P3-15 | CH₂C≡CH |
| P3-16 | OCHF₃ |
| P3-17 | OCH₃ |
| P3-18 | OCHF₂ |
| P3-19 | OC₂H₅ |
| P3-20 | CN |
| P3-21 | F |
| P3-22 | Cl |
| P3-23 | Br |
| P3-24 | 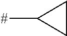 |
| P3-25 | 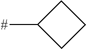 |
| P3-26 | 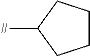 |
| P3-27 | 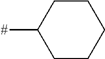 |

In one further embodiment of the invention R² and R³ together with the carbon atoms they are attached to can form a substituted ring of the formula II:

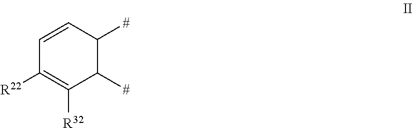

wherein
# means the connection to the pyridine ring of the formula I:
R²² is selected from the group consisting of H, halogen, C₁-C₆-alkyl, C₁-C₆-halogenalkyl.
R³² is selected from the group consisting of halogen, C₁-C₆-alkyl, C₁-C₆-halogenalkyl.

In one embodiment of the invention R²² is preferably selected from the group consisting of: H, F, most preferred H.

In one embodiment of the invention R³² is preferably selected from the group consisting of: F, Cl, CH₃, most preferred F.

According to one embodiment of formula I, R⁴ is H, halogen, C₁-C₆-alkyl or C₁-C₆-halogenalkyl, in particular H, F, Cl, CH₃, C₂H₅, CF₃ more specifically H, CH₃, F or Cl most preferred H, F or Cl, especially R⁴ is hydrogen.

Particularly preferred embodiments of R⁴ according to the invention are in Table P4 below, wherein each line of lines P4-1 to P4-10 corresponds to one particular embodiment of the invention. Thereby, for every R⁴ that is present in the inventive compounds, these specific embodiments and preferences apply independently of the meaning of any other R⁴ that may be present in the ring:

TABLE P4

| No. | R⁴ |
|---|---|
| P4-1 | H |
| P4-2 | Cl |
| P4-3 | F |
| P4-4 | Br |
| P4-5 | CN |
| P4-6 | CH₃ |
| P4-7 | CH₂OH₃ |
| P4-8 | CF₃ |
| P4-9 | CHF₂ |
| P4-10 | OCH₃ |
| P4-11 | OCH2CH3 |
| P4-12 | OCF3 |
| P4-13 | OCHF2 |

Y is NR⁵, CR⁶R⁷;
R⁵ is in each case independently selected from CN, C₁-C₆-alkyl, C₂-C₆-alkenyl, C₂-C₆-alkynyl, O—C₁-C₆-alkyl, O—C₂-C₆-alkenyl, O—C₂-C₆-alkynyl, C₃-C₆-cycloalkyl, CH₂—C₃-C₆-cycloalkyl, O—C₃-C₆-cycloalkyl, C₃-C₆-cycloalkenyl, O—C₃-C₆-cycloalkenyl, C(R″)═N—OR′, phenyl, —CH₂-phenyl, a five- or six-membered heteroaryl, or CH₂-heteroaryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
   wherein the acyclic and cyclic moieties of R⁵ are unsubstituted or substituted by one to six groups R^{5a} which independently of one another are selected from:
   halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
   the acyclic and cyclic moieties of R^{5a} are unsubstituted or substituted by one to six groups halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C(R″)═N—OR′;
   wherein the groups R^{5a} are unsubstituted or substituted by one to six halogen or CN;
R′ is in each case independently selected from C₁-C₆-alkyl, C₂-C₆-alkenyl, C₂-C₆-alkynyl, wherein the acyclic moieties of R′ are unsubstituted or substituted by one to six groups R^{R′} independently of one another are selected from:
   halogen, CN, O—C₁-C₆-alkyl, C₃-C₆-cycloalkyl, O—C₃-C₆-cycloalkyl, phenyl, phenoxy;
   wherein the acyclic and cyclic moieties of R^{R′} are unsubstituted or substituted by one to six groups selected from halogen, CN, C₁-C₆-alkyl, C₁-C₆-halogenalkyl, C₃-C₆-cycloalkyl, O—C₁-C₆-alkyl, O—C₁-C₆-halogenalkyl;
R″ is in each case independently selected from hydrogen, C₁-C₆-alkyl, C₂-C₆-alkenyl, C₂-C₆-alkynyl, C₃-C₆-cycloalkyl, C₃-C₆-cycloalkenyl, C(R″)═N—OR′, phenyl, a five- or six-membered heteroaryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
   the acyclic and cyclic moieties of R″ are unsubstituted or substituted by one to six groups halogen, CN, C₁-C₆-alkyl, C₁-C₆-halogenalkyl, C₃-C₆-cycloalkyl, O—C₁-C₆—O-alkyl, O—C₁-C₆-halogenalkyl.

According to one embodiment of formula I, R⁵ is CN, C₁-C₆-alkyl, O—C₁-C₆-alkyl, C₃-C₆-cycloalkyl, O—C₃-C₆-cycloalkyl, C(R″)═N—OR′, phenyl, wherein the acyclic and cyclic moieties of R⁵ are unsubstituted or substituted by one to six groups R^{5a} which independently of one another are selected from:

halogen, C(R″)═N—OR′, phenyl; and wherein
the acyclic and cyclic moieties of R^{5a} are unsubstituted or substituted by one to six groups R^{5b} halogen, C₁-C₆-alkyl, C₁-C₆-halogenalkyl, O—C₁-C₆-alkyl, C(R″)═N—OR′;
wherein the groups R^{5b} are unsubstituted or substituted by one to six halogen or CN.

According to one further preferred embodiment of formula I, R⁵ is C₁-C₆-alkyl, phenyl, wherein the acyclic and cyclic moieties of R⁵ are unsubstituted or substituted by one to six groups R^{5a} which independently of one another are selected from:

halogen, phenyl; and wherein
the phenyl is unsubstituted or substituted by one to six halogen.

According to one further preferred embodiment of formula I, R⁵ is CH₃, C₂H₅, phenyl, wherein the acyclic and cyclic moieties of R⁵ are unsubstituted or substituted by one to six groups R^{5a} which independently of one another are selected from:

halogen, phenyl; and wherein
the phenyl is unsubstituted or substituted by one to six halogen.

According to one further preferred embodiment of formula I, R⁵ is CH₃, C₂H₅, phenyl, CH₂-phenyl, wherein the acyclic and cyclic moieties of R⁵ are unsubstituted or substituted by one to six groups R⁵¹ which independently of one another are selected from:

halogen.

According to one embodiment of formula I, R⁵ is CN. According to still another embodiment of formula I, R⁵ is C₁-C₆-alkyl, such as CH₃, C₂H₅, n-propyl, i-propyl, n-butyl, i-butyl, tert-butyl, n-pentyl or i-pentyl. Most preferably is i-propyl, tert-butyl.

According to still another embodiment of formula I, R⁵ is C₁-C₆-halogenalkyl, in particular C₁-C₄-halogenalkyl, such as CF₃, CCl₃, FCH₂, ClCH₂, F₂CH, Cl₂CH, CF₃CH₂, CCl₃CH₂ or CF₂CHF₂.

According to still another embodiment of formula I R⁵ is C₃-C₆-cycloalkyl, in particular cyclopropyl.

According to still another embodiment of formula I, R⁵ is C₃-C₆-halogencycloalkyl. In a special embodiment R⁵ is fully or partially halogenated cyclopropyl, such as 1-F-cyclopropyl, 1-Cl-cyclopropyl, 2,2-F₂-cyclopropyl, 2,2-C₂-cyclopropyl.

According to still a further embodiment of formula I, R⁵ is C₂-C₆-alkenyl, in particular C₂-C₄-alkenyl, such as C(CH₃)═CH₂, CH₂CH═CH₂, CH₂—CH₂—CH═CH₂.

According to a further specific embodiment of formula I, R⁵ is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CH₂CH═CHF, CH₂CH═CHCl, CH₂CH═CF₂, CH₂CH═CCl₂, CF₂CH═CF₂, CCl₂CH═CCl₂, CF₂CF═CF₂, CCl₂CCl═CCl₂.

According to still a further embodiment of formula I, R⁶ is C₂-C₆-alkynyl or C₂-C₆-halogenalkynyl, in particular C₂-C₄-alkynyl or C₂-C₄-halogenalkynyl, such as C≡CH, CH₂C≡CH, C≡CCl, CH₂C≡CCl, or CCl₂C≡CCl.

According to a further specific embodiment of formula I, R⁵ is O—C₁-C₆-alkanyl in particular C₂-C₄-alkanyl, more specifically C₁-C₂-alkanyl. R⁵ is such as OCH₃, OC₂H₅.

According to a further specific embodiment of formula I, R⁵ is O—C₂-C₆-alkenyl in particular C₂-C₄-alkenyl, more specifically C₂-C₃-alkenyl. R³ is such as OCH═CH₂, OCH₂CH═CH₂.

According to a further specific embodiment of formula I, R⁵ is O—C₂-C₆-alkynyl, in particular C₂-C₆-alkynyl, in particular C₂-C₄-alkynyl, more specifically C₂-C₃-alkynyl. R³ is such as OCH₂C≡CH,

According to still another embodiment of formula I, R⁵ is C₃-C₆-cycloalkyl, in particular cyclopropyl, cyclopentyl and cyclohexyl.

According to still another embodiment of formula I, R⁵ is CH₂—C₃-C₆-cycloalkyl, in particular CH₂-cyclopropyl, CH₂-cyclopentyl and CH₂-cyclohexyl.

According to still another embodiment of formula I, R⁵ is C₃-C₆-halogencycloalkyl. In a special embodiment R⁵ is fully or partially halogenated cyclopropyl, such as 1-F-cyclopropyl, 1-Cl-cyclopropyl, 2,2-F₂-cyclopropyl, 2,2-Cl₂-cyclopropyl.

According to a further specific embodiment of formula I, R⁵ is CR″═NOR′ such as

C(CH₃)═NOCH₃, C(CH₃)═NO—CH₂CH₃, C(CH₃)═NO—CH₂CF₃, C(CH₃)═NO—CH₂—CH═CH₂, C(CH₃)═NO—CH₂—CCH, C(CH₃)═NO—CH₂—C₆H₅;
C(CH₂CH₃)═NOCH₃, C(CH₂CH₃)═NO—CH₂CH₃, C(CH₂CH₃)═NO—CH₂CF₃, C(CH₂CH₃)═NO—CH₂—CH═CH₂, C(CH₂CH₃)═NO—CH₂—CCH, C(CH₂CH₃)═NO—CH₂—C₆H₅;
C(CF₃)═NOCH₃, C(CF₃)═NO—CH₂CH₃, C(CF₃)═NO—CH₂CF₃, C(CF₃)═NO—CH₂—CH═CH₂, C(CF₃)═NO—CH₂—CCH, C(CF₃)═NO—CH₂—C₆H₅;
C(C₃H₅)═NOCH₃, C(C₃H₅)═NO—CH₂CH₃, C(C₃H₅)═NO—CH₂CF₃, C(C₃H₅)═NO—CH₂—CH═CH₂, C(C₃H₅)═NO—CH₂—CCH, C(C₃H₅)═NO—CH₂—C₆H₅;
C(C₆H₅)═NOCH₃, C(C₆H₅)═NO—CH₂CH₃, C(C₆H₅)═NO—CH₂CF₃, C(C₆H₅)═NO—CH₂CH═CH₂, C(C₆H₅)═NO—CH₂—CCH, C(C₆H₅)═NO—CH₂—C₆H₅.

According to still another embodiment of formula I, R⁵ is phenyl or, wherein phenyl in each case is unsubstituted or substituted by identical or different groups R^{5a} which independently of one another are selected from halogen, C₁-C₂-alkyl, CR″═NOR′.

According to still another embodiment of formula I, R⁵ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl, preferred are pyrazol-1-yl, pyrazol-3-yl, pyrazol-4-yl, pyrazol-5-yl, 1,2,4-triazolyl-1-yl, 1,2,4-triazol-3-yl 1,2,4-triazol-5-yl, isoxazol-3-yl, isoxazol-4-yl, isoxazol-5-yl. The 5-membered heteroaryl in each case is unsubstituted or substituted by identical or different groups R^{5a} which independently of one another are selected from halogen, C₁-C₂-alkyl, CR″═NOR′.

According to still another embodiment of formula I, R⁵ is a 6-membered heteroaryl such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl, preferred are pyridin-2-yl, pyridin-3-yl, pyridin-4-yl. The 5-membered heteroaryl in each case is unsubstituted or substituted by identical or different groups R^{5a} which independently of one another are selected from halogen, C₁-C₂-alkyl, CR″═NOR′.

Particularly preferred embodiments of R⁵ according to the invention are in Table P5 below, wherein each line of lines P5-1 to P5-106 corresponds to one particular embodiment of the invention, wherein P5-1 to P5-106 are also in any combination with one another a preferred embodiment of the present invention. The connection point to the carbon atom, to which R⁵ is bound is marked with “#” in the drawings.

TABLE P5

| No. | R⁵ |
|---|---|
| P5-1 | CN |
| P5-2 | CH₃ |
| P5-3 | CH₂F |
| P5-4 | CHF₂ |
| P5-5 | CF₃ |
| P5-6 | C₂H₅ |
| P5-7 | C₃H₇ |
| P5-8 | CH(CH₃)₂ |
| P5-9 | CH₂CH₂CH₂CH₃ |
| P5-10 | CH₂CH(CH₃)₂ |
| P5-11 | C(CH₃)₃ |
| P5-12 | CH₂CH₂CH₂CH₂CH₃ |
| P5-13 | CH═CH₂ |
| P5-14 | CH₂CH═CH₂ |
| P5-15 | C≡CH |
| P5-16 | CH₂C≡CH |
| P5-17 | CH₂CH₂CH(CH₃)₂ |
| P5-18 | OCH₃ |
| P5-19 | OC₂H₅ |
| P5-20 | OCH₂CH₂CH₃ |
| P5-21 | OCH(CH₃)₂ |
| P5-22 | OCH₂—CH═CH₂ |
| P5-23 | CH₂—C≡CH |
| P5-24 | C(CH₃)═N—OCH₃ |
| P5-25 | C(CH₃)═N—OCH₂—C₆H₅ |
| P5-26 | C(C₆H₅)═N—OCH₃ |
| P5-27 | 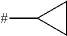 |
| P5-28 | 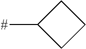 |
| P5-29 | 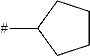 |
| P5-30 | 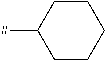 |
| P5-31 | 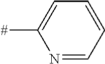 |
| P5-32 | 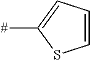 |
| P5-33 | C₆H₅ |
| P5-34 | 2-F—C₆H₄ |
| P5-35 | 3-F—C₆H₄ |
| P5-36 | 4-F—C₆H₄ |
| P5-37 | 2-Cl—C₆H₄ |
| P5-38 | 3-Cl—C₆H₄ |
| P5-39 | 4-Cl—C₆H₄ |
| P5-40 | 2-OCH₃—C₆H₄ |
| P5-41 | 3-OCH₃—C₆H₄ |
| P5-42 | 4-OCH₄—C₆H₄ |
| P5-43 | 2-OCHF₂—C₆H₄ |
| P5-44 | 3-OCHF₂—C₆H₄ |
| P5-45 | 4-OCHF₂—C₆H₄ |
| P5-46 | 2-CH₃—C₆H₄ |
| P5-47 | 3-CH₃—C₆H₄ |
| P5-48 | 4-CH₃—C₆H₄ |
| P5-49 | 2-CF₃—C₆H₄ |
| P5-50 | 3-CF₃—C₆H₄ |
| P5-51 | 4-CF₃—C₆H₄ |
| P5-52 | 2,3-F₂—C₆H₃ |
| P5-53 | 2,4-F₂—C₆H₃ |
| P5-54 | 2,5-F₂—C₆H₃ |
| P5-55 | 2,6-F₂—C₆H₃ |
| P5-56 | 3,4-F₂—C₆H₃ |
| P5-57 | 3,5-F₂—C₆H₃ |
| P5-58 | 2,3-Cl₂—C₆H₃ |
| P5-59 | 2,4-Cl₂—C₆H₃ |
| P5-60 | 2,5-Cl₂—C₆H₃ |
| P5-61 | 2,6-Cl₂—C₆H₃ |
| P5-62 | 3,4-Cl₂—C₆H₃ |
| P5-63 | 3,5-Cl₂—C₆H₃ |
| P5-64 | 2,3-(CH₃)₂—C₆H₃ |
| P5-65 | 2,4-(CH₃)₂—C₆H₃ |
| P5-66 | 2,5-(CH₃)₂—C₆H₃ |
| P5-67 | 2,6-(CH₃)₂—C₆H₃ |
| P5-68 | 3,4-(CH₃)₂—C₆H₃ |
| P5-69 | 3,5-(CH₃)₂—C₆H₃ |
| P5-70 | C₆H₅—CH₂ |
| P5-71 | 2-F—C₆H₄—CH₂ |
| P5-72 | 3-F—C₆H₄—CH₂ |
| P5-73 | 4-F—C₆H₄—CH₂ |
| P5-74 | 2-Cl—C₆H₄—CH₂ |
| P5-75 | 3-Cl—C₆H₄—CH₂ |
| P5-76 | 4-Cl—C₆H₄—CH₂ |
| P5-77 | 2-CH₃—C₆H₄—CH₂ |
| P5-78 | 3-CH₃—C₆H₄—CH₂ |
| P5-79 | 4-CH₃—C₆H₄—CH₂ |
| P5-80 | 2-CF₃—C₆H₄—CH₂ |
| P5-81 | 3-CF₃—C₆H₄—CH₂ |
| P5-82 | 4-CF₃—C₆H₄—CH₂ |
| P5-83 | 2-OCH₃—C₆H₄—CH₂ |
| P5-84 | 3-OCH₃—C₆H₄—CH₂ |
| P5-85 | 4-OCH₃—C₆H₄—CH₂ |
| P5-86 | 2-OCHF₂—C₆H₄—CH₂ |
| P5-87 | 3-OCHF₂—C₆H₄—CH₂ |
| P5-88 | 4-OCHF₂—C₆H₄—CH₂ |
| P5-89 | 2,3-F₂—C₆H₃—CH₂ |
| P5-90 | 2,4-F₂—C₆H₃—CH₂ |
| P5-91 | 2,5-F₂—C₆H₃—CH₂ |
| P5-92 | 2,6-F₂—C₆H₃—CH₂ |
| P5-93 | 3,4-F₂—C₆H₃—CH₂ |
| P5-94 | 3,5-F₂—C₆H₃—CH₂ |
| P5-95 | 2,3-Cl₂—C₆H₃—CH₂ |
| P5-96 | 2,4-Cl₂—C₆H₃—CH₂ |
| P5-97 | 2,5-Cl₂—C₆H₃—CH₂ |
| P5-98 | 2,6-Cl₂—C₆H₃—CH₂ |
| P5-99 | 3,4-Cl₂—C₆H₃—CH₂ |
| P5-100 | 3,5-Cl₂—C₆H₃—CH₂ |
| P5-101 | 2,3-(CH₃)₂—C₆H₃—CH₂ |
| P5-102 | 2,4-(CH₃)₂—C₆H₃—CH₂ |
| P5-103 | 2,5-(CH₃)₂—C₆H₃—CH₂ |
| P5-104 | 2,6-(CH₃)₂—C₆H₃—CH₂ |
| P5-105 | 3,4-(CH₃)₂—C₆H₃—CH₂ |
| P5-106 | 3,5-(CH₃)₂—C₆H₃—CH₂ |

R⁶ is in each case independently selected from halogen, ON, C₁-C₆-alkyl, C₂-C₆-alkenyl, C₂-C₆-alkynyl, C₁-C₆-halogenalkyl, C₂-C₆-halogenalkenyl, C₂-C₆-halogenalkynyl, O—C₁-C₆-alkyl, O—C₂-C₆-alkenyl, O—C₂-C₆-alkynyl, C₃-C₆-cycloalkyl, CH₂—C₃-C₆-cycloalkyl, O—C₃-C₆-cycloalkyl, C₃-C₆-cycloalkenyl, CH₂—C₃-C₆-cycloalkenyl, O—C₃-C₆-cycloalkenyl, C(R″)═N—OR′, phenyl, phenoxy, —CH₂-phenyl, a five- or six-membered heteroaryl, heteroaryloxy or CH₂-heteroaryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
   wherein the acyclic and cyclic moieties of R⁶ are unsubstituted or substituted by one to six groups R^{6a} which independently of one another are selected from:
   halogen, ON, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
   the acyclic and cyclic moieties of R^{6a} are unsubstituted or substituted by one to six groups R^{6b} halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C(R″)═N—OR′;
   wherein the groups R^{6b} are unsubstituted or substituted by one to six halogen or CN;

According to one embodiment of formula I, R⁶ is CN, halogen, C₁-C₆-alkyl, C₁-C₆-halogenalkyl, C₂-C₆-alkenyl, C₂-C₆-halogenalkenyl, O—C₁-C₆-alkyl, C₃-C₆-cycloalkyl, CH₂—C₃-C₆-cycloalkyl, wherein the acyclic and cyclic moieties of R⁶ are unsubstituted or substituted by one to six groups R^{6a} which independently of one another are selected from:

halogen, C(R″)═N—OR′, phenyl; and wherein
the acyclic and cyclic moieties of R^{6a} are unsubstituted or substituted by one to six groups R^{6b} halogen, C₁-C₆-alkyl, C₁-C₆-halogenalkyl, O—C₁-C₆-alkyl, C(R″)═N—OR′;
wherein the groups R^{6b} are unsubstituted or substituted by one to six halogen or CN.

According to one further preferred embodiment of formula I, R⁶ is C₁-C₆-alkyl, phenyl, phenoxy, wherein the acyclic and cyclic moieties of R⁶ are unsubstituted or substituted by one to six groups R^{6a} which independently of one another are selected from:

halogen, phenyl; and wherein
the phenyl is unsubstituted or substituted by one to six halogen.

According to one further preferred embodiment of formula I, R⁶ is CH₃, C₂H₅, phenyl, phenoxy, wherein the acyclic and cyclic moieties of R⁶ are unsubstituted or substituted by one to six groups R^{6a} which independently of one another are selected from:

halogen, phenyl; and wherein
the phenyl is unsubstituted or substituted by one to six halogen.

According to one further preferred embodiment of formula I, R⁶ is CH₃, C₂H₅, phenyl, CH₂-phenyl, phenoxy, wherein the acyclic and cyclic moieties of R⁶ are unsubstituted or substituted by one to six groups R^{6a} which independently of one another are selected from:

halogen.

According to still another embodiment of formula I, R⁶ is halogen, preferably F or C, most preferably F.

According to one embodiment of formula I, R⁶ is CN.

According to still another embodiment of formula I, R⁶ is C₁-C₆-alkyl, such as CH₃, C₂H₅, n-propyl, i-propyl, n-butyl, i-butyl, tert-butyl, n-pentyl, CH(CH₃)—(CH₂)₂—CH₃, (CH₂)₂—CH(CH₃)₂, or CH₂—C(CH₃)₃. Most preferably is i-propyl, tert-butyl, CH₂—C(CH₃)₃.

According to still another embodiment of formula I, R⁶ is C₁-C₆-halogenalkyl, in particular C₁-C₄-halogenalkyl, such as CF₃, CCl₃, FCH₂, ClCH₂, F₂CH, Cl₂CH, CF₃CH₂, CCl₃CH₂ or CF₂CHF₂.

According to still another embodiment of formula I R⁶ is C₃-C₆-cycloalkyl, in particular cyclopropyl.

According to still another embodiment of formula I, R⁶ is C₃-C₆-halogencycloalkyl. In a special embodiment R^{6b} is fully or partially halogenated cyclopropyl, such as 1-F-cyclopropyl, 1-Cl-cyclopropyl, 2,2-F₂-cyclopropyl, 2,2-Cl₂-cyclopropyl.

According to still a further embodiment of formula I, R⁶ is C₂-C₆-alkenyl, in particular C₂-C₄-alkenyl, such as C(CH₃)═CH₂, CH₂CH═CH₂, CH₂—CH₂—CH═CH₂.

According to a further specific embodiment of formula I, R⁶ is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CH₂CH═CHF, CH₂CH═CHCl, CH₂CH═CF₂, CH₂CH═CCl₂, CF₂CH═CF₂, CCl₂CH═CCl₂, CF₂CF═CF₂, CCl₂CCl═CCl₂.

According to still a further embodiment of formula I, R⁶ is C₂-C₆-alkynyl or C₂-C₆-halogenalkynyl, in particular C₂-C₄-alkynyl or C₂-C₄-halogenalkynyl, such as C≡CH, CH₂C≡CH, C≡CCl, CH₂C≡CCl, or CCl₂C≡CCl.

According to a further specific embodiment of formula I, R⁶ is O—C₁-C₆-alkanyl in particular C₂-C₄-alkanyl, more specifically C₁-C₂-alkanyl. R⁶ is such as OCH₃, OC₂H₅.

According to a further specific embodiment of formula I, R⁶ is O—C₂-C₆-alkenyl in particular C₂-C₄-alkenyl, more specifically C₂-C₃-alkenyl. R³ is such as OCH═CH₂, OCH₂CH═CH₂.

According to a further specific embodiment of formula I, R⁶ is O—C₂-C₆-alkynyl, in particular C₂-C₆-alkynyl, in particular C₂-C₄-alkynyl, more specifically C₂-C₃-alkynyl. R³ is such as OCH₂C≡CH,

According to still another embodiment of formula I, R⁶ is C₃-C₆-cycloalkyl, in particular cyclopropyl, cyclopentyl and cyclohexyl.

According to still another embodiment of formula I, R⁶ is CH₂—C₃-C₆-cycloalkyl, in particular CH₂-cyclopropyl, CH₂-cyclopentyl and CH₂-cyclohexyl.

According to still another embodiment of formula I, R⁶ is C₃-C₆-halogencycloalkyl. In a special embodiment R⁶ is fully or partially halogenated cyclopropyl, such as 1-F-cyclopropyl, 1-Cl-cyclopropyl, 2,2-F₂-cyclopropyl, 2,2-Cl₂-cyclopropyl.

According to a further specific embodiment of formula I, R⁶ is CR″═NOR′ such as

C(CH₃)═NOCH₃, C(CH₃)═NO—CH₂CH₃, C(CH₃)═NO—CH₂CF₃, C(CH₃)═NO—CH₂—CH═CH₂, C(CH₃)═NO—CH₂—CCH, C(CH₃)═NO—CH₂—C₆H₅;
C(CH₂CH₃)═NOCH₃, C(CH₂CH₃)═NO—CH₂CH₃, C(CH₂CH₃)═NO—CH₂CF₃, C(CH₂CH₃)═NO—CH₂—CH═CH₂, C(CH₂CH₃)═NO—CH₂—CCH, C(CH₂CH₃)═NO—CH₂—C₆H₅;
C(CF₃)═NOCH₃, C(CF₃)═NO—CH₂CH₃, C(CF₃)═NO—CH₂CF₃, C(CF₃)═NO—CH₂—CH═CH₂, C(CF₃)═NO—CH₂—CCH, C(CF₃)═NO—CH₂—C₆H₅;
C(C₃H₅)═NOCH₃, C(C₃H₅)═NO—CH₂CH₃, C(C₃H₅)═NO—CH₂CF₃, C(C₃H₅)═NO—CH₂—CH═CH₂, C(C₃H₅)═NO—CH₂—CCH, C(C₃H₅)═NO—CH₂—C₆H₅;
C(C₆H₅)═NOCH₃, C(C₆H₅)═NO—CH₂CH₃, C(C₆H₅)═NO—CH₂CF₃, C(C₆H₅)═NO—CH₂CH═CH₂, C(C₆H₅)═NO—CH₂—CCH, C(C₆H₅)═NO—CH₂—C₆H₅.

According to still another embodiment of formula I, R⁶ is phenyl or O-phenyl, wherein phenyl in each case is unsubstituted or substituted by identical or different groups R^{6a} which independently of one another are selected from halogen, C₁-C₂-alkyl, CR″═NOR′.

According to still another embodiment of formula I, R⁶ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl, preferred are pyrazol-1-yl, pyrazol-3-yl, pyrazol-4-yl, pyrazol-5-yl, 1,2,4-triazolyl-1-yl, 1,2,4-triazol-3-yl 1,2,4-triazol-5-yl, isoxazol-3-yl, isoxazol-4-yl, isoxazol-5-yl. The 5-membered heteroaryl in each case is unsubstituted or substituted by identical or different groups R^{6a} which independently of one another are selected from halogen, C₁-C₂-alkyl, CR″═NOR′.

According to still another embodiment of formula I, R⁶ is a 6-membered heteroaryl such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl, preferred are pyridin-2-yl, pyridin-3-yl, pyridin-4-yl. The 5-membered heteroaryl in each case is unsubstituted or substituted by identical or different groups R^{6a} which independently of one another are selected from halogen, C₁-C₂-alkyl, CR″═NOR′.

Particularly preferred embodiments of R⁶ according to the invention are in Table P6 below, wherein each line of lines P6-1 to P6-146 corresponds to one particular embodiment of the invention, wherein P6-1 to P6-146 are also in any combination with one another a preferred embodiment of the present invention. The connection point to the carbon atom, to which R⁶ is bound is marked with “#” in the drawings.

TABLE P6

| No. | R⁶ |
|---|---|
| P6-1 | CN |
| P6-2 | F |
| P6-3 | Cl |
| P6-4 | CH₃ |
| P6-5 | CH₂F |
| P6-6 | CHF₂ |
| P6-7 | CF₃ |
| P6-8 | C₂H₅ |
| P6-9 | CH₂CHF₂ |
| P6-10 | CH₂CF₃ |
| P6-11 | CH(CH₃)₂ |
| P6-12 | CH₂CH₂CH₃ |
| P6-13 | CH₂CH₂CH₂CH₃ |
| P6-14 | CH₂CH(CH₃)₂ |
| P6-15 | C(CH₃)₃ |
| P6-16 | CH₂CH₂CH₂CH₂CH₃ |
| P6-17 | CH═CH₂ |
| P6-18 | CH₂CH═CH₂ |
| P6-19 | C≡CH |
| P6-20 | CH₂C≡CH |
| P6-21 | CH₂CH₂CH(CH₃)₂ |
| P6-22 | OCH₃ |
| P6-23 | OC₂H₅ |
| P6-24 | OCH₂CH₂CH₃ |
| P6-25 | OCH(CH₃)₂ |
| P6-26 | OCH₂—CH═CH₂ |
| P6-27 | CH₂—C≡CH |
| P6-28 | C(CH₃)═N—OCH₃ |
| P6-29 | C(CH₃)═N—OCH₂—C₆H₅ |
| P6-30 | C(C₆H₅)═N—OCH₃ |
| P6-31 | 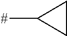 |
| P6-32 | 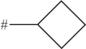 |
| P6-33 | 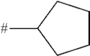 |
| P6-34 | 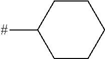 |
| P6-35 | 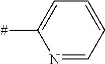 |
| P6-36 | 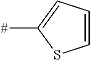 |
| P6-37 | C₆H₅ |
| P6-38 | 2-F—C₆H₄ |
| P6-39 | 3-F—C₆H₄ |
| P6-40 | 4-F—C₆H₄ |
| P6-41 | 2-Cl—C₆H₄ |
| P6-42 | 3-Cl—C₆H₄ |
| P6-43 | 4-Cl—C₆H₄ |
| P6-44 | 2-OCH₃—C₆H₄ |
| P6-45 | 3-OCH₃—C₆H₄ |
| P6-46 | 4-OCH₄—C₆H₄ |
| P6-47 | 2-OCHF₂—C₆H₄ |
| P6-48 | 3-OCHF₂—C₆H₄ |
| P6-49 | 4-OCHF₂—C₆H₄ |
| P6-50 | 2-CH₃—C₆H₄ |
| P6-51 | 3-CH₃—C₆H₄ |
| P6-52 | 4-CH₃—C₆H₄ |
| P6-53 | 2-CF₃—C₆H₄ |
| P6-54 | 3-CF₃—C₆H₄ |
| P6-55 | 4-CF₃—C₆H₄ |
| P6-56 | 2,3-F₂—C₆H₃ |
| P6-57 | 2,4-F₂—C₆H₃ |
| P6-58 | 2,5-F₂—C₆H₃ |
| P6-59 | 2,6-F₂—C₆H₃ |
| P6-60 | 3,4-F₂—C₆H₃ |
| P6-61 | 3,5-F₂—C₆H₃ |
| P6-62 | 2,3-Cl₂—C₆H₃ |
| P6-63 | 2,4-Cl₂—C₆H₃ |
| P6-64 | 2,5-Cl₂—C₆H₃ |
| P6-65 | 2,6-Cl₂—C₆H₃ |
| P6-66 | 3,4-Cl₂—C₆H₃ |
| P6-67 | 3,5-Cl₂—C₆H₃ |
| P6-68 | 2,3-(CH₃)₂—C₆H₃ |
| P6-69 | 2,4-(CH₃)₂—C₆H₃ |
| P6-70 | 2,5-(CH₃)₂—C₆H₃ |
| P6-71 | 2,6-(CH₃)₂—C₆H₃ |
| P6-72 | 3,4-(CH₃)₂—C₆H₃ |
| P6-73 | 3,5-(CH₃)₂—C₆H₃ |
| P6-74 | C₆H₅O |
| P6-75 | 2-F—C₆H₄O |
| P6-76 | 3-F—C₆H₄O |
| P6-77 | 4-F—C₆H₄O |
| P6-78 | 2-Cl—C₆H₄O |
| P6-79 | 3-Cl—C₆H₄O |
| P6-80 | 4-Cl—C₆H₄O |
| P6-81 | 2-CH₃—C₆H₄O |
| P6-82 | 3-CH₃—C₆H₄O |
| P6-83 | 4-CH₃—C₆H₄O |
| P6-84 | 2-CF₃—C₆H₄O |
| P6-85 | 3-CF₃—C₆H₄O |
| P6-86 | 4-CF₃—C₆H₄O |
| P6-87 | 2-OCH₃—C₆H₄O |
| P6-88 | 3-OCH₃—C₆H₄O |
| P6-89 | 4-OCH₃—C₆H₄O |
| P6-90 | 2-OCHF₂—C₆H₄O |
| P6-91 | 3-OCHF₂—C₆H₄O |
| P6-92 | 4-OCHF₂—C₆H₄O |
| P6-93 | 2,3-F₂—C₆H₃O |
| P6-94 | 2,4-F₂—C₆H₃O |
| P6-95 | 2,5-F₂—C₆H₃O |
| P6-96 | 2,6-F₂—C₆H₃O |
| P6-97 | 3,4-F₂—C₆H₃O |
| P6-98 | 3,5-F₂—C₆H₃O |
| P6-99 | 2,3-Cl₂—C₆H₃O |
| P6-100 | 2,4-Cl₂—C₆H₃O |
| P6-101 | 2,5-Cl₂—C₆H₃O |
| P6-102 | 2,6-Cl₂—C₆H₃O |
| P6-103 | 3,4-Cl₂—C₆H₃O |
| P6-104 | 3,5-Cl₂—C₆H₃O |
| P6-105 | 2,3-(CH₃)₂—C₆H₃O |
| P6-106 | 2,4-(CH₃)₂—C₆H₃O |
| P6-107 | 2,5-(CH₃)₂—C₆H₃O |
| P6-108 | 2,6-(CH₃)₂—C₆H₃O |
| P6-109 | 3,4-(CH₃)₂—C₆H₃O |
| P6-110 | 3,5-(CH₃)₂—C₆H₃O |
| P6-111 | C₆H₅—CH₂ |
| P6-112 | 2-F—C₆H₄—CH₂ |
| P6-113 | 3-F—C₆H₄—CH₂ |
| P6-114 | 4-F—C₆H₄—CH₂ |
| P6-115 | 2-Cl—C₆H₄—CH₂ |
| P6-116 | 3-Cl—C₆H₄—CH₂ |
| P6-117 | 4-Cl—C₆H₄—CH₂ |
| P6-118 | 2-CH₃—C₆H₄—CH₂ |
| P6-119 | 3-CH₃—C₆H₄—CH₂ |
| P6-120 | 4-CH₃—C₆H₄—CH₂ |
| P6-121 | 2-CF₃—C₆H₄—CH₂ |
| P6-122 | 3-CF₃—C₆H₄—CH₂ |
| P6-123 | 4-CF₃—C₆H₄—CH₂ |
| P6-124 | 2-OCH₃—C₆H₄—CH₂ |
| P6-125 | 3-OCH₃—C₆H₄—CH₂ |
| P6-126 | 4-OCH₃—C₆H₄—CH₂ |
| P6-127 | 2-OCHF₂—C₆H₄—CH₂ |
| P6-128 | 3-OCHF₂—C₆H₄—CH₂ |
| P6-129 | 4-OCHF₂—C₆H₄—CH₂ |
| P6-130 | 2,3-F₂—C₆H₃—CH₂ |
| P6-131 | 2,4-F₂—C₆H₃—CH₂ |
| P6-132 | 2,5-F₂—C₆H₃—CH₂ |
| P6-133 | 2,6-F₂—C₆H₃—CH₂ |
| P6-134 | 3,4-F₂—C₆H₃—CH₂ |
| P6-135 | 3,5-F₂—C₆H₃—CH₂ |
| P6-136 | 2,3-Cl₂—C₆H₃—CH₂ |
| P6-137 | 2,4-Cl₂—C₆H₃—CH₂ |
| P6-138 | 2,5-Cl₂—C₆H₃—CH₂ |
| P6-139 | 2,6-Cl₂—C₆H₃—CH₂ |
| P6-140 | 3,4-Cl₂—C₆H₃—CH₂ |
| P6-141 | 3,5-Cl₂—C₆H₃—CH₂ |
| P6-142 | 2,3-(CH₃)₂—C₆H₃—CH₂ |
| P6-143 | 2,4-(CH₃)₂—C₆H₃—CH₂ |
| P6-144 | 2,5-(CH₃)₂—C₆H₃—CH₂ |
| P6-145 | 2,6-(CH₃)₂—C₆H₃—CH₂ |
| P6-146 | 3,4-(CH₃)₂—C₆H₃—CH₂ |
| P6-147 | 3,5-(CH₃)₂—C₆H₃—CH₂ |

R⁷ is in each case independently selected from hydrogen, OH, halogen, CN, C₁-C₆-alkyl, C₂-C₆-alkenyl, C₂-C₆-alkynyl, C₁-C₆-halogenalkyl, C₂-C₆-halogenalkenyl, C₂-C₆-halogenalkynyl, O—C₁-C₆-alkyl, O—C₂-C₆-alkenyl, O—C₂-C₆-alkynyl, C₃-C₆-cycloalkyl, CH₂—C₃-C₆-cycloalkyl, O—C₃-C₆-cycloalkyl, C₃-C₆-cycloalkenyl, CH₂—C₃-C₆-cycloalkenyl, O—C₃-C₆-cycloalkenyl, C(R″)═N—OR′, phenyl, phenoxy, —CH₂-phenyl, a five- or six-membered heteroaryl, heteroaryloxy or CH₂-heteroaryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
   wherein the acyclic and cyclic moieties of R⁷ are unsubstituted or substituted by one to six groups R^{7a} which independently of one another are selected from:
   halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
   the acyclic and cyclic moieties of R^{7a} are unsubstituted or substituted by one to six groups halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C(R″)═N—OR′;
   wherein the groups R^{7b} are unsubstituted or substituted by one to six halogen or CN.

According to one embodiment of formula I, R⁷ is H, OH, CN, halogen, C₁-C₆-alkyl, O—C₁-C₆-alkyl, C₃-C₆-cycloalkyl, O—C₃-C₆-cycloalkyl, C(R″)═N—OR′, phenyl, phenoxy, CH₂-phenyl, wherein the acyclic and cyclic moieties of R⁷ are unsubstituted or substituted by one to six groups R^{7a} which independently of one another are selected from:
halogen, C(R″)═N—OR′, phenyl; and wherein the acyclic and cyclic moieties of R^{7a} are unsubstituted or substituted by one to six groups R^{7b} halogen, C₁-C₆-alkyl, C₁-C₆-halogenalkyl, O—C₁-C₆-alkyl, C(R″)═N—OR′;
wherein the groups R^{7b} are unsubstituted or substituted by one to six halogen or CN.

According to one further preferred embodiment of formula I, R⁷ is H, OH, halogen, CN, C₁-C₆-alkyl, phenyl, phenoxy, CH₂-phenyl, wherein the acyclic and cyclic moieties of R⁷ are unsubstituted or substituted by one to six groups R^{7a} which independently of one another are selected from:

halogen, phenyl; and wherein
the phenyl is unsubstituted or substituted by one to six halogen.

According to one further preferred embodiment of formula I, R⁷ is H, F, Cl, CN, CH₃, C₂H₅.

According to one further preferred embodiment of formula I, R⁷ is H, OH, CH₃, C₂H₅, phenyl, CH₂-phenyl, phenoxy, wherein the acyclic and cyclic moieties of R⁷ are unsubstituted or substituted by one to six groups R^{7a} which independently of one another are selected from:

halogen.

According to one further preferred embodiment of formula I, R⁷ is H, OH, CH₃.

According to one embodiment of formula I, R⁷ is H.

According to one embodiment of formula I, R⁷ is OH.

According to still another embodiment of formula I, R⁷ is halogen, preferably F or Cl, most preferably F.

According to one embodiment of formula I, R⁷ is CN.

According to still another embodiment of formula I, R⁷ is C₁-C₆-alkyl, such as CH₃, C₂H₅, n-propyl, i-propyl, n-butyl, i-butyl, tert-butyl, n-pentyl or i-pentyl. Most preferably is CH₃, C₂H₅.

According to still another embodiment of formula I, R⁷ is C₁-C₆-halogenalkyl, in particular C₁-C₄-halogenalkyl, such as CF₃, CCl₃, FCH₂, ClCH₂, F₂CH, Cl₂CH, CF₃CH₂, CCl₃CH₂ or CF₂CHF₂.

According to still another embodiment of formula I R⁷ is C₃-C₆-cycloalkyl, in particular cyclopropyl.

According to still another embodiment of formula I, R⁷ is C₃-C₆-halogencycloalkyl. In a special embodiment R^{7b} is fully or partially halogenated cyclopropyl, such as 1-F-cyclopropyl, 1-Cl-cyclopropyl, 2,2-F₂-cyclopropyl, 2,2-Cl₂-cyclopropyl.

According to still a further embodiment of formula I, R⁷ is C₂-C₆-alkenyl, in particular C₂-C₄-alkenyl, such as C(CH₃)═CH₂, CH₂CH═CH₂, CH₂—CH₂—CH═CH₂.

According to a further specific embodiment of formula I, R⁷ is C₂-C₆-halogenalkenyl, in particular C₂-C₄-halogenalkenyl, more specifically C₂-C₃-halogenalkenyl such as CH═CHF, CH═CHCl, CH═CF₂, CH═CCl₂, CH₂CH═CHF, CH₂CH═CHCl, CH₂CH═CF₂, CH₂CH═CCl₂, CF₂CH═CF₂, CCl₂CH═CCl₂, CF₂CF═CF₂, CCl₂CCl═CCl₂.

According to still a further embodiment of formula I, R⁶ is C₂-C₆-alkynyl or C₂-C₆-halogenalkynyl, in particular C₂-C₄-alkynyl or C₂-C₄-halogenalkynyl, such as C≡CH, CH₂C≡CH, C≡CCl, CH₂C≡CCl, or CCl₂C≡CCl.

According to a further specific embodiment of formula I, R⁷ is O—C₁-C₆-alkanyl in particular C₂-C₄-alkanyl, more specifically C₁-C₂-alkanyl. R⁷ is such as OCH₃, OC₂H₅.

According to a further specific embodiment of formula I, R⁷ is O—C₂-C₆-alkenyl in particular C₂-C₄-alkenyl, more specifically C₂-C₃-alkenyl. R³ is such as OCH═CH₂, OCH₂CH═CH₂.

According to a further specific embodiment of formula I, R⁷ is O—C₂-C₆-alkynyl, in particular C₂-C₆-alkynyl, in particular C₂-C₄-alkynyl, more specifically C₂-C₃-alkynyl. R³ is such as OCH₂C≡CH,

According to still another embodiment of formula I, R⁷ is C₃-C₆-cycloalkyl, in particular cyclopropyl, cyclopentyl and cyclohexyl.

According to still another embodiment of formula I, R⁷ is CH₂—C₃-C₆-cycloalkyl, in particular CH₂-cyclopropyl, CH₂-cyclopentyl and CH₂-cyclohexyl.

According to still another embodiment of formula I, R⁷ is C₃-C₆-halogencycloalkyl. In a special embodiment R⁷ is fully or partially halogenated cyclopropyl, such as 1-F-cyclopropyl, 1-Cl-cyclopropyl, 2,2-F₂-cyclopropyl, 2,2-Cl₂-cyclopropyl.

According to a further specific embodiment of formula I, R⁷ is CR″═NOR′ such as

C(CH₃)═NOCH₃, C(CH₃)═NO—CH₂CH₃, C(CH₃)═NO—CH₂CF₃, C(CH₃)═NO—CH₂—CH═CH₂, C(CH₃)═NO—CH₂—CCH, C(CH₃)═NO—CH₂—C₆H₅;
C(CH₂CH₃)═NOCH₃, C(CH₂CH₃)═NO—CH₂CH₃, C(CH₂CH₃)═NO—CH₂CF₃, C(CH₂CH₃)═NO—CH₂—CH═CH₂, C(CH₂CH₃)═NO—CH₂—CCH, C(CH₂CH₃)═NO—CH₂—C₆H₅;
C(CF₃)═NOCH₃, C(CF₃)═NO—CH₂CH₃, C(CF₃)═NO—CH₂CF₃, C(CF₃)═NO—CH₂—CH═CH₂, C(CF₃)═NO—CH₂—CCH, C(CF₃)═NO—CH₂—C₆H₅;
C(C₃H₅)═NOCH₃, C(C₃H₅)═NO—CH₂CH₃, C(C₃H₅)═NO—CH₂CF₃, C(C₃H₅)═NO—CH₂—CH═CH₂, C(C₃H₅)═NO—CH₂—CCH, C(C₃H₅)═NO—CH₂—C₆H₅;
C(C₆H₅)═NOCH₃, C(C₆H₅)═NO—CH₂CH₃, C(C₆H₅)═NO—CH₂CF₃, C(C₆H₅)═NO—CH₂CH═CH₂, C(C₆H₅)═NO—CH₂—CCH, C(C₆H₅)═NO—CH₂—C₆H₅.

According to still another embodiment of formula I, R⁷ is phenyl or O-phenyl, wherein phenyl in each case is unsubstituted or substituted by identical or different groups R^{7a} which independently of one another are selected from halogen, C₁-C₂-alkyl, CR″═NOR′.

According to still another embodiment of formula I, R⁷ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl, preferred are pyrazol-1-yl, pyrazol-3-yl, pyrazol-4-yl, pyrazol-5-yl, 1,2,4-triazolyl-1-yl, 1,2,4-triazol-3-yl 1,2,4-triazol-5-yl, isoxazol-3-yl, isoxazol-4-yl, isoxazol-5-yl. The 5-membered heteroaryl in each case is unsubstituted or substituted by identical or different groups R^{7a} which independently of one another are selected from halogen, C₁-C₂-alkyl, CR″═NOR′.

According to still another embodiment of formula I, R⁷ is a 6-membered heteroaryl such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl, preferred are pyridin-2-yl, pyridin-3-yl, pyridin-4-yl. The 5-membered heteroaryl in each case is unsubstituted or substituted by identical or different groups R^{7a} which independently of one another are selected from halogen, C₁-C₂-alkyl, CR″═NOR′.

Particularly preferred embodiments of R⁷ according to the invention are in Table P7 below, wherein each line of lines P7-1 to P7-146 corresponds to one particular embodiment of the invention, wherein P7-1 to P7-146 are also in any combination with one another a preferred embodiment of the present invention. The connection point to the carbon atom, to which R⁷ is bound is marked with “#” in the drawings.

TABLE P7

| No. | R⁷ |
|---|---|
| P7-1 | H |
| P7-2 | OH |
| P7-3 | CN |
| P7-4 | F |
| P7-5 | Cl |
| P7-6 | CH₃ |
| P7-7 | CH₂F |
| P7-8 | CHF₂ |
| P7-9 | CF₃ |
| P7-10 | C₂H₅ |
| P7-11 | CH(CH₃)₂ |
| P7-12 | CH₂CH₂CH₃ |
| P7-13 | CH₂CH₂CH₂CH₃ |
| P7-14 | CH₂CH(CH₃)₂ |
| P7-15 | C(CH₃)₃ |
| P7-16 | CH₂CH₂CH₂CH₂CH₃ |
| P7-17 | CH═CH₂ |
| P7-18 | CH₂CH═CH₂ |
| P7-19 | C≡CH |
| P7-20 | CH₂C≡CH |
| P7-21 | CH₂CH₂CH(CH₃)₂ |
| P7-22 | OCH₃ |
| P7-23 | OC₂H₅ |
| P7-24 | OCH₂CH₂CH₃ |
| P7-25 | OCH(CH₃)₂ |
| P7-26 | OCH₂—CH═CH₂ |
| P7-27 | CH₂—C≡CH |
| P7-28 | C(CH₃)═N—OCH₃ |
| P7-29 | C(CH₃)═N—OCH₂—C₆H₅ |
| P7-30 | C(C₆H₅)═N—OCH₃ |
| P7-31 | 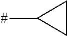 |
| P7-32 | 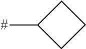 |
| P7-33 | 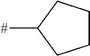 |
| P7-34 | 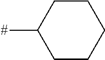 |
| P7-35 | 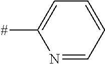 |
| P7-36 | 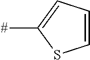 |
| P7-37 | C₆H₅ |
| P7-38 | 2-F—C₆H₄ |
| P7-39 | 3-F—C₆H₄ |
| P7-40 | 4-F—C₆H₄ |
| P7-41 | 2-Cl—C₆H₄ |
| P7-42 | 3-Cl—C₆H₄ |
| P7-43 | 4-Cl—C₆H₄ |
| P7-44 | 2-OCH₃—C₆H₄ |
| P7-45 | 3-OCH₃—C₆H₄ |
| P7-46 | 4-OCH₄—C₆H₄ |
| P7-47 | 2-OCHF₂—C₆H₄ |
| P7-48 | 3-OCHF₂—C₆H₄ |
| P7-49 | 4-OCHF₂—C₆H₄ |
| P7-50 | 2-CH₃—C₆H₄ |
| P7-51 | 3-CH₃—C₆H₄ |
| P7-52 | 4-CH₃—C₆H₄ |
| P7-53 | 2-CF₃—C₆H₄ |
| P7-54 | 3-CF₃—C₆H₄ |
| P7-55 | 4-CF₃—C₆H₄ |
| P7-56 | 2,3-F₂—C₆H₃ |
| P7-57 | 2,4-F₂—C₆H₃ |
| P7-58 | 2,5-F₂—C₆H₃ |
| P7-59 | 2,6-F₂—C₆H₃ |
| P7-60 | 3,4-F₂—C₆H₃ |
| P7-61 | 3,5-F₂—C₆H₃ |
| P7-62 | 2,3-Cl₂—C₆H₃ |
| P7-63 | 2,4-Cl₂—C₆H₃ |
| P7-64 | 2,5-Cl₂—C₆H₃ |
| P7-65 | 2,6-Cl₂—C₆H₃ |
| P7-66 | 3,4-Cl₂—C₆H₃ |
| P7-67 | 3,5-Cl₂—C₆H₃ |
| P7-68 | 2,3-(CH₃)₂—C₆H₃ |
| P7-69 | 2,4-(CH₃)₂—C₆H₃ |
| P7-70 | 2,5-(CH₃)₂—C₆H₃ |
| P7-71 | 2,6-(CH₃)₂—C₆H₃ |
| P7-72 | 3,4-(CH₃)₂—C₆H₃ |
| P7-73 | 3,5-(CH₃)₂—C₆H₃ |
| P7-74 | C₆H₅O |
| P7-75 | 2-F—C₆H₄O |
| P7-76 | 3-F—C₆H₄O |
| P7-77 | 4-F—C₆H₄O |
| P7-78 | 2-Cl—C₆H₄O |
| P7-79 | 3-Cl—C₆H₄O |
| P7-80 | 4-Cl—C₆H₄O |
| P7-81 | 2-CH₃—C₆H₄O |
| P7-82 | 3-CH₃—C₆H₄O |
| P7-83 | 4-CH₃—C₆H₄O |
| P7-84 | 2-CF₃—C₆H₄O |
| P7-85 | 3-CF₃—C₆H₄O |
| P7-86 | 4-CF₃—C₆H₄O |
| P7-87 | 2-OCH₃—C₆H₄O |
| P7-88 | 3-OCH₃—C₆H₄O |
| P7-89 | 4-OCH₃—C₆H₄O |
| P7-90 | 2-OCHF₂—C₆H₄O |
| P7-91 | 3-OCHF₂—C₆H₄O |
| P7-92 | 4-OCHF₂—C₆H₄O |
| P7-93 | 2,3-F₂—C₆H₃O |
| P7-94 | 2,4-F₂—C₆H₃O |
| P7-95 | 2,5-F₂—C₆H₃O |
| P7-96 | 2,6-F₂—C₆H₃O |
| P7-97 | 3,4-F₂—C₆H₃O |
| P7-98 | 3,5-F₂—C₆H₃O |
| P7-99 | 2,3-Cl₂—C₆H₃O |
| P7-100 | 2,4-Cl₂—C₆H₃O |
| P7-101 | 2,5-Cl₂—C₆H₃O |
| P7-102 | 2,6-Cl₂—C₆H₃O |
| P7-103 | 3,4-Cl₂—C₆H₃O |
| P7-104 | 3,5-Cl₂—C₆H₃O |
| P7-105 | 2,3-(CH₃)₂—C₆H₃O |
| P7-106 | 2,4-(CH₃)₂—C₆H₃O |
| P7-107 | 2,5-(CH₃)₂—C₆H₃O |
| P7-108 | 2,6-(CH₃)₂—C₆H₃O |
| P7-109 | 3,4-(CH₃)₂—C₆H₃O |
| P7-110 | 3,5-(CH₃)₂—C₆H₃O |
| P7-111 | C₆H₅—CH₂ |
| P7-112 | 2-F—C₆H₄—CH₂ |
| P7-113 | 3-F—C₆H₄—CH₂ |
| P7-114 | 4-F—C₆H₄—CH₂ |
| P7-115 | 2-Cl—C₆H₄—CH₂ |
| P7-116 | 3-Cl—C₆H₄—CH₂ |
| P7-117 | 4-Cl—C₆H₄—CH₂ |
| P7-118 | 2-CH₃—C₆H₄—CH₂ |
| P7-119 | 3-CH₃—C₆H₄—CH₂ |
| P7-120 | 4-CH₃—C₆H₄—CH₂ |
| P7-121 | 2-CF₃—C₆H₄—CH₂ |
| P7-122 | 3-CF₃—C₆H₄—CH₂ |
| P7-123 | 4-CF₃—C₆H₄—CH₂ |
| P7-124 | 2-OCH₃—C₆H₄—CH₂ |
| P7-125 | 3-OCH₃—C₆H₄—CH₂ |
| P7-126 | 4-OCH₃—C₆H₄—CH₂ |
| P7-127 | 2-OCHF₂—C₆H₄—CH₂ |
| P7-128 | 3-OCHF₂—C₆H₄—CH₂ |
| P7-129 | 4-OCHF₂—C₆H₄—CH₂ |
| P7-130 | 2,3-F₂—C₆H₃—CH₂ |
| P7-131 | 2,4-F₂—C₆H₃—CH₂ |
| P7-132 | 2,5-F₂—C₆H₃—CH₂ |
| P7-133 | 2,6-F₂—C₆H₃—CH₂ |
| P7-134 | 3,4-F₂—C₆H₃—CH₂ |
| P7-135 | 3,5-F₂—C₆H₃—CH₂ |
| P7-136 | 2,3-Cl₂—C₆H₃—CH₂ |
| P7-137 | 2,4-Cl₂—C₆H₃—CH₂ |
| P7-138 | 2,5-Cl₂—C₆H₃—CH₂ |
| P7-139 | 2,6-Cl₂—C₆H₃—CH₂ |
| P7-140 | 3,4-Cl₂—C₆H₃—CH₂ |
| P7-141 | 3,5-Cl₂—C₆H₃—CH₂ |
| P7-142 | 2,3-(CH₃)₂—C₆H₃—CH₂ |
| P7-143 | 2,4-(CH₃)₂—C₆H₃—CH₂ |
| P7-144 | 2,5-(CH₃)₂—C₆H₃—CH₂ |
| P7-145 | 2,6-(CH₃)₂—C₆H₃—CH₂ |
| P7-146 | 3,4-(CH₃)₂—C₆H₃—CH₂ |
| P7-147 | 3,5-(CH₃)₂—C₆H₃—CH₂ |

According to one embodiment of formula I, R⁶ and R⁷ form: ═N—O—CH₃, ═N—O—C₂H₅, ═N—O—CH₂—CF₃, ═N—O—CH₂—CH₂—CH₃, ═N—O—CH(CH₃)₂, ═N—O—CH₂—CH₂—CH₂—CH₃, ═N—O—CH(CH₃)—CH₂—CH₃, ═N—O—CH₂—CH(CH₃)₂, ═N—O—C(CH₃)₃

═N—O—CH₂—C₆H₅.R⁶ and R⁷ may join to form a group NO—R′
R′ CH₃, C₂H₅, n-C₃H₇, CH(CH₃)₂, n-C₄H₉, CH(CH₃)—CH₂—CH₃, CH₂—CH(CH₃)₂, n-C₅H₁₁, CH(CH₃)—CH₂—CH₂—CH₃, CH₂—CH(CH₃)—CH₂—CH₃, CH₂—CH₂—CH(CH₃)₂, CH₂—CHF₂, CH₂—CF₃, CH₂—CH═CH₂, CH₂—CH═CHCl, CH₂—C≡CH, CH₂-cyclo-C₃H₅, CH₂-cyclo-C₄H₇, CH₂-cyclo-C₅H₉, CH₂-cyclo-C₆H₁₁
benzyl, 2-F-benzyl, 3-F-benzyl, 4-F-benzyl, 2,3-F₂-benzyl, 2,4-F₂-benzyl, 2,5-F2-benzyl, 2,6-F₂-benzyl, 3,4-F₂-benzyl, 3,5-F₂-benzyl
2-Cl-benzyl, 3-Cl-benzyl, 4-Cl-benzyl, 2,3-Cl₂-benzyl, 2,4-Cl₂-benzyl, 2,5-Cl₂-benzyl, 2,6-Cl₂-benzyl, 3,4-Cl₂-benzyl, 3,5-Cl₂-benzyl
2-CH₃-benzyl, 3-CH₃-benzyl, 4-CH₃-benzyl, 2,3-(CH₃)₂-benzyl, 2,4-(CH₃)₂-benzyl, 2,5-(CH₃)₂-benzyl, 2,6-(CH₃)₂-benzyl, 3,4-(CH₃)₂-benzyl, 3,5-(CH₃)₂-benzyl
2-CF₃-benzyl, 3-CF₃-benzyl, 4-CF₃-benzyl,
2-OCH₃-benzyl, 3-OCH₃-benzyl, 4-OCH₃-benzyl,
2-OCHF₂-benzyl, 3-OCHF₂-benzyl, 4-OCHF₂-benzyl.
R⁸ is in each case independently selected from C₃-C₁₀-cycloalkyl, C₃-C₁₀-cycloalkenyl, phenyl or a five- or six-membered heteroaryl, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
   wherein the acyclic and cyclic moieties of R⁸ are unsubstituted or substituted by one to six groups R^{3a} which independently of one another are selected from:
   halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C₃-C₆-cycloalkenyl, O—C₃-C₆-cycloalkenyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
   the acyclic and cyclic moieties of R^{3a} are unsubstituted or substituted by one to six groups R^{8b} halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C₃-C₆-cycloalkenyl, O—C₃-C₆-cycloalkenyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein

the groups R^{8b} are unsubstituted or substituted by one to six halogen or CN.

According to one embodiment of formula I, R³ is phenyl, pyridyl-2, pyridyl-3, pyridyl-4, cyclopentyl or cyclohexyl.

According to still another embodiment of formula I, R⁸ is C₃-C₁₀-cycloalkyl, in particular cyclopropyl, cyclopentyl and cyclohexyl.

According to still another embodiment of formula I, R⁸ is C₃-C₆-halogencycloalkyl. In a special embodiment R⁸ is fully or partially halogenated cyclopropyl, such as 1-F-cyclopropyl, 1-Cl-cyclopropyl, 2,2-F₂-cyclopropyl, 2,2-Cl₂-cyclopropyl.

According to still another embodiment of formula I, R⁸ is phenyl, wherein phenyl in each case is unsubstituted or substituted by identical or different groups R^{3a} which independently of one another are selected from halogen, C₁-C₆-alkyl, C₁-C₆-halogenalkyl, O—C₁-C₆-alkyl, O—C₁-C₆-halogenalkyl, CR″═NOR′.

According to still another embodiment of formula I, R⁸ is a 5-membered heteroaryl such as pyrrol-1-yl, pyrrol-2-yl, pyrrol-3-yl, thien-2-yl, thien-3-yl, furan-2-yl, furan-3-yl, pyrazol-1-yl, pyrazol-3-yl, pyrazol-4-yl, pyrazol-5-yl, imidazol-1-yl, imidazol-2-yl, imidazol-4-yl, imidazol-5-yl, oxazol-2-yl, oxazol4-yl, oxazol-5-yl, isoxazol-3-yl, isoxazol-4-yl, isoxazol-5-yl, thiazol-2-yl, thiazol-4-yl, thiazol-5-yl, isothiazol-3-yl, isothiazol-4-yl, isothiazol-5-yl, 1,2,4-triazolyl-1-yl, 1,2,4-triazol-3-yl 1,2,4-triazol-5-yl, 1,2,4-oxadiazol-3-yl, 1,2,4-oxadiazol-5-yl and 1,2,4-thiadiazol-3-yl, 1,2,4-thiadiazol-5-yl, preferred are pyrazol-1-yl, pyrazol-3-yl, pyrazol-4-yl, pyrazol-5-yl, 1,2,4-triazolyl-1-yl, 1,2,4-triazol-3-yl 1,2,4-triazol-5-yl, isoxazol-3-yl, isoxazol-4-yl, isoxazol-5-yl. The 5-membered heteroaryl in each case is unsubstituted or substituted by identical or different groups R^{3a} which independently of one another are selected from halogen, C₁-C₆-alkyl, C₁-C₆-halogenalkyl, O—C₁-C₆-alkyl, O—C₁-C₆-halogenalkyl, CR″═NOR′.

According to still another embodiment of formula I, R⁸ is a 6-membered heteroaryl such as pyridin-2-yl, pyridin-3-yl, pyridin-4-yl, pyridazin-3-yl, pyridazin-4-yl, pyrimidin-2-yl, pyrimidin-4-yl, pyrimidin-5-yl, pyrazin-2-yl and 1,3,5-triazin-2-yl and 1,2,4-triazin-3-yl, preferred are pyridin-2-yl, pyridin-3-yl, pyridin-4-yl. The 5-membered heteroaryl in each case is unsubstituted or substituted by identical or different groups R^{3a} which independently of one another are selected from halogen, C₁-C₆-alkyl, C₁-C₆-halogenalkyl, O—C₁-C₆-alkyl, O—C₁-C₆-halogenalkyl, CR″═NOR′.

Particularly preferred embodiments of R⁸ according to the invention are in Table P8 below, wherein each line of lines P8-1 to P8-45 corresponds to one particular embodiment of the invention, wherein P8-1 to P8-45 are also in any combination with one another a preferred embodiment of the present invention. The connection point to the carbon atom, to which R⁸ is bound is marked with “#” in the drawings.

TABLE P8

| No. | R⁸ |
|---|---|
| P8-1 | 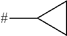 |
| P8-2 | 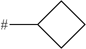 |
| P8-3 | 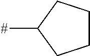 |
| P8-4 | 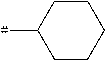 |
| P8-5 | 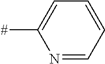 |
| P8-6 | 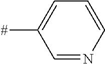 |
| P8-7 | 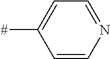 |
| P8-8 | 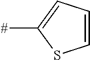 |
| P8-9 | C₆H₅ |
| P8-10 | 2-F—C₆H₄ |
| P8-11 | 3-F—C₆H₄ |
| P8-12 | 4-F—C₆H₄ |
| P8-13 | 2-Cl—C₆H₄ |
| P8-14 | 3-Cl—C₆H₄ |
| P8-15 | 4-Cl—C₆H₄ |
| P8-16 | 2-OCH₃—C₆H₄ |
| P8-17 | 3-OCH₃—C₆H₄ |
| P8-18 | 4-OCH₄—C₆H₄ |
| P8-19 | 2-OCHF₂—C₆H₄ |
| P8-20 | 3-OCHF₂—C₆H₄ |
| P8-21 | 4-OCHF₂—C₆H₄ |
| P8-22 | 2-CH₃—C₆H₄ |
| P8-23 | 3-CH₃—C₆H₄ |
| P8-24 | 4-CH₃—C₆H₄ |
| P8-25 | 2-CF₃—C₆H₄ |
| P8-26 | 3-CF₃—C₆H₄ |
| P8-27 | 4-CF₃—C₆H₄ |
| P8-28 | 2,3-F₂—C₆H₃ |
| P8-29 | 2,4-F₂—C₆H₃ |
| P8-30 | 2,5-F₂—C₆H₃ |
| P8-31 | 2,6-F₂—C₆H₃ |
| P8-32 | 3,4-F₂—C₆H₃ |
| P8-33 | 3,5-F₂—C₆H₃ |
| P8-34 | 2,3-Cl₂—C₆H₃ |
| P8-35 | 2,4-Cl₂—C₆H₃ |
| P8-36 | 2,5-Cl₂—C₆H₃ |
| P8-37 | 2,6-Cl₂—C₆H₃ |
| P8-38 | 3,4-Cl₂—C₆H₃ |
| P8-39 | 3,5-Cl₂—C₆H₃ |
| P8-40 | 2,3-(CH₃)₂—C₆H₃ |
| P8-41 | 2,4-(CH₃)₂—C₆H₃ |
| P8-42 | 2,5-(CH₃)₂—C₆H₃ |
| P8-43 | 2,6-(CH₃)₂—C₆H₃ |
| P8-44 | 3,4-(CH₃)₂—C₆H₃ |
| P8-45 | 3,5-(CH₃)₂—C₆H₃ |

In one embodiment, the invention relates to compounds of the formula I, or the N-oxides, or the agriculturally acceptable salts thereof, wherein

### X is O;

R¹ is H;
R² is selected from the group consisting of halogen, C₁-C₆-alkyl or O—C₁-C₆-alkyl;
R³ is selected from the group consisting of halogen, C₁-C₆-alkyl, C₁-C₆-halogenalkyl;
R⁴ is H;
Y is NR⁵;
R⁵ is selected from CH₃, C₂H₅, n-C₃H₇, CH(CH₃)₂, (CH₂)₃—CH₃, CH(CH₃)—CH₂—CH₃, CH₂—CH(CH₃)₂, C(CH₃)₃, CH₂—C(CH₃)₃, phenyl, CH₂-phenyl, wherein the acyclic and cyclic moieties of R⁵ are unsubstituted or substituted by one to six groups R^{5a} which independently of one another are selected from:
halogen;
R⁸ is selected from phenyl, pyridyl-2, pyridyl-3, pyridyl-4, cyclopentyl or cyclohexyl.

In one further embodiment, the invention relates to compounds of the formula I, or the N-oxides, or the agriculturally acceptable salts thereof, wherein
R¹ is H;
R² is selected from the group consisting of CH₃, C₂H₅, OCH₃.
R³ is selected from the group consisting of F, Cl, CH₃, C₂H₅, CF₃, CH₂F, CHF₂;
R⁴ is H;
Y is NR⁵;
R⁵ is selected from n-C₃H₇, CH(CH₃)₂, CH₂—CH(CH₃)₂, C(CH₃)₃, CH₂—C(CH₃)₃, phenyl, CH₂-phenyl, wherein the acyclic and cyclic moieties of R⁵ are unsubstituted or substituted by one to six groups R⁵¹ which independently of one another are selected from:
halogen;
R⁸ is selected from phenyl, pyridyl-2, pyridyl-3, pyridyl-4, cyclopentyl or cyclohexyl.

In one further embodiment, the invention relates to compounds of the formula I, or the N-oxides, or the agriculturally acceptable salts thereof, wherein R¹, R², R³, R⁴, R⁶, R⁷, R⁸ are defined above
with the proviso that:
if Y is CR⁶R⁷, R¹ and R⁴ are H and
R⁶ is C₁-C₄-alkyl and
R⁷ is H, OH, CH₃, C₂H₅,
R⁸ is not an unsubstituted phenyl;
if Y is CR⁶R⁷, R¹ and R⁴ are H and
R⁶ is halogen and
R⁷ is halogen,
R⁸ is not an unsubstituted phenyl;
if Y is CR⁶R⁷, R¹ and R⁴ are H and
R⁶ is C₃-C₆-alkyl and
R⁷ is CH₃,
R⁸ is not cyclopropyl;
if Y is CR⁶R⁷, R¹ and R⁴ are H and
R⁶ is CH₂-phenyl and
R⁷ is H,
R⁸ is not cyclopropyl;
if Y is CR⁶R⁷, R¹ and R⁴ are H and
R⁶ is i-propyl and
R⁷ is H,
R⁸ is not 4-methoxyphenyl;
and following compounds are excluded

I.A-1

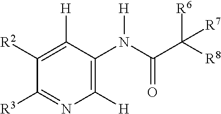

| R² | R³ | R⁶ | R⁷ | R⁸ |
|---|---|---|---|---|
| —CH₃ | —CH₃ | cyclopentyl | H | phenyl |
| —CH₃ | —CH₃ | CF₃ | —OCH₃ | phenyl |
| —CH₃ | —CH₃ | O—C(CH₃)₃ | H | phenyl |

In one embodiment, the invention relates to compounds of the formula I, or the N-oxides, or the agriculturally acceptable salts thereof, wherein

### X is O;

R¹ is H;
R² is selected from the group consisting of halogen, C₁-C₆-alkyl or O—C₁-C₆-alkyl;
R³ is selected from the group consisting of halogen, C₁-C₆-alkyl, C₁-C₆-halogenalkyl;
R⁴ is H;
Y is CR⁶R⁷;
R⁶ is selected from CH₃, C₂H₅, n-C₃H₇, CH(CH₃)₂, CH₂—CH(CH₃)₂, C(CH₃)₃, CH₂—C(CH₃)₃, CH₂—CH═CH₂, CH₂—C(CH₃)═CH₂, CH₂—CH═CH—CH₃, CH₂—CH═C(CH₃)₂, CH₂—C(CH₃)═C(CH₃)₂, cyclopropyl, CH₂-cyclopropyl, phenyl, CH₂-phenyl, wherein the acyclic and cyclic moieties of R⁶ are unsubstituted or substituted by one to six groups R^{6a} which independently of one another are selected from:
halogen;
R⁷ is selected from H, OH, halogen, CN, C₁-C₆-alkyl;
R⁸ is selected from phenyl, pyridyl-2, pyridyl-3, pyridyl-4, cyclopentyl or cyclohexyl.

In one further embodiment, the invention relates to compounds of the formula I, or the N-oxides, or the agriculturally acceptable salts thereof, wherein
R¹ is H;
R² is selected from the group consisting of CH₃, C₂H₅, OCH₃.
R³ is selected from the group consisting of F, C, CH₃, C₂H₅, CF₃, CH₂F, CHF₂;
R⁴ is H;
Y is CR⁶R⁷;
R⁶ is selected from n-C₃H₇, CH(CH₃)₂, CH₂—CH(CH₃)₂, C(CH₃)₃, CH₂—C(CH₃)₃, CH₂—CH═CH₂, CH₂—C(CH₃)═CH₂, CH₂—CH═CH—CH₃, CH₂—CH═C(CH₃)₂, CH₂—C(CH₃)═C(CH₃)₂, cyclopropyl, CH₂-cyclopropyl, wherein the acyclic and cyclic moieties of R⁶ are unsubstituted or substituted by one to six groups R^{6a} which independently of one another are selected from:
halogen;
R⁷ is selected from H, halogen, CN, CH₃;
R⁸ is selected from phenyl, pyridyl-2, pyridyl-3, pyridyl-4, cyclopentyl or cyclohexyl.

In one further embodiment, the invention relates to compounds of the formula I, or the N-oxides, or the agriculturally acceptable salts thereof, wherein
R¹ is H;
R² and R³ together with the carbon atoms they are attached to can form a substituted ring of the formula II:

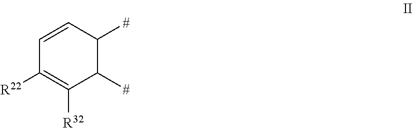

R²² is selected from the group consisting of H, halogen, C₁-C₂-alkyl, C₁-C₂-halogenalkyl.
R³² is selected from the group consisting of halogen, C₁-C₂-alkyl, C₁-C₂-halogenalkyl;
R⁴ is H;
Y is CR⁶R⁷;
R⁶ is selected from n-C₃H₇, CH(CH₃)₂, CH₂—CH(CH₃)₂, C(CH₃)₃, CH₂—C(CH₃)₃, CH₂—CH═CH₂, CH₂—C(CH₃)═CH₂, CH₂—CH═CH—CH₃, CH₂—CH═C(CH₃)₂, CH₂—C(CH₃)═C(CH₃)₂, cyclopropyl, CH₂-cyclopropyl, wherein the acyclic and cyclic moieties of R⁶ are unsubstituted or substituted by one to six groups R^{6a} which independently of one another are selected from:
halogen;
R⁷ is selected from H, halogen, CN, CH₃;
R⁸ is selected from phenyl, pyridyl-2, pyridyl-3, pyridyl-4, cyclopentyl or cyclohexyl.

Preferred embodiments of the present invention are the following compounds I.A-1, I.A-2, I.A-3, I.A-4, I.A-5, I.A-6; compounds I.B-1, I.B-2, I.B-3, I.B-4, I.B-5, I.B-6. In these formulae, the substituents R⁵, R⁸ are independently as defined above or preferably defined herein:

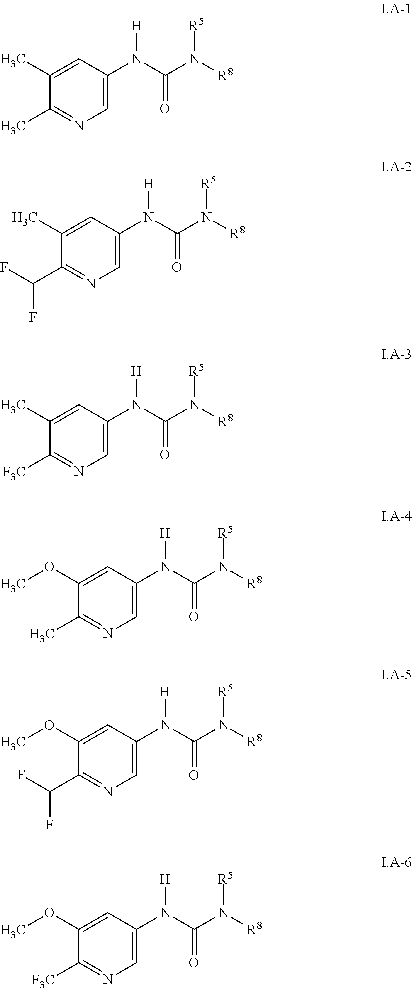

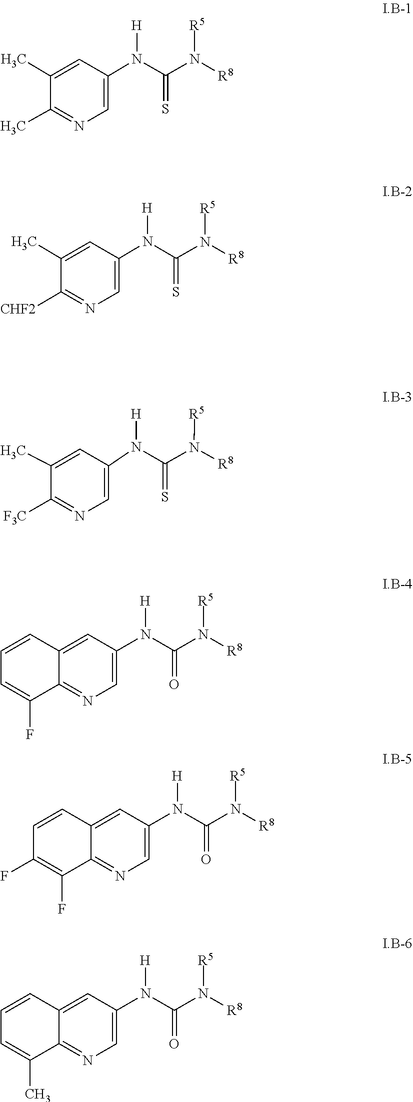

In particular with a view to their use, according to one embodiment, preference is given to the compounds of the compounds I.A-1, I.A-2, I.A-3, I.A-4, I.A-5, I.A-6; compounds I.B-1, I.B-2, I.B-3, I.B-4, I.B-5, I.B-6, that are compiled in the Tables 1a to 43 a. Each of the groups mentioned for a substituent in the tables is furthermore per se, independently of the combination in which it is mentioned, a particularly preferred aspect of the substituent in question.

Table 1a Compounds of the formula I.A-1, I.A-2, I.A-3, I.A-4, I.A-5, I.A-6; I.B-1, I.B-2, I.B-3, I.B-4, I.B-5, I.B-6 in which R⁸ is cyclo-C₃H₅ and the meaning for R⁵ for each individual compound corresponds in each case to one line of Table B (compounds I.A-1.1a.B-1 to I.A-1.1a.B-94, I.A-2.1a.B-1 to I.A-2.1a.B-94, I.A-3.1a.B-1 to I.A-3.1a.B-94, I.A-4.1a.B-1 to I.A-5.1a.B-94, I.A-5.1a.B-1 to I.A-3.1a.B-94, I.A-6.1a.B-1 to I.A-6.1a.B-94; I.E-1a.B-1 to I.B-1.1a.B-94, I.B-2.1a.B-1 to I.B-2.1a.B-94, I.B-3.1a.B-1 to I.B-3.1a.B-94, I.B-4.1a.B-1 to I.B-4.1a.B-94, I.B-5.1a.B-1 to I.B-5.1a.B-94, I.B-6.1a.B-1 to I.B-6.1a.B-94).
Table 2a Compounds of the formula I.A-1, I.A-2, I.A-3, I.A-4, I.A-5, I.A-6; I.B-1, I.B-2, I.B-3, I.B-4, I.B-5, I.B-6 in which R⁸ is cyclo-C₄H₇ and the meaning for R⁵ for each individual compound corresponds in each case to one line of Table B (compounds I.A-1.2a.B-1 to I.A-1.2a.B-94, I.A-2.2a.B-1 to I.A-2.2a.B-94, I.A-3.2a.B-1 to I.A-3.2a.B-94, I.A-4.2a.B-1 to I.A-5.2a.B-94, I.A-5.2a.B-1 to I.A-3.2a.B-94, I.A-6.2a.B-1 to I.A-6.2a.B-94; I.B-2a.B-1 to I.B-1.2a.B-94, I.B-2.2a.B-1 to I.B-2.2a.B-94, I.B-3.2a.B-1 to I.B-3.2a.B-94, I.B-4.2a.B-1 to I.B-4.2a.B-94, I.B-5.2a.B-1 to I.B-5.2a.B-94, I.B-6.2a.B-1 to I.B-6.2a.B-94).
Table 3a Compounds of the formula I.A-1, I.A-2, I.A-3, I.A-4, I.A-5, I.A-6; I.B-1, I.B-2, I.B-3, I.B-4, I.B-5, I.B-6 in which R⁸ is cyclo-C₅H₉ and the meaning for R⁵ for each individual compound corresponds in each case to one line of Table B (compounds I.A-1.3a.B-1 to I.A-1.3a.B-94, I.A-2.3a.B-1 to I.A-2.3a.B-94, I.A-3.3a.B-1 to I.A-3.3a.B-94, I.A-4.3a.B-1 to I.A-5.3a.B-94, I.A-5.3a.B-1 to I.A-3.3a.B-94, I.A-6.3a.B-1 to I.A-6.3a.B-94; I.B-3a.B-1 to I.B-1.3a.B-94, I.B-2.3a.B-1 to I.B-2.3a.B-94, I.B-3.3a.B-1 to I.B-3.3a.B-94, I.B-4.3a.B-1 to I.B-4.3a.B-94, I.B-5.3a.B-1 to I.B-5.3a.B-94, I.B-6.3a.B-1 to I.B-6.3a.B-94).
Table 4a Compounds of the formula I.A-1, I.A-2, I.A-3, I.A-4, I.A-5, I.A-6; I.B-1, I.B-2, I.B-3, I.B-4, I.B-5, I.B-6 in which R⁸ is cyclo-C₆H₁₁ and the meaning for R⁵ for each individual compound corresponds in each case to one line of Table B (compounds I.A-1.4a.B-1 to I.A-1.4a.B-94, I.A-2.4a.B-1 to I.A-2.4a.B-94, I.A-3.4a.B-1 to I.A-3.4a.B-94, I.A-4.4a.B-1 to I.A-5.4a.B-94, I.A-5.4a.B-1 to I.A-3.4a.B-94, I.A-6.4a.B-1 to I.A-6.4a.B-94; I.B-4a.B-1 to I.B-1.4a.B-94, I.B-2.4a.B-1 to I.B-2.4a.B-94, I.B-3.4a.B-1 to I.B-3.4a.B-94, I.B-4.4a.B-1 to I.B-4.4a.B-94, I.B-5.4a.B-1 to I.B-5.4a.B-94, I.B-6.4a.B-1 to I.B-6.4a.B-94).
Table 5a Compounds of the formula I.A-1, I.A-2, I.A-3, I.A-4, I.A-5, I.A-6; I.B-1, I.B-2, I.B-3, I.B-4, I.B-5, I.B-6 in which R⁸ is

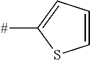

and the meaning for R⁵ for each individual compound corresponds in each case to one line of Table B (compounds I.A-1.5a.B-1 to I.A-1.5a.B-94, I.A-2.5a.B-1 to I.A-2.5a.B-94, I.A-3.5a.B-1 to I.A-3.5a.B-94, I.A-4.5a.B-1 to I.A-5.5a.B-94, I.A-5.5a.B-1 to I.A-3.5a.B-94, I.A-6.5a.B-1 to I.A-6.5a.B-94; I.B-5a.B-1 to I.B-1.5a.B-94, I.B-2.5a.B-1 to I.B-2.5a.B-94, I.B-3.5a.B-1 to I.B-3.5a.B-94, I.B-4.5a.B-1 to I.B-4.5a.B-94, I.B-5.5a.B-1 to I.B-5.5a.B-94, I.B-6.5a.B-1 to I.B-6.5a.B-94).
Table 6a Compounds of the formula I.A-1, I.A-2, I.A-3, I.A-4, I.A-5, I.A-6; I.B-1, I.B-2, I.B-3, I.B-4, I.B-5, I.B-6 in which R⁸ is C₆H₅ and the meaning for R⁵ for each individual compound corresponds in each case to one line of Table B (compounds I.A-1.6a.B-1 to I.A-1.6a.B-94, I.A-2.6a.B-1 to I.A-2.6a.B-94, I.A-3.6a.B-1 to I.A-3.6a.B-94, I.A-4.6a.B-1 to I.A-5.6a.B-94, I.A-5.6a.B-1 to I.A-3.6a.B-94, I.A-6.6a.B-1 to I.A-6.6a.B-94; I.B-6a.B-1 to I.B-1.6a.B-94, I.B-2.6a.B-1 to I.B-2.6a.B-94, I.B-3.6a.B-1 to I.B-3.6a.B-94, I.B-4.6a.B-1 to I.B-4.6a.B-94, I.B-5.6a.B-1 to I.B-5.6a.B-94, I.B-6.6a.B-1 to I.B-6.6a.B-94).
Table 7a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 2-F—C₆H₄ and the meaning for R⁵ for each individual compound corresponds in each case to one line of Table B (compounds I.A-1.7a.B-1 to I.A-1.7a.B-94, I.A-2.7a.B-1 to I.A-2.7a.B-94, I.A-3.7a.B-1 to I.A-3.7a.B-94, I.A-4.7a.B-1 to I.A-5.7a.B-94, I.A-5.7a.B-1 to I.A-3.7a.B-94, I.A-6.7a.B-1 to I.A-6.7a.B-94; I.B-7a.B-1 to I.B-1.7a.B-94, I.B-2.7a.B-1 to I.B-2.7a.B-94, I.B-3.7a.B-1 to I.B-3.7a.B-94, I.B-4.7a.B-1 to I.B-4.7a.B-94, I.B-5.7a.B-1 to I.B-5.7a.B-94, I.B-6.7a.B-1 to I.B-6.7a.B-94).
Table 8a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 3-F—C₆H₄ and the meaning for R⁵ for each individual compound corresponds in each case to one line of Table B (compounds I.A-1.8a.B-1 to I.A-1.8a.B-94, I.A-2.8a.B-1 to I.A-2.8a.B-94, I.A-3.8a.B-1 to I.A-3.8a.B-94, I.A-4.8a.B-1 to I.A-5.8a.B-94, I.A-5.8a.B-1 to I.A-3.8a.B-94, I.A-6.8a.B-1 to I.A-6.8a.B-94; I.B-8a.B-1 to I.B-1.8a.B-94, I.B-2.8a.B-1 to I.B-2.8a.B-94, I.B-3.8a.B-1 to I.B-3.8a.B-94, I.B-4.8a.B-1 to I.B-4.8a.B-94, I.B-5.8a.B-1 to I.B-5.8a.B-94, I.B-6.8a.B-1 to I.B-6.8a.B-94).
Table 9a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 4-F—C₆H₄ and the meaning for R⁵ for each individual compound corresponds in each case to one line of Table B (compounds I.A-1.9a.B-1 to I.A-1.9a.B-94, I.A-2.9a.B-1 to I.A-2.9a.B-94, I.A-3.9a.B-1 to I.A-3.9a.B-94, I.A-4.9a.B-1 to I.A-5.9a.B-94, I.A-5.9a.B-1 to I.A-3.9a.B-94, I.A-6.9a.B-1 to I.A-6.9a.B-94; I.B-9a.B-1 to I.B-1.9a.B-94, I.B-2.9a.B-1 to I.B-2.9a.B-94, I.B-3.9a.B-1 to I.B-3.9a.B-94, I.B-4.9a.B-1 to I.B-4.9a.B-94, I.B-5.9a.B-1 to I.B-5.9a.B-94, I.B-6.9a.B-1 to I.B-6.9a.B-94).
Table 10a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 2-Cl—C₆H₄ and the meaning for R⁵ for each individual compound corresponds in each case to one line of Table B (compounds I.A-1.10a.B-1 to I.A-1.10a.B-94, I.A-2.10a.B-1 to I.A-2.10a.B-94, I.A-3.10a.B-1 to I.A-3.10a.B-94, I.A-4.10a.B-1 to I.A-5.10a.B-94, I.A-5.10a.B-1 to I.A-3.10a.B-94, I.A-6.10a.B-1 to I.A-6.10a.B-94; I.B-10a.B-1 to I.B-1.10a.B-94, I.B-2.10a.B-1 to I.B-2.10a.B-94, I.B-3.10a.B-1 to I.B-3.10a.B-94, I.B-4.10a.B-1 to I.B-4.10a.B-94, I.B-5.10a.B-1 to I.B-5.10a.B-94, I.B-6.10a.B-1 to I.B-6.10a.B-94).
Table 11a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 3-Cl—C₆H₄ and the meaning for R⁵ for each individual compound corresponds in each case to one line of Table B (compounds I.A-1.11a.B-1 to I.A-1.11a.B-94, I.A-2.11a.B-1 to I.A-2.11a.B-94, I.A-3.11a.B-1 to I.A-3.11a.B-94, I.A-4.11a.B-1 to I.A-5.11a.B-94, I.A-5.11a.B-1 to I.A-3.11a.B-94, I.A-6.11a.B-1 to I.A-6.11a.B-94; I.B-11a.B-1 to I.B-1.11a.B-94, I.B-2.11a.B-1 to I.B-2.11a.B-94, I.B-3.11a.B-1 to I.B-3.11a.B-94, I.B-4.11a.B-1 to I.B-4.11a.B-94, I.B-5.11a.B-1 to I.B-5.11a.B-94, I.B-6.11a.B-1 to I.B-6.11a.B-94).
Table 12a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 4-Cl—C₆H₄ and the meaning for R⁵ for each individual compound corresponds in each case to one line of Table B (compounds I.A-1.12a.B-1 to I.A-1.12a.B-94, I.A-2.12a.B-1 to I.A-2.12a.B-94, I.A-3.12a.B-1 to I.A-3.12a.B-94, I.A-4.12a.B-1 to I.A-5.12a.B-94, I.A-5.12a.B-1 to I.A-3.12a.B-94, I.A-6.12a.B-1 to I.A-6.12a.B-94; I.B-12a.B-1 to I.B-1.12a.B-94, I.B-2.12a.B-1 to I.B-2.12a.B-94, I.B-3.12a.B-1 to I.B-3.12a.B-94, I.B-4.12a.B-1 to I.B-4.12a.B-94, I.B-5.12a.B-1 to I.B-5.12a.B-94, I.B-6.12a.B-1 to I.B-6.12a.B-94).
Table 13a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 2-OCH₃—C₆H₄ and the meaning for R⁵ for each individual compound corresponds in each case to one line of Table B (compounds I.A-1.13a.B-1 to I.A-1.13a.B-94, I.A-2.13a.B-1 to I.A-2.13a.B-94, I.A-3.13a.B-1 to I.A-3.13a.B-94, I.A-4.13a.B-1 to I.A-5.13a.B-94, I.A-5.13a.B-1 to I.A-3.13a.B-94, I.A-6.13a.B-1 to I.A-6.13a.B-94; I.B-13a.B-1 to I.B-1.13a.B-94, I.B-2.13a.B-1 to I.B-2.13a.B-94, I.B-3.13a.B-1 to I.B-3.13a.B-94, I.B-4.13a.B-1 to I.B-4.13a.B-94, I.B-5.13a.B-1 to I.B-5.13a.B-94, I.B-6.13a.B-1 to I.B-6.13a.B-94).
Table 14a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 3-OCH₃—C₆H₄ and the meaning for R⁵ for each individual compound corresponds in each case to one line of Table B (compounds I.A-1.14a.B-1 to I.A-1.14a.B-94, I.A-2.14a.B-1 to I.A-2.14a.B-94, I.A-3.14a.B-1 to I.A-3.14a.B-94, I.A-4.14a.B-1 to I.A-5.14a.B-94, I.A-5.14a.B-1 to I.A-3.14a.B-94, I.A-6.14a.B-1 to I.A-6.14a.B-94; I.B-14a.B-1 to I.B-1.14a.B-94, I.B-2.14a.B-1 to I.B-2.14a.B-94, I.B-3.14a.B-1 to I.B-3.14a.B-94, I.B-4.14a.B-1 to I.B-4.14a.B-94, I.B-5.14a.B-1 to I.B-5.14a.B-94, I.B-6.14a.B-1 to I.B-6.14a.B-94).
Table 15a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 4-OCH₃—C₆H₄ and the meaning for R⁵ for each individual compound corresponds in each case to one line of Table B (compounds I.A-1.15a.B-1 to I.A-1.15a.B-94, I.A-2.15a.B-1 to I.A-2.15a.B-94, I.A-3.15a.B-1 to I.A-3.15a.B-94, I.A-4.15a.B-1 to I.A-5.15a.B-94, I.A-5.15a.B-1 to I.A-3.15a.B-94, I.A-6.15a.B-1 to I.A-6.15a.B-94; I.E-15a.B-1 to I.B-1.15a.B-94, I.B-2.15a.B-1 to I.B-2.15a.B-94, I.B-3.15a.B-1 to I.B-3.15a.B-94, I.B-4.15a.B-1 to I.B-4.15a.B-94, I.B-5.15a.B-1 to I.B-5.15a.B-94, I.B-6.15a.B-1 to I.B-6.15a.B-94).
Table 16a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 2-OCHF₂—C₆H₄ and the meaning for R⁵ for each individual compound corresponds in each case to one line of Table B (compounds I.A-1.16a.B-1 to I.A-1.16a.B-94, I.A-2.16a.B-1 to I.A-2.16a.B-94, I.A-3.16a.B-1 to I.A-3.16a.B-94, I.A-4.16a.B-1 to I.A-5.16a.B-94, I.A-5.16a.B-1 to I.A-3.16a.B-94, I.A-6.16a.B-1 to I.A-6.16a.B-94; I.B-16a.B-1 to I.B-1.16a.B-94, I.B-2.16a.B-1 to I.B-2.16a.B-94, I.B-3.16a.B-1 to I.B-3.16a.B-94, I.B-4.16a.B-1 to I.B-4.16a.B-94, I.B-5.16a.B-1 to I.B-5.16a.B-94, I.B-6.16a.B-1 to I.B-6.16a.B-94).
Table 17a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 3-OCHF₂—C₆H₄ and the meaning for R⁵ for each individual compound corresponds in each case to one line of Table B (compounds I.A-1.17a.B-1 to I.A-1.17a.B-94, I.A-2.17a.B-1 to I.A-2.17a.B-94, I.A-3.17a.B-1 to I.A-3.17a.B-94, I.A-4.17a.B-1 to I.A-5.17a.B-94, I.A-5.17a.B-1 to I.A-3.17a.B-94, I.A-6.17a.B-1 to I.A-6.17a.B-94; I.B-17a.B-1 to I.B-1.17a.B-94, I.B-2.17a.B-1 to I.B-2.17a.B-94, I.B-3.17a.B-1 to I.B-3.17a.B-94, I.B-4.17a.B-1 to I.B-4.17a.B-94, I.B-5.17a.B-1 to I.B-5.17a.B-94, I.B-6.17a.B-1 to I.B-6.17a.B-94).
Table 18a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 4-OCHF₂—C₆H₄ and the meaning for R⁵ for each individual compound corresponds in each case to one line of Table B (compounds I.A-1.18a.B-1 to I.A-1.18a.B-94, I.A-2.18a.B-1 to I.A-2.18a.B-94, I.A-3.18a.B-1 to I.A-3.18a.B-94, I.A-4.18a.B-1 to I.A-5.18a.B-94, I.A-5.18a.B-1 to I.A-3.18a.B-94, I.A-6.18a.B-1 to I.A-6.18a.B-94; I.B-18a.B-1 to I.B-1.18a.B-94, I.B-2.18a.B-1 to I.B-2.18a.B-94, I.B-3.18a.B-1 to I.B-3.18a.B-94, I.B-4.18a.B-1 to I.B-4.18a.B-94, I.B-5.18a.B-1 to I.B-5.18a.B-94, I.B-6.18a.B-1 to I.B-6.18a.B-94).
Table 19a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 2-CH₃—C₆H₄ and the meaning for R⁵ for each individual compound corresponds in each case to one line of Table B (compounds I.A-1.19a.B-1 to I.A-1.19a.B-94, I.A-2.19a.B-1 to I.A-2.19a.B-94, I.A-3.19a.B-1 to I.A-3.19a.B-94, I.A-4.19a.B-1 to I.A-5.19a.B-94, I.A-5.19a.B-1 to I.A-3.19a.B-94, I.A-6.19a.B-1 to I.A-6.19a.B-94; I.E-19a.B-1 to I.B-1.19a.B-94, I.B-2.19a.B-1 to I.B-2.19a.B-94, I.B-3.19a.B-1 to I.B-3.19a.B-94, I.B-4.19a.B-1 to I.B-4.19a.B-94, I.B-5.19a.B-1 to I.B-5.19a.B-94, I.B-6.19a.B-1 to I.B-6.19a.B-94).
Table 20a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 3-CH₃—C₆H₄ and the meaning for R⁵ for each individual compound corresponds in each case to one line of Table B (compounds I.A-1.20a.B-1 to I.A-1.20a.B-94, I.A-2.20a.B-1 to I.A-2.20a.B-94, I.A-3.20a.B-1 to I.A-3.20a.B-94, I.A-4.20a.B-1 to I.A-5.20a.B-94, I.A-5.20a.B-1 to I.A-3.20a.B-94, I.A-6.20a.B-1 to I.A-6.20a.B-94; I.B-20a.B-1 to I.B-1.20a.B-94, I.B-2.20a.B-1 to I.B-2.20a.B-94, I.B-3.20a.B-1 to I.B-3.20a.B-94, I.B-4.20a.B-1 to I.B-4.20a.B-94, I.B-5.20a.B-1 to I.B-5.20a.B-94, I.B-6.20a.B-1 to I.B-6.20a.B-94).
Table 21a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 4-CH₃—C₆H₄ and the meaning for R⁵ for each individual compound corresponds in each case to one line of Table B (compounds I.A-1.21a.B-1 to I.A-1.21a.B-94, I.A-2.21a.B-1 to I.A-2.21a.B-94, I.A-3.21a.B-1 to I.A-3.21a.B-94, I.A-4.21a.B-1 to I.A-5.21a.B-94, I.A-5.21a.B-1 to I.A-3.21a.B-94, I.A-6.21a.B-1 to I.A-6.21a.B-94; I.B-21a.B-1 to I.B-1.21a.B-94, I.B-2.21a.B-1 to I.B-2.21a.B-94, I.B-3.21a.B-1 to I.B-3.21a.B-94, I.B-4.21a.B-1 to I.B-4.21a.B-94, I.B-5.21a.B-1 to I.B-5.21a.B-94, I.B-6.21a.B-1 to I.B-6.21a.B-94).
Table 22a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 2-CF₃—C₆H₄ and the meaning for R⁵ for each individual compound corresponds in each case to one line of Table B (compounds I.A-1.22a.B-1 to I.A-1.22a.B-94, I.A-2.22a.B-1 to I.A-2.22a.B-94, I.A-3.22a.B-1 to I.A-3.22a.B-94, I.A-4.22a.B-1 to I.A-5.22a.B-94, I.A-5.22a.B-1 to I.A-3.22a.B-94, I.A-6.22a.B-1 to I.A-6.22a.B-94; I.B-22a.B-1 to I.B-1.22a.B-94, I.B-2.22a.B-1 to I.B-2.22a.B-94, I.B-3.22a.B-1 to I.B-3.22a.B-94, I.B-4.22a.B-1 to I.B-4.22a.B-94, I.B-5.22a.B-1 to I.B-5.22a.B-94, I.B-6.22a.B-1 to I.B-6.22a.B-94).
Table 23a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 3-CF₃—C₆H₄ and the meaning for R⁵ for each individual compound corresponds in each case to one line of Table B (compounds I.A-1.23a.B-1 to I.A-1.23a.B-94, I.A-2.23a.B-1 to I.A-2.23a.B-94, I.A-3.23a.B-1 to I.A-3.23a.B-94, I.A-4.23a.B-1 to I.A-5.23a.B-94, I.A-5.23a.B-1 to I.A-3.23a.B-94, I.A-6.23a.B-1 to I.A-6.23a.B-94; I.B-23a.B-1 to I.B-1.23a.B-94, I.B-2.23a.B-1 to I.B-2.23a.B-94, I.B-3.23a.B-1 to I.B-3.23a.B-94, I.B-4.23a.B-1 to I.B-4.23a.B-94, I.B-5.23a.B-1 to I.B-5.23a.B-94, I.B-6.23a.B-1 to I.B-6.23a.B-94).
Table 24a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 4-CF₃—C₆H₄ and the meaning for R⁵ for each individual compound corresponds in each case to one line of Table B (compounds I.A-1.24a.B-1 to I.A-1.24a.B-94, I.A-2.24a.B-1 to I.A-2.24a.B-94, I.A-3.24a.B-1 to I.A-3.24a.B-94, I.A-4.24a.B-1 to I.A-5.24a.B-94, I.A-5.24a.B-1 to I.A-3.24a.B-94, I.A-6.24a.B-1 to I.A-6.24a.B-94; I.B-24a.B-1 to I.B-1.24a.B-94, I.B-2.24a.B-1 to I.B-2.24a.B-94, I.B-3.24a.B-1 to I.B-3.24a.B-94, I.B-4.24a.B-1 to I.B-4.24a.B-94, I.B-5.24a.B-1 to I.B-5.24a.B-94, I.B-6.24a.B-1 to I.B-6.24a.B-94).
Table 25a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 2,3-F₂-C₆H₃ and the meaning for R⁵ for each individual compound corresponds in each case to one line of Table B (compounds I.A-1.25a.B-1 to I.A-1.25a.B-94, I.A-2.25a.B-1 to I.A-2.25a.B-94, I.A-3.25a.B-1 to I.A-3.25a.B-94, I.A-4.25a.B-1 to I.A-5.25a.B-94, I.A-5.25a.B-1 to I.A-3.25a.B-94, I.A-6.25a.B-1 to I.A-6.25a.B-94; I.B-25a.B-1 to I.B-1.25a.B-94, I.B-2.25a.B-1 to I.B-2.25a.B-94, I.B-3.25a.B-1 to I.B-3.25a.B-94, I.B-4.25a.B-1 to I.B-4.25a.B-94, I.B-5.25a.B-1 to I.B-5.25a.B-94, I.B-6.25a.B-1 to I.B-6.25a.B-94).
Table 26a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 2,4-F₂-C₆H₃ and the meaning for R⁵ for each individual compound corresponds in each case to one line of Table B (compounds I.A-1.26a.B-1 to I.A-1.26a.B-94, I.A-2.26a.B-1 to I.A-2.26a.B-94, I.A-3.26a.B-1 to I.A-3.26a.B-94, I.A-4.26a.B-1 to I.A-5.26a.B-94, I.A-5.26a.B-1 to I.A-3.26a.B-94, I.A-6.26a.B-1 to I.A-6.26a.B-94; I.B-26a.B-1 to I.B-1.26a.B-94, I.B-2.26a.B-1 to I.B-2.26a.B-94, I.B-3.26a.B-1 to I.B-3.26a.B-94, I.B-4.26a.B-1 to I.B-4.26a.B-94, I.B-5.26a.B-1 to I.B-5.26a.B-94, I.B-6.26a.B-1 to I.B-6.26a.B-94).
Table 27a Compounds of the formula I.A-1, I.A-2, I.A-3, I.A-4, I.A-5, I.A-6; I.E-1, I.B-2, I.B-3, I.B-4, I.B-5, I.B-6 in which R⁸ is 2,5-F₂-C₆H₃ and the meaning for R⁵ for each individual compound corresponds in each case to one line of Table B (compounds I.A-1.27a.B-1 to I.A-1.27a.B-94, I.A-2.27a.B-1 to I.A-2.27a.B-94, I.A-3.27a.B-1 to I.A-3.27a.B-94, I.A-4.27a.B-1 to I.A-5.27a.B-94, I.A-5.27a.B-1 to I.A-3.27a.B-94, I.A-6.27a.B-1 to I.A-6.27a.B-94; I.B-27a.B-1 to I.B-1.27a.B-94, I.B-2.27a.B-1 to I.B-2.27a.B-94, I.B-3.27a.B-1 to I.B-3.27a.B-94, I.B-4.27a.B-1 to I.B-4.27a.B-94, I.B-5.27a.B-1 to I.B-5.27a.B-94, I.B-6.27a.B-1 to I.B-6.27a.B-94).
Table 28a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 2,6-F₂-C₆H₃ and the meaning for R⁵ for each individual compound corresponds in each case to one line of Table B (compounds I.A-1.28a.B-1 to I.A-1.28a.B-94, I.A-2.28a.B-1 to I.A-2.28a.B-94, I.A-3.28a.B-1 to I.A-3.28a.B-94, I.A-4.28a.B-1 to I.A-5.28a.B-94, I.A-5.28a.B-1 to I.A-3.28a.B-94, I.A-6.28a.B-1 to I.A-6.28a.B-94; I.B-28a.B-1 to I.B-1.28a.B-94, I.B-2.28a.B-1 to I.B-2.28a.B-94, I.B-3.28a.B-1 to I.B-3.28a.B-94, I.B-4.28a.B-1 to I.B-4.28a.B-94, I.B-5.28a.B-1 to I.B-5.28a.B-94, I.B-6.28a.B-1 to I.B-6.28a.B-94).
Table 29a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 3,4-F₂-C₆H₃ and the meaning for R⁵ for each individual compound corresponds in each case to one line of Table B (compounds I.A-1.29a.B-1 to I.A-1.29a.B-94, I.A-2.29a.B-1 to I.A-2.29a.B-94, I.A-3.29a.B-1 to I.A-3.29a.B-94, I.A-4.29a.B-1 to I.A-5.29a.B-94, I.A-5.29a.B-1 to I.A-3.29a.B-94, I.A-6.29a.B-1 to I.A-6.29a.B-94; I.B-29a.B-1 to I.B-1.29a.B-94, I.B-2.29a.B-1 to I.B-2.29a.B-94, I.B-3.29a.B-1 to I.B-3.29a.B-94, I.B-4.29a.B-1 to I.B-4.29a.B-94, I.B-5.29a.B-1 to I.B-5.29a.B-94, I.B-6.29a.B-1 to I.B-6.29a.B-94).
Table 30a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 3,5-F₂-C₆H₃ and the meaning for R⁵ for each individual compound corresponds in each case to one line of Table B (compounds I.A-1.30a.B-1 to I.A-1.30a.B-94, I.A-2.30a.B-1 to I.A-2.30a.B-94, I.A-3.30a.B-1 to I.A-3.30a.B-94, I.A-4.30a.B-1 to I.A-5.30a.B-94, I.A-5.30a.B-1 to I.A-3.30a.B-94, I.A-6.30a.B-1 to I.A-6.30a.B-94; I.B-30a.B-1 to I.B-1.30a.B-94, I.B-2.30a.B-1 to I.B-2.30a.B-94, I.B-3.30a.B-1 to I.B-3.30a.B-94, I.B-4.30a.B-1 to I.B-4.30a.B-94, I.B-5.30a.B-1 to I.B-5.30a.B-94, I.B-6.30a.B-1 to I.B-6.30a.B-94).
Table 31a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 2,3-Cl₂—C₆H₃ and the meaning for R⁵ for each individual compound corresponds in each case to one line of Table B (compounds I.A-1.31a.B-1 to I.A-1.31a.B-94, I.A-2.31a.B-1 to I.A-2.31a.B-94, I.A-3.31a.B-1 to I.A-3.31a.B-94, I.A-4.31a.B-1 to I.A-5.31a.B-94, I.A-5.31a.B-1 to I.A-3.31a.B-94, I.A-6.31a.B-1 to I.A-6.31a.B-94; I.B-31a.B-1 to I.B-1.31a.B-94, I.B-2.31a.B-1 to I.B-2.31a.B-94, I.B-3.31a.B-1 to I.B-3.31a.B-94, I.B-4.31a.B-1 to I.B-4.31a.B-94, I.B-5.31a.B-1 to I.B-5.31a.B-94, I.B-6.31a.B-1 to I.B-6.31a.B-94).
Table 32a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 2,4-Cl₂—C₆H₃ and the meaning for R⁵ for each individual compound corresponds in each case to one line of Table B (compounds I.A-1.32a.B-1 to I.A-1.32a.B-94, I.A-2.32a.B-1 to I.A-2.32a.B-94, I.A-3.32a.B-1 to I.A-3.32a.B-94, I.A-4.32a.B-1 to I.A-5.32a.B-94, I.A-5.32a.B-1 to I.A-3.32a.B-94, I.A-6.32a.B-1 to I.A-6.32a.B-94; I.B-32a.B-1 to I.B-1.32a.B-94, I.B-2.32a.B-1 to I.B-2.32a.B-94, I.B-3.32a.B-1 to I.B-3.32a.B-94, I.B-4.32a.B-1 to I.B-4.32a.B-94, I.B-5.32a.B-1 to I.B-5.32a.B-94, I.B-6.32a.B-1 to I.B-6.32a.B-94).
Table 33a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 2,5-Cl₂—C₆H₃ and the meaning for R⁵ for each individual compound corresponds in each case to one line of Table B (compounds I.A-1.33a.B-1 to I.A-1.33a.B-94, I.A-2.33a.B-1 to I.A-2.33a.B-94, I.A-3.33a.B-1 to I.A-3.33a.B-94, I.A-4.33a.B-1 to I.A-5.33a.B-94, I.A-5.33a.B-1 to I.A-3.33a.B-94, I.A-6.33a.B-1 to I.A-6.33a.B-94; I.B-33a.B-1 to I.B-1.33a.B-94, I.B-2.33a.B-1 to I.B-2.33a.B-94, I.B-3.33a.B-1 to I.B-3.33a.B-94, I.B-4.33a.B-1 to I.B-4.33a.B-94, I.B-5.33a.B-1 to I.B-5.33a.B-94, I.B-6.33a.B-1 to I.B-6.33a.B-94).
Table 34a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 2,6-Cl₂—C₆H₃ and the meaning for R⁵ for each individual compound corresponds in each case to one line of Table B (compounds I.A-1.34a.B-1 to I.A-1.34a.B-94, I.A-2.34a.B-1 to I.A-2.34a.B-94, I.A-3.34a.B-1 to I.A-3.34a.B-94, I.A-4.34a.B-1 to I.A-5.34a.B-94, I.A-5.34a.B-1 to I.A-3.34a.B-94, I.A-6.34a.B-1 to I.A-6.34a.B-94; I.B-34a.B-1 to I.B-1.34a.B-94, I.B-2.34a.B-1 to I.B-2.34a.B-94, I.B-3.34a.B-1 to I.B-3.34a.B-94, I.B-4.34a.B-1 to I.B-4.34a.B-94, I.B-5.34a.B-1 to I.B-5.34a.B-94, I.B-6.34a.B-1 to I.B-6.34a.B-94).
Table 35a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 3,4-Cl₂—C₆H₃ and the meaning for R⁵ for each individual compound corresponds in each case to one line of Table B (compounds I.A-1.35a.B-1 to I.A-1.35a.B-94, I.A-2.35a.B-1 to I.A-2.35a.B-94, I.A-3.35a.B-1 to I.A-3.35a.B-94, I.A-4.35a.B-1 to I.A-5.35a.B-94, I.A-5.35a.B-1 to I.A-3.35a.B-94, I.A-6.35a.B-1 to I.A-6.35a.B-94; I.B-35a.B-1 to I.B-1.35a.B-94, I.B-2.35a.B-1 to I.B-2.35a.B-94, I.B-3.35a.B-1 to I.B-3.35a.B-94, I.B-4.35a.B-1 to I.B-4.35a.B-94, I.B-5.35a.B-1 to I.B-5.35a.B-94, I.B-6.35a.B-1 to I.B-6.35a.B-94).
Table 36a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 3,5-Cl₂—C₆H₃ and the meaning for R⁵ for each individual compound corresponds in each case to one line of Table B (compounds I.A-1.36a.B-1 to I.A-1.36a.B-94, I.A-2.36a.B-1 to I.A-2.36a.B-94, I.A-3.36a.B-1 to I.A-3.36a.B-94, I.A-4.36a.B-1 to I.A-5.36a.B-94, I.A-5.36a.B-1 to I.A-3.36a.B-94, I.A-6.36a.B-1 to I.A-6.36a.B-94; I.B-36a.B-1 to I.B-1.36a.B-94, I.B-2.36a.B-1 to I.B-2.36a.B-94, I.B-3.36a.B-1 to I.B-3.36a.B-94, I.B-4.36a.B-1 to I.B-4.36a.B-94, I.B-5.36a.B-1 to I.B-5.36a.B-94, I.B-6.36a.B-1 to I.B-6.36a.B-94).
Table 37a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 2,3-(CH₃)₂-C₆H₃ and the meaning for R⁵ for each individual compound corresponds in each case to one line of Table B (compounds I.A-1.37a.B-1 to I.A-1.37a.B-94, I.A-2.37a.B-1 to I.A-2.37a.B-94, I.A-3.37a.B-1 to I.A-3.37a.B-94, I.A-4.37a.B-1 to I.A-5.37a.B-94, I.A-5.37a.B-1 to I.A-3.37a.B-94, I.A-6.37a.B-1 to I.A-6.37a.B-94; I.B-37a.B-1 to I.B-1.37a.B-94, I.B-2.37a.B-1 to I.B-2.37a.B-94, I.B-3.37a.B-1 to I.B-3.37a.B-94, I.B-4.37a.B-1 to I.B-4.37a.B-94, I.B-5.37a.B-1 to I.B-5.37a.B-94, I.B-6.37a.B-1 to I.B-6.37a.B-94).
Table 38a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 2,4-(CH₃)₂-C₆H₃ and the meaning for R⁵ for each individual compound corresponds in each case to one line of Table B (compounds I.A-1.38a.B-1 to I.A-1.38a.B-94, I.A-2.38a.B-1 to I.A-2.38a.B-94, I.A-3.38a.B-1 to I.A-3.38a.B-94, I.A-4.38a.B-1 to I.A-5.38a.B-94, I.A-5.38a.B-1 to I.A-3.38a.B-94, I.A-6.38a.B-1 to I.A-6.38a.B-94; I.B-38a.B-1 to I.B-1.38a.B-94, I.B-2.38a.B-1 to I.B-2.38a.B-94, I.B-3.38a.B-1 to I.B-3.38a.B-94, I.B-4.38a.B-1 to I.B-4.38a.B-94, I.B-5.38a.B-1 to I.B-5.38a.B-94, I.B-6.38a.B-1 to I.B-6.38a.B-94).
Table 39a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 2,5-(CH₃)₂-C₆H₃ and the meaning for R⁵ for each individual compound corresponds in each case to one line of Table B (compounds I.A-1.39a.B-1 to I.A-1.39a.B-94, I.A-2.39a.B-1 to I.A-2.39a.B-94, I.A-3.39a.B-1 to I.A-3.39a.B-94, I.A-4.39a.B-1 to I.A-5.39a.B-94, I.A-5.39a.B-1 to I.A-3.39a.B-94, I.A-6.39a.B-1 to I.A-6.39a.B-94; I.B-39a.B-1 to I.B-1.39a.B-94, I.B-2.39a.B-1 to I.B-2.39a.B-94, I.B-3.39a.B-1 to I.B-3.39a.B-94, I.B-4.39a.B-1 to I.B-4.39a.B-94, I.B-5.39a.B-1 to I.B-5.39a.B-94, I.B-6.39a.B-1 to I.B-6.39a.B-94).
Table 40a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 2,6-(CH₃)₂-C₆H₃ and the meaning for R⁵ for each individual compound corresponds in each case to one line of Table B (compounds I.A-1.40a.B-1 to I.A-1.40a.B-94, I.A-2.40a.B-1 to I.A-2.40a.B-94, I.A-3.40a.B-1 to I.A-3.40a.B-94, I.A-4.40a.B-1 to I.A-5.40a.B-94, I.A-5.40a.B-1 to I.A-3.40a.B-94, I.A-6.40a.B-1 to I.A-6.40a.B-94; I.B-40a.B-1 to I.B-1.40a.B-94, I.B-2.40a.B-1 to I.B-2.40a.B-94, I.B-3.40a.B-1 to I.B-3.40a.B-94, I.B-4.40a.B-1 to I.B-4.40a.B-94, I.B-5.40a.B-1 to I.B-5.40a.B-94, I.B-6.40a.B-1 to I.B-6.40a.B-94).
Table 41a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 3,4-(CH₃)₂-C₆H₃ and the meaning for R⁵ for each individual compound corresponds in each case to one line of Table B (compounds I.A-1.41a.B-1 to I.A-1.41a.B-94, I.A-2.41a.B-1 to I.A-2.41a.B-94, I.A-3.41a.B-1 to I.A-3.41a.B-94, I.A-4.41a.B-1 to I.A-5.41a.B-94, I.A-5.41a.B-1 to I.A-3.41a.B-94, I.A-6.41a.B-1 to I.A-6.41a.B-94; I.B-41a.B-1 to I.B-1.41a.B-94, I.B-2.41a.B-1 to I.B-2.41a.B-94, I.B-3.41a.B-1 to I.B-3.41a.B-94, I.B-4.41a.B-1 to I.B-4.41a.B-94, I.B-5.41a.B-1 to I.B-5.41a.B-94, I.B-6.41a.B-1 to I.B-6.41a.B-94).
Table 42a Compounds of the formula I.A-1, I.A-2, I.A-3, I.A-4, I.A-5, I.A-6; I.B-1, I.B-2, I.B-3, I.B-4, I.B-5, I.B-6 in which R⁸ is 3,5-(CH₃)₂-C₆H₃ and the meaning for R⁵ for each individual compound corresponds in each case to one line of Table B (compounds I.A-1.42a.B-1 to I.A-1.42a.B-94, I.A-2.42a.B-1 to I.A-2.42a.B-94, I.A-3.42a.B-1 to I.A-3.42a.B-94, I.A-4.42a.B-1 to I.A-5.42a.B-94, I.A-5.42a.B-1 to I.A-3.42a.B-94, I.A-6.42a.B-1 to I.A-6.42a.B-94; I.B-42a.B-1 to I.B-1.42a.B-94, I.B-2.42a.B-1 to I.B-2.42a.B-94, I.B-3.42a.B-1 to I.B-3.42a.B-94, I.B-4.42a.B-1 to I.B-4.42a.B-94, I.B-5.42a.B-1 to I.B-5.42a.B-94, I.B-6.42a.B-1 to I.B-6.42a.B-94).
Table 43a Compounds of the formula I.A-1, I.A-2, I.A-3, I.A-4, I.A-5, I.A-6; I.B-1, I.B-2, I.B-3, I.B-4, I.B-5, I.B-6 in which R⁸ is

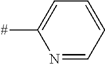

and the meaning for R⁵ for each individual compound corresponds in each case to one line of Table B (compounds I.A-1.43a.B-1 to I.A-1.43a.B-94, I.A-2.43a.B-1 to I.A-2.43a.B-94, I.A-3.43a.B-1 to I.A-3.43a.B-94, I.A-4.43a.B-1 to I.A-5.43a.B-94, I.A-5.43a.B-1 to I.A-3.43a.B-94, I.A-6.43a.B-1 to I.A-6.43a.B-94; I.B-43a.B-1 to I.B-1.43a.B-94, I.B-2.43a.B-1 to I.B-2.43a.B-94, I.B-3.43a.B-1 to I.B-3.43a.B-94, I.B-4.43a.B-1 to I.B-4.43a.B-94, I.B-5.43a.B-1 to I.B-5.43a.B-94, I.B-6.43a.B-1 to I.B-6.43a.B-94).

TABLE B

| No. | R⁵ |
|---|---|
| B-1 | CH₃ |
| B-2 | C₂H₅ |
| B-3 | n-C₃H₇ |
| B-4 | CH(CH₃)₂ |
| B-5 | n-C₄H₉ |
| B-6 | CH(CH₃)—CH₂—CH₃ |
| B-7 | CH₂—CH(CH₃)₂ |
| B-8 | n-C₅H₁₁ |
| B-9 | CH(CH₃)—CH₂—CH₂—CH₃ |
| B-10 | CH₂—CH(CH₃)—CH₂—CH₃ |
| B-11 | CH₂—CH₂—CH(CH₃)₂ |
| B-12 | CH₂—CHF₂ |
| B-13 | CH₂—CF₃ |
| B-14 | CH₂—CH═CH₂ |
| B-15 | CH₂—CH═CHCl |
| B-16 | CH₂—C≡CH |
| B-17 | CH₂-cyclo-C₃H₅ |
| B-18 | CH₂-cyclo-C₄H₇ |
| B-19 | CH₂-cyclo-C₅H₉ |
| B-20 | CH₂-cyclo-C₆H₁₁ |
| B-21 | C₆H₅ |
| B-22 | 2-F—C₆H₄ |
| B-23 | 3-F—C₆H₄ |
| B-24 | 4-F—C₆H₄ |
| B-25 | 2-Cl—C₆H₄ |
| B-26 | 3-Cl—C₆H₄ |
| B-27 | 4-Cl—C₆H₄ |
| B-28 | 2-OCH₃—C₆H₄ |
| B-29 | 3-OCH₃—C₆H₄ |
| B-30 | 4-OCH₄—C₆H₄ |
| B-31 | 2-OCHF₂—C₆H₄ |
| B-32 | 3-OCHF₂—C₆H₄ |
| B-33 | 4-OCHF₂—C₆H₄ |
| B-34 | 2-CH₃—C₆H₄ |
| B-35 | 3-CH₃—C₆H₄ |
| B-36 | 4-CH₃—C₆H₄ |
| B-37 | 2-CF₃—C₆H₄ |
| B-38 | 3-CF₃—C₆H₄ |
| B-39 | 4-CF₃—C₆H₄ |
| B-40 | 2,3-F₂—C₆H₃ |
| B-41 | 2,4-F₂—C₆H₃ |
| B-42 | 2,5-F₂—C₆H₃ |
| B-43 | 2,6-F₂—C₆H₃ |
| B-44 | 3,4-F₂—C₆H₃ |
| B-45 | 3,5-F₂—C₆H₃ |
| B-46 | 2,3-Cl₂—C₆H₃ |
| B-47 | 2,4-Cl₂—C₆H₃ |
| B-48 | 2,5-Cl₂—C₆H₃ |
| B-49 | 2,6-Cl₂—C₆H₃ |
| B-50 | 3,4-Cl₂—C₆H₃ |
| B-51 | 3,5-Cl₂—C₆H₃ |
| B-52 | 2,3-(CH₃)₂—C₆H₃ |
| B-53 | 2,4-(CH₃)₂—C₆H₃ |
| B-54 | 2,5-(CH₃)₂—C₆H₃ |
| B-55 | 2,6-(CH₃)₂—C₆H₃ |
| B-56 | 3,4-(CH₃)₂—C₆H₃ |
| B-57 | 3,5-(CH₃)₂—C₆H₃ |
| B-58 | C₆H₅—CH₂ |
| B-59 | 2-F—C₆H₄—CH₂ |
| B-60 | 3-F—C₆H₄—CH₂ |
| B-61 | 4-F—C₆H₄—CH₂ |
| B-62 | 2-Cl—C₆H₄—CH₂ |
| B-63 | 3-Cl—C₆H₄—CH₂ |
| B-64 | 4-Cl—C₆H₄—CH₂ |
| B-65 | 2-CH₃—C₆H₄—CH₂ |
| B-66 | 3-CH₃—C₆H₄—CH₂ |
| B-67 | 4-CH₃—C₆H₄—CH₂ |
| B-68 | 2-CF₃—C₆H₄—CH₂ |
| B-69 | 3-CF₃—C₆H₄—CH₂ |
| B-70 | 4-CF₃—C₆H₄—CH₂ |
| B-71 | 2-OCH₃—C₆H₄—CH₂ |
| B-72 | 3-OCH₃—C₆H₄—CH₂ |
| B-73 | 4-OCH₃—C₆H₄—CH₂ |
| B-74 | 2-OCHF₂—C₆H₄—CH₂ |
| B-75 | 3-OCHF₂—C₆H₄—CH₂ |
| B-76 | 4-OCHF₂—C₆H₄—CH₂ |
| B-77 | 2,3-F₂—C₆H₃—CH₂ |
| B-78 | 2,4-F₂—C₆H₃—CH₂ |
| B-79 | 2,5-F₂—C₆H₃—CH₂ |
| B-80 | 2,6-F₂—C₆H₃—CH₂ |
| B-81 | 3,4-F₂—C₆H₃—CH₂ |
| B-82 | 3,5-F₂—C₆H₃—CH₂ |
| B-83 | 2,3-Cl₂—C₆H₃—CH₂ |
| B-84 | 2,4-Cl₂—C₆H₃—CH₂ |
| B-85 | 2,5-Cl₂—C₆H₃—CH₂ |
| B-86 | 2,6-Cl₂—C₆H₃—CH₂ |
| B-87 | 3,4-Cl₂—C₆H₃—CH₂ |
| B-88 | 3,5-Cl₂—C₆H₃—CH₂ |
| B-89 | 2,3-(CH₃)₂—C₆H₃—CH₂ |
| B-90 | 2,4-(CH₃)₂—C₆H₃—CH₂ |
| B-91 | 2,5-(CH₃)₂—C₆H₃—CH₂ |
| B-92 | 2,6-(CH₃)₂—C₆H₃—CH₂ |
| B-93 | 3,4-(CH₃)₂—C₆H₃—CH₂ |
| B-94 | 3,5-(CH₃)₂—C₆H₃—CH₂ |

Preferred embodiments of the present invention are the following compounds I.C-1, I.C-2, I.C-3, I.C-4, I.C-5, I.C-6; compounds I.D-1, I.D-2, I.D-3, I.D-4, I.D-5, I.D-6. In these formulae, the substituents R⁵, R⁸ are independently as defined above or preferably defined herein:

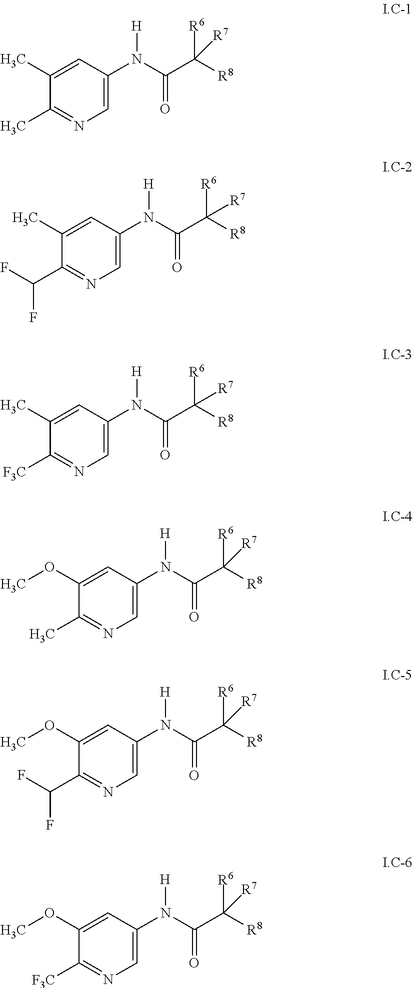

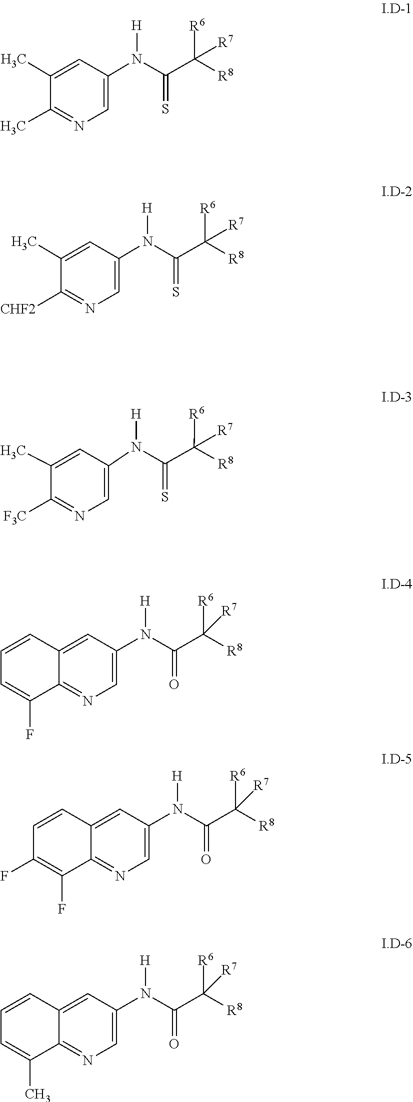

In particular with a view to their use, according to one embodiment, preference is given to the compounds of the compounds I.C-1, I.C-2, I.C-3, I.C-4, I.C-5, I.C-6; compounds I.D-1, I.D-2, I.D-3, I.D-4, I.D-5, I.D-6, that are compiled in the Tables 1b to 43 b. Each of the groups mentioned for a substituent in the tables is furthermore per se, independently of the combination in which it is mentioned, a particularly preferred aspect of the substituent in question.

Table 1b Compounds of the formula I.C-1, I.C-2, I.C-3, I.C-4, I.C-5, I.C-6; I.D-1, I.D-2, I.D-3, I.D-4, I.D-5, I.D-6 in which R⁸ is cyclo-C₃H₅ and the meaning for the combination of R⁶ and R⁷ for each individual compound corresponds in each case to one line of Table X (compounds I.C-1b.X-1 to I.D-1.1b.X-384, I.D-2.1b.X-1 to I.D-2.1b.X-384, I.D-3.1b.X-1 to I.D-3.1b.X-384, I.D-4.1b.X-1 to I.D-4.1b.X-384, I.D-5.1b.X-1 to I.D-5.1b.X-384, I.D-6.1b.X-1 to I.D-6.1b.X-384).
Table 2b Compounds of the formula I.C-1, I.C-2, I.C-3, I.C-4, I.C-5, I.C-6; I.D-1, I.D-2, I.D-3, I.D-4, I.D-5, I.D-6 in which R⁸ is cyclo-C₄H₇ and the meaning for the combination of R⁶ and R⁷ for each individual compound corresponds in each case to one line of Table X (compounds I.C-1.2b.X-1 to I.C-1.2b.X-384, I.C-2.2b.X-1 to I.C-2.2b.X-384, I.C-3.2b.X-1 to I.C-3.2b.X-384, I.C-4.2b.X-1 to I.C-5.2b.X-384, I.C-5.2b.X-1 to I.C-3.2b.X-384, I.C-6.2b.X-1 to I.C-6.2b.X-384; I.D-2b.X-1 to I.D-1.2b.X-384, I.D-2.2b.X-1 to I.D-2.2b.X-384, I.D-3.2b.X-1 to I.D-3.2b.X-384, I.D-4.2b.X-1 to I.D-4.2b.X-384, I.D-5.2b.X-1 to I.D-5.2b.X-384, I.D-6.2b.X-1 to I.D-6.2b.X-384).
Table 3b Compounds of the formula I.C-1, I.C-2, I.C-3, I.C-4, I.C-5, I.C-6; I.D-1, I.D-2, I.D-3, I.D-4, I.D-5, I.D-6 in which R⁸ is cyclo-C₅H₉ and the meaning for the combination of R⁶ and R⁷ for each individual compound corresponds in each case to one line of Table X (compounds I.C-1.3b.X-1 to I.C-1.3b.X-384, I.C-2.3b.X-1 to I.C-2.3b.X-384, I.C-3.3b.X-1 to I.C-3.3b.X-384, I.C-4.3b.X-1 to I.C-5.3b.X-384, I.C-5.3b.X-1 to I.C-3.3b.X-384, I.C-6.3b.X-1 to I.C-6.3b.X-384; I.D-3b.X-1 to I.D-1.3b.X-384, I.D-2.3b.X-1 to I.D-2.3b.X-384, I.D-3.3b.X-1 to I.D-3.3b.X-384, I.D-4.3b.X-1 to I.D-4.3b.X-384, I.D-5.3b.X-1 to I.D-5.3b.X-384, I.D-6.3b.X-1 to I.D-6.3b.X-384).
Table 4b Compounds of the formula I.C-1, I.C-2, I.C-3, I.C-4, I.C-5, I.C-6; I.D-1, I.D-2, I.D-3, I.D-4, I.D-5, I.D-6 in which R⁸ is cyclo-C₆H₁₁ and the meaning for the combination of R⁶ and R⁷ for each individual compound corresponds in each case to one line of Table X (compounds I.C-1.4b.X-1 to I.C-1.4b.X-384, I.C-2.4b.X-1 to I.C-2.4b.X-384, I.C-3.4b.X-1 to I.C-3.4b.X-384, I.C-4.4b.X-1 to I.C-5.4b.X-384, I.C-5.4b.X-1 to I.C-3.4b.X-384, I.C-6.4b.X-1 to I.C-6.4b.X-384; I.D-4b.X-1 to I.D-1.4b.X-384, I.D-2.4b.X-1 to I.D-2.4b.X-384, I.D-3.4b.X-1 to I.D-3.4b.X-384, I.D-4.4b.X-1 to I.D-4.4b.X-384, I.D-5.4b.X-1 to I.D-5.4b.X-384, I.D-6.4b.X-1 to I.D-6.4b.X-384).
Table 5b Compounds of the formula I.C-1, I.C-2, I.C-3, I.C-4, I.C-5, I.C-6; I.D-1, I.D-2, I.D-3, I.D-4, I.D-5, I.D-6 in which R⁸ is

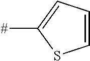

and the meaning for the combination of R⁶ and R⁷ for each individual compound corresponds in each case to one line of Table X (compounds I.C-1.5b.X-1 to I.C-1.5b.X-384, I.C-2.5b.X-1 to I.C-2.5b.X-384, I.C-3.5b.X-1 to I.C-3.5b.X-384, I.C-4.5b.X-1 to I.C-5.5b.X-384, I.C-5.5b.X-1 to I.C-3.5b.X-384, I.C-6.5b.X-1 to I.C-6.5b.X-384; I.D-5b.X-1 to I.D-1.5b.X-384, I.D-2.5b.X-1 to I.D-2.5b.X-384, I.D-3.5b.X-1 to I.D-3.5b.X-384, I.D-4.5b.X-1 to I.D-4.5b.X-384, I.D-5.5b.X-1 to I.D-5.5b.X-384, I.D-6.5b.X-1 to I.D-6.5b.X-384).
Table 6b Compounds of the formula I.C-1, I.C-2, I.C-3, I.C-4, I.C-5, I.C-6; I.D-1, I.D-2, I.D-3, I.D-4, I.D-5, I.D-6 in which R⁸ is C₆H₅ and the meaning for the combination of R⁶ and R⁷ for each individual compound corresponds in each case to one line of Table X (compounds I.C-1.6b.X-1 to I.C-1.6b.X-384, I.C-2.6b.X-1 to I.C-2.6b.X-384, I.C-3.6b.X-1 to I.C-3.6b.X-384, I.C-4.6b.X-1 to I.C-5.6b.X-384, I.C-5.6b.X-1 to I.C-3.6b.X-384, I.C-6.6b.X-1 to I.C-6.6b.X-384; I.D-6b.X-1 to I.D-1.6b.X-384, I.D-2.6b.X-1 to I.D-2.6b.X-384, I.D-3.6b.X-1 to I.D-3.6b.X-384, I.D-4.6b.X-1 to I.D-4.6b.X-384, I.D-5.6b.X-1 to I.D-5.6b.X-384, I.D-6.6b.X-1 to I.D-6.6b.X-384).
Table 7b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2-F—C₆H₄ and the meaning for the combination of R⁶ and R⁷ for each individual compound corresponds in each case to one line of Table X (compounds I.C-1.7b.X-1 to I.C-1.7b.X-384, I.C-2.7b.X-1 to I.C-2.7b.X-384, I.C-3.7b.X-1 to I.C-3.7b.X-384, I.C-4.7b.X-1 to I.C-5.7b.X-384, I.C-5.7b.X-1 to I.C-3.7b.X-384, I.C-6.7b.X-1 to I.C-6.7b.X-384; I.D-7b.X-1 to I.D-1.7b.X-384, I.D-2.7b.X-1 to I.D-2.7b.X-384, I.D-3.7b.X-1 to I.D-3.7b.X-384, I.D-4.7b.X-1 to I.D-4.7b.X-384, I.D-5.7b.X-1 to I.D-5.7b.X-384, I.D-6.7b.X-1 to I.D-6.7b.X-384).
Table 8b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 3-F—C₆H₄ and the meaning for the combination of R⁶ and R⁷ for each individual compound corresponds in each case to one line of Table X (compounds I.C-1.8b.X-1 to I.C-1.8b.X-384, I.C-2.8b.X-1 to I.C-2.8b.X-384, I.C-3.8b.X-1 to I.C-3.8b.X-384, I.C-4.8b.X-1 to I.C-5.8b.X-384, I.C-5.8b.X-1 to I.C-3.8b.X-384, I.C-6.8b.X-1 to I.C-6.8b.X-384; I.D-8b.X-1 to I.D-1.8b.X-384, I.D-2.8b.X-1 to I.D-2.8b.X-384, I.D-3.8b.X-1 to I.D-3.8b.X-384, I.D-4.8b.X-1 to I.D-4.8b.X-384, I.D-5.8b.X-1 to I.D-5.8b.X-384, I.D-6.8b.X-1 to I.D-6.8b.X-384).
Table 9b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 4-F—C₆H₄ and the meaning for the combination of R⁶ and R⁷ for each individual compound corresponds in each case to one line of Table X (compounds I.C-1.9b.X-1 to I.C-1.9b.X-384, I.C-2.9b.X-1 to I.C-2.9b.X-384, I.C-3.9b.X-1 to I.C-3.9b.X-384, I.C-4.9b.X-1 to I.C-5.9b.X-384, I.C-5.9b.X-1 to I.C-3.9b.X-384, I.C-6.9b.X-1 to I.C-6.9b.X-384; I.D-9b.X-1 to I.D-1.9b.X-384, I.D-2.9b.X-1 to I.D-2.9b.X-384, I.D-3.9b.X-1 to I.D-3.9b.X-384, I.D-4.9b.X-1 to I.D-4.9b.X-384, I.D-5.9b.X-1 to I.D-5.9b.X-384, I.D-6.9b.X-1 to I.D-6.9b.X-384).
Table 10b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2-Cl—C₆H₄ and the meaning for the combination of R⁶ and R⁷ for each individual compound corresponds in each case to one line of Table X (compounds I.C-1.10b.X-1 to I.C-1.10b.X-384, I.C-2.10b.X-1 to I.C-2.10b.X-384, I.C-3.10b.X-1 to I.C-3.10b.X-384, I.C-4.10b.X-1 to I.C-5.10b.X-384, I.C-5.10b.X-1 to I.C-3.10b.X-384, I.C-6.10b.X-1 to I.C-6.10b.X-384; I.D-10b.X-1 to I.D-1.10b.X-384, I.D-2.10b.X-1 to I.D-2.10b.X-384, I.D-3.10b.X-1 to I.D-3.10b.X-384, I.D-4.10b.X-1 to I.D-4.10b.X-384, I.D-5.10b.X-1 to I.D-5.10b.X-384, I.D-6.10b.X-1 to I.D-6.10b.X-384).
Table 11b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 3-Cl—C₆H₄ and the meaning for the combination of R⁶ and R⁷ for each individual compound corresponds in each case to one line of Table X (compounds I.C-1.11b.X-1 to I.C-1.11b.X-384, I.C-2.11b.X-1 to I.C-2.11b.X-384, I.C-3.11b.X-1 to I.C-3.11b.X-384, I.C-4.11b.X-1 to I.C-5.11b.X-384, I.C-5.11b.X-1 to I.C-3.11b.X-384, I.C-6.11b.X-1 to I.C-6.11b.X-384; I.D-11b.X-1 to I.D-1.11b.X-384, I.D-2.11b.X-1 to I.D-2.11b.X-384, I.D-3.11b.X-1 to I.D-3.11b.X-384, I.D-4.11b.X-1 to I.D-4.11b.X-384, I.D-5.11b.X-1 to I.D-5.11b.X-384, I.D-6.11b.X-1 to I.D-6.11b.X-384).
Table 12b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 4-Cl—C₆H₄ and the meaning for the combination of R⁶ and R⁷ for each individual compound corresponds in each case to one line of Table X (compounds I.C-1.12b.X-1 to I.C-1.12b.X-384, I.C-2.12b.X-1 to I.C-2.12b.X-384, I.C-3.12b.X-1 to I.C-3.12b.X-384, I.C-4.12b.X-1 to I.C-5.12b.X-384, I.C-5.12b.X-1 to I.C-3.12b.X-384, I.C-6.12b.X-1 to I.C-6.12b.X-384; I.D-12b.X-1 to I.D-1.12b.X-384, I.D-2.12b.X-1 to I.D-2.12b.X-384, I.D-3.12b.X-1 to I.D-3.12b.X-384, I.D-4.12b.X-1 to I.D-4.12b.X-384, I.D-5.12b.X-1 to I.D-5.12b.X-384, I.D-6.12b.X-1 to I.D-6.12b.X-384).
Table 13b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2-OCH₃—C₆H₄ and the meaning for the combination of R⁶ and R⁷ for each individual compound corresponds in each case to one line of Table X (compounds I.C-1.13b.X-1 to I.C-1.13b.X-384, I.C-2.13b.X-1 to I.C-2.13b.X-384, I.C-3.13b.X-1 to I.C-3.13b.X-384, I.C-4.13b.X-1 to I.C-5.13b.X-384, I.C-5.13b.X-1 to I.C-3.13b.X-384, I.C-6.13b.X-1 to I.C-6.13b.X-384; I.D-13b.X-1 to I.D-1.13b.X-384, I.D-2.13b.X-1 to I.D-2.13b.X-384, I.D-3.13b.X-1 to I.D-3.13b.X-384, I.D-4.13b.X-1 to I.D-4.13b.X-384, I.D-5.13b.X-1 to I.D-5.13b.X-384, I.D-6.13b.X-1 to I.D-6.13b.X-384).
Table 14b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 3-OCH₃—C₆H₄ and the meaning for the combination of R⁶ and R⁷ for each individual compound corresponds in each case to one line of Table X (compounds I.C-1.14b.X-1 to I.C-1.14b.X-384, I.C-2.14b.X-1 to I.C-2.14b.X-384, I.C-3.14b.X-1 to I.C-3.14b.X-384, I.C-4.14b.X-1 to I.C-5.14b.X-384, I.C-5.14b.X-1 to I.C-3.14b.X-384, I.C-6.14b.X-1 to I.C-6.14b.X-384; I.D-14b.X-1 to I.D-1.14b.X-384, I.D-2.14b.X-1 to I.D-2.14b.X-384, I.D-3.14b.X-1 to I.D-3.14b.X-384, I.D-4.14b.X-1 to I.D-4.14b.X-384, I.D-5.14b.X-1 to I.D-5.14b.X-384, I.D-6.14b.X-1 to I.D-6.14b.X-384).
Table 15b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 4-OCH₃—C₆H₄ and the meaning for the combination of R⁶ and R⁷ for each individual compound corresponds in each case to one line of Table X (compounds I.C-1.15b.X-1 to I.C-1.15b.X-384, I.C-2.15b.X-1 to I.C-2.15b.X-384, I.C-3.15b.X-1 to I.C-3.15b.X-384, I.C-4.15b.X-1 to I.C-5.15b.X-384, I.C-5.15b.X-1 to I.C-3.15b.X-384, I.C-6.15b.X-1 to I.C-6.15b.X-384; I.D-15b.X-1 to I.D-1.15b.X-384, I.D-2.15b.X-1 to I.D-2.15b.X-384, I.D-3.15b.X-1 to I.D-3.15b.X-384, I.D-4.15b.X-1 to I.D-4.15b.X-384, I.D-5.15b.X-1 to I.D-5.15b.X-384, I.D-6.15b.X-1 to I.D-6.15b.X-384).
Table 16b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2-OCHF₂—C₆H₄ and the meaning for the combination of R⁶ and R⁷ for each individual compound corresponds in each case to one line of Table X (compounds I.C-1.16b.X-1 to I.C-1.16b.X-384, I.C-2.16b.X-1 to I.C-2.16b.X-384, I.C-3.16b.X-1 to I.C-3.16b.X-384, I.C-4.16b.X-1 to I.C-5.16b.X-384, I.C-5.16b.X-1 to I.C-3.16b.X-384, I.C-6.16b.X-1 to I.C-6.16b.X-384; I.D-16b.X-1 to I.D-1.16b.X-384, I.D-2.16b.X-1 to I.D-2.16b.X-384, I.D-3.16b.X-1 to I.D-3.16b.X-384, I.D-4.16b.X-1 to I.D-4.16b.X-384, I.D-5.16b.X-1 to I.D-5.16b.X-384, I.D-6.16b.X-1 to I.D-6.16b.X-384).
Table 17b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 3-OCHF₂—C₆H₄ and the meaning for the combination of R⁶ and R⁷ for each individual compound corresponds in each case to one line of Table X (compounds I.C-1.17b.X-1 to I.C-1.17b.X-384, I.C-2.17b.X-1 to I.C-2.17b.X-384, I.C-3.17b.X-1 to I.C-3.17b.X-384, I.C-4.17b.X-1 to I.C-5.17b.X-384, I.C-5.17b.X-1 to I.C-3.17b.X-384, I.C-6.17b.X-1 to I.C-6.17b.X-384; I.D-17b.X-1 to I.D-1.17b.X-384, I.D-2.17b.X-1 to I.D-2.17b.X-384, I.D-3.17b.X-1 to I.D-3.17b.X-384, I.D-4.17b.X-1 to I.D-4.17b.X-384, I.D-5.17b.X-1 to I.D-5.17b.X-384, I.D-6.17b.X-1 to I.D-6.17b.X-384).
Table 18b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 4-OCHF₂—C₆H₄ and the meaning for the combination of R⁶ and R⁷ for each individual compound corresponds in each case to one line of Table X (compounds I.C-1.18b.X-1 to I.C-1.18b.X-384, I.C-2.18b.X-1 to I.C-2.18b.X-384, I.C-3.18b.X-1 to I.C-3.18b.X-384, I.C-4.18b.X-1 to I.C-5.18b.X-384, I.C-5.18b.X-1 to I.C-3.18b.X-384, I.C-6.18b.X-1 to I.C-6.18b.X-384; I.D-18b.X-1 to I.D-1.18b.X-384, I.D-2.18b.X-1 to I.D-2.18b.X-384, I.D-3.18b.X-1 to I.D-3.18b.X-384, I.D-4.18b.X-1 to I.D-4.18b.X-384, I.D-5.18b.X-1 to I.D-5.18b.X-384, I.D-6.18b.X-1 to I.D-6.18b.X-384).
Table 19b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2-CH₃—C₆H₄ and the meaning for the combination of R⁶ and R⁷ for each individual compound corresponds in each case to one line of Table X (compounds I.C-1.19b.X-1 to I.C-1.19b.X-384, I.C-2.19b.X-1 to I.C-2.19b.X-384, I.C-3.19b.X-1 to I.C-3.19b.X-384, I.C-4.19b.X-1 to I.C-5.19b.X-384, I.C-5.19b.X-1 to I.C-3.19b.X-384, I.C-6.19b.X-1 to I.C-6.19b.X-384; I.D-19b.X-1 to I.D-1.19b.X-384, I.D-2.19b.X-1 to I.D-2.19b.X-384, I.D-3.19b.X-1 to I.D-3.19b.X-384, I.D-4.19b.X-1 to I.D-4.19b.X-384, I.D-5.19b.X-1 to I.D-5.19b.X-384, I.D-6.19b.X-1 to I.D-6.19b.X-384).
Table 20b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 3-CH₃—C₆H₄ and the meaning for the combination of R⁶ and R⁷ for each individual compound corresponds in each case to one line of Table X (compounds I.C-1.20b.X-1 to I.C-1.20b.X-384, I.C-2.20b.X-1 to I.C-2.20b.X-384, I.C-3.20b.X-1 to I.C-3.20b.X-384, I.C-4.20b.X-1 to I.C-5.20b.X-384, I.C-5.20b.X-1 to I.C-3.20b.X-384, I.C-6.20b.X-1 to I.C-6.20b.X-384; I.D-20b.X-1 to I.D-1.20b.X-384, I.D-2.20b.X-1 to I.D-2.20b.X-384, I.D-3.20b.X-1 to I.D-3.20b.X-384, I.D-4.20b.X-1 to I.D-4.20b.X-384, I.D-5.20b.X-1 to I.D-5.20b.X-384, I.D-6.20b.X-1 to I.D-6.20b.X-384).
Table 21b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 4-CH₃—C₆H₄ and the meaning for the combination of R⁶ and R⁷ for each individual compound corresponds in each case to one line of Table X (compounds I.C-1.21b.X-1 to I.C-1.21b.X-384, I.C-2.21b.X-1 to I.C-2.21b.X-384, I.C-3.21b.X-1 to I.C-3.21b.X-384, I.C-4.21b.X-1 to I.C-5.21b.X-384, I.C-5.21b.X-1 to I.C-3.21b.X-384, I.C-6.21b.X-1 to I.C-6.21b.X-384; I.D-21b.X-1 to I.D-1.21b.X-384, I.D-2.21b.X-1 to I.D-2.21b.X-384, I.D-3.21b.X-1 to I.D-3.21b.X-384, I.D-4.21b.X-1 to I.D-4.21b.X-384, I.D-5.21b.X-1 to I.D-5.21b.X-384, I.D-6.21b.X-1 to I.D-6.21b.X-384).
Table 22b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2-CF₃—C₆H₄ and the meaning for the combination of R⁶ and R⁷ for each individual compound corresponds in each case to one line of Table X (compounds I.C-1.22b.X-1 to I.C-1.22b.X-384, I.C-2.22b.X-1 to I.C-2.22b.X-384, I.C-3.22b.X-1 to I.C-3.22b.X-384, I.C-4.22b.X-1 to I.C-5.22b.X-384, I.C-5.22b.X-1 to I.C-3.22b.X-384, I.C-6.22b.X-1 to I.C-6.22b.X-384; I.D-22b.X-1 to I.D-1.22b.X-384, I.D-2.22b.X-1 to I.D-2.22b.X-384, I.D-3.22b.X-1 to I.D-3.22b.X-384, I.D-4.22b.X-1 to I.D-4.22b.X-384, I.D-5.22b.X-1 to I.D-5.22b.X-384, I.D-6.22b.X-1 to I.D-6.22b.X-384).
Table 23b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 3-CF₃—C₆H₄ and the meaning for the combination of R⁶ and R⁷ for each individual compound corresponds in each case to one line of Table X (compounds I.C-1.23b.X-1 to I.C-1.23b.X-384, I.C-2.23b.X-1 to I.C-2.23b.X-384, I.C-3.23b.X-1 to I.C-3.23b.X-384, I.C-4.23b.X-1 to I.C-5.23b.X-384, I.C-5.23b.X-1 to I.C-3.23b.X-384, I.C-6.23b.X-1 to I.C-6.23b.X-384; I.D-23b.X-1 to I.D-1.23b.X-384, I.D-2.23b.X-1 to I.D-2.23b.X-384, I.D-3.23b.X-1 to I.D-3.23b.X-384, I.D-4.23b.X-1 to I.D-4.23b.X-384, I.D-5.23b.X-1 to I.D-5.23b.X-384, I.D-6.23b.X-1 to I.D-6.23b.X-384).
Table 24b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 4-CF₃—C₆H₄ and the meaning for the combination of R⁶ and R⁷ for each individual compound corresponds in each case to one line of Table X (compounds I.C-1.24b.X-1 to I.C-1.24b.X-384, I.C-2.24b.X-1 to I.C-2.24b.X-384, I.C-3.24b.X-1 to I.C-3.24b.X-384, I.C-4.24b.X-1 to I.C-5.24b.X-384, I.C-5.24b.X-1 to I.C-3.24b.X-384, I.C-6.24b.X-1 to I.C-6.24b.X-384; I.D-24b.X-1 to I.D-1.24b.X-384, I.D-2.24b.X-1 to I.D-2.24b.X-384, I.D-3.24b.X-1 to I.D-3.24b.X-384, I.D-4.24b.X-1 to I.D-4.24b.X-384, I.D-5.24b.X-1 to I.D-5.24b.X-384, I.D-6.24b.X-1 to I.D-6.24b.X-384).
Table 25b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2,3-F₂-C₆H₃ and the meaning for the combination of R⁶ and R⁷ for each individual compound corresponds in each case to one line of Table X (compounds I.C-1.25b.X-1 to I.C-1.25b.X-384, I.C-2.25b.X-1 to I.C-2.25b.X-384, I.C-3.25b.X-1 to I.C-3.25b.X-384, I.C-4.25b.X-1 to I.C-5.25b.X-384, I.C-5.25b.X-1 to I.C-3.25b.X-384, I.C-6.25b.X-1 to I.C-6.25b.X-384; I.D-25b.X-1 to I.D-1.25b.X-384, I.D-2.25b.X-1 to I.D-2.25b.X-384, I.D-3.25b.X-1 to I.D-3.25b.X-384, I.D-4.25b.X-1 to I.D-4.25b.X-384, I.D-5.25b.X-1 to I.D-5.25b.X-384, I.D-6.25b.X-1 to I.D-6.25b.X-384).
Table 26b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2,4-F₂-C₆H₃ and the meaning for the combination of R⁶ and R⁷ for each individual compound corresponds in each case to one line of Table X (compounds I.C-1.26b.X-1 to I.C-1.26b.X-384, I.C-2.26b.X-1 to I.C-2.26b.X-384, I.C-3.26b.X-1 to I.C-3.26b.X-384, I.C-4.26b.X-1 to I.C-5.26b.X-384, I.C-5.26b.X-1 to I.C-3.26b.X-384, I.C-6.26b.X-1 to I.C-6.26b.X-384; I.D-26b.X-1 to I.D-1.26b.X-384, I.D-2.26b.X-1 to I.D-2.26b.X-384, I.D-3.26b.X-1 to I.D-3.26b.X-384, I.D-4.26b.X-1 to I.D-4.26b.X-384, I.D-5.26b.X-1 to I.D-5.26b.X-384, I.D-6.26b.X-1 to I.D-6.26b.X-384).
Table 27b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2,5-F₂-C₆H₃ and the meaning for the combination of R⁶ and R⁷ for each individual compound corresponds in each case to one line of Table X (compounds I.C-1.27b.X-1 to I.C-1.27b.X-384, I.C-2.27b.X-1 to I.C-2.27b.X-384, I.C-3.27b.X-1 to I.C-3.27b.X-384, I.C-4.27b.X-1 to I.C-5.27b.X-384, I.C-5.27b.X-1 to I.C-3.27b.X-384, I.C-6.27b.X-1 to I.C-6.27b.X-384; I.D-27b.X-1 to I.D-1.27b.X-384, I.D-2.27b.X-1 to I.D-2.27b.X-384, I.D-3.27b.X-1 to I.D-3.27b.X-384, I.D-4.27b.X-1 to I.D-4.27b.X-384, I.D-5.27b.X-1 to I.D-5.27b.X-384, I.D-6.27b.X-1 to I.D-6.27b.X-384).
Table 28b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2,6-F₂-C₆H₃ and the meaning for the combination of R⁶ and R⁷ for each individual compound corresponds in each case to one line of Table X (compounds I.C-1.28b.X-1 to I.C-1.28b.X-384, I.C-2.28b.X-1 to I.C-2.28b.X-384, I.C-3.28b.X-1 to I.C-3.28b.X-384, I.C-4.28b.X-1 to I.C-5.28b.X-384, I.C-5.28b.X-1 to I.C-3.28b.X-384, I.C-6.28b.X-1 to I.C-6.28b.X-384; I.D-28b.X-1 to I.D-1.28b.X-384, I.D-2.28b.X-1 to I.D-2.28b.X-384, I.D-3.28b.X-1 to I.D-3.28b.X-384, I.D-4.28b.X-1 to I.D-4.28b.X-384, I.D-5.28b.X-1 to I.D-5.28b.X-384, I.D-6.28b.X-1 to I.D-6.28b.X-384).
Table 29b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 3,4-F₂-C₆H₃ and the meaning for the combination of R⁶ and R⁷ for each individual compound corresponds in each case to one line of Table X (compounds I.C-1.29b.X-1 to I.C-1.29b.X-384, I.C-2.29b.X-1 to I.C-2.29b.X-384, I.C-3.29b.X-1 to I.C-3.29b.X-384, I.C-4.29b.X-1 to I.C-5.29b.X-384, I.C-5.29b.X-1 to I.C-3.29b.X-384, I.C-6.29b.X-1 to I.C-6.29b.X-384; I.D-29b.X-1 to I.D-1.29b.X-384, I.D-2.29b.X-1 to I.D-2.29b.X-384, I.D-3.29b.X-1 to I.D-3.29b.X-384, I.D-4.29b.X-1 to I.D-4.29b.X-384, I.D-5.29b.X-1 to I.D-5.29b.X-384, I.D-6.29b.X-1 to I.D-6.29b.X-384).
Table 30b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 3,5-F₂-C₆H₃ and the meaning for the combination of R⁶ and R⁷ for each individual compound corresponds in each case to one line of Table X (compounds I.C-1.30b.X-1 to I.C-1.30b.X-384, I.C-2.30b.X-1 to I.C-2.30b.X-384, I.C-3.30b.X-1 to I.C-3.30b.X-384, I.C-4.30b.X-1 to I.C-5.30b.X-384, I.C-5.30b.X-1 to I.C-3.30b.X-384, I.C-6.30b.X-1 to I.C-6.30b.X-384; I.D-30b.X-1 to I.D-1.30b.X-384, I.D-2.30b.X-1 to I.D-2.30b.X-384, I.D-3.30b.X-1 to I.D-3.30b.X-384, I.D-4.30b.X-1 to I.D-4.30b.X-384, I.D-5.30b.X-1 to I.D-5.30b.X-384, I.D-6.30b.X-1 to I.D-6.30b.X-384).
Table 31b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2,3-Cl₂—C₆H₃ and the meaning for the combination of R⁶ and R⁷ for each individual compound corresponds in each case to one line of Table X (compounds I.C-1.31b.X-1 to I.C-1.31b.X-384, I.C-2.31b.X-1 to I.C-2.31b.X-384, I.C-3.31b.X-1 to I.C-3.31b.X-384, I.C-4.31b.X-1 to I.C-5.31b.X-384, I.C-5.31b.X-1 to I.C-3.31b.X-384, I.C-6.31b.X-1 to I.C-6.31b.X-384; I.D-31b.X-1 to I.D-1.31b.X-384, I.D-2.31b.X-1 to I.D-2.31b.X-384, I.D-3.31b.X-1 to I.D-3.31b.X-384, I.D-4.31b.X-1 to I.D-4.31b.X-384, I.D-5.31b.X-1 to I.D-5.31b.X-384, I.D-6.31b.X-1 to I.D-6.31b.X-384).
Table 32b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2,4-Cl₂—C₆H₃ and the meaning for the combination of R⁶ and R⁷ for each individual compound corresponds in each case to one line of Table X (compounds I.C-1.32b.X-1 to I.C-1.32b.X-384, I.C-2.32b.X-1 to I.C-2.32b.X-384, I.C-3.32b.X-1 to I.C-3.32b.X-384, I.C-4.32b.X-1 to I.C-5.32b.X-384, I.C-5.32b.X-1 to I.C-3.32b.X-384, I.C-6.32b.X-1 to I.C-6.32b.X-384; I.D-32b.X-1 to I.D-1.32b.X-384, I.D-2.32b.X-1 to I.D-2.32b.X-384, I.D-3.32b.X-1 to I.D-3.32b.X-384, I.D-4.32b.X-1 to I.D-4.32b.X-384, I.D-5.32b.X-1 to I.D-5.32b.X-384, I.D-6.32b.X-1 to I.D-6.32b.X-384).
Table 33b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2,5-Cl₂—C₆H₃ and the meaning for the combination of R⁶ and R⁷ for each individual compound corresponds in each case to one line of Table X (compounds I.C-1.33b.X-1 to I.C-1.33b.X-384, I.C-2.33b.X-1 to I.C-2.33b.X-384, I.C-3.33b.X-1 to I.C-3.33b.X-384, I.C-4.33b.X-1 to I.C-5.33b.X-384, I.C-5.33b.X-1 to I.C-3.33b.X-384, I.C-6.33b.X-1 to I.C-6.33b.X-384; I.D-33b.X-1 to I.D-1.33b.X-384, I.D-2.33b.X-1 to I.D-2.33b.X-384, I.D-3.33b.X-1 to I.D-3.33b.X-384, I.D-4.33b.X-1 to I.D-4.33b.X-384, I.D-5.33b.X-1 to I.D-5.33b.X-384, I.D-6.33b.X-1 to I.D-6.33b.X-384).
Table 34b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2,6-Cl₂—C₆H₃ and the meaning for the combination of R⁶ and R⁷ for each individual compound corresponds in each case to one line of Table X (compounds I.C-1.34b.X-1 to I.C-1.34b.X-384, I.C-2.34b.X-1 to I.C-2.34b.X-384, I.C-3.34b.X-1 to I.C-3.34b.X-384, I.C-4.34b.X-1 to I.C-5.34b.X-384, I.C-5.34b.X-1 to I.C-3.34b.X-384, I.C-6.34b.X-1 to I.C-6.34b.X-384; I.D-34b.X-1 to I.D-1.34b.X-384, I.D-2.34b.X-1 to I.D-2.34b.X-384, I.D-3.34b.X-1 to I.D-3.34b.X-384, I.D-4.34b.X-1 to I.D-4.34b.X-384, I.D-5.34b.X-1 to I.D-5.34b.X-384, I.D-6.34b.X-1 to I.D-6.34b.X-384).
Table 35b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 3,4-Cl₂—C₆H₃ and the meaning for the combination of R⁶ and R⁷ for each individual compound corresponds in each case to one line of Table X (compounds I.C-1.35b.X-1 to I.C-1.35b.X-384, I.C-2.35b.X-1 to I.C-2.35b.X-384, I.C-3.35b.X-1 to I.C-3.35b.X-384, I.C-4.35b.X-1 to I.C-5.35b.X-384, I.C-5.35b.X-1 to I.C-3.35b.X-384, I.C-6.35b.X-1 to I.C-6.35b.X-384; I.D-35b.X-1 to I.D-1.35b.X-384, I.D-2.35b.X-1 to I.D-2.35b.X-384, I.D-3.35b.X-1 to I.D-3.35b.X-384, I.D-4.35b.X-1 to I.D-4.35b.X-384, I.D-5.35b.X-1 to I.D-5.35b.X-384, I.D-6.35b.X-1 to I.D-6.35b.X-384).
Table 36b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 3,5-Cl₂—C₆H₃ and the meaning for the combination of R⁶ and R⁷ for each individual compound corresponds in each case to one line of Table X (compounds I.C-1.36b.X-1 to I.C-1.36b.X-384, I.C-2.36b.X-1 to I.C-2.36b.X-384, I.C-3.36b.X-1 to I.C-3.36b.X-384, I.C-4.36b.X-1 to I.C-5.36b.X-384, I.C-5.36b.X-1 to I.C-3.36b.X-384, I.C-6.36b.X-1 to I.C-6.36b.X-384; I.D-36b.X-1 to I.D-1.36b.X-384, I.D-2.36b.X-1 to I.D-2.36b.X-384, I.D-3.36b.X-1 to I.D-3.36b.X-384, I.D-4.36b.X-1 to I.D-4.36b.X-384, I.D-5.36b.X-1 to I.D-5.36b.X-384, I.D-6.36b.X-1 to I.D-6.36b.X-384).
Table 37b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2,3-(CH₃)₂-C₆H₃ and the meaning for the combination of R⁶ and R⁷ for each individual compound corresponds in each case to one line of Table X (compounds I.C-1.37b.X-1 to I.C-1.37b.X-384, I.C-2.37b.X-1 to I.C-2.37b.X-384, I.C-3.37b.X-1 to I.C-3.37b.X-384, I.C-4.37b.X-1 to I.C-5.37b.X-384, I.C-5.37b.X-1 to I.C-3.37b.X-384, I.C-6.37b.X-1 to I.C-6.37b.X-384; I.D-37b.X-1 to I.D-1.37b.X-384, I.D-2.37b.X-1 to I.D-2.37b.X-384, I.D-3.37b.X-1 to I.D-3.37b.X-384, I.D-4.37b.X-1 to I.D-4.37b.X-384, I.D-5.37b.X-1 to I.D-5.37b.X-384, I.D-6.37b.X-1 to I.D-6.37b.X-384).
Table 38b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2,4-(CH₃)₂-C₆H₃ and the meaning for the combination of R⁶ and R⁷ for each individual compound corresponds in each case to one line of Table X (compounds I.C-1.38b.X-1 to I.C-1.38b.X-384, I.C-2.38b.X-1 to I.C-2.38b.X-384, I.C-3.38b.X-1 to I.C-3.38b.X-384, I.C-4.38b.X-1 to I.C-5.38b.X-384, I.C-5.38b.X-1 to I.C-3.38b.X-384, I.C-6.38b.X-1 to I.C-6.38b.X-384; I.D-38b.X-1 to I.D-1.38b.X-384, I.D-2.38b.X-1 to I.D-2.38b.X-384, I.D-3.38b.X-1 to I.D-3.38b.X-384, I.D-4.38b.X-1 to I.D-4.38b.X-384, I.D-5.38b.X-1 to I.D-5.38b.X-384, I.D-6.38b.X-1 to I.D-6.38b.X-384).
Table 39b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2,5-(CH₃)₂-C₆H₃ and the meaning for the combination of R⁶ and R⁷ for each individual compound corresponds in each case to one line of Table X (compounds I.C-1.39b.X-1 to I.C-1.39b.X-384, I.C-2.39b.X-1 to I.C-2.39b.X-384, I.C-3.39b.X-1 to I.C-3.39b.X-384, I.C-4.39b.X-1 to I.C-5.39b.X-384, I.C-5.39b.X-1 to I.C-3.39b.X-384, I.C-6.39b.X-1 to I.C-6.39b.X-384; I.D-39b.X-1 to I.D-1.39b.X-384, I.D-2.39b.X-1 to I.D-2.39b.X-384, I.D-3.39b.X-1 to I.D-3.39b.X-384, I.D-4.39b.X-1 to I.D-4.39b.X-384, I.D-5.39b.X-1 to I.D-5.39b.X-384, I.D-6.39b.X-1 to I.D-6.39b.X-384).
Table 40b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 2,6-(CH₃)₂-C₆H₃ and the meaning for the combination of R⁶ and R⁷ for each individual compound corresponds in each case to one line of Table X (compounds I.C-1.40b.X-1 to I.C-1.40b.X-384, I.C-2.40b.X-1 to I.C-2.40b.X-384, I.C-3.40b.X-1 to I.C-3.40b.X-384, I.C-4.40b.X-1 to I.C-5.40b.X-384, I.C-5.40b.X-1 to I.C-3.40b.X-384, I.C-6.40b.X-1 to I.C-6.40b.X-384; I.D-40b.X-1 to I.D-1.40b.X-384, I.D-2.40b.X-1 to I.D-2.40b.X-384, I.D-3.40b.X-1 to I.D-3.40b.X-384, I.D-4.40b.X-1 to I.D-4.40b.X-384, I.D-5.40b.X-1 to I.D-5.40b.X-384, I.D-6.40b.X-1 to I.D-6.40b.X-384).
Table 41b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 3,4-(CH₃)₂-C₆H₃ and the meaning for the combination of R⁶ and R⁷ for each individual compound corresponds in each case to one line of Table X (compounds I.C-1.41b.X-1 to I.C-1.41b.X-384, I.C-2.41b.X-1 to I.C-2.41b.X-384, I.C-3.41b.X-1 to I.C-3.41b.X-384, I.C-4.41b.X-1 to I.C-5.41b.X-384, I.C-5.41b.X-1 to I.C-3.41b.X-384, I.C-6.41b.X-1 to I.C-6.41b.X-384; I.D-41b.X-1 to I.D-1.41b.X-384, I.D-2.41b.X-1 to I.D-2.41b.X-384, I.D-3.41b.X-1 to I.D-3.41b.X-384, I.D-4.41b.X-1 to I.D-4.41b.X-384, I.D-5.41b.X-1 to I.D-5.41b.X-384, I.D-6.41b.X-1 to I.D-6.41b.X-384).
Table 42b Compounds of the formula I.C-1, I.C-2, I.C-3, I.C-4, I.C-5, I.C-6; I.D-1, I.D-2, I.D-3, I.D-4, I.D-5, I.D-6 in which R⁸ is 3,5-(CH₃)₂-C₆H₃ and the meaning for the combination of R⁶ and R⁷ for each individual compound corresponds in each case to one line of Table X (compounds I.C-1.42b.X-1 to I.C-1.42b.X-384, I.C-2.42b.X-1 to I.C-2.42b.X-384, I.C-3.42b.X-1 to I.C-3.42b.X-384, I.C-4.42b.X-1 to I.C-5.42b.X-384, I.C-5.42b.X-1 to I.C-3.42b.X-384, I.C-6.42b.X-1 to I.C-6.42b.X-384; I.D-42b.X-1 to I.D-1.42b.X-384, I.D-2.42b.X-1 to I.D-2.42b.X-384, I.D-3.42b.X-1 to I.D-3.42b.X-384, I.D-4.42b.X-1 to I.D-4.42b.X-384, I.D-5.42b.X-1 to I.D-5.42b.X-384, I.D-6.42b.X-1 to I.D-6.42b.X-384).
Table 43b Compounds of the formula I.C-1, I.C-2, I.C-3, I.C-4, I.C-5, I.C-6; I.D-1, I.D-2, I.D-3, I.D-4, I.D-5, I.D-6 in which R⁸ is

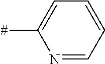

and the meaning for the combination of R⁶ and R⁷ for each individual compound corresponds in each case to one line of Table X (compounds I.C-1.43b.X-1 to I.C-1.43b.X-384, I.C-2.43b.X-1 to I.C-2.43b.X-384, I.C-3.43b.X-1 to I.C-3.43b.X-384, I.C-4.43b.X-1 to I.C-5.43b.X-384, I.C-5.43b.X-1 to I.C-3.43b.X-384, I.C-6.43b.X-1 to I.C-6.43b.X-384; I.D-43b.X-1 to I.D-1.43b.X-384, I.D-2.43b.X-1 to I.D-2.43b.X-384, I.D-3.43b.X-1 to I.D-3.43b.X-384, I. D-4.43b.X-1 to I. D-4.43b.X-384, I. D-5.43b.X-1 to I. D-5.43b.X-384, I.D-6.43b.X-1 to I.D-6.43b.X-384).

TABLE X

| No. | R⁶ | R⁷ |
|---|---|---|
| X-1 | CN | H |
| X-2 | CH₃ | H |
| X-3 | C₂H₅ | H |
| X-4 | n-C₃H₇ | H |
| X-5 | CH(CH₃)₂ | H |
| X-6 | n-C₄H₉ | H |
| X-7 | CH(CH₃)—CH₂—CH₃ | H |
| X-8 | CH₂—CH(CH₃)₂ | H |
| X-9 | n-C₅H₁₁ | H |
| X-10 | CH(CH₃)—CH₂—CH₂—CH₃ | H |
| X-11 | CH₂—CH(CH₃)—CH₂—CH₃ | H |
| X-12 | CH₂—CH₂—CH(CH₃)₂ | H |
| X-13 | CHF₂ | H |
| X-14 | CF₃ | H |
| X-15 | CH₂—CHF₂ | H |
| X-16 | CH₂—CF₃ | H |
| X-17 | CH₂—CH═CH₂ | H |
| X-18 | CH₂—CH═CHCl | H |
| X-19 | CH₂—C≡CH | H |
| X-20 | CH₂-cyclo-C₃H₅ | H |
| X-21 | CH₂-cyclo-C₄H₇ | H |
| X-22 | CH₂-cyclo-C₅H₉ | H |
| X-23 | CH₂-cyclo-C₆H₁₁ | H |
| X-24 | cyclo-C₃H₅ | H |
| X-25 | cyclo-C₄H₇ | H |
| X-26 | cyclo-C₅H₉ | H |
| X-27 | cyclo-C₆H₁₁ | H |
| X-28 | C(CH₃)═NOCH₃ | H |
| X-29 | C(C₂H₅)═NOCH₃ | H |
| X-30 | C(n-C₂H₇)═NOCH₃ | H |
| X-31 | C(CH(CH₃)₂)═NOCH₃ | H |
| X-32 | C(C₆H₅)═NOCH₃ | H |
| X-33 | C(CH₃)═NO—CH₂—C₆H₅ | H |
| X-34 | C₆H₅ | H |
| X-35 | 2-F—C₆H₄ | H |
| X-36 | 3-F—C₆H₄ | H |
| X-37 | 4-F—C₆H₄ | H |
| X-38 | 2-Cl—C₆H₄ | H |
| X-39 | 3-Cl—C₆H₄ | H |
| X-40 | 4-Cl—C₆H₄ | H |
| X-41 | 2-OCH₃—C₆H₄ | H |
| X-42 | 3-OCH₃—C₆H₄ | H |
| X-43 | 4-OCH₄—C₆H₄ | H |
| X-44 | 2-OCHF₂—C₆H₄ | H |
| X-45 | 3-OCHF₂—C₆H₄ | H |
| X-46 | 4-OCHF₂—C₆H₄ | H |
| X-47 | 2-CH₃—C₆H₄ | H |
| X-48 | 3-CH₃—C₆H₄ | H |
| X-49 | 4-CH₃—C₆H₄ | H |
| X-50 | 2-CF₃—C₆H₄ | H |
| X-51 | 3-CF₃—C₆H₄ | H |
| X-52 | 4-CF₃—C₆H₄ | H |
| X-53 | 2,3-F₂—C₆H₃ | H |
| X-54 | 2,4-F₂—C₆H₃ | H |
| X-55 | 2,5-F₂—C₆H₃ | H |
| X-56 | 2,6-F₂—C₆H₃ | H |
| X-57 | 3,4-F₂—C₆H₃ | H |
| X-58 | 3,5-F₂—C₆H₃ | H |
| X-59 | 2,3-Cl₂—C₆H₃ | H |
| X-60 | 2,4-Cl₂—C₆H₃ | H |
| X-61 | 2,5-Cl₂—C₆H₃ | H |
| X-62 | 2,6-Cl₂—C₆H₃ | H |
| X-63 | 3,4-Cl₂—C₆H₃ | H |
| X-64 | 3,5-Cl₂—C₆H₃ | H |
| X-65 | 2,3-(CH₃)₂—C₆H₃ | H |
| X-66 | 2,4-(CH₃)₂—C₆H₃ | H |
| X-67 | 2,5-(CH₃)₂—C₆H₃ | H |
| X-68 | 2,6-(CH₃)₂—C₆H₃ | H |
| X-69 | 3,4-(CH₃)₂—C₆H₃ | H |
| X-70 | 3,5-(CH₃)₂—C₆H₃ | H |
| X-71 | C₆H₅—CH₂ | H |
| X-72 | 2-F—C₆H₄—CH₂ | H |
| X-73 | 3-F—C₆H₄—CH₂ | H |
| X-74 | 4-F—C₆H₄—CH₂ | H |
| X-75 | 2-Cl—C₆H₄—CH₂ | H |
| X-76 | 3-Cl—C₆H₄—CH₂ | H |
| X-77 | 4-Cl—C₆H₄—CH₂ | H |
| X-78 | 2-CH₃—C₆H₄—CH₂ | H |
| X-79 | 3-CH₃—C₆H₄—CH₂ | H |
| X-80 | 4-CH₃—C₆H₄—CH₂ | H |
| X-81 | 2-CF₃—C₆H₄—CH₂ | H |
| X-82 | 3-CF₃—C₆H₄—CH₂ | H |
| X-83 | 4-CF₃—C₆H₄—CH₂ | H |
| X-84 | 2-OCH₃—C₆H₄—CH₂ | H |
| X-85 | 3-OCH₃—C₆H₄—CH₂ | H |
| X-86 | 4-OCH₃—C₆H₄—CH₂ | H |
| X-87 | 2-OCHF₂—C₆H₄—CH₂ | H |
| X-88 | 3-OCHF₂—C₆H₄—CH₂ | H |
| X-89 | 4-OCHF₂—C₆H₄—CH₂ | H |
| X-90 | 2,3-F₂—C₆H₃—CH₂ | H |
| X-91 | 2,4-F₂—C₆H₃—CH₂ | H |
| X-92 | 2,5-F₂—C₆H₃—CH₂ | H |
| X-93 | 2,6-F₂—C₆H₃—CH₂ | H |
| X-94 | 3,4-F₂—C₆H₃—CH₂ | H |
| X-95 | 3,5-F₂—C₆H₃—CH₂ | H |
| X-96 | 2,3-Cl₂—C₆H₃—CH₂ | H |
| X-97 | 2,4-Cl₂—C₆H₃—CH₂ | H |
| X-98 | 2,5-Cl₂—C₆H₃—CH₂ | H |
| X-99 | 2,6-Cl₂—C₆H₃—CH₂ | H |
| X-100 | 3,4-Cl₂—C₆H₃—CH₂ | H |
| X-101 | 3,5-Cl₂—C₆H₃—CH₂ | H |
| X-102 | 2,3-(CH₃)₂—C₆H₃—CH₂ | H |
| X-103 | 2,4-(CH₃)₂—C₆H₃—CH₂ | H |
| X-104 | 2,5-(CH₃)₂—C₆H₃—CH₂ | H |
| X-105 | 2,6-(CH₃)₂—C₆H₃—CH₂ | H |
| X-106 | 3,4-(CH₃)₂—C₆H₃—CH₂ | H |
| X-107 | 3,5-(CH₃)₂—C₆H₃—CH₂ | H |
| X-108 | 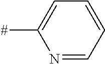 | H |
| X-109 | 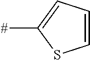 | H |
| X-110 | CN | OH |
| X-111 | CH₃ | OH |
| X-112 | C₂H₅ | OH |
| X-113 | n-C₃H₇ | OH |
| X-114 | CH(CH₃)₂ | OH |
| X-115 | n-C₄H₉ | OH |
| X-116 | CH(CH₃)—CH₂—CH₃ | OH |
| X-117 | CH₂—CH(CH₃)₂ | OH |
| X-118 | n-C₅H₁₁ | OH |
| X-119 | CH(CH₃)—CH₂—CH₂—CH₃ | OH |
| X-120 | CH₂—CH(CH₃)—CH₂—CH₃ | OH |
| X-121 | CH₂—CH₂—CH(CH₃)₂ | OH |
| X-122 | CHF₂ | OH |
| X-123 | CF₃ | OH |
| X-124 | CH₂—CHF₂ | OH |
| X-125 | CH₂—CF₃ | OH |
| X-126 | CH₂—CH═CH₂ | OH |
| X-127 | CH₂—CH═CHCl | OH |
| X-128 | CH₂—C≡CH | OH |
| X-129 | CH₂-cyclo-C₃H₅ | OH |
| X-130 | CH₂-cyclo-C₄H₇ | OH |
| X-131 | CH₂-cyclo-C₅H₉ | OH |
| X-132 | CH₂-cyclo-C₆H₁₁ | OH |
| X-133 | cyclo-C₃H₅ | OH |
| X-134 | cyclo-C₄H₇ | OH |
| X-135 | cyclo-C₅H₉ | OH |
| X-136 | cyclo-C₆H₁₁ | OH |
| X-137 | C(CH₃)═NOCH₃ | OH |
| X-138 | C(C₂H₅)═NOCH₃ | OH |
| X-139 | C(n-C₂H₇)═NOCH₃ | OH |
| X-140 | C(CH(CH₃)₂)═NOCH₃ | OH |
| X-141 | C(C₆H₅)═NOCH₃ | OH |
| X-142 | C(CH₃)═NO—CH₂—C₆H₅ | OH |
| X-143 | C₆H₅ | OH |
| X-144 | 2-F—C₆H₄ | OH |
| X-145 | 3-F—C₆H₄ | OH |
| X-146 | 4-F—C₆H₄ | OH |
| X-147 | 2-Cl—C₆H₄ | OH |
| X-148 | 3-Cl—C₆H₄ | OH |
| X-149 | 4-Cl—C₆H₄ | OH |
| X-150 | 2-OCH₃—C₆H₄ | OH |
| X-151 | 3-OCH₃—C₆H₄ | OH |
| X-152 | 4-OCH₄—C₆H₄ | OH |
| X-153 | 2-OCHF₂—C₆H₄ | OH |
| X-154 | 3-OCHF₂—C₆H₄ | OH |
| X-155 | 4-OCHF₂—C₆H₄ | OH |
| X-156 | 2-CH₃—C₆H₄ | OH |
| X-157 | 3-CH₃—C₆H₄ | OH |
| X-158 | 4-CH₃—C₆H₄ | OH |
| X-159 | 2-CF₃—C₆H₄ | OH |
| X-160 | 3-CF₃—C₆H₄ | OH |
| X-161 | 4-CF₃—C₆H₄ | OH |
| X-162 | 2,3-F₂—C₆H₃ | OH |
| X-163 | 2,4-F₂—C₆H₃ | OH |
| X-164 | 2,5-F₂—C₆H₃ | OH |
| X-165 | 2,6-F₂—C₆H₃ | OH |
| X-166 | 3,4-F₂—C₆H₃ | OH |
| X-167 | 3,5-F₂—C₆H₃ | OH |
| X-168 | 2,3-Cl₂—C₆H₃ | OH |
| X-169 | 2,4-Cl₂—C₆H₃ | OH |
| X-170 | 2,5-Cl₂—C₆H₃ | OH |
| X-171 | 2,6-Cl₂—C₆H₃ | OH |
| X-172 | 3,4-Cl₂—C₆H₃ | OH |
| X-173 | 3,5-Cl₂—C₆H₃ | OH |
| X-174 | 2,3-(CH₃)₂—C₆H₃ | OH |
| X-175 | 2,4-(CH₃)₂—C₆H₃ | OH |
| X-176 | 2,5-(CH₃)₂—C₆H₃ | OH |
| X-177 | 2,6-(CH₃)₂—C₆H₃ | OH |
| X-178 | 3,4-(CH₃)₂—C₆H₃ | OH |
| X-179 | 3,5-(CH₃)₂—C₆H₃ | OH |
| X-180 | C₆H₅—CH₂ | OH |
| X-181 | 2-F—C₆H₄—CH₂ | OH |
| X-182 | 3-F—C₆H₄—CH₂ | OH |
| X-183 | 4-F—C₆H₄—CH₂ | OH |
| X-184 | 2-Cl—C₆H₄—CH₂ | OH |
| X-185 | 3-Cl—C₆H₄—CH₂ | OH |
| X-186 | 4-Cl—C₆H₄—CH₂ | OH |
| X-187 | 2-CH₃—C₆H₄—CH₂ | OH |
| X-188 | 3-CH₃—C₆H₄—CH₂ | OH |
| X-189 | 4-CH₃—C₆H₄—CH₂ | OH |
| X-190 | 2-CF₃—C₆H₄—CH₂ | OH |
| X-191 | 3-CF₃—C₆H₄—CH₂ | OH |
| X-192 | 4-CF₃—C₆H₄—CH₂ | OH |
| X-193 | 2-OCH₃—C₆H₄—CH₂ | OH |
| X-194 | 3-OCH₃—C₆H₄—CH₂ | OH |
| X-195 | 4-OCH₃—C₆H₄—CH₂ | OH |
| X-196 | 2-OCHF₂—C₆H₄—CH₂ | OH |
| X-197 | 3-OCHF₂—C₆H₄—CH₂ | OH |
| X-198 | 4-OCHF₂—C₆H₄—CH₂ | OH |
| X-199 | 2,3-F₂—C₆H₃—CH₂ | OH |
| X-200 | 2,4-F₂—C₆H₃—CH₂ | OH |
| X-201 | 2,5-F₂—C₆H₃—CH₂ | OH |
| X-202 | 2,6-F₂—C₆H₃—CH₂ | OH |
| X-203 | 3,4-F₂—C₆H₃—CH₂ | OH |
| X-204 | 3,5-F₂—C₆H₃—CH₂ | OH |
| X-205 | 2,3-Cl₂—C₆H₃—CH₂ | OH |
| X-206 | 2,4-Cl₂—C₆H₃—CH₂ | OH |
| X-207 | 2,5-Cl₂—C₆H₃—CH₂ | OH |
| X-208 | 2,6-Cl₂—C₆H₃—CH₂ | OH |
| X-209 | 3,4-Cl₂—C₆H₃—CH₂ | OH |
| X-210 | 3,5-Cl₂—C₆H₃—CH₂ | OH |
| X-211 | 2,3-(CH₃)₂—C₆H₃—CH₂ | OH |
| X-212 | 2,4-(CH₃)₂—C₆H₃—CH₂ | OH |
| X-213 | 2,5-(CH₃)₂—C₆H₃—CH₂ | OH |
| X-214 | 2,6-(CH₃)₂—C₆H₃—CH₂ | OH |
| X-215 | 3,4-(CH₃)₂—C₆H₃—CH₂ | OH |
| X-216 | 3,5-(CH₃)₂—C₆H₃—CH₂ | OH |
| X-217 | 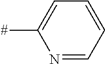 | OH |
| X-218 | 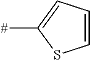 | OH |
| X-219 | CN | CH₃ |
| X-220 | CH₃ | CH₃ |
| X-221 | C₂H₅ | CH₃ |
| X-222 | n-C₃H₇ | CH₃ |
| X-223 | CH(CH₃)₂ | CH₃ |
| X-224 | n-C₄H₉ | CH₃ |
| X-225 | CH(CH₃)—CH₂—CH₃ | CH₃ |
| X-226 | CH₂—CH(CH₃)₂ | CH₃ |
| X-227 | n-C₅H₁₁ | CH₃ |
| X-228 | CH(CH₃)—CH₂—CH₂—CH₃ | CH₃ |
| X-229 | CH₂—CH(CH₃)—CH₂—CH₃ | CH₃ |
| X-230 | CH₂—CH₂—CH(CH₃)₂ | CH₃ |
| X-231 | CHF₂ | CH₃ |
| X-232 | CF₃ | CH₃ |
| X-233 | CH₂—CHF₂ | CH₃ |
| X-234 | CH₂—CF₃ | CH₃ |
| X-235 | CH₂—CH═CH₂ | CH₃ |
| X-236 | CH₂—CH═CHCl | CH₃ |
| X-237 | CH₂—C≡CH | CH₃ |
| X-238 | CH₂-cyclo-C₃H₅ | CH₃ |
| X-239 | CH₂-cyclo-C₄H₇ | CH₃ |
| X-240 | CH₂-cyclo-C₅H₉ | CH₃ |
| X-241 | CH₂-cyclo-C₆H₁₁ | CH₃ |
| X-242 | cyclo-C₃H₅ | CH₃ |
| X-243 | cyclo-C₄H₇ | CH₃ |
| X-244 | cyclo-C₅H₉ | CH₃ |
| X-245 | cyclo-C₆H₁₁ | CH₃ |
| X-246 | C(CH₃)═NOCH₃ | CH₃ |
| X-247 | C(C₂H₅)═NOCH₃ | CH₃ |
| X-248 | C(n-C₂H₇)═NOCH₃ | CH₃ |
| X-249 | C(CH(CH₃)₂)═NOCH₃ | CH₃ |
| X-250 | C(C₆H₅)═NOCH₃ | CH₃ |
| X-251 | C(CH₃)═NO—CH₂—C₆H₅ | CH₃ |
| X-252 | C₆H₅ | CH₃ |
| X-253 | 2-F—C₆H₄ | CH₃ |
| X-254 | 3-F—C₆H₄ | CH₃ |
| X-255 | 4-F—C₆H₄ | CH₃ |
| X-256 | 2-Cl—C₆H₄ | CH₃ |
| X-257 | 3-Cl—C₆H₄ | CH₃ |
| X-258 | 4-Cl—C₆H₄ | CH₃ |
| X-259 | 2-OCH₃—C₆H₄ | CH₃ |
| X-260 | 3-OCH₃—C₆H₄ | CH₃ |
| X-261 | 4-OCH₄—C₆H₄ | CH₃ |
| X-262 | 2-OCHF₂—C₆H₄ | CH₃ |
| X-263 | 3-OCHF₂—C₆H₄ | CH₃ |
| X-264 | 4-OCHF₂—C₆H₄ | CH₃ |
| X-265 | 2-CH₃—C₆H₄ | CH₃ |
| X-266 | 3-CH₃—C₆H₄ | CH₃ |
| X-267 | 4-CH₃—C₆H₄ | CH₃ |
| X-268 | 2-CF₃—C₆H₄ | CH₃ |
| X-269 | 3-CF₃—C₆H₄ | CH₃ |
| X-270 | 4-CF₃—C₆H₄ | CH₃ |
| X-271 | 2,3-F₂—C₆H₃ | CH₃ |
| X-272 | 2,4-F₂—C₆H₃ | CH₃ |
| X-273 | 2,5-F₂—C₆H₃ | CH₃ |
| X-274 | 2,6-F₂—C₆H₃ | CH₃ |
| X-275 | 3,4-F₂—C₆H₃ | CH₃ |
| X-276 | 3,5-F₂—C₆H₃ | CH₃ |
| X-277 | 2,3-Cl₂—C₆H₃ | CH₃ |
| X-278 | 2,4-Cl₂—C₆H₃ | CH₃ |
| X-279 | 2,5-Cl₂—C₆H₃ | CH₃ |
| X-280 | 2,6-Cl₂—C₆H₃ | CH₃ |
| X-281 | 3,4-Cl₂—C₆H₃ | CH₃ |
| X-282 | 3,5-Cl₂—C₆H₃ | CH₃ |
| X-283 | 2,3-(CH₃)₂—C₆H₃ | CH₃ |
| X-284 | 2,4-(CH₃)₂—C₆H₃ | CH₃ |
| X-285 | 2,5-(CH₃)₂—C₆H₃ | CH₃ |
| X-286 | 2,6-(CH₃)₂—C₆H₃ | CH₃ |
| X-287 | 3,4-(CH₃)₂—C₆H₃ | CH₃ |
| X-288 | 3,5-(CH₃)₂—C₆H₃ | CH₃ |
| X-289 | C₆H₅—CH₂ | CH₃ |
| X-290 | 2-F—C₆H₄—CH₂ | CH₃ |
| X-291 | 3-F—C₆H₄—CH₂ | CH₃ |
| X-292 | 4-F—C₆H₄—CH₂ | CH₃ |
| X-293 | 2-Cl—C₆H₄—CH₂ | CH₃ |
| X-294 | 3-Cl—C₆H₄—CH₂ | CH₃ |
| X-295 | 4-Cl—C₆H₄—CH₂ | CH₃ |
| X-296 | 2-CH₃—C₆H₄—CH₂ | CH₃ |
| X-297 | 3-CH₃—C₆H₄—CH₂ | CH₃ |
| X-298 | 4-CH₃—C₆H₄—CH₂ | CH₃ |
| X-299 | 2-CF₃—C₆H₄—CH₂ | CH₃ |
| X-300 | 3-CF₃—C₆H₄—CH₂ | CH₃ |
| X-301 | 4-CF₃—C₆H₄—CH₂ | CH₃ |
| X-302 | 2-OCH₃—C₆H₄—CH₂ | CH₃ |
| X-303 | 3-OCH₃—C₆H₄—CH₂ | CH₃ |
| X-304 | 4-OCH₃—C₆H₄—CH₂ | CH₃ |
| X-305 | 2-OCHF₂—C₆H₄—CH₂ | CH₃ |
| X-306 | 3-OCHF₂—C₆H₄—CH₂ | CH₃ |
| X-307 | 4-OCHF₂—C₆H₄—CH₂ | CH₃ |
| X-308 | 2,3-F₂—C₆H₃—CH₂ | CH₃ |
| X-309 | 2,4-F₂—C₆H₃—CH₂ | CH₃ |
| X-310 | 2,5-F₂—C₆H₃—CH₂ | CH₃ |
| X-311 | 2,6-F₂—C₆H₃—CH₂ | CH₃ |
| X-312 | 3,4-F₂—C₆H₃—CH₂ | CH₃ |
| X-313 | 3,5-F₂—C₆H₃—CH₂ | CH₃ |
| X-314 | 2,3-Cl₂—C₆H₃—CH₂ | CH₃ |
| X-315 | 2,4-Cl₂—C₆H₃—CH₂ | CH₃ |
| X-316 | 2,5-Cl₂—C₆H₃—CH₂ | CH₃ |
| X-317 | 2,6-Cl₂—C₆H₃—CH₂ | CH₃ |
| X-318 | 3,4-Cl₂—C₆H₃—CH₂ | CH₃ |
| X-319 | 3,5-Cl₂—C₆H₃—CH₂ | CH₃ |
| X-320 | 2,3-(CH₃)₂—C₆H₃—CH₂ | CH₃ |
| X-321 | 2,4-(CH₃)₂—C₆H₃—CH₂ | CH₃ |
| X-322 | 2,5-(CH₃)₂—C₆H₃—CH₂ | CH₃ |
| X-323 | 2,6-(CH₃)₂—C₆H₃—CH₂ | CH₃ |
| X-324 | 3,4-(CH₃)₂—C₆H₃—CH₂ | CH₃ |
| X-325 | 3,5-(CH₃)₂—C₆H₃—CH₂ | CH₃ |
| X-326 | 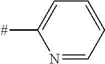 | CH₃ |
| X-327 | 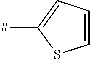 | CH₃ |
| X-328 | ═NO—CH₃ | |
| X-329 | ═NO—C₂H₅ | |
| X-330 | ═NO-n-C₃H₇ | |
| X-331 | ═NO—CH(CH₃)₂ | |
| X-332 | ═NO-n-C₄H₉ | |
| X-333 | ═NO—CH(CH₃)—CH₂—CH₃ | |
| X-334 | ═NO—CH₂—CH(CH₃)₂ | |
| X-335 | ═NO-n-C₅H₁₁ | |
| X-336 | ═NO—CH(CH₃)—CH₂—CH₂—CH₃ | |
| X-337 | ═NO—CH₂—CH(CH₃)—CH₂—CH₃ | |
| X-338 | ═NO—CH₂—CH₂—CH(CH₃)₂ | |
| X-339 | ═NO—CH₂—CHF₂ | |
| X-340 | ═NO—CH₂—CF₃ | |
| X-341 | ═NO—CH₂—CH═CH₂ | |
| X-342 | ═NO—CH₂—CH═CHCl | |
| X-343 | ═NO—CH₂—C≡CH | |
| X-344 | ═NO-cyclo-C₃H₅ | |
| X-345 | ═NO-cyclo-C₄H₇ | |
| X-346 | ═NO-cyclo-C₅H₉ | |
| X-347 | ═NO-cyclo-C₆H₁₁ | |
| X-348 | C₆H₅—CH₂—ON═ | |
| X-349 | 2-F—C₆H₄—CH₂—ON═ | |
| X-350 | 3-F—C₆H₄—CH₂—ON═ | |
| X-351 | 4-F—C₆H₄—CH₂—ON═ | |
| X-352 | 2-Cl—C₆H₄—CH₂—ON═ | |
| X-353 | 3-Cl—C₆H₄—CH₂—ON═ | |
| X-354 | 4-Cl—C₆H₄—CH₂—ON═ | |
| X-355 | 2-CH₃—C₆H₄—CH₂—ON═ | |
| X-356 | 3-CH₃—C₆H₄—CH₂—ON═ | |
| X-357 | 4-CH₃—C₆H₄—CH₂—ON═ | |
| X-358 | 2-CF₃—C₆H₄—CH₂—ON═ | |
| X-359 | 3-CF₃—C₆H₄—CH₂—ON═ | |
| X-360 | 4-CF₃—C₆H₄—CH₂—ON═ | |
| X-361 | 2-OCH₃—C₆H₄—CH₂—ON═ | |
| X-362 | 3-OCH₃—C₆H₄—CH₂—ON═ | |
| X-363 | 4-OCH₃—C₆H₄—CH₂—ON═ | |
| X-364 | 2-OCHF₂—C₆H₄—CH₂—ON═ | |
| X-365 | 3-OCHF₂—C₆H₄—CH₂—ON═ | |
| X-366 | 4-OCHF₂—C₆H₄—CH₂—ON═ | |
| X-367 | 2,3-F₂—C₆H₃—CH₂—ON═ | |
| X-368 | 2,4-F₂—C₆H₃—CH₂—ON═ | |
| X-369 | 2,5-F₂—C₆H₃—CH₂—ON═ | |
| X-370 | 2,6-F₂—C₆H₃—CH₂—ON═ | |
| X-371 | 3,4-F₂—C₆H₃—CH₂—ON═ | |
| X-372 | 3,5-F₂—C₆H₃—CH₂—ON═ | |
| X-373 | 2,3-Cl₂—C₆H₃—CH₂—ON═ | |
| X-374 | 2,4-Cl₂—C₆H₃—CH₂—ON═ | |
| X-375 | 2,5-Cl₂—C₆H₃—CH₂—ON═ | |
| X-376 | 2,6-Cl₂—C₆H₃—CH₂—ON═ | |
| X-377 | 3,4-Cl₂—C₆H₃—CH₂—ON═ | |
| X-378 | 3,5-Cl₂—C₆H₃—CH₂—ON═ | |
| X-379 | 2,3-(CH₃)₂—C₆H₃—CH₂—ON═ | |
| X-380 | 2,4-(CH₃)₂—C₆H₃—CH₂—ON═ | |
| X-381 | 2,5-(CH₃)₂—C₆H₃—CH₂—ON═ | |
| X-382 | 2,6-(CH₃)₂—C₆H₃—CH₂—ON═ | |
| X-383 | 3,4-(CH₃)₂—C₆H₃—CH₂—ON═ | |
| X-384 | 3,5-(CH₃)₂—C₆H₃—CH₂—ON═ | |

Preferred embodiments of the present invention are the following compounds I.E-1, I.E-2, I.E-3, I.E-4, I.E-5, I.E-6; compounds I.F-1, I.F-2, I.F-3, I.F-4, I.F-5, I.F-6. In these formulae, the substituents R⁵, R⁸ are independently as defined above or preferably defined herein:

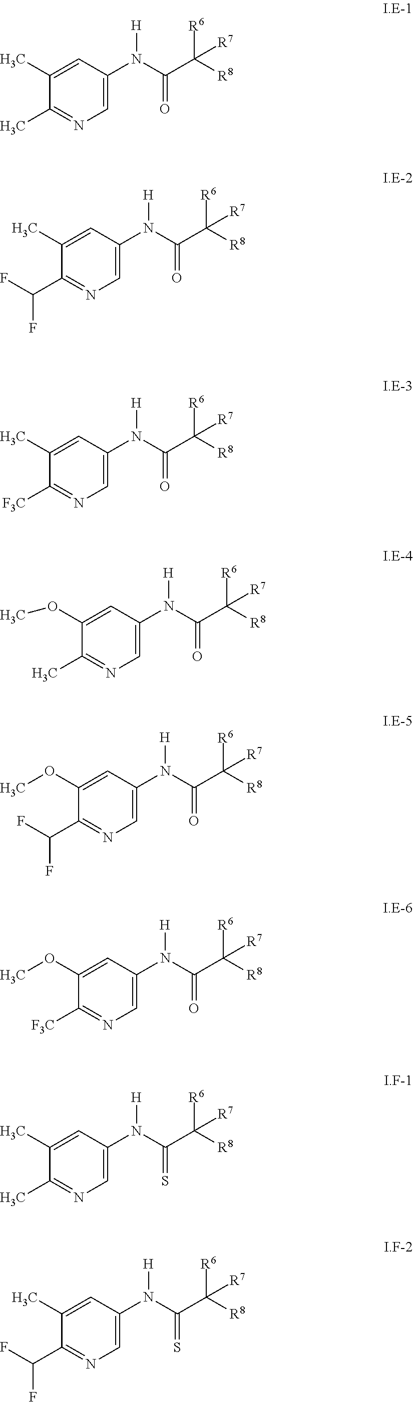

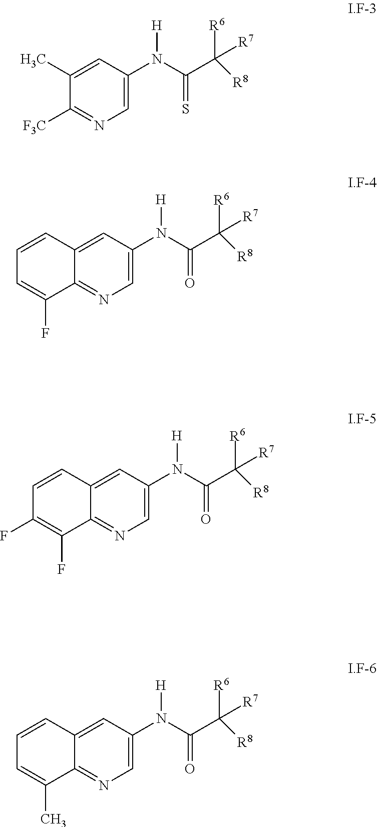

In particular with a view to their use, according to one embodiment, preference is given to the compounds of the compounds I.E-1, I.E-2, I.E-3, I.E-4, I.E-5, I.E-6; compounds I.F-1, I.F-2, I.F-3, I.F-4, I.F-5, I.F-6, that are compiled in the Tables 1c to 143c. Each of the groups mentioned for a substituent in the tables is furthermore per se, independently of the combination in which it is mentioned, a particularly preferred aspect of the substituent in question.

Table 1c Compounds of the formula I.E-1, I.E-2, I.E-3, I.E-4, I.E-5, I.E-6; I.F-1, I.F-2, I.F-3, I.F-4, I.F-5, I.F-6 in which R⁸ is cyclo-C₃H₅ and the meaning for the combination of R⁶ and R⁷ for each individual compound corresponds in each case to one line of Table Y (compounds I.E-1.1c.Y-1 to I.E-1.1c.Y-480, I.E-2.1c.Y-1 to I.E-2.1c.Y-480, I.E-3.1c.Y-1 to I.E-3.1c.Y-480, I.E-4.1c.Y-1 to I.E-5.1c.Y-480, I.E-5.1c.Y-1 to I.E-3.1c.Y-480, I.E-6.1c.Y-1 to I.E-6.1c.Y-480; I.F-1c.Y-1 to I.F-1.1c.Y-480, I.F-2.1c.Y-1 to I.F-2.1c.Y-480, I.F-3.1c.Y-1 to I.F-3.1c.Y-480, I.F-4.1c.Y-1 to I.F-4.1c.Y-480, I.F-5.1c.Y-1 to I.F-5.1c.Y-480, I.F-6.1c.Y-1 to I.F-6.1c.Y-480).
Table 2c Compounds of the formula I.E-1, I.E-2, I.E-3, I.E-4, I.E-5, I.E-6; I.F-1, I.F-2, I.F-3, I.F-4, I.F-5, I.F-6 in which R⁸ is cyclo-C₄H₇ and the meaning for the combination of R⁶ and R⁷ for each individual compound corresponds in each case to one line of Table Y (compounds I.E-1.2c.Y-1 to I.E-1.2c.Y-480, I.E-2.2c.Y-1 to I.E-2.2c.Y-480, I.E-3.2c.Y-1 to I.E-3.2c.Y-480, I.E-4.2c.Y-1 to I.E-5.2c.Y-480, I.E-5.2c.Y-1 to I.E-3.2c.Y-480, I.E-6.2c.Y-1 to I.E-6.2c.Y-480; I.F-2c.Y-1 to I.F-1.2c.Y-480, I.F-2.2c.Y-1 to I.F-2.2c.Y-480, I.F-3.2c.Y-1 to I.F-3.2c.Y-480, I.F-4.2c.Y-1 to I.F-4.2c.Y-480, I.F-5.2c.Y-1 to I.F-5.2c.Y-480, I.F-6.2c.Y-1 to I.F-6.2c.Y-480).
Table 3c Compounds of the formula I.E-1, I.E-2, I.E-3, I.E-4, I.E-5, I.E-6; I.F-1, I.F-2, I.F-3, I.F-4, I.F-5, I.F-6 in which R⁸ is cyclo-C₅H₉ and the meaning for the combination of R⁶ and R⁷ for each individual compound corresponds in each case to one line of Table Y (compounds I.E-1.3c.Y-1 to I.E-1.3c.Y-480, I.E-2.3c.Y-1 to I.E-2.3c.Y-480, I.E-3.3c.Y-1 to I.E-3.3c.Y-480, I.E-4.3c.Y-1 to I.E-5.3c.Y-480, I.E-5.3c.Y-1 to I.E-3.3c.Y-480, I.E-6.3c.Y-1 to I.E-6.3c.Y-480; I.F-3c.Y-1 to I.F-1.3c.Y-480, I.F-2.3c.Y-1 to I.F-2.3c.Y-480, I.F-3.3c.Y-1 to I.F-3.3c.Y-480, I.F-4.3c.Y-1 to I.F-4.3c.Y-480, I.F-5.3c.Y-1 to I.F-5.3c.Y-480, I.F-6.3c.Y-1 to I.F-6.3c.Y-480).
Table 4c Compounds of the formula I.E-1, I.E-2, I.E-3, I.E-4, I.E-5, I.E-6; I.F-1, I.F-2, I.F-3, I.F-4, I.F-5, I.F-6 in which R⁸ is cyclo-C₆H₁₁ and the meaning for the combination of R⁶ and R⁷ for each individual compound corresponds in each case to one line of Table Y (compounds I.E-1.4c.Y-1 to I.E-1.4c.Y-480, I.E-2.4c.Y-1 to I.E-2.4c.Y-480, I.E-3.4c.Y-1 to I.E-3.4c.Y-480, I.E-4.4c.Y-1 to I.E-5.4c.Y-480, I.E-5.4c.Y-1 to I.E-3.4c.Y-480, I.E-6.4c.Y-1 to I.E-6.4c.Y-480; I.F-4c.Y-1 to I.F-1.4c.Y-480, I.F-2.4c.Y-1 to I.F-2.4c.Y-480, I.F-3.4c.Y-1 to I.F-3.4c.Y-480, I.F-4.4c.Y-1 to I.F-4.4c.Y-480, I.F-5.4c.Y-1 to I.F-5.4c.Y-480, I.F-6.4c.Y-1 to I.F-6.4c.Y-480).
Table 5c Compounds of the formula I.E-1, I.E-2, I.E-3, I.E-4, I.E-5, I.E-6; I.F-1, I.F-2, I.F-3, I.F-4, I.F-5, I.F-6 in which R³ is

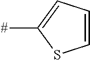

and the meaning for the combination of R⁶ and R⁷ for each individual compound corresponds in each case to one line of Table Y (compounds I.E-1.5c.Y-1 to I.E-1.5c.Y-480, I.E-2.5c.Y-1 to I.E-2.5c.Y-480, I.E-3.5c.Y-1 to I.E-3.5c.Y-480, I.E-4.5c.Y-1 to I.E-5.5c.Y-480, I.E-5.5c.Y-1 to I.E-3.5c.Y-480, I.E-6.5c.Y-1 to I.E-6.5c.Y-480; I.F-5c.Y-1 to I.F-1.5c.Y-480, I.F-2.5c.Y-1 to I.F-2.5c.Y-480, I.F-3.5c.Y-1 to I.F-3.5c.Y-480, I.F-4.5c.Y-1 to I.F-4.5c.Y-480, I.F-5.5c.Y-1 to I.F-5.5c.Y-480, I.F-6.5c.Y-1 to I.F-6.5c.Y-480).
Table 6c Compounds of the formula I.E-1, I.E-2, I.E-3, I.E-4, I.E-5, I.E-6; I.F-1, I.F-2, I.F-3, I.F-4, I.F-5, I.F-6 in which R⁸ is C₆H₅ and the meaning for the combination of R⁶ and R⁷ for each individual compound corresponds in each case to one line of Table Y (compounds I.E-1.6c.Y-1 to I.E-1.6c.Y-480, I.E-2.6c.Y-1 to I.E-2.6c.Y-480, I.E-3.6c.Y-1 to I.E-3.6c.Y-480, I.E-4.6c.Y-1 to I.E-5.6c.Y-480, I.E-5.6c.Y-1 to I.E-3.6c.Y-480, I.E-6.6c.Y-1 to I.E-6.6c.Y-480; I.F-6c.Y-1 to I.F-1.6c.Y-480, I.F-2.6c.Y-1 to I.F-2.6c.Y-480, I.F-3.6c.Y-1 to I.F-3.6c.Y-480, I.F-4.6c.Y-1 to I.F-4.6c.Y-480, I.F-5.6c.Y-1 to I.F-5.6c.Y-480, I.F-6.6c.Y-1 to I.F-6.6c.Y-480).
Table 7c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-F—C₆H₄ and the meaning for the combination of R⁶ and R⁷ for each individual compound corresponds in each case to one line of Table Y (compounds I.E-1.7c.Y-1 to I.E-1.7c.Y-480, I.E-2.7c.Y-1 to I.E-2.7c.Y-480, I.E-3.7c.Y-1 to I.E-3.7c.Y-480, I.E-4.7c.Y-1 to I.E-5.7c.Y-480, I.E-5.7c.Y-1 to I.E-3.7c.Y-480, I.E-6.7c.Y-1 to I.E-6.7c.Y-480; I.F-7c.Y-1 to I.F-1.7c.Y-480, I.F-2.7c.Y-1 to I.F-2.7c.Y-480, I.F-3.7c.Y-1 to I.F-3.7c.Y-480, I.F-4.7c.Y-1 to I.F-4.7c.Y-480, I.F-5.7c.Y-1 to I.F-5.7c.Y-480, I.F-6.7c.Y-1 to I.F-6.7c.Y-480).
Table 8c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-F—C₆H₄ and the meaning for the combination of R⁶ and R⁷ for each individual compound corresponds in each case to one line of Table Y (compounds I.E-1.8c.Y-1 to I.E-1.8c.Y-480, I.E-2.8c.Y-1 to I.E-2.8c.Y-480, I.E-3.8c.Y-1 to I.E-3.8c.Y-480, I.E-4.8c.Y-1 to I.E-5.8c.Y-480, I.E-5.8c.Y-1 to I.E-3.8c.Y-480, I.E-6.8c.Y-1 to I.E-6.8c.Y-480; I.F-8c.Y-1 to I.F-1.8c.Y-480, I.F-2.8c.Y-1 to I.F-2.8c.Y-480, I.F-3.8c.Y-1 to I.F-3.8c.Y-480, I.F-4.8c.Y-1 to I.F-4.8c.Y-480, I.F-5.8c.Y-1 to I.F-5.8c.Y-480, I.F-6.8c.Y-1 to I.F-6.8c.Y-480).
Table 9c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-F—C₆H₄ and the meaning for the combination of R⁶ and R⁷ for each individual compound corresponds in each case to one line of Table Y (compounds I.E-1.9c.Y-1 to I.E-1.9c.Y-480, I.E-2.9c.Y-1 to I.E-2.9c.Y-480, I.E-3.9c.Y-1 to I.E-3.9c.Y-480, I.E-4.9c.Y-1 to I.E-5.9c.Y-480, I.E-5.9c.Y-1 to I.E-3.9c.Y-480, I.E-6.9c.Y-1 to I.E-6.9c.Y-480; I.F-9c.Y-1 to I.F-1.9c.Y-480, I.F-2.9c.Y-1 to I.F-2.9c.Y-480, I.F-3.9c.Y-1 to I.F-3.9c.Y-480, I.F-4.9c.Y-1 to I.F-4.9c.Y-480, I.F-5.9c.Y-1 to I.F-5.9c.Y-480, I.F-6.9c.Y-1 to I.F-6.9c.Y-480).
Table 10c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-Cl—C₆H₄ and the meaning for the combination of R⁶ and R⁷ for each individual compound corresponds in each case to one line of Table Y (compounds I.E-1.10c.Y-1 to I.E-1.10c.Y-480, I.E-2.10c.Y-1 to I.E-2.10c.Y-480, I.E-3.10c.Y-1 to I.E-3.10c.Y-480, I.E-4.10c.Y-1 to I.E-5.10c.Y-480, I.E-5.10c.Y-1 to I.E-3.10c.Y-480, I.E-6.10c.Y-1 to I.E-6.10c.Y-480; I.F-10c.Y-1 to I.F-1.10c.Y-480, I.F-2.10c.Y-1 to I.F-2.10c.Y-480, I.F-3.10c.Y-1 to I.F-3.10c.Y-480, I.F-4.10c.Y-1 to I.F-4.10c.Y-480, I.F-5.10c.Y-1 to I.F-5.10c.Y-480, I.F-6.10c.Y-1 to I.F-6.10c.Y-480).
Table 11c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-Cl—C₆H₄ and the meaning for the combination of R⁶ and R⁷ for each individual compound corresponds in each case to one line of Table Y (compounds I.E-1.11c.Y-1 to I.E-1.11c.Y-480, I.E-2.11c.Y-1 to I.E-2.11c.Y-480, I.E-3.11c.Y-1 to I.E-3.11c.Y-480, I.E-4.11c.Y-1 to I.E-5.11c.Y-480, I.E-5.11c.Y-1 to I.E-3.11c.Y-480, I.E-6.11c.Y-1 to I.E-6.11c.Y-480; I.F-11c.Y-1 to I.F-1.11c.Y-480, I.F-2.11c.Y-1 to I.F-2.11c.Y-480, I.F-3.11c.Y-1 to I.F-3.11c.Y-480, I.F-4.11c.Y-1 to I.F-4.11c.Y-480, I.F-5.11c.Y-1 to I.F-5.11c.Y-480, I.F-6.11c.Y-1 to I.F-6.11c.Y-480).
Table 12c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-Cl—C₆H₄ and the meaning for the combination of R⁶ and R⁷ for each individual compound corresponds in each case to one line of Table Y (compounds I.E-1.12c.Y-1 to I.E-1.12c.Y-480, I.E-2.12c.Y-1 to I.E-2.12c.Y-480, I.E-3.12c.Y-1 to I.E-3.12c.Y-480, I.E-4.12c.Y-1 to I.E-5.12c.Y-480, I.E-5.12c.Y-1 to I.E-3.12c.Y-480, I.E-6.12c.Y-1 to I.E-6.12c.Y-480; I.F-12c.Y-1 to I.F-1.12c.Y-480, I.F-2.12c.Y-1 to I.F-2.12c.Y-480, I.F-3.12c.Y-1 to I.F-3.12c.Y-480, I.F-4.12c.Y-1 to I.F-4.12c.Y-480, I.F-5.12c.Y-1 to I.F-5.12c.Y-480, I.F-6.12c.Y-1 to I.F-6.12c.Y-480).
Table 13c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-OCH₃—C₆H₄ and the meaning for the combination of R⁶ and R⁷ for each individual compound corresponds in each case to one line of Table Y (compounds I.E-1.13c.Y-1 to I.E-1.13c.Y-480, I.E-2.13c.Y-1 to I.E-2.13c.Y-480, I.E-3.13c.Y-1 to I.E-3.13c.Y-480, I.E-4.13c.Y-1 to I.E-5.13c.Y-480, I.E-5.13c.Y-1 to I.E-3.13c.Y-480, I.E-6.13c.Y-1 to I.E-6.13c.Y-480; I.F-13c.Y-1 to I.F-1.13c.Y-480, I.F-2.13c.Y-1 to I.F-2.13c.Y-480, I.F-3.13c.Y-1 to I.F-3.13c.Y-480, I.F-4.13c.Y-1 to I.F-4.13c.Y-480, I.F-5.13c.Y-1 to I.F-5.13c.Y-480, I.F-6.13c.Y-1 to I.F-6.13c.Y-480).
Table 14c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-OCH₃—C₆H₄ and the meaning for the combination of R⁶ and R⁷ for each individual compound corresponds in each case to one line of Table Y (compounds I.E-1.14c.Y-1 to I.E-1.14c.Y-480, I.E-2.14c.Y-1 to I.E-2.14c.Y-480, I.E-3.14c.Y-1 to I.E-3.14c.Y-480, I.E-4.14c.Y-1 to I.E-5.14c.Y-480, I.E-5.14c.Y-1 to I.E-3.14c.Y-480, I.E-6.14c.Y-1 to I.E-6.14c.Y-480; I.F-14c.Y-1 to I.F-1.14c.Y-480, I.F-2.14c.Y-1 to I.F-2.14c.Y-480, I.F-3.14c.Y-1 to I.F-3.14c.Y-480, I.F-4.14c.Y-1 to I.F-4.14c.Y-480, I.F-5.14c.Y-1 to I.F-5.14c.Y-480, I.F-6.14c.Y-1 to I.F-6.14c.Y-480).
Table 15c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-OCH₃—C₆H₄ and the meaning for the combination of R⁶ and R⁷ for each individual compound corresponds in each case to one line of Table Y (compounds I.E-1.15c.Y-1 to I.E-1.15c.Y-480, I.E-2.15c.Y-1 to I.E-2.15c.Y-480, I.E-3.15c.Y-1 to I.E-3.15c.Y-480, I.E-4.15c.Y-1 to I.E-5.15c.Y-480, I.E-5.15c.Y-1 to I.E-3.15c.Y-480, I.E-6.15c.Y-1 to I.E-6.15c.Y-480; I.F-15c.Y-1 to I.F-1.15c.Y-480, I.F-2.15c.Y-1 to I.F-2.15c.Y-480, I.F-3.15c.Y-1 to I.F-3.15c.Y-480, I.F-4.15c.Y-1 to I.F-4.15c.Y-480, I.F-5.15c.Y-1 to I.F-5.15c.Y-480, I.F-6.15c.Y-1 to I.F-6.15c.Y-480).
Table 16c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-OCHF₂—C₆H₄ and the meaning for the combination of R⁶ and R⁷ for each individual compound corresponds in each case to one line of Table Y (compounds I.E-1.16c.Y-1 to I.E-1.16c.Y-480, I.E-2.16c.Y-1 to I.E-2.16c.Y-480, I.E-3.16c.Y-1 to I.E-3.16c.Y-480, I.E-4.16c.Y-1 to I.E-5.16c.Y-480, I.E-5.16c.Y-1 to I.E-3.16c.Y-480, I.E-6.16c.Y-1 to I.E-6.16c.Y-480; I.F-16c.Y-1 to I.F-1.16c.Y-480, I.F-2.16c.Y-1 to I.F-2.16c.Y-480, I.F-3.16c.Y-1 to I.F-3.16c.Y-480, I.F-4.16c.Y-1 to I.F-4.16c.Y-480, I.F-5.16c.Y-1 to I.F-5.16c.Y-480, I.F-6.16c.Y-1 to I.F-6.16c.Y-480).
Table 17c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-OCHF₂—C₆H₄ and the meaning for the combination of R⁶ and R⁷ for each individual compound corresponds in each case to one line of Table Y (compounds I.E-1.17c.Y-1 to I.E-1.17c.Y-480, I.E-2.17c.Y-1 to I.E-2.17c.Y-480, I.E-3.17c.Y-1 to I.E-3.17c.Y-480, I.E-4.17c.Y-1 to I.E-5.17c.Y-480, I.E-5.17c.Y-1 to I.E-3.17c.Y-480, I.E-6.17c.Y-1 to I.E-6.17c.Y-480; I.F-17c.Y-1 to I.F-1.17c.Y-480, I.F-2.17c.Y-1 to I.F-2.17c.Y-480, I.F-3.17c.Y-1 to I.F-3.17c.Y-480, I.F-4.17c.Y-1 to I.F-4.17c.Y-480, I.F-5.17c.Y-1 to I.F-5.17c.Y-480, I.F-6.17c.Y-1 to I.F-6.17c.Y-480).
Table 18c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-OCHF₂—C₆H₄ and the meaning for the combination of R⁶ and R⁷ for each individual compound corresponds in each case to one line of Table Y (compounds I.E-1.18c.Y-1 to I.E-1.18c.Y-480, I.E-2.18c.Y-1 to I.E-2.18c.Y-480, I.E-3.18c.Y-1 to I.E-3.18c.Y-480, I.E-4.18c.Y-1 to I.E-5.18c.Y-480, I.E-5.18c.Y-1 to I.E-3.18c.Y-480, I.E-6.18c.Y-1 to I.E-6.18c.Y-480; I.F-18c.Y-1 to I.F-1.18c.Y-480, I.F-2.18c.Y-1 to I.F-2.18c.Y-480, I.F-3.18c.Y-1 to I.F-3.18c.Y-480, I.F-4.18c.Y-1 to I.F-4.18c.Y-480, I.F-5.18c.Y-1 to I.F-5.18c.Y-480, I.F-6.18c.Y-1 to I.F-6.18c.Y-480).
Table 19c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-CH₃—C₆H₄ and the meaning for the combination of R⁶ and R⁷ for each individual compound corresponds in each case to one line of Table Y (compounds I.E-1.19c.Y-1 to I.E-1.19c.Y-480, I.E-2.19c.Y-1 to I.E-2.19c.Y-480, I.E-3.19c.Y-1 to I.E-3.19c.Y-480, I.E-4.19c.Y-1 to I.E-5.19c.Y-480, I.E-5.19c.Y-1 to I.E-3.19c.Y-480, I.E-6.19c.Y-1 to I.E-6.19c.Y-480; I.F-19c.Y-1 to I.F-1.19c.Y-480, I.F-2.19c.Y-1 to I.F-2.19c.Y-480, I.F-3.19c.Y-1 to I.F-3.19c.Y-480, I.F-4.19c.Y-1 to I.F-4.19c.Y-480, I.F-5.19c.Y-1 to I.F-5.19c.Y-480, I.F-6.19c.Y-1 to I.F-6.19c.Y-480).
Table 20c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-CH₃—C₆H₄ and the meaning for the combination of R⁶ and R⁷ for each individual compound corresponds in each case to one line of Table Y (compounds I.E-1.20c.Y-1 to I.E-1.20c.Y-480, I.E-2.20c.Y-1 to I.E-2.20c.Y-480, I.E-3.20c.Y-1 to I.E-3.20c.Y-480, I.E-4.20c.Y-1 to I.E-5.20c.Y-480, I.E-5.20c.Y-1 to I.E-3.20c.Y-480, I.E-6.20c.Y-1 to I.E-6.20c.Y-480; I.F-20c.Y-1 to I.F-1.20c.Y-480, I.F-2.20c.Y-1 to I.F-2.20c.Y-480, I.F-3.20c.Y-1 to I.F-3.20c.Y-480, I.F-4.20c.Y-1 to I.F-4.20c.Y-480, I.F-5.20c.Y-1 to I.F-5.20c.Y-480, I.F-6.20c.Y-1 to I.F-6.20c.Y-480).
Table 21c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-CH₃—C₆H₄ and the meaning for the combination of R⁶ and R⁷ for each individual compound corresponds in each case to one line of Table Y (compounds I.E-1.21c.Y-1 to I.E-1.21c.Y-480, I.E-2.21c.Y-1 to I.E-2.21c.Y-480, I.E-3.21c.Y-1 to I.E-3.21c.Y-480, I.E-4.21c.Y-1 to I.E-5.21c.Y-480, I.E-5.21c.Y-1 to I.E-3.21c.Y-480, I.E-6.21c.Y-1 to I.E-6.21c.Y-480; I.F-21c.Y-1 to I.F-1.21c.Y-480, I.F-2.21c.Y-1 to I.F-2.21c.Y-480, I.F-3.21c.Y-1 to I.F-3.21c.Y-480, I.F-4.21c.Y-1 to I.F-4.21c.Y-480, I.F-5.21c.Y-1 to I.F-5.21c.Y-480, I.F-6.21c.Y-1 to I.F-6.21c.Y-480).
Table 22c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-CF₃—C₆H₄ and the meaning for the combination of R⁶ and R⁷ for each individual compound corresponds in each case to one line of Table Y (compounds I.E-1.22c.Y-1 to I.E-1.22c.Y-480, I.E-2.22c.Y-1 to I.E-2.22c.Y-480, I.E-3.22c.Y-1 to I.E-3.22c.Y-480, I.E-4.22c.Y-1 to I.E-5.22c.Y-480, I.E-5.22c.Y-1 to I.E-3.22c.Y-480, I.E-6.22c.Y-1 to I.E-6.22c.Y-480; I.F-22c.Y-1 to I.F-1.22c.Y-480, I.F-2.22c.Y-1 to I.F-2.22c.Y-480, I.F-3.22c.Y-1 to I.F-3.22c.Y-480, I.F-4.22c.Y-1 to I.F-4.22c.Y-480, I.F-5.22c.Y-1 to I.F-5.22c.Y-480, I.F-6.22c.Y-1 to I.F-6.22c.Y-480).
Table 23c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-CF₃—C₆H₄ and the meaning for the combination of R⁶ and R⁷ for each individual compound corresponds in each case to one line of Table Y (compounds I.E-1.23c.Y-1 to I.E-1.23c.Y-480, I.E-2.23c.Y-1 to I.E-2.23c.Y-480, I.E-3.23c.Y-1 to I.E-3.23c.Y-480, I.E-4.23c.Y-1 to I.E-5.23c.Y-480, I.E-5.23c.Y-1 to I.E-3.23c.Y-480, I.E-6.23c.Y-1 to I.E-6.23c.Y-480; I.F-23c.Y-1 to I.F-1.23c.Y-480, I.F-2.23c.Y-1 to I.F-2.23c.Y-480, I.F-3.23c.Y-1 to I.F-3.23c.Y-480, I.F-4.23c.Y-1 to I.F-4.23c.Y-480, I.F-5.23c.Y-1 to I.F-5.23c.Y-480, I.F-6.23c.Y-1 to I.F-6.23c.Y-480).
Table 24c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-CF₃—C₆H₄ and the meaning for the combination of R⁶ and R⁷ for each individual compound corresponds in each case to one line of Table Y (compounds I.E-1.24c.Y-1 to I.E-1.24c.Y-480, I.E-2.24c.Y-1 to I.E-2.24c.Y-480, I.E-3.24c.Y-1 to I.E-3.24c.Y-480, I.E-4.24c.Y-1 to I.E-5.24c.Y-480, I.E-5.24c.Y-1 to I.E-3.24c.Y-480, I.E-6.24c.Y-1 to I.E-6.24c.Y-480; I.F-24c.Y-1 to I.F-1.24c.Y-480, I.F-2.24c.Y-1 to I.F-2.24c.Y-480, I.F-3.24c.Y-1 to I.F-3.24c.Y-480, I.F-4.24c.Y-1 to I.F-4.24c.Y-480, I.F-5.24c.Y-1 to I.F-5.24c.Y-480, I.F-6.24c.Y-1 to I.F-6.24c.Y-480).
Table 25c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2,3-F₂-C₆H₃ and the meaning for the combination of R⁶ and R⁷ for each individual compound corresponds in each case to one line of Table Y (compounds I.E-1.25c.Y-1 to I.E-1.25c.Y-480, I.E-2.25c.Y-1 to I.E-2.25c.Y-480, I.E-3.25c.Y-1 to I.E-3.25c.Y-480, I.E-4.25c.Y-1 to I.E-5.25c.Y-480, I.E-5.25c.Y-1 to I.E-3.25c.Y-480, I.E-6.25c.Y-1 to I.E-6.25c.Y-480; I.F-25c.Y-1 to I.F-1.25c.Y-480, I.F-2.25c.Y-1 to I.F-2.25c.Y-480, I.F-3.25c.Y-1 to I.F-3.25c.Y-480, I.F-4.25c.Y-1 to I.F-4.25c.Y-480, I.F-5.25c.Y-1 to I.F-5.25c.Y-480, I.F-6.25c.Y-1 to I.F-6.25c.Y-480).
Table 26c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2,4-F₂-C₆H₃ and the meaning for the combination of R⁶ and R⁷ for each individual compound corresponds in each case to one line of Table Y (compounds I.E-1.26c.Y-1 to I.E-1.26c.Y-480, I.E-2.26c.Y-1 to I.E-2.26c.Y-480, I.E-3.26c.Y-1 to I.E-3.26c.Y-480, I.E-4.26c.Y-1 to I.E-5.26c.Y-480, I.E-5.26c.Y-1 to I.E-3.26c.Y-480, I.E-6.26c.Y-1 to I.E-6.26c.Y-480; I.F-26c.Y-1 to I.F-1.26c.Y-480, I.F-2.26c.Y-1 to I.F-2.26c.Y-480, I.F-3.26c.Y-1 to I.F-3.26c.Y-480, I.F-4.26c.Y-1 to I.F-4.26c.Y-480, I.F-5.26c.Y-1 to I.F-5.26c.Y-480, I.F-6.26c.Y-1 to I.F-6.26c.Y-480).
Table 27c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2,5-F₂-C₆H₃ and the meaning for the combination of R⁶ and R⁷ for each individual compound corresponds in each case to one line of Table Y (compounds I.E-1.27c.Y-1 to I.E-1.27c.Y-480, I.E-2.27c.Y-1 to I.E-2.27c.Y-480, I.E-3.27c.Y-1 to I.E-3.27c.Y-480, I.E-4.27c.Y-1 to I.E-5.27c.Y-480, I.E-5.27c.Y-1 to I.E-3.27c.Y-480, I.E-6.27c.Y-1 to I.E-6.27c.Y-480; I.F-27c.Y-1 to I.F-1.27c.Y-480, I.F-2.27c.Y-1 to I.F-2.27c.Y-480, I.F-3.27c.Y-1 to I.F-3.27c.Y-480, I.F-4.27c.Y-1 to I.F-4.27c.Y-480, I.F-5.27c.Y-1 to I.F-5.27c.Y-480, I.F-6.27c.Y-1 to I.F-6.27c.Y-480).
Table 28c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2,6-F₂-C₆H₃ and the meaning for the combination of R⁶ and R⁷ for each individual compound corresponds in each case to one line of Table Y (compounds I.E-1.28c.Y-1 to I.E-1.28c.Y-480, I.E-2.28c.Y-1 to I.E-2.28c.Y-480, I.E-3.28c.Y-1 to I.E-3.28c.Y-480, I.E-4.28c.Y-1 to I.E-5.28c.Y-480, I.E-5.28c.Y-1 to I.E-3.28c.Y-480, I.E-6.28c.Y-1 to I.E-6.28c.Y-480; I.F-28c.Y-1 to I.F-1.28c.Y-480, I.F-2.28c.Y-1 to I.F-2.28c.Y-480, I.F-3.28c.Y-1 to I.F-3.28c.Y-480, I.F-4.28c.Y-1 to I.F-4.28c.Y-480, I.F-5.28c.Y-1 to I.F-5.28c.Y-480, I.F-6.28c.Y-1 to I.F-6.28c.Y-480).
Table 29c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3,4-F₂-C₆H₃ and the meaning for the combination of R⁶ and R⁷ for each individual compound corresponds in each case to one line of Table Y (compounds I.E-1.29c.Y-1 to I.E-1.29c.Y-480, I.E-2.29c.Y-1 to I.E-2.29c.Y-480, I.E-3.29c.Y-1 to I.E-3.29c.Y-480, I.E-4.29c.Y-1 to I.E-5.29c.Y-480, I.E-5.29c.Y-1 to I.E-3.29c.Y-480, I.E-6.29c.Y-1 to I.E-6.29c.Y-480; I.F-29c.Y-1 to I.F-1.29c.Y-480, I.F-2.29c.Y-1 to I.F-2.29c.Y-480, I.F-3.29c.Y-1 to I.F-3.29c.Y-480, I.F-4.29c.Y-1 to I.F-4.29c.Y-480, I.F-5.29c.Y-1 to I.F-5.29c.Y-480, I.F-6.29c.Y-1 to I.F-6.29c.Y-480).
Table 30c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3,5-F₂-C₆H₃ and the meaning for the combination of R⁶ and R⁷ for each individual compound corresponds in each case to one line of Table Y (compounds I.E-1.30c.Y-1 to I.E-1.30c.Y-480, I.E-2.30c.Y-1 to I.E-2.30c.Y-480, I.E-3.30c.Y-1 to I.E-3.30c.Y-480, I.E-4.30c.Y-1 to I.E-5.30c.Y-480, I.E-5.30c.Y-1 to I.E-3.30c.Y-480, I.E-6.30c.Y-1 to I.E-6.30c.Y-480; I.F-30c.Y-1 to I.F-1.30c.Y-480, I.F-2.30c.Y-1 to I.F-2.30c.Y-480, I.F-3.30c.Y-1 to I.F-3.30c.Y-480, I.F-4.30c.Y-1 to I.F-4.30c.Y-480, I.F-5.30c.Y-1 to I.F-5.30c.Y-480, I.F-6.30c.Y-1 to I.F-6.30c.Y-480).
Table 31c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2,3-Cl₂—C₆H₃ and the meaning for the combination of R⁶ and R⁷ for each individual compound corresponds in each case to one line of Table Y (compounds I.E-1.31c.Y-1 to I.E-1.31c.Y-480, I.E-2.31c.Y-1 to I.E-2.31c.Y-480, I.E-3.31c.Y-1 to I.E-3.31c.Y-480, I.E-4.31c.Y-1 to I.E-5.31c.Y-480, I.E-5.31c.Y-1 to I.E-3.31c.Y-480, I.E-6.31c.Y-1 to I.E-6.31c.Y-480; I.F-31c.Y-1 to I.F-1.31c.Y-480, I.F-2.31c.Y-1 to I.F-2.31c.Y-480, I.F-3.31c.Y-1 to I.F-3.31c.Y-480, I.F-4.31c.Y-1 to I.F-4.31c.Y-480, I.F-5.31c.Y-1 to I.F-5.31c.Y-480, I.F-6.31c.Y-1 to I.F-6.31c.Y-480).
Table 32c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2,4-Cl₂—C₆H₃ and the meaning for the combination of R⁶ and R⁷ for each individual compound corresponds in each case to one line of Table Y (compounds I.E-1.32c.Y-1 to I.E-1.32c.Y-480, I.E-2.32c.Y-1 to I.E-2.32c.Y-480, I.E-3.32c.Y-1 to I.E-3.32c.Y-480, I.E-4.32c.Y-1 to I.E-5.32c.Y-480, I.E-5.32c.Y-1 to I.E-3.32c.Y-480, I.E-6.32c.Y-1 to I.E-6.32c.Y-480; I.F-32c.Y-1 to I.F-1.32c.Y-480, I.F-2.32c.Y-1 to I.F-2.32c.Y-480, I.F-3.32c.Y-1 to I.F-3.32c.Y-480, I.F-4.32c.Y-1 to I.F-4.32c.Y-480, I.F-5.32c.Y-1 to I.F-5.32c.Y-480, I.F-6.32c.Y-1 to I.F-6.32c.Y-480).
Table 33c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2,5-Cl₂—C₆H₃ and the meaning for the combination of R⁶ and R⁷ for each individual compound corresponds in each case to one line of Table Y (compounds I.E-1.33c.Y-1 to I.E-1.33c.Y-480, I.E-2.33c.Y-1 to I.E-2.33c.Y-480, I.E-3.33c.Y-1 to I.E-3.33c.Y-480, I.E-4.33c.Y-1 to I.E-5.33c.Y-480, I.E-5.33c.Y-1 to I.E-3.33c.Y-480, I.E-6.33c.Y-1 to I.E-6.33c.Y-480; I.F-33c.Y-1 to I.F-1.33c.Y-480, I.F-2.33c.Y-1 to I.F-2.33c.Y-480, I.F-3.33c.Y-1 to I.F-3.33c.Y-480, I.F-4.33c.Y-1 to I.F-4.33c.Y-480, I.F-5.33c.Y-1 to I.F-5.33c.Y-480, I.F-6.33c.Y-1 to I.F-6.33c.Y-480).
Table 34c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2,6-Cl₂—C₆H₃ and the meaning for the combination of R⁶ and R⁷ for each individual compound corresponds in each case to one line of Table Y (compounds I.E-1.34c.Y-1 to I.E-1.34c.Y-480, I.E-2.34c.Y-1 to I.E-2.34c.Y-480, I.E-3.34c.Y-1 to I.E-3.34c.Y-480, I.E-4.34c.Y-1 to I.E-5.34c.Y-480, I.E-5.34c.Y-1 to I.E-3.34c.Y-480, I.E-6.34c.Y-1 to I.E-6.34c.Y-480; I.F-34c.Y-1 to I.F-1.34c.Y-480, I.F-2.34c.Y-1 to I.F-2.34c.Y-480, I.F-3.34c.Y-1 to I.F-3.34c.Y-480, I.F-4.34c.Y-1 to I.F-4.34c.Y-480, I.F-5.34c.Y-1 to I.F-5.34c.Y-480, I.F-6.34c.Y-1 to I.F-6.34c.Y-480).
Table 35c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3,4-Cl₂—C₆H₃ and the meaning for the combination of R⁶ and R⁷ for each individual compound corresponds in each case to one line of Table Y (compounds I.E-1.35c.Y-1 to I.E-1.35c.Y-480, I.E-2.35c.Y-1 to I.E-2.35c.Y-480, I.E-3.35c.Y-1 to I.E-3.35c.Y-480, I.E-4.35c.Y-1 to I.E-5.35c.Y-480, I.E-5.35c.Y-1 to I.E-3.35c.Y-480, I.E-6.35c.Y-1 to I.E-6.35c.Y-480; I.F-35c.Y-1 to I.F-1.35c.Y-480, I.F-2.35c.Y-1 to I.F-2.35c.Y-480, I.F-3.35c.Y-1 to I.F-3.35c.Y-480, I.F-4.35c.Y-1 to I.F-4.35c.Y-480, I.F-5.35c.Y-1 to I.F-5.35c.Y-480, I.F-6.35c.Y-1 to I.F-6.35c.Y-480).
Table 36c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3,5-Cl₂—C₆H₃ and the meaning for the combination of R⁶ and R⁷ for each individual compound corresponds in each case to one line of Table Y (compounds I.E-1.36c.Y-1 to I.E-1.36c.Y-480, I.E-2.36c.Y-1 to I.E-2.36c.Y-480, I.E-3.36c.Y-1 to I.E-3.36c.Y-480, I.E-4.36c.Y-1 to I.E-5.36c.Y-480, I.E-5.36c.Y-1 to I.E-3.36c.Y-480, I.E-6.36c.Y-1 to I.E-6.36c.Y-480; I.F-36c.Y-1 to I.F-1.36c.Y-480, I.F-2.36c.Y-1 to I.F-2.36c.Y-480, I.F-3.36c.Y-1 to I.F-3.36c.Y-480, I.F-4.36c.Y-1 to I.F-4.36c.Y-480, I.F-5.36c.Y-1 to I.F-5.36c.Y-480, I.F-6.36c.Y-1 to I.F-6.36c.Y-480).
Table 37c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2,3-(CH₃)₂-C₆H₃ and the meaning for the combination of R⁶ and R⁷ for each individual compound corresponds in each case to one line of Table Y (compounds I.E-1.37c.Y-1 to I.E-1.37c.Y-480, I.E-2.37c.Y-1 to I.E-2.37c.Y-480, I.E-3.37c.Y-1 to I.E-3.37c.Y-480, I.E-4.37c.Y-1 to I.E-5.37c.Y-480, I.E-5.37c.Y-1 to I.E-3.37c.Y-480, I.E-6.37c.Y-1 to I.E-6.37c.Y-480; I.F-37c.Y-1 to I.F-1.37c.Y-480, I.F-2.37c.Y-1 to I.F-2.37c.Y-480, I.F-3.37c.Y-1 to I.F-3.37c.Y-480, I.F-4.37c.Y-1 to I.F-4.37c.Y-480, I.F-5.37c.Y-1 to I.F-5.37c.Y-480, I.F-6.37c.Y-1 to I.F-6.37c.Y-480).
Table 38c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2,4-(CH₃)₂-C₆H₃ and the meaning for the combination of R⁶ and R⁷ for each individual compound corresponds in each case to one line of Table Y (compounds I.E-1.38c.Y-1 to I.E-1.38c.Y-480, I.E-2.38c.Y-1 to I.E-2.38c.Y-480, I.E-3.38c.Y-1 to I.E-3.38c.Y-480, I.E-4.38c.Y-1 to I.E-5.38c.Y-480, I.E-5.38c.Y-1 to I.E-3.38c.Y-480, I.E-6.38c.Y-1 to I.E-6.38c.Y-480; I.F-38c.Y-1 to I.F-1.38c.Y-480, I.F-2.38c.Y-1 to I.F-2.38c.Y-480, I.F-3.38c.Y-1 to I.F-3.38c.Y-480, I.F-4.38c.Y-1 to I.F-4.38c.Y-480, I.F-5.38c.Y-1 to I.F-5.38c.Y-480, I.F-6.38c.Y-1 to I.F-6.38c.Y-480).
Table 39c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2,5-(CH₃)₂-C₆H₃ and the meaning for the combination of R⁶ and R⁷ for each individual compound corresponds in each case to one line of Table Y (compounds I.E-1.39c.Y-1 to I.E-1.39c.Y-480, I.E-2.39c.Y-1 to I.E-2.39c.Y-480, I.E-3.39c.Y-1 to I.E-3.39c.Y-480, I.E-4.39c.Y-1 to I.E-5.39c.Y-480, I.E-5.39c.Y-1 to I.E-3.39c.Y-480, I.E-6.39c.Y-1 to I.E-6.39c.Y-480; I.F-39c.Y-1 to I.F-1.39c.Y-480, I.F-2.39c.Y-1 to I.F-2.39c.Y-480, I.F-3.39c.Y-1 to I.F-3.39c.Y-480, I.F-4.39c.Y-1 to I.F-4.39c.Y-480, I.F-5.39c.Y-1 to I.F-5.39c.Y-480, I.F-6.39c.Y-1 to I.F-6.39c.Y-480).
Table 40c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2,6-(CH₃)₂-C₆H₃ and the meaning for the combination of R⁶ and R⁷ for each individual compound corresponds in each case to one line of Table Y (compounds I.E-1.40c.Y-1 to I.E-1.40c.Y-480, I.E-2.40c.Y-1 to I.E-2.40c.Y-480, I.E-3.40c.Y-1 to I.E-3.40c.Y-480, I.E-4.40c.Y-1 to I.E-5.40c.Y-480, I.E-5.40c.Y-1 to I.E-3.40c.Y-480, I.E-6.40c.Y-1 to I.E-6.40c.Y-480; I.F-40c.Y-1 to I.F-1.40c.Y-480, I.F-2.40c.Y-1 to I.F-2.40c.Y-480, I.F-3.40c.Y-1 to I.F-3.40c.Y-480, I.F-4.40c.Y-1 to I.F-4.40c.Y-480, I.F-5.40c.Y-1 to I.F-5.40c.Y-480, I.F-6.40c.Y-1 to I.F-6.40c.Y-480).
Table 41c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3,4-(CH₃)₂-C₆H₃ and the meaning for the combination of R⁶ and R⁷ for each individual compound corresponds in each case to one line of Table Y (compounds I.E-1.41c.Y-1 to I.E-1.41c.Y-480, I.E-2.41c.Y-1 to I.E-2.41c.Y-480, I.E-3.41c.Y-1 to I.E-3.41c.Y-480, I.E-4.41c.Y-1 to I.E-5.41c.Y-480, I.E-5.41c.Y-1 to I.E-3.41c.Y-480, I.E-6.41c.Y-1 to I.E-6.41c.Y-480; I.F-41c.Y-1 to I.F-1.41c.Y-480, I.F-2.41c.Y-1 to I.F-2.41c.Y-480, I.F-3.41c.Y-1 to I.F-3.41c.Y-480, I.F-4.41c.Y-1 to I.F-4.41c.Y-480, I.F-5.41c.Y-1 to I.F-5.41c.Y-480, I.F-6.41c.Y-1 to I.F-6.41c.Y-480).
Table 42c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3,5-(CH₃)₂-C₆H₃ and the meaning for the combination of R⁶ and R⁷ for each individual compound corresponds in each case to one line of Table Y (compounds I.E-1.42c.Y-1 to I.E-1.42c.Y-480, I.E-2.42c.Y-1 to I.E-2.42c.Y-480, I.E-3.42c.Y-1 to I.E-3.42c.Y-480, I.E-4.42c.Y-1 to I.E-5.42c.Y-480, I.E-5.42c.Y-1 to I.E-3.42c.Y-480, I.E-6.42c.Y-1 to I.E-6.42c.Y-480; I.F-42c.Y-1 to I.F-1.42c.Y-480, I.F-2.42c.Y-1 to I.F-2.42c.Y-480, I.F-3.42c.Y-1 to I.F-3.42c.Y-480, I.F-4.42c.Y-1 to I.F-4.42c.Y-480, I.F-5.42c.Y-1 to I.F-5.42c.Y-480, I.F-6.42c.Y-1 to I.F-6.42c.Y-480).
Table 43c Compounds of the formula I.E-1, I.E-2, I.E-3, I.E-4, I.E-5, I.E-6; I.F-1, I.F-2, I.F-3, I.F-4, I.F-5, I.F-6 in which R⁸ is thien-3-yl and the meaning for the combination of R⁶ and R⁷ for each individual compound corresponds in each case to one line of Table Y (compounds I.E-1.43c.Y-1 to I.E-1.43c.Y-480, I.E-2.43c.Y-1 to I.E-2.43c.Y-480, I.E-3.43c.Y-1 to I.E-3.43c.Y-480, I.E-4.43c.Y-1 to I.E-5.43c.Y-480, I.E-5.43c.Y-1 to I.E-3.43c.Y-480, I.E-6.43c.Y-1 to I.E-6.43c.Y-480; I.F-43c.Y-1 to I.F-1.43c.Y-480, I.F-2.43c.Y-1 to I.F-2.43c.Y-480, I.F-3.43c.Y-1 to I.F-3.43c.Y-480, I.F-4.43c.Y-1 to I.F-4.43c.Y-480, I.F-5.43c.Y-1 to I.F-5.43c.Y-480, I.F-6.43c.Y-1 to I.F-6.43c.Y-480).
Table 44c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-CHF₂—C₆H₄ and the meaning for the combination of R⁶ and R⁷ for each individual compound corresponds in each case to one line of Table Y (compounds I.E-1.44c.Y-1 to I.E-1.44c.Y-480, I.E-2.44c.Y-1 to I.E-2.44c.Y-480, I.E-3.44c.Y-1 to I.E-3.44c.Y-480, I.E-4.44c.Y-1 to I.E-5.44c.Y-480, I.E-5.44c.Y-1 to I.E-3.44c.Y-480, I.E-6.44c.Y-1 to I.E-6.44c.Y-480; I.F-19c.Y-1 to I.F-1.44c.Y-480, I.F-2.44c.Y-1 to I.F-2.44c.Y-480, I.F-3.44c.Y-1 to I.F-3.44c.Y-480, I.F-4.44c.Y-1 to I.F-4.44c.Y-480, I.F-5.44c.Y-1 to I.F-5.44c.Y-480, I.F-6.44c.Y-1 to I.F-6.44c.Y-480).
Table 45c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-CHF₂—C₆H₄ and the meaning for the combination of R⁶ and R⁷ for each individual compound corresponds in each case to one line of Table Y (compounds I.E-1.45c.Y-1 to I.E-1.45c.Y-480, I.E-2.45c.Y-1 to I.E-2.45c.Y-480, I.E-3.45c.Y-1 to I.E-3.45c.Y-480, I.E-4.45c.Y-1 to I.E-5.45c.Y-480, I.E-5.45c.Y-1 to I.E-3.45c.Y-480, I.E-6.45c.Y-1 to I.E-6.45c.Y-480; I.F-19c.Y-1 to I.F-1.45c.Y-480, I.F-2.45c.Y-1 to I.F-2.45c.Y-480, I.F-3.45c.Y-1 to I.F-3.45c.Y-480, I.F-4.45c.Y-1 to I.F-4.45c.Y-480, I.F-5.45c.Y-1 to I.F-5.45c.Y-480, I.F-6.45c.Y-1 to I.F-6.45c.Y-480).
Table 46c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-CHF₂—C₆H₄ and the meaning for the combination of R⁶ and R⁷ for each individual compound corresponds in each case to one line of Table Y (compounds I.E-1.46c.Y-1 to I.E-1.46c.Y-480, I.E-2.46c.Y-1 to I.E-2.46c.Y-480, I.E-3.46c.Y-1 to I.E-3.46c.Y-480, I.E-4.46c.Y-1 to I.E-5.46c.Y-480, I.E-5.46c.Y-1 to I.E-3.46c.Y-480, I.E-6.46c.Y-1 to I.E-6.46c.Y-480; I.F-19c.Y-1 to I.F-1.46c.Y-480, I.F-2.46c.Y-1 to I.F-2.46c.Y-480, I.F-3.46c.Y-1 to I.F-3.46c.Y-480, I.F-4.46c.Y-1 to I.F-4.46c.Y-480, I.F-5.46c.Y-1 to I.F-5.46c.Y-480, I.F-6.46c.Y-1 to I.F-6.46c.Y-480).
Table 47c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-OCF₃—C₆H₄ and the meaning for the combination of R⁶ and R⁷ for each individual compound corresponds in each case to one line of Table Y (compounds I.E-1.47c.Y-1 to I.E-1.47c.Y-480, I.E-2.47c.Y-1 to I.E-2.47c.Y-480, I.E-3.47c.Y-1 to I.E-3.47c.Y-480, I.E-4.47c.Y-1 to I.E-5.47c.Y-480, I.E-5.47c.Y-1 to I.E-3.47c.Y-480, I.E-6.47c.Y-1 to I.E-6.47c.Y-480; I.F-19c.Y-1 to I.F-1.47c.Y-480, I.F-2.47c.Y-1 to I.F-2.47c.Y-480, I.F-3.47c.Y-1 to I.F-3.47c.Y-480, I.F-4.47c.Y-1 to I.F-4.47c.Y-480, I.F-5.47c.Y-1 to I.F-5.47c.Y-480, I.F-6.47c.Y-1 to I.F-6.47c.Y-480).
Table 48c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-OCF₃—C₆H₄ and the meaning for the combination of R⁶ and R⁷ for each individual compound corresponds in each case to one line of Table Y (compounds I.E-1.48c.Y-1 to I.E-1.48c.Y-480, I.E-2.48c.Y-1 to I.E-2.48c.Y-480, I.E-3.48c.Y-1 to I.E-3.48c.Y-480, I.E-4.48c.Y-1 to I.E-5.48c.Y-480, I.E-5.48c.Y-1 to I.E-3.48c.Y-480, I.E-6.48c.Y-1 to I.E-6.48c.Y-480; I.F-19c.Y-1 to I.F-1.48c.Y-480, I.F-2.48c.Y-1 to I.F-2.48c.Y-480, I.F-3.48c.Y-1 to I.F-3.48c.Y-480, I.F-4.48c.Y-1 to I.F-4.48c.Y-480, I.F-5.48c.Y-1 to I.F-5.48c.Y-480, I.F-6.48c.Y-1 to I.F-6.48c.Y-480).
Table 49c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-OCF₃—C₆H₄ and the meaning for the combination of R⁶ and R⁷ for each individual compound corresponds in each case to one line of Table Y (compounds I.E-1.49c.Y-1 to I.E-1.49c.Y-480, I.E-2.49c.Y-1 to I.E-2.49c.Y-480, I.E-3.49c.Y-1 to I.E-3.49c.Y-480, I.E-4.49c.Y-1 to I.E-5.49c.Y-480, I.E-5.49c.Y-1 to I.E-3.49c.Y-480, I.E-6.49c.Y-1 to I.E-6.49c.Y-480; I.F-19c.Y-1 to I.F-1.49c.Y-480, I.F-2.49c.Y-1 to I.F-2.49c.Y-480, I.F-3.49c.Y-1 to I.F-3.49c.Y-480, I.F-4.49c.Y-1 to I.F-4.49c.Y-480, I.F-5.49c.Y-1 to I.F-5.49c.Y-480, I.F-6.49c.Y-1 to I.F-6.49c.Y-480).
Table 50c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-CN—C₆H₄ and the meaning for the combination of R⁶ and R⁷ for each individual compound corresponds in each case to one line of Table Y (compounds I.E-1.50c.Y-1 to I.E-1.50c.Y-480, I.E-2.50c.Y-1 to I.E-2.50c.Y-480, I.E-3.50c.Y-1 to I.E-3.50c.Y-480, I.E-4.50c.Y-1 to I.E-5.50c.Y-480, I.E-5.50c.Y-1 to I.E-3.50c.Y-480, I.E-6.50c.Y-1 to I.E-6.50c.Y-480; I.F-19c.Y-1 to I.F-1.50c.Y-480, I.F-2.50c.Y-1 to I.F-2.50c.Y-480, I.F-3.50c.Y-1 to I.F-3.50c.Y-480, I.F-4.50c.Y-1 to I.F-4.50c.Y-480, I.F-5.50c.Y-1 to I.F-5.50c.Y-480, I.F-6.50c.Y-1 to I.F-6.50c.Y-480).
Table 51c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-CN—C₆H₄ and the meaning for the combination of R⁶ and R⁷ for each individual compound corresponds in each case to one line of Table Y (compounds I.E-1.51c.Y-1 to I.E-1.51c.Y-480, I.E-2.51c.Y-1 to I.E-2.51c.Y-480, I.E-3.51c.Y-1 to I.E-3.51c.Y-480, I.E-4.51c.Y-1 to I.E-5.51c.Y-480, I.E-5.51c.Y-1 to I.E-3.51c.Y-480, I.E-6.51c.Y-1 to I.E-6.51c.Y-480; I.F-19c.Y-1 to I.F-1.51c.Y-480, I.F-2.51c.Y-1 to I.F-2.51c.Y-480, I.F-3.51c.Y-1 to I.F-3.51c.Y-480, I.F-4.51c.Y-1 to I.F-4.51c.Y-480, I.F-5.51c.Y-1 to I.F-5.51c.Y-480, I.F-6.51c.Y-1 to I.F-6.51c.Y-480).
Table 52c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-CN—C₆H₄ and the meaning for the combination of R⁶ and R⁷ for each individual compound corresponds in each case to one line of Table Y (compounds I.E-1.52c.Y-1 to I.E-1.52c.Y-480, I.E-2.52c.Y-1 to I.E-2.52c.Y-480, I.E-3.52c.Y-1 to I.E-3.52c.Y-480, I.E-4.52c.Y-1 to I.E-5.52c.Y-480, I.E-5.52c.Y-1 to I.E-3.52c.Y-480, I.E-6.52c.Y-1 to I.E-6.52c.Y-480; I.F-19c.Y-1 to I.F-1.52c.Y-480, I.F-2.52c.Y-1 to I.F-2.52c.Y-480, I.F-3.52c.Y-1 to I.F-3.52c.Y-480, I.F-4.52c.Y-1 to I.F-4.52c.Y-480, I.F-5.52c.Y-1 to I.F-5.52c.Y-480, I.F-6.52c.Y-1 to I.F-6.52c.Y-480).
Table 53c Compounds of the formula I.E-1, I.E-2, I.E-3, I.E-4, I.E-5, I.E-6; I.F-1, I.F-2, I.F-3, I.F-4, I.F-5, I.F-6 in which R⁸ is pyridin-2-yl and the meaning for the combination of R⁶ and R⁷ for each individual compound corresponds in each case to one line of Table Y (compounds I.E-1.53c.Y-1 to I.E-1.53c.Y-480, I.E-2.53c.Y-1 to I.E-2.53c.Y-480, I.E-3.53c.Y-1 to I.E-3.53c.Y-480, I.E-4.53c.Y-1 to I.E-5.53c.Y-480, I.E-5.53c.Y-1 to I.E-3.53c.Y-480, I.E-6.53c.Y-1 to I.E-6.53c.Y-480; I.F-19c.Y-1 to I.F-1.53c.Y-480, I.F-2.53c.Y-1 to I.F-2.53c.Y-480, I.F-3.53c.Y-1 to I.F-3.53c.Y-480, I.F-4.53c.Y-1 to I.F-4.53c.Y-480, I.F-5.53c.Y-1 to I.F-5.53c.Y-480, I.F-6.53c.Y-1 to I.F-6.53c.Y-480).
Table 54c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-F-pyridin-2-yl and the meaning for the combination of R⁶ and R⁷ for each individual compound corresponds in each case to one line of Table Y (compounds I.E-1.54c.Y-1 to I.E-1.54c.Y-480, I.E-2.54c.Y-1 to I.E-2.54c.Y-480, I.E-3.54c.Y-1 to I.E-3.54c.Y-480, I.E-4.54c.Y-1 to I.E-5.54c.Y-480, I.E-5.54c.Y-1 to I.E-3.54c.Y-480, I.E-6.54c.Y-1 to I.E-6.54c.Y-480; I.F-19c.Y-1 to I.F-1.54c.Y-480, I.F-2.54c.Y-1 to I.F-2.54c.Y-480, I.F-3.54c.Y-1 to I.F-3.54c.Y-480, I.F-4.54c.Y-1 to I.F-4.54c.Y-480, I.F-5.54c.Y-1 to I.F-5.54c.Y-480, I.F-6.54c.Y-1 to I.F-6.54c.Y-480).
Table 55c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-F-pyridin-2-yl and the meaning for the combination of R⁶ and R⁷ for each individual compound corresponds in each case to one line of Table Y (compounds I.E-1.55c.Y-1 to I.E-1.55c.Y-480, I.E-2.55c.Y-1 to I.E-2.55c.Y-480, I.E-3.55c.Y-1 to I.E-3.55c.Y-480, I.E-4.55c.Y-1 to I.E-5.55c.Y-480, I.E-5.55c.Y-1 to I.E-3.55c.Y-480, I.E-6.55c.Y-1 to I.E-6.55c.Y-480; I.F-19c.Y-1 to I.F-1.55c.Y-480, I.F-2.55c.Y-1 to I.F-2.55c.Y-480, I.F-3.55c.Y-1 to I.F-3.55c.Y-480, I.F-4.55c.Y-1 to I.F-4.55c.Y-480, I.F-5.55c.Y-1 to I.F-5.55c.Y-480, I.F-6.55c.Y-1 to I.F-6.55c.Y-480).
Table 56c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-F-pyridin-2-yl and the meaning for the combination of R⁶ and R⁷ for each individual compound corresponds in each case to one line of Table Y (compounds I.E-1.56c.Y-1 to I.E-1.56c.Y-480, I.E-2.56c.Y-1 to I.E-2.56c.Y-480, I.E-3.56c.Y-1 to I.E-3.56c.Y-480, I.E-4.56c.Y-1 to I.E-5.56c.Y-480, I.E-5.56c.Y-1 to I.E-3.56c.Y-480, I.E-6.56c.Y-1 to I.E-6.56c.Y-480; I.F-19c.Y-1 to I.F-1.56c.Y-480, I.F-2.56c.Y-1 to I.F-2.56c.Y-480, I.F-3.56c.Y-1 to I.F-3.56c.Y-480, I.F-4.56c.Y-1 to I.F-4.56c.Y-480, I.F-5.56c.Y-1 to I.F-5.56c.Y-480, I.F-6.56c.Y-1 to I.F-6.56c.Y-480).
Table 57c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-F-pyridin-2-yl and the meaning for the combination of R⁶ and R⁷ for each individual compound corresponds in each case to one line of Table Y (compounds I.E-1.57c.Y-1 to I.E-1.57c.Y-480, I.E-2.57c.Y-1 to I.E-2.57c.Y-480, I.E-3.57c.Y-1 to I.E-3.57c.Y-480, I.E-4.57c.Y-1 to I.E-5.57c.Y-480, I.E-5.57c.Y-1 to I.E-3.57c.Y-480, I.E-6.57c.Y-1 to I.E-6.57c.Y-480; I.F-19c.Y-1 to I.F-1.57c.Y-480, I.F-2.57c.Y-1 to I.F-2.57c.Y-480, I.F-3.57c.Y-1 to I.F-3.57c.Y-480, I.F-4.57c.Y-1 to I.F-4.57c.Y-480, I.F-5.57c.Y-1 to I.F-5.57c.Y-480, I.F-6.57c.Y-1 to I.F-6.57c.Y-480).
Table 58c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-Cl-pyridin-2-yl and the meaning for the combination of R⁶ and R⁷ for each individual compound corresponds in each case to one line of Table Y (compounds I.E-1.58c.Y-1 to I.E-1.58c.Y-480, I.E-2.58c.Y-1 to I.E-2.58c.Y-480, I.E-3.58c.Y-1 to I.E-3.58c.Y-480, I.E-4.58c.Y-1 to I.E-5.58c.Y-480, I.E-5.58c.Y-1 to I.E-3.58c.Y-480, I.E-6.58c.Y-1 to I.E-6.58c.Y-480; I.F-19c.Y-1 to I.F-1.58c.Y-480, I.F-2.58c.Y-1 to I.F-2.58c.Y-480, I.F-3.58c.Y-1 to I.F-3.58c.Y-480, I.F-4.58c.Y-1 to I.F-4.58c.Y-480, I.F-5.58c.Y-1 to I.F-5.58c.Y-480, I.F-6.58c.Y-1 to I.F-6.58c.Y-480).
Table 59c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-Cl-pyridin-2-yl and the meaning for the combination of R⁶ and R⁷ for each individual compound corresponds in each case to one line of Table Y (compounds I.E-1.59c.Y-1 to I.E-1.59c.Y-480, I.E-2.59c.Y-1 to I.E-2.59c.Y-480, I.E-3.59c.Y-1 to I.E-3.59c.Y-480, I.E-4.59c.Y-1 to I.E-5.59c.Y-480, I.E-5.59c.Y-1 to I.E-3.59c.Y-480, I.E-6.59c.Y-1 to I.E-6.59c.Y-480; I.F-19c.Y-1 to I.F-1.59c.Y-480, I.F-2.59c.Y-1 to I.F-2.59c.Y-480, I.F-3.59c.Y-1 to I.F-3.59c.Y-480, I.F-4.59c.Y-1 to I.F-4.59c.Y-480, I.F-5.59c.Y-1 to I.F-5.59c.Y-480, I.F-6.59c.Y-1 to I.F-6.59c.Y-480).
Table 60c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-Cl-pyridin-2-yl and the meaning for the combination of R⁶ and R⁷ for each individual compound corresponds in each case to one line of Table Y (compounds I.E-1.60c.Y-1 to I.E-1.60c.Y-480, I.E-2.60c.Y-1 to I.E-2.60c.Y-480, I.E-3.60c.Y-1 to I.E-3.60c.Y-480, I.E-4.60c.Y-1 to I.E-5.60c.Y-480, I.E-5.60c.Y-1 to I.E-3.60c.Y-480, I.E-6.60c.Y-1 to I.E-6.60c.Y-480; I.F-19c.Y-1 to I.F-1.60c.Y-480, I.F-2.60c.Y-1 to I.F-2.60c.Y-480, I.F-3.60c.Y-1 to I.F-3.60c.Y-480, I.F-4.60c.Y-1 to I.F-4.60c.Y-480, I.F-5.60c.Y-1 to I.F-5.60c.Y-480, I.F-6.60c.Y-1 to I.F-6.60c.Y-480).
Table 61c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-Cl-pyridin-2-yl and the meaning for the combination of R⁶ and R⁷ for each individual compound corresponds in each case to one line of Table Y (compounds I.E-1.61c.Y-1 to I.E-1.61c.Y-480, I.E-2.61c.Y-1 to I.E-2.61c.Y-480, I.E-3.61c.Y-1 to I.E-3.61c.Y-480, I.E-4.61c.Y-1 to I.E-5.61c.Y-480, I.E-5.61c.Y-1 to I.E-3.61c.Y-480, I.E-6.61c.Y-1 to I.E-6.61c.Y-480; I.F-19c.Y-1 to I.F-1.61c.Y-480, I.F-2.61c.Y-1 to I.F-2.61c.Y-480, I.F-3.61c.Y-1 to I.F-3.61c.Y-480, I.F-4.61c.Y-1 to I.F-4.61c.Y-480, I.F-5.61c.Y-1 to I.F-5.61c.Y-480, I.F-6.61c.Y-1 to I.F-6.61c.Y-480).
Table 62c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-CH₃-pyridin-2-yl and the meaning for the combination of R⁶ and R⁷ for each individual compound corresponds in each case to one line of Table Y (compounds I.E-1.62c.Y-1 to I.E-1.62c.Y-480, I.E-2.62c.Y-1 to I.E-2.62c.Y-480, I.E-3.62c.Y-1 to I.E-3.62c.Y-480, I.E-4.62c.Y-1 to I.E-5.62c.Y-480, I.E-5.62c.Y-1 to I.E-3.62c.Y-480, I.E-6.62c.Y-1 to I.E-6.62c.Y-480; I.F-19c.Y-1 to I.F-1.62c.Y-480, I.F-2.62c.Y-1 to I.F-2.62c.Y-480, I.F-3.62c.Y-1 to I.F-3.62c.Y-480, I.F-4.62c.Y-1 to I.F-4.62c.Y-480, I.F-5.62c.Y-1 to I.F-5.62c.Y-480, I.F-6.62c.Y-1 to I.F-6.62c.Y-480).
Table 63c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-CH₃-pyridin-2-yl and the meaning for the combination of R⁶ and R⁷ for each individual compound corresponds in each case to one line of Table Y (compounds I.E-1.63c.Y-1 to I.E-1.63c.Y-480, I.E-2.63c.Y-1 to I.E-2.63c.Y-480, I.E-3.63c.Y-1 to I.E-3.63c.Y-480, I.E-4.63c.Y-1 to I.E-5.63c.Y-480, I.E-5.63c.Y-1 to I.E-3.63c.Y-480, I.E-6.63c.Y-1 to I.E-6.63c.Y-480; I.F-19c.Y-1 to I.F-1.63c.Y-480, I.F-2.63c.Y-1 to I.F-2.63c.Y-480, I.F-3.63c.Y-1 to I.F-3.63c.Y-480, I.F-4.63c.Y-1 to I.F-4.63c.Y-480, I.F-5.63c.Y-1 to I.F-5.63c.Y-480, I.F-6.63c.Y-1 to I.F-6.63c.Y-480).
Table 64c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-CH₃-pyridin-2-yl and the meaning for the combination of R⁶ and R⁷ for each individual compound corresponds in each case to one line of Table Y (compounds I.E-1.64c.Y-1 to I.E-1.64c.Y-480, I.E-2.64c.Y-1 to I.E-2.64c.Y-480, I.E-3.64c.Y-1 to I.E-3.64c.Y-480, I.E-4.64c.Y-1 to I.E-5.64c.Y-480, I.E-5.64c.Y-1 to I.E-3.64c.Y-480, I.E-6.64c.Y-1 to I.E-6.64c.Y-480; I.F-19c.Y-1 to I.F-1.64c.Y-480, I.F-2.64c.Y-1 to I.F-2.64c.Y-480, I.F-3.64c.Y-1 to I.F-3.64c.Y-480, I.F-4.64c.Y-1 to I.F-4.64c.Y-480, I.F-5.64c.Y-1 to I.F-5.64c.Y-480, I.F-6.64c.Y-1 to I.F-6.64c.Y-480).
Table 65c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-CH₃-pyridin-2-yl and the meaning for the combination of R⁶ and R⁷ for each individual compound corresponds in each case to one line of Table Y (compounds I.E-1.65c.Y-1 to I.E-1.65c.Y-480, I.E-2.65c.Y-1 to I.E-2.65c.Y-480, I.E-3.65c.Y-1 to I.E-3.65c.Y-480, I.E-4.65c.Y-1 to I.E-5.65c.Y-480, I.E-5.65c.Y-1 to I.E-3.65c.Y-480, I.E-6.65c.Y-1 to I.E-6.65c.Y-480; I.F-19c.Y-1 to I.F-1.65c.Y-480, I.F-2.65c.Y-1 to I.F-2.65c.Y-480, I.F-3.65c.Y-1 to I.F-3.65c.Y-480, I.F-4.65c.Y-1 to I.F-4.65c.Y-480, I.F-5.65c.Y-1 to I.F-5.65c.Y-480, I.F-6.65c.Y-1 to I.F-6.65c.Y-480).
Table 66c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-OCH₃-pyridin-2-yl and the meaning for the combination of R⁶ and R⁷ for each individual compound corresponds in each case to one line of Table Y (compounds I.E-1.66c.Y-1 to I.E-1.66c.Y-480, I.E-2.66c.Y-1 to I.E-2.66c.Y-480, I.E-3.66c.Y-1 to I.E-3.66c.Y-480, I.E-4.66c.Y-1 to I.E-5.66c.Y-480, I.E-5.66c.Y-1 to I.E-3.66c.Y-480, I.E-6.66c.Y-1 to I.E-6.66c.Y-480; I.F-19c.Y-1 to I.F-1.66c.Y-480, I.F-2.66c.Y-1 to I.F-2.66c.Y-480, I.F-3.66c.Y-1 to I.F-3.66c.Y-480, I.F-4.66c.Y-1 to I.F-4.66c.Y-480, I.F-5.66c.Y-1 to I.F-5.66c.Y-480, I.F-6.66c.Y-1 to I.F-6.66c.Y-480).
Table 67c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-OCH₃-pyridin-2-yl and the meaning for the combination of R⁶ and R⁷ for each individual compound corresponds in each case to one line of Table Y (compounds I.E-1.67c.Y-1 to I.E-1.67c.Y-480, I.E-2.67c.Y-1 to I.E-2.67c.Y-480, I.E-3.67c.Y-1 to I.E-3.67c.Y-480, I.E-4.67c.Y-1 to I.E-5.67c.Y-480, I.E-5.67c.Y-1 to I.E-3.67c.Y-480, I.E-6.67c.Y-1 to I.E-6.67c.Y-480; I.F-19c.Y-1 to I.F-1.67c.Y-480, I.F-2.67c.Y-1 to I.F-2.67c.Y-480, I.F-3.67c.Y-1 to I.F-3.67c.Y-480, I.F-4.67c.Y-1 to I.F-4.67c.Y-480, I.F-5.67c.Y-1 to I.F-5.67c.Y-480, I.F-6.67c.Y-1 to I.F-6.67c.Y-480).
Table 68c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-OCH₃-pyridin-2-yl and the meaning for the combination of R⁶ and R⁷ for each individual compound corresponds in each case to one line of Table Y (compounds I.E-1.68c.Y-1 to I.E-1.68c.Y-480, I.E-2.68c.Y-1 to I.E-2.68c.Y-480, I.E-3.68c.Y-1 to I.E-3.68c.Y-480, I.E-4.68c.Y-1 to I.E-5.68c.Y-480, I.E-5.68c.Y-1 to I.E-3.68c.Y-480, I.E-6.68c.Y-1 to I.E-6.68c.Y-480; I.F-19c.Y-1 to I.F-1.68c.Y-480, I.F-2.68c.Y-1 to I.F-2.68c.Y-480, I.F-3.68c.Y-1 to I.F-3.68c.Y-480, I.F-4.68c.Y-1 to I.F-4.68c.Y-480, I.F-5.68c.Y-1 to I.F-5.68c.Y-480, I.F-6.68c.Y-1 to I.F-6.68c.Y-480).
Table 69c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-OCH₃-pyridin-2-yl and the meaning for the combination of R⁶ and R⁷ for each individual compound corresponds in each case to one line of Table Y (compounds I.E-1.69c.Y-1 to I.E-1.69c.Y-480, I.E-2.69c.Y-1 to I.E-2.69c.Y-480, I.E-3.69c.Y-1 to I.E-3.69c.Y-480, I.E-4.69c.Y-1 to I.E-5.69c.Y-480, I.E-5.69c.Y-1 to I.E-3.69c.Y-480, I.E-6.69c.Y-1 to I.E-6.69c.Y-480; I.F-19c.Y-1 to I.F-1.69c.Y-480, I.F-2.69c.Y-1 to I.F-2.69c.Y-480, I.F-3.69c.Y-1 to I.F-3.69c.Y-480, I.F-4.69c.Y-1 to I.F-4.69c.Y-480, I.F-5.69c.Y-1 to I.F-5.69c.Y-480, I.F-6.69c.Y-1 to I.F-6.69c.Y-480).
Table 70c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-CHF₂-pyridin-2-yl and the meaning for the combination of R⁶ and R⁷ for each individual compound corresponds in each case to one line of Table Y (compounds I.E-1.70c.Y-1 to I.E-1.70c.Y-480, I.E-2.70c.Y-1 to I.E-2.70c.Y-480, I.E-3.70c.Y-1 to I.E-3.70c.Y-480, I.E-4.70c.Y-1 to I.E-5.70c.Y-480, I.E-5.70c.Y-1 to I.E-3.70c.Y-480, I.E-6.70c.Y-1 to I.E-6.70c.Y-480; I.F-19c.Y-1 to I.F-1.70c.Y-480, I.F-2.70c.Y-1 to I.F-2.70c.Y-480, I.F-3.70c.Y-1 to I.F-3.70c.Y-480, I.F-4.70c.Y-1 to I.F-4.70c.Y-480, I.F-5.70c.Y-1 to I.F-5.70c.Y-480, I.F-6.70c.Y-1 to I.F-6.70c.Y-480).
Table 71c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-CHF₂-pyridin-2-yl and the meaning for the combination of R⁶ and R⁷ for each individual compound corresponds in each case to one line of Table Y (compounds I.E-1.71c.Y-1 to I.E-1.71c.Y-480, I.E-2.71c.Y-1 to I.E-2.71c.Y-480, I.E-3.71c.Y-1 to I.E-3.71c.Y-480, I.E-4.71c.Y-1 to I.E-5.71c.Y-480, I.E-5.71c.Y-1 to I.E-3.71c.Y-480, I.E-6.71c.Y-1 to I.E-6.71c.Y-480; I.F-19c.Y-1 to I.F-1.71c.Y-480, I.F-2.71c.Y-1 to I.F-2.71c.Y-480, I.F-3.71c.Y-1 to I.F-3.71c.Y-480, I.F-4.71c.Y-1 to I.F-4.71c.Y-480, I.F-5.71c.Y-1 to I.F-5.71c.Y-480, I.F-6.71c.Y-1 to I.F-6.71c.Y-480).
Table 72c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-CHF₂-pyridin-2-yl and the meaning for the combination of R⁶ and R⁷ for each individual compound corresponds in each case to one line of Table Y (compounds I.E-1.72c.Y-1 to I.E-1.72c.Y-480, I.E-2.72c.Y-1 to I.E-2.72c.Y-480, I.E-3.72c.Y-1 to I.E-3.72c.Y-480, I.E-4.72c.Y-1 to I.E-5.72c.Y-480, I.E-5.72c.Y-1 to I.E-3.72c.Y-480, I.E-6.72c.Y-1 to I.E-6.72c.Y-480; I.F-19c.Y-1 to I.F-1.72c.Y-480, I.F-2.72c.Y-1 to I.F-2.72c.Y-480, I.F-3.72c.Y-1 to I.F-3.72c.Y-480, I.F-4.72c.Y-1 to I.F-4.72c.Y-480, I.F-5.72c.Y-1 to I.F-5.72c.Y-480, I.F-6.72c.Y-1 to I.F-6.72c.Y-480).
Table 73c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-CHF₂-pyridin-2-yl and the meaning for the combination of R⁶ and R⁷ for each individual compound corresponds in each case to one line of Table Y (compounds I.E-1.73c.Y-1 to I.E-1.73c.Y-480, I.E-2.73c.Y-1 to I.E-2.73c.Y-480, I.E-3.73c.Y-1 to I.E-3.73c.Y-480, I.E-4.73c.Y-1 to I.E-5.73c.Y-480, I.E-5.73c.Y-1 to I.E-3.73c.Y-480, I.E-6.73c.Y-1 to I.E-6.73c.Y-480; I.F-19c.Y-1 to I.F-1.73c.Y-480, I.F-2.73c.Y-1 to I.F-2.73c.Y-480, I.F-3.73c.Y-1 to I.F-3.73c.Y-480, I.F-4.73c.Y-1 to I.F-4.73c.Y-480, I.F-5.73c.Y-1 to I.F-5.73c.Y-480, I.F-6.73c.Y-1 to I.F-6.73c.Y-480).
Table 74c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-CF₃-pyridin-2-yl and the meaning for the combination of R⁶ and R⁷ for each individual compound corresponds in each case to one line of Table Y (compounds I.E-1.74c.Y-1 to I.E-1.74c.Y-480, I.E-2.74c.Y-1 to I.E-2.74c.Y-480, I.E-3.74c.Y-1 to I.E-3.74c.Y-480, I.E-4.74c.Y-1 to I.E-5.74c.Y-480, I.E-5.74c.Y-1 to I.E-3.74c.Y-480, I.E-6.74c.Y-1 to I.E-6.74c.Y-480; I.F-19c.Y-1 to I.F-1.74c.Y-480, I.F-2.74c.Y-1 to I.F-2.74c.Y-480, I.F-3.74c.Y-1 to I.F-3.74c.Y-480, I.F-4.74c.Y-1 to I.F-4.74c.Y-480, I.F-5.74c.Y-1 to I.F-5.74c.Y-480, I.F-6.74c.Y-1 to I.F-6.74c.Y-480).
Table 75c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-CF₃-pyridin-2-yl and the meaning for the combination of R⁶ and R⁷ for each individual compound corresponds in each case to one line of Table Y (compounds I.E-1.75c.Y-1 to I.E-1.75c.Y-480, I.E-2.75c.Y-1 to I.E-2.75c.Y-480, I.E-3.75c.Y-1 to I.E-3.75c.Y-480, I.E-4.75c.Y-1 to I.E-5.75c.Y-480, I.E-5.75c.Y-1 to I.E-3.75c.Y-480, I.E-6.75c.Y-1 to I.E-6.75c.Y-480; I.F-19c.Y-1 to I.F-1.75c.Y-480, I.F-2.75c.Y-1 to I.F-2.75c.Y-480, I.F-3.75c.Y-1 to I.F-3.75c.Y-480, I.F-4.75c.Y-1 to I.F-4.75c.Y-480, I.F-5.75c.Y-1 to I.F-5.75c.Y-480, I.F-6.75c.Y-1 to I.F-6.75c.Y-480).
Table 76c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-CF₃-pyridin-2-yl and the meaning for the combination of R⁶ and R⁷ for each individual compound corresponds in each case to one line of Table Y (compounds I.E-1.76c.Y-1 to I.E-1.76c.Y-480, I.E-2.76c.Y-1 to I.E-2.76c.Y-480, I.E-3.76c.Y-1 to I.E-3.76c.Y-480, I.E-4.76c.Y-1 to I.E-5.76c.Y-480, I.E-5.76c.Y-1 to I.E-3.76c.Y-480, I.E-6.76c.Y-1 to I.E-6.76c.Y-480; I.F-19c.Y-1 to I.F-1.76c.Y-480, I.F-2.76c.Y-1 to I.F-2.76c.Y-480, I.F-3.76c.Y-1 to I.F-3.76c.Y-480, I.F-4.76c.Y-1 to I.F-4.76c.Y-480, I.F-5.76c.Y-1 to I.F-5.76c.Y-480, I.F-6.76c.Y-1 to I.F-6.76c.Y-480).
Table 77c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-CF₃-pyridin-2-yl and the meaning for the combination of R⁶ and R⁷ for each individual compound corresponds in each case to one line of Table Y (compounds I.E-1.77c.Y-1 to I.E-1.77c.Y-480, I.E-2.77c.Y-1 to I.E-2.77c.Y-480, I.E-3.77c.Y-1 to I.E-3.77c.Y-480, I.E-4.77c.Y-1 to I.E-5.77c.Y-480, I.E-5.77c.Y-1 to I.E-3.77c.Y-480, I.E-6.77c.Y-1 to I.E-6.77c.Y-480; I.F-19c.Y-1 to I.F-1.77c.Y-480, I.F-2.77c.Y-1 to I.F-2.77c.Y-480, I.F-3.77c.Y-1 to I.F-3.77c.Y-480, I.F-4.77c.Y-1 to I.F-4.77c.Y-480, I.F-5.77c.Y-1 to I.F-5.77c.Y-480, I.F-6.77c.Y-1 to I.F-6.77c.Y-480).
Table 78c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-OCF₃-pyridin-2-yl and the meaning for the combination of R⁶ and R⁷ for each individual compound corresponds in each case to one line of Table Y (compounds I.E-1.78c.Y-1 to I.E-1.78c.Y-480, I.E-2.78c.Y-1 to I.E-2.78c.Y-480, I.E-3.78c.Y-1 to I.E-3.78c.Y-480, I.E-4.78c.Y-1 to I.E-5.78c.Y-480, I.E-5.78c.Y-1 to I.E-3.78c.Y-480, I.E-6.78c.Y-1 to I.E-6.78c.Y-480; I.F-19c.Y-1 to I.F-1.78c.Y-480, I.F-2.78c.Y-1 to I.F-2.78c.Y-480, I.F-3.78c.Y-1 to I.F-3.78c.Y-480, I.F-4.78c.Y-1 to I.F-4.78c.Y-480, I.F-5.78c.Y-1 to I.F-5.78c.Y-480, I.F-6.78c.Y-1 to I.F-6.78c.Y-480).
Table 79c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-OCF₃-pyridin-2-yl and the meaning for the combination of R⁶ and R⁷ for each individual compound corresponds in each case to one line of Table Y (compounds I.E-1.79c.Y-1 to I.E-1.79c.Y-480, I.E-2.79c.Y-1 to I.E-2.79c.Y-480, I.E-3.79c.Y-1 to I.E-3.79c.Y-480, I.E-4.79c.Y-1 to I.E-5.79c.Y-480, I.E-5.79c.Y-1 to I.E-3.79c.Y-480, I.E-6.79c.Y-1 to I.E-6.79c.Y-480; I.F-19c.Y-1 to I.F-1.79c.Y-480, I.F-2.79c.Y-1 to I.F-2.79c.Y-480, I.F-3.79c.Y-1 to I.F-3.79c.Y-480, I.F-4.79c.Y-1 to I.F-4.79c.Y-480, I.F-5.79c.Y-1 to I.F-5.79c.Y-480, I.F-6.79c.Y-1 to I.F-6.79c.Y-480).
Table 80c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-OCF₃-pyridin-2-yl and the meaning for the combination of R⁶ and R⁷ for each individual compound corresponds in each case to one line of Table Y (compounds I.E-1.80c.Y-1 to I.E-1.80c.Y-480, I.E-2.80c.Y-1 to I.E-2.80c.Y-480, I.E-3.80c.Y-1 to I.E-3.80c.Y-480, I.E-4.80c.Y-1 to I.E-5.80c.Y-480, I.E-5.80c.Y-1 to I.E-3.80c.Y-480, I.E-6.80c.Y-1 to I.E-6.80c.Y-480; I.F-19c.Y-1 to I.F-1.80c.Y-480, I.F-2.80c.Y-1 to I.F-2.80c.Y-480, I.F-3.80c.Y-1 to I.F-3.80c.Y-480, I.F-4.80c.Y-1 to I.F-4.80c.Y-480, I.F-5.80c.Y-1 to I.F-5.80c.Y-480, I.F-6.80c.Y-1 to I.F-6.80c.Y-480).
Table 81c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-OCF₃-pyridin-2-yl and the meaning for the combination of R⁶ and R⁷ for each individual compound corresponds in each case to one line of Table Y (compounds I.E-1.81c.Y-1 to I.E-1.81c.Y-480, I.E-2.81c.Y-1 to I.E-2.81c.Y-480, I.E-3.81c.Y-1 to I.E-3.81c.Y-480, I.E-4.81c.Y-1 to I.E-5.81c.Y-480, I.E-5.81c.Y-1 to I.E-3.81c.Y-480, I.E-6.81c.Y-1 to I.E-6.81c.Y-480; I.F-19c.Y-1 to I.F-1.81c.Y-480, I.F-2.81c.Y-1 to I.F-2.81c.Y-480, I.F-3.81c.Y-1 to I.F-3.81c.Y-480, I.F-4.81c.Y-1 to I.F-4.81c.Y-480, I.F-5.81c.Y-1 to I.F-5.81c.Y-480, I.F-6.81c.Y-1 to I.F-6.81c.Y-480).
Table 82c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-OCHF₂-pyridin-2-yl and the meaning for the combination of R⁶ and R⁷ for each individual compound corresponds in each case to one line of Table Y (compounds I.E-1.82c.Y-1 to I.E-1.82c.Y-480, I.E-2.82c.Y-1 to I.E-2.82c.Y-480, I.E-3.82c.Y-1 to I.E-3.82c.Y-480, I.E-4.82c.Y-1 to I.E-5.82c.Y-480, I.E-5.82c.Y-1 to I.E-3.82c.Y-480, I.E-6.82c.Y-1 to I.E-6.82c.Y-480; I.F-19c.Y-1 to I.F-1.82c.Y-480, I.F-2.82c.Y-1 to I.F-2.82c.Y-480, I.F-3.82c.Y-1 to I.F-3.82c.Y-480, I.F-4.82c.Y-1 to I.F-4.82c.Y-480, I.F-5.82c.Y-1 to I.F-5.82c.Y-480, I.F-6.82c.Y-1 to I.F-6.82c.Y-480).
Table 83c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-OCHF₂-pyridin-2-yl and the meaning for the combination of R⁶ and R⁷ for each individual compound corresponds in each case to one line of Table Y (compounds I.E-1.83c.Y-1 to I.E-1.83c.Y-480, I.E-2.83c.Y-1 to I.E-2.83c.Y-480, I.E-3.83c.Y-1 to I.E-3.83c.Y-480, I.E-4.83c.Y-1 to I.E-5.83c.Y-480, I.E-5.83c.Y-1 to I.E-3.83c.Y-480, I.E-6.83c.Y-1 to I.E-6.83c.Y-480; I.F-19c.Y-1 to I.F-1.83c.Y-480, I.F-2.83c.Y-1 to I.F-2.83c.Y-480, I.F-3.83c.Y-1 to I.F-3.83c.Y-480, I.F-4.83c.Y-1 to I.F-4.83c.Y-480, I.F-5.83c.Y-1 to I.F-5.83c.Y-480, I.F-6.83c.Y-1 to I.F-6.83c.Y-480).
Table 84c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-OCHF₂-pyridin-2-yl and the meaning for the combination of R⁶ and R⁷ for each individual compound corresponds in each case to one line of Table Y (compounds I.E-1.84c.Y-1 to I.E-1.84c.Y-480, I.E-2.84c.Y-1 to I.E-2.84c.Y-480, I.E-3.84c.Y-1 to I.E-3.84c.Y-480, I.E-4.84c.Y-1 to I.E-5.84c.Y-480, I.E-5.84c.Y-1 to I.E-3.84c.Y-480, I.E-6.84c.Y-1 to I.E-6.84c.Y-480; I.F-19c.Y-1 to I.F-1.84c.Y-480, I.F-2.84c.Y-1 to I.F-2.84c.Y-480, I.F-3.84c.Y-1 to I.F-3.84c.Y-480, I.F-4.84c.Y-1 to I.F-4.84c.Y-480, I.F-5.84c.Y-1 to I.F-5.84c.Y-480, I.F-6.84c.Y-1 to I.F-6.84c.Y-480).
Table 85c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-OCHF₂-pyridin-2-yl and the meaning for the combination of R⁶ and R⁷ for each individual compound corresponds in each case to one line of Table Y (compounds I.E-1.85c.Y-1 to I.E-1.85c.Y-480, I.E-2.85c.Y-1 to I.E-2.85c.Y-480, I.E-3.85c.Y-1 to I.E-3.85c.Y-480, I.E-4.85c.Y-1 to I.E-5.85c.Y-480, I.E-5.85c.Y-1 to I.E-3.85c.Y-480, I.E-6.85c.Y-1 to I.E-6.85c.Y-480; I.F-19c.Y-1 to I.F-1.85c.Y-480, I.F-2.85c.Y-1 to I.F-2.85c.Y-480, I.F-3.85c.Y-1 to I.F-3.85c.Y-480, I.F-4.85c.Y-1 to I.F-4.85c.Y-480, I.F-5.85c.Y-1 to I.F-5.85c.Y-480, I.F-6.85c.Y-1 to I.F-6.85c.Y-480).
Table 86c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-CN-pyridin-2-yl and the meaning for the combination of R⁶ and R⁷ for each individual compound corresponds in each case to one line of Table Y (compounds I.E-1.86c.Y-1 to I.E-1.86c.Y-480, I.E-2.86c.Y-1 to I.E-2.86c.Y-480, I.E-3.86c.Y-1 to I.E-3.86c.Y-480, I.E-4.86c.Y-1 to I.E-5.86c.Y-480, I.E-5.86c.Y-1 to I.E-3.86c.Y-480, I.E-6.86c.Y-1 to I.E-6.86c.Y-480; I.F-19c.Y-1 to I.F-1.86c.Y-480, I.F-2.86c.Y-1 to I.F-2.86c.Y-480, I.F-3.86c.Y-1 to I.F-3.86c.Y-480, I.F-4.86c.Y-1 to I.F-4.86c.Y-480, I.F-5.86c.Y-1 to I.F-5.86c.Y-480, I.F-6.86c.Y-1 to I.F-6.86c.Y-480).
Table 87c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-CN-pyridin-2-yl and the meaning for the combination of R⁶ and R⁷ for each individual compound corresponds in each case to one line of Table Y (compounds I.E-1.87c.Y-1 to I.E-1.87c.Y-480, I.E-2.87c.Y-1 to I.E-2.87c.Y-480, I.E-3.87c.Y-1 to I.E-3.87c.Y-480, I.E-4.87c.Y-1 to I.E-5.87c.Y-480, I.E-5.87c.Y-1 to I.E-3.87c.Y-480, I.E-6.87c.Y-1 to I.E-6.87c.Y-480; I.F-19c.Y-1 to I.F-1.87c.Y-480, I.F-2.87c.Y-1 to I.F-2.87c.Y-480, I.F-3.87c.Y-1 to I.F-3.87c.Y-480, I.F-4.87c.Y-1 to I.F-4.87c.Y-480, I.F-5.87c.Y-1 to I.F-5.87c.Y-480, I.F-6.87c.Y-1 to I.F-6.87c.Y-480).
Table 88c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-CN-pyridin-2-yl and the meaning for the combination of R⁶ and R⁷ for each individual compound corresponds in each case to one line of Table Y (compounds I.E-1.88c.Y-1 to I.E-1.88c.Y-480, I.E-2.88c.Y-1 to I.E-2.88c.Y-480, I.E-3.88c.Y-1 to I.E-3.88c.Y-480, I.E-4.88c.Y-1 to I.E-5.88c.Y-480, I.E-5.88c.Y-1 to I.E-3.88c.Y-480, I.E-6.88c.Y-1 to I.E-6.88c.Y-480; I.F-19c.Y-1 to I.F-1.88c.Y-480, I.F-2.88c.Y-1 to I.F-2.88c.Y-480, I.F-3.88c.Y-1 to I.F-3.88c.Y-480, I.F-4.88c.Y-1 to I.F-4.88c.Y-480, I.F-5.88c.Y-1 to I.F-5.88c.Y-480, I.F-6.88c.Y-1 to I.F-6.88c.Y-480).
Table 89c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-CN-pyridin-2-yl and the meaning for the combination of R⁶ and R⁷ for each individual compound corresponds in each case to one line of Table Y (compounds I.E-1.89c.Y-1 to I.E-1.89c.Y-480, I.E-2.89c.Y-1 to I.E-2.89c.Y-480, I.E-3.89c.Y-1 to I.E-3.89c.Y-480, I.E-4.89c.Y-1 to I.E-5.89c.Y-480, I.E-5.89c.Y-1 to I.E-3.89c.Y-480, I.E-6.89c.Y-1 to I.E-6.89c.Y-480; I.F-19c.Y-1 to I.F-1.89c.Y-480, I.F-2.89c.Y-1 to I.F-2.89c.Y-480, I.F-3.89c.Y-1 to I.F-3.89c.Y-480, I.F-4.89c.Y-1 to I.F-4.89c.Y-480, I.F-5.89c.Y-1 to I.F-5.89c.Y-480, I.F-6.89c.Y-1 to I.F-6.89c.Y-480).
Table 90c Compounds of the formula I.E-1, I.E-2, I.E-3, I.E-4, I.E-5, I.E-6; I.F-1, I.F-2, I.F-3, I.F-4, I.F-5, I.F-6 in which R⁸ is pyridin-3-yl and the meaning for the combination of R⁶ and R⁷ for each individual compound corresponds in each case to one line of Table Y (compounds I.E-1.90c.Y-1 to I.E-1.90c.Y-480, I.E-2.90c.Y-1 to I.E-2.90c.Y-480, I.E-3.90c.Y-1 to I.E-3.90c.Y-480, I.E-4.90c.Y-1 to I.E-5.90c.Y-480, I.E-5.90c.Y-1 to I.E-3.90c.Y-480, I.E-6.90c.Y-1 to I.E-6.90c.Y-480; I.F-19c.Y-1 to I.F-1.90c.Y-480, I.F-2.90c.Y-1 to I.F-2.90c.Y-480, I.F-3.90c.Y-1 to I.F-3.90c.Y-480, I.F-4.90c.Y-1 to I.F-4.90c.Y-480, I.F-5.90c.Y-1 to I.F-5.90c.Y-480, I.F-6.90c.Y-1 to I.F-6.90c.Y-480).
Table 91c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-F-pyridin-3-yl and the meaning for the combination of R⁶ and R⁷ for each individual compound corresponds in each case to one line of Table Y (compounds I.E-1.91c.Y-1 to I.E-1.91c.Y-480, I.E-2.91c.Y-1 to I.E-2.91c.Y-480, I.E-3.91c.Y-1 to I.E-3.91c.Y-480, I.E-4.91c.Y-1 to I.E-5.91c.Y-480, I.E-5.91c.Y-1 to I.E-3.91c.Y-480, I.E-6.91c.Y-1 to I.E-6.91c.Y-480; I.F-19c.Y-1 to I.F-1.91c.Y-480, I.F-2.91c.Y-1 to I.F-2.91c.Y-480, I.F-3.91c.Y-1 to I.F-3.91c.Y-480, I.F-4.91c.Y-1 to I.F-4.91c.Y-480, I.F-5.91c.Y-1 to I.F-5.91c.Y-480, I.F-6.91c.Y-1 to I.F-6.91c.Y-480).
Table 92c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-F-pyridin-3-yl and the meaning for the combination of R⁶ and R⁷ for each individual compound corresponds in each case to one line of Table Y (compounds I.E-1.92c.Y-1 to I.E-1.92c.Y-480, I.E-2.92c.Y-1 to I.E-2.92c.Y-480, I.E-3.92c.Y-1 to I.E-3.92c.Y-480, I.E-4.92c.Y-1 to I.E-5.92c.Y-480, I.E-5.92c.Y-1 to I.E-3.92c.Y-480, I.E-6.92c.Y-1 to I.E-6.92c.Y-480; I.F-19c.Y-1 to I.F-1.92c.Y-480, I.F-2.92c.Y-1 to I.F-2.92c.Y-480, I.F-3.92c.Y-1 to I.F-3.92c.Y-480, I.F-4.92c.Y-1 to I.F-4.92c.Y-480, I.F-5.92c.Y-1 to I.F-5.92c.Y-480, I.F-6.92c.Y-1 to I.F-6.92c.Y-480).
Table 93c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-F-pyridin-3-yl and the meaning for the combination of R⁶ and R⁷ for each individual compound corresponds in each case to one line of Table Y (compounds I.E-1.93c.Y-1 to I.E-1.93c.Y-480, I.E-2.93c.Y-1 to I.E-2.93c.Y-480, I.E-3.93c.Y-1 to I.E-3.93c.Y-480, I.E-4.93c.Y-1 to I.E-5.93c.Y-480, I.E-5.93c.Y-1 to I.E-3.93c.Y-480, I.E-6.93c.Y-1 to I.E-6.93c.Y-480; I.F-19c.Y-1 to I.F-1.93c.Y-480, I.F-2.93c.Y-1 to I.F-2.93c.Y-480, I.F-3.93c.Y-1 to I.F-3.93c.Y-480, I.F-4.93c.Y-1 to I.F-4.93c.Y-480, I.F-5.93c.Y-1 to I.F-5.93c.Y-480, I.F-6.93c.Y-1 to I.F-6.93c.Y-480).
Table 94c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-F-pyridin-3-yl and the meaning for the combination of R⁶ and R⁷ for each individual compound corresponds in each case to one line of Table Y (compounds I.E-1.94c.Y-1 to I.E-1.94c.Y-480, I.E-2.94c.Y-1 to I.E-2.94c.Y-480, I.E-3.94c.Y-1 to I.E-3.94c.Y-480, I.E-4.94c.Y-1 to I.E-5.94c.Y-480, I.E-5.94c.Y-1 to I.E-3.94c.Y-480, I.E-6.94c.Y-1 to I.E-6.94c.Y-480; I.F-19c.Y-1 to I.F-1.94c.Y-480, I.F-2.94c.Y-1 to I.F-2.94c.Y-480, I.F-3.94c.Y-1 to I.F-3.94c.Y-480, I.F-4.94c.Y-1 to I.F-4.94c.Y-480, I.F-5.94c.Y-1 to I.F-5.94c.Y-480, I.F-6.94c.Y-1 to I.F-6.94c.Y-480).
Table 95c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-Cl-pyridin-3-yl and the meaning for the combination of R⁶ and R⁷ for each individual compound corresponds in each case to one line of Table Y (compounds I.E-1.95c.Y-1 to I.E-1.95c.Y-480, I.E-2.95c.Y-1 to I.E-2.95c.Y-480, I.E-3.95c.Y-1 to I.E-3.95c.Y-480, I.E-4.95c.Y-1 to I.E-5.95c.Y-480, I.E-5.95c.Y-1 to I.E-3.95c.Y-480, I.E-6.95c.Y-1 to I.E-6.95c.Y-480; I.F-19c.Y-1 to I.F-1.95c.Y-480, I.F-2.95c.Y-1 to I.F-2.95c.Y-480, I.F-3.95c.Y-1 to I.F-3.95c.Y-480, I.F-4.95c.Y-1 to I.F-4.95c.Y-480, I.F-5.95c.Y-1 to I.F-5.95c.Y-480, I.F-6.95c.Y-1 to I.F-6.95c.Y-480).
Table 96c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-Cl-pyridin-3-yl and the meaning for the combination of R⁶ and R⁷ for each individual compound corresponds in each case to one line of Table Y (compounds I.E-1.96c.Y-1 to I.E-1.96c.Y-480, I.E-2.96c.Y-1 to I.E-2.96c.Y-480, I.E-3.96c.Y-1 to I.E-3.96c.Y-480, I.E-4.96c.Y-1 to I.E-5.96c.Y-480, I.E-5.96c.Y-1 to I.E-3.96c.Y-480, I.E-6.96c.Y-1 to I.E-6.96c.Y-480; I.F-19c.Y-1 to I.F-1.96c.Y-480, I.F-2.96c.Y-1 to I.F-2.96c.Y-480, I.F-3.96c.Y-1 to I.F-3.96c.Y-480, I.F-4.96c.Y-1 to I.F-4.96c.Y-480, I.F-5.96c.Y-1 to I.F-5.96c.Y-480, I.F-6.96c.Y-1 to I.F-6.96c.Y-480).
Table 97c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-Cl-pyridin-3-yl and the meaning for the combination of R⁶ and R⁷ for each individual compound corresponds in each case to one line of Table Y (compounds I.E-1.97c.Y-1 to I.E-1.97c.Y-480, I.E-2.97c.Y-1 to I.E-2.97c.Y-480, I.E-3.97c.Y-1 to I.E-3.97c.Y-480, I.E-4.97c.Y-1 to I.E-5.97c.Y-480, I.E-5.97c.Y-1 to I.E-3.97c.Y-480, I.E-6.97c.Y-1 to I.E-6.97c.Y-480; I.F-19c.Y-1 to I.F-1.97c.Y-480, I.F-2.97c.Y-1 to I.F-2.97c.Y-480, I.F-3.97c.Y-1 to I.F-3.97c.Y-480, I.F-4.97c.Y-1 to I.F-4.97c.Y-480, I.F-5.97c.Y-1 to I.F-5.97c.Y-480, I.F-6.97c.Y-1 to I.F-6.97c.Y-480).
Table 98c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-Cl-pyridin-3-yl and the meaning for the combination of R⁶ and R⁷ for each individual compound corresponds in each case to one line of Table Y (compounds I.E-1.98c.Y-1 to I.E-1.98c.Y-480, I.E-2.98c.Y-1 to I.E-2.98c.Y-480, I.E-3.98c.Y-1 to I.E-3.98c.Y-480, I.E-4.98c.Y-1 to I.E-5.98c.Y-480, I.E-5.98c.Y-1 to I.E-3.98c.Y-480, I.E-6.98c.Y-1 to I.E-6.98c.Y-480; I.F-19c.Y-1 to I.F-1.98c.Y-480, I.F-2.98c.Y-1 to I.F-2.98c.Y-480, I.F-3.98c.Y-1 to I.F-3.98c.Y-480, I.F-4.98c.Y-1 to I.F-4.98c.Y-480, I.F-5.98c.Y-1 to I.F-5.98c.Y-480, I.F-6.98c.Y-1 to I.F-6.98c.Y-480).
Table 99c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-CH₃-pyridin-3-yl and the meaning for the combination of R⁶ and R⁷ for each individual compound corresponds in each case to one line of Table Y (compounds I.E-1.99c.Y-1 to I.E-1.99c.Y-480, I.E-2.99c.Y-1 to I.E-2.99c.Y-480, I.E-3.99c.Y-1 to I.E-3.99c.Y-480, I.E-4.99c.Y-1 to I.E-5.99c.Y-480, I.E-5.99c.Y-1 to I.E-3.99c.Y-480, I.E-6.99c.Y-1 to I.E-6.99c.Y-480; I.F-19c.Y-1 to I.F-1.99c.Y-480, I.F-2.99c.Y-1 to I.F-2.99c.Y-480, I.F-3.99c.Y-1 to I.F-3.99c.Y-480, I.F-4.99c.Y-1 to I.F-4.99c.Y-480, I.F-5.99c.Y-1 to I.F-5.99c.Y-480, I.F-6.99c.Y-1 to I.F-6.99c.Y-480).
Table 100c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-CH₃-pyridin-3-yl and the meaning for the combination of R⁶ and R⁷ for each individual compound corresponds in each case to one line of Table Y (compounds I.E-1.100c.Y-1 to I.E-1.100c.Y-480, I.E-2.100c.Y-1 to I.E-2.100c.Y-480, I.E-3.100c.Y-1 to I.E-3.100c.Y-480, I.E-4.100c.Y-1 to I.E-5.100c.Y-480, I.E-5.100c.Y-1 to I.E-3.100c.Y-480, I.E-6.100c.Y-1 to I.E-6.100c.Y-480; I.F-19c.Y-1 to I.F-1.100c.Y-480, I.F-2.100c.Y-1 to I.F-2.100c.Y-480, I.F-3.100c.Y-1 to I.F-3.100c.Y-480, I.F-4.100c.Y-1 to I.F-4.100c.Y-480, I.F-5.100c.Y-1 to I.F-5.100c.Y-480, I.F-6.100c.Y-1 to I.F-6.100c.Y-480).
Table 101c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-CH₃-pyridin-3-yl and the meaning for the combination of R⁶ and R⁷ for each individual compound corresponds in each case to one line of Table Y (compounds I.E-1.101c.Y-1 to I.E-1.101c.Y-480, I.E-2.101c.Y-1 to I.E-2.101c.Y-480, I.E-3.101c.Y-1 to I.E-3.101c.Y-480, I.E-4.101c.Y-1 to I.E-5.101c.Y-480, I.E-5.101c.Y-1 to I.E-3.101c.Y-480, I.E-6.101c.Y-1 to I.E-6.101c.Y-480; I.F-19c.Y-1 to I.F-1.101c.Y-480, I.F-2.101c.Y-1 to I.F-2.101c.Y-480, I.F-3.101c.Y-1 to I.F-3.101c.Y-480, I.F-4.101c.Y-1 to I.F-4.101c.Y-480, I.F-5.101c.Y-1 to I.F-5.101c.Y-480, I.F-6.101c.Y-1 to I.F-6.101c.Y-480).
Table 102c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-CH₃-pyridin-3-yl and the meaning for the combination of R⁶ and R⁷ for each individual compound corresponds in each case to one line of Table Y (compounds I.E-1.102c.Y-1 to I.E-1.102c.Y-480, I.E-2.102c.Y-1 to I.E-2.102c.Y-480, I.E-3.102c.Y-1 to I.E-3.102c.Y-480, I.E-4.102c.Y-1 to I.E-5.102c.Y-480, I.E-5.102c.Y-1 to I.E-3.102c.Y-480, I.E-6.102c.Y-1 to I.E-6.102c.Y-480; I.F-19c.Y-1 to I.F-1.102c.Y-480, I.F-2.102c.Y-1 to I.F-2.102c.Y-480, I.F-3.102c.Y-1 to I.F-3.102c.Y-480, I.F-4.102c.Y-1 to I.F-4.102c.Y-480, I.F-5.102c.Y-1 to I.F-5.102c.Y-480, I.F-6.102c.Y-1 to I.F-6.102c.Y-480).
Table 103c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-OCH₃-pyridin-3-yl and the meaning for the combination of R⁶ and R⁷ for each individual compound corresponds in each case to one line of Table Y (compounds I.E-1.103c.Y-1 to I.E-1.103c.Y-480, I.E-2.103c.Y-1 to I.E-2.103c.Y-480, I.E-3.103c.Y-1 to I.E-3.103c.Y-480, I.E-4.103c.Y-1 to I.E-5.103c.Y-480, I.E-5.103c.Y-1 to I.E-3.103c.Y-480, I.E-6.103c.Y-1 to I.E-6.103c.Y-480; I.F-19c.Y-1 to I.F-1.103c.Y-480, I.F-2.103c.Y-1 to I.F-2.103c.Y-480, I.F-3.103c.Y-1 to I.F-3.103c.Y-480, I.F-4.103c.Y-1 to I.F-4.103c.Y-480, I.F-5.103c.Y-1 to I.F-5.103c.Y-480, I.F-6.103c.Y-1 to I.F-6.103c.Y-480).
Table 104c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-OCH₃-pyridin-3-yl and the meaning for the combination of R⁶ and R⁷ for each individual compound corresponds in each case to one line of Table Y (compounds I.E-1.104c.Y-1 to I.E-1.104c.Y-480, I.E-2.104c.Y-1 to I.E-2.104c.Y-480, I.E-3.104c.Y-1 to I.E-3.104c.Y-480, I.E-4.104c.Y-1 to I.E-5.104c.Y-480, I.E-5.104c.Y-1 to I.E-3.104c.Y-480, I.E-6.104c.Y-1 to I.E-6.104c.Y-480; I.F-19c.Y-1 to I.F-1.104c.Y-480, I.F-2.104c.Y-1 to I.F-2.104c.Y-480, I.F-3.104c.Y-1 to I.F-3.104c.Y-480, I.F-4.104c.Y-1 to I.F-4.104c.Y-480, I.F-5.104c.Y-1 to I.F-5.104c.Y-480, I.F-6.104c.Y-1 to I.F-6.104c.Y-480).
Table 105c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-OCH₃-pyridin-3-yl and the meaning for the combination of R⁶ and R⁷ for each individual compound corresponds in each case to one line of Table Y (compounds I.E-1.105c.Y-1 to I.E-1.105c.Y-480, I.E-2.105c.Y-1 to I.E-2.105c.Y-480, I.E-3.105c.Y-1 to I.E-3.105c.Y-480, I.E-4.105c.Y-1 to I.E-5.105c.Y-480, I.E-5.105c.Y-1 to I.E-3.105c.Y-480, I.E-6.105c.Y-1 to I.E-6.105c.Y-480; I.F-19c.Y-1 to I.F-1.105c.Y-480, I.F-2.105c.Y-1 to I.F-2.105c.Y-480, I.F-3.105c.Y-1 to I.F-3.105c.Y-480, I.F-4.105c.Y-1 to I.F-4.105c.Y-480, I.F-5.105c.Y-1 to I.F-5.105c.Y-480, I.F-6.105c.Y-1 to I.F-6.105c.Y-480).
Table 106c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-OCH₃-pyridin-3-yl and the meaning for the combination of R⁶ and R⁷ for each individual compound corresponds in each case to one line of Table Y (compounds I.E-1.106c.Y-1 to I.E-1.106c.Y-480, I.E-2.106c.Y-1 to I.E-2.106c.Y-480, I.E-3.106c.Y-1 to I.E-3.106c.Y-480, I.E-4.106c.Y-1 to I.E-5.106c.Y-480, I.E-5.106c.Y-1 to I.E-3.106c.Y-480, I.E-6.106c.Y-1 to I.E-6.106c.Y-480; I.F-19c.Y-1 to I.F-1.106c.Y-480, I.F-2.106c.Y-1 to I.F-2.106c.Y-480, I.F-3.106c.Y-1 to I.F-3.106c.Y-480, I.F-4.106c.Y-1 to I.F-4.106c.Y-480, I.F-5.106c.Y-1 to I.F-5.106c.Y-480, I.F-6.106c.Y-1 to I.F-6.106c.Y-480).
Table 107c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-CHF₂-pyridin-3-yl and the meaning for the combination of R⁶ and R⁷ for each individual compound corresponds in each case to one line of Table Y (compounds I.E-1.107c.Y-1 to I.E-1.107c.Y-480, I.E-2.107c.Y-1 to I.E-2.107c.Y-480, I.E-3.107c.Y-1 to I.E-3.107c.Y-480, I.E-4.107c.Y-1 to I.E-5.107c.Y-480, I.E-5.107c.Y-1 to I.E-3.107c.Y-480, I.E-6.107c.Y-1 to I.E-6.107c.Y-480; I.F-19c.Y-1 to I.F-1.107c.Y-480, I.F-2.107c.Y-1 to I.F-2.107c.Y-480, I.F-3.107c.Y-1 to I.F-3.107c.Y-480, I.F-4.107c.Y-1 to I.F-4.107c.Y-480, I.F-5.107c.Y-1 to I.F-5.107c.Y-480, I.F-6.107c.Y-1 to I.F-6.107c.Y-480).
Table 108c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-CHF₂-pyridin-3-yl and the meaning for the combination of R⁶ and R⁷ for each individual compound corresponds in each case to one line of Table Y (compounds I.E-1.108c.Y-1 to I.E-1.108c.Y-480, I.E-2.108c.Y-1 to I.E-2.108c.Y-480, I.E-3.108c.Y-1 to I.E-3.108c.Y-480, I.E-4.108c.Y-1 to I.E-5.108c.Y-480, I.E-5.108c.Y-1 to I.E-3.108c.Y-480, I.E-6.108c.Y-1 to I.E-6.108c.Y-480; I.F-19c.Y-1 to I.F-1.108c.Y-480, I.F-2.108c.Y-1 to I.F-2.108c.Y-480, I.F-3.108c.Y-1 to I.F-3.108c.Y-480, I.F-4.108c.Y-1 to I.F-4.108c.Y-480, I.F-5.108c.Y-1 to I.F-5.108c.Y-480, I.F-6.108c.Y-1 to I.F-6.108c.Y-480).
Table 109c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-CHF₂-pyridin-3-yl and the meaning for the combination of R⁶ and R⁷ for each individual compound corresponds in each case to one line of Table Y (compounds I.E-1.109c.Y-1 to I.E-1.109c.Y-480, I.E-2.109c.Y-1 to I.E-2.109c.Y-480, I.E-3.109c.Y-1 to I.E-3.109c.Y-480, I.E-4.109c.Y-1 to I.E-5.109c.Y-480, I.E-5.109c.Y-1 to I.E-3.109c.Y-480, I.E-6.109c.Y-1 to I.E-6.109c.Y-480; I.F-19c.Y-1 to I.F-1.109c.Y-480, I.F-2.109c.Y-1 to I.F-2.109c.Y-480, I.F-3.109c.Y-1 to I.F-3.109c.Y-480, I.F-4.109c.Y-1 to I.F-4.109c.Y-480, I.F-5.109c.Y-1 to I.F-5.109c.Y-480, I.F-6.109c.Y-1 to I.F-6.109c.Y-480).
Table 110c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-CHF₂-pyridin-3-yl and the meaning for the combination of R⁶ and R⁷ for each individual compound corresponds in each case to one line of Table Y (compounds I.E-1.110c.Y-1 to I.E-1.110c.Y-480, I.E-2.110c.Y-1 to I.E-2.110c.Y-480, I.E-3.110c.Y-1 to I.E-3.110c.Y-480, I.E-4.110c.Y-1 to I.E-5.110c.Y-480, I.E-5.110c.Y-1 to I.E-3.110c.Y-480, I.E-6.110c.Y-1 to I.E-6.110c.Y-480; I.F-19c.Y-1 to I.F-1.110c.Y-480, I.F-2.110c.Y-1 to I.F-2.110c.Y-480, I.F-3.110c.Y-1 to I.F-3.110c.Y-480, I.F-4.110c.Y-1 to I.F-4.110c.Y-480, I.F-5.110c.Y-1 to I.F-5.110c.Y-480, I.F-6.110c.Y-1 to I.F-6.110c.Y-480).
Table 111c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-OCHF₂-pyridin-3-yl and the meaning for the combination of R⁶ and R⁷ for each individual compound corresponds in each case to one line of Table Y (compounds I.E-1.111c.Y-1 to I.E-1.111c.Y-480, I.E-2.111c.Y-1 to I.E-2.111c.Y-480, I.E-3.111c.Y-1 to I.E-3.111c.Y-480, I.E-4.111c.Y-1 to I.E-5.111c.Y-480, I.E-5.111c.Y-1 to I.E-3.111c.Y-480, I.E-6.111c.Y-1 to I.E-6.111c.Y-480; I.F-19c.Y-1 to I.F-1.111c.Y-480, I.F-2.111c.Y-1 to I.F-2.111c.Y-480, I.F-3.111c.Y-1 to I.F-3.111c.Y-480, I.F-4.111c.Y-1 to I.F-4.111c.Y-480, I.F-5.111c.Y-1 to I.F-5.111c.Y-480, I.F-6.111c.Y-1 to I.F-6.111c.Y-480).
Table 112c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-OCHF₂-pyridin-3-yl and the meaning for the combination of R⁶ and R⁷ for each individual compound corresponds in each case to one line of Table Y (compounds I.E-1.112c.Y-1 to I.E-1.112c.Y-480, I.E-2.112c.Y-1 to I.E-2.112c.Y-480, I.E-3.112c.Y-1 to I.E-3.112c.Y-480, I.E-4.112c.Y-1 to I.E-5.112c.Y-480, I.E-5.112c.Y-1 to I.E-3.112c.Y-480, I.E-6.112c.Y-1 to I.E-6.112c.Y-480; I.F-19c.Y-1 to I.F-1.112c.Y-480, I.F-2.112c.Y-1 to I.F-2.112c.Y-480, I.F-3.112c.Y-1 to I.F-3.112c.Y-480, I.F-4.112c.Y-1 to I.F-4.112c.Y-480, I.F-5.112c.Y-1 to I.F-5.112c.Y-480, I.F-6.112c.Y-1 to I.F-6.112c.Y-480).
Table 113c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-OCHF₂-pyridin-3-yl and the meaning for the combination of R⁶ and R⁷ for each individual compound corresponds in each case to one line of Table Y (compounds I.E-1.113c.Y-1 to I.E-1.113c.Y-480, I.E-2.113c.Y-1 to I.E-2.113c.Y-480, I.E-3.113c.Y-1 to I.E-3.113c.Y-480, I.E-4.113c.Y-1 to I.E-5.113c.Y-480, I.E-5.113c.Y-1 to I.E-3.113c.Y-480, I.E-6.113c.Y-1 to I.E-6.113c.Y-480; I.F-19c.Y-1 to I.F-1.113c.Y-480, I.F-2.113c.Y-1 to I.F-2.113c.Y-480, I.F-3.113c.Y-1 to I.F-3.113c.Y-480, I.F-4.113c.Y-1 to I.F-4.113c.Y-480, I.F-5.113c.Y-1 to I.F-5.113c.Y-480, I.F-6.113c.Y-1 to I.F-6.113c.Y-480).
Table 114c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-OCHF₂-pyridin-3-yl and the meaning for the combination of R⁶ and R⁷ for each individual compound corresponds in each case to one line of Table Y (compounds I.E-1.114c.Y-1 to I.E-1.114c.Y-480, I.E-2.114c.Y-1 to I.E-2.114c.Y-480, I.E-3.114c.Y-1 to I.E-3.114c.Y-480, I.E-4.114c.Y-1 to I.E-5.114c.Y-480, I.E-5.114c.Y-1 to I.E-3.114c.Y-480, I.E-6.114c.Y-1 to I.E-6.114c.Y-480; I.F-19c.Y-1 to I.F-1.114c.Y-480, I.F-2.114c.Y-1 to I.F-2.114c.Y-480, I.F-3.114c.Y-1 to I.F-3.114c.Y-480, I.F-4.114c.Y-1 to I.F-4.114c.Y-480, I.F-5.114c.Y-1 to I.F-5.114c.Y-480, I.F-6.114c.Y-1 to I.F-6.114c.Y-480).
Table 115c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-OCHF₂-pyridin-3-yl and the meaning for the combination of R⁶ and R⁷ for each individual compound corresponds in each case to one line of Table Y (compounds I.E-1.115c.Y-1 to I.E-1.115c.Y-480, I.E-2.115c.Y-1 to I.E-2.115c.Y-480, I.E-3.115c.Y-1 to I.E-3.115c.Y-480, I.E-4.115c.Y-1 to I.E-5.115c.Y-480, I.E-5.115c.Y-1 to I.E-3.115c.Y-480, I.E-6.115c.Y-1 to I.E-6.115c.Y-480; I.F-19c.Y-1 to I.F-1.115c.Y-480, I.F-2.115c.Y-1 to I.F-2.115c.Y-480, I.F-3.115c.Y-1 to I.F-3.115c.Y-480, I.F-4.115c.Y-1 to I.F-4.115c.Y-480, I.F-5.115c.Y-1 to I.F-5.115c.Y-480, I.F-6.115c.Y-1 to I.F-6.115c.Y-480).
Table 116c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-CF₃-pyridin-3-yl and the meaning for the combination of R⁶ and R⁷ for each individual compound corresponds in each case to one line of Table Y (compounds I.E-1.116c.Y-1 to I.E-1.116c.Y-480, I.E-2.116c.Y-1 to I.E-2.116c.Y-480, I.E-3.116c.Y-1 to I.E-3.116c.Y-480, I.E-4.116c.Y-1 to I.E-5.116c.Y-480, I.E-5.116c.Y-1 to I.E-3.116c.Y-480, I.E-6.116c.Y-1 to I.E-6.116c.Y-480; I.F-19c.Y-1 to I.F-1.116c.Y-480, I.F-2.116c.Y-1 to I.F-2.116c.Y-480, I.F-3.116c.Y-1 to I.F-3.116c.Y-480, I.F-4.116c.Y-1 to I.F-4.116c.Y-480, I.F-5.116c.Y-1 to I.F-5.116c.Y-480, I.F-6.116c.Y-1 to I.F-6.116c.Y-480).
Table 117c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-CF₃-pyridin-3-yl and the meaning for the combination of R⁶ and R⁷ for each individual compound corresponds in each case to one line of Table Y (compounds I.E-1.117c.Y-1 to I.E-1.117c.Y-480, I.E-2.117c.Y-1 to I.E-2.117c.Y-480, I.E-3.117c.Y-1 to I.E-3.117c.Y-480, I.E-4.117c.Y-1 to I.E-5.117c.Y-480, I.E-5.117c.Y-1 to I.E-3.117c.Y-480, I.E-6.117c.Y-1 to I.E-6.117c.Y-480; I.F-19c.Y-1 to I.F-1.117c.Y-480, I.F-2.117c.Y-1 to I.F-2.117c.Y-480, I.F-3.117c.Y-1 to I.F-3.117c.Y-480, I.F-4.117c.Y-1 to I.F-4.117c.Y-480, I.F-5.117c.Y-1 to I.F-5.117c.Y-480, I.F-6.117c.Y-1 to I.F-6.117c.Y-480).
Table 118c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-CF₃-pyridin-3-yl and the meaning for the combination of R⁶ and R⁷ for each individual compound corresponds in each case to one line of Table Y (compounds I.E-1.118c.Y-1 to I.E-1.118c.Y-480, I.E-2.118c.Y-1 to I.E-2.118c.Y-480, I.E-3.118c.Y-1 to I.E-3.118c.Y-480, I.E-4.118c.Y-1 to I.E-5.118c.Y-480, I.E-5.118c.Y-1 to I.E-3.118c.Y-480, I.E-6.118c.Y-1 to I.E-6.118c.Y-480; I.F-19c.Y-1 to I.F-1.118c.Y-480, I.F-2.118c.Y-1 to I.F-2.118c.Y-480, I.F-3.118c.Y-1 to I.F-3.118c.Y-480, I.F-4.118c.Y-1 to I.F-4.118c.Y-480, I.F-5.118c.Y-1 to I.F-5.118c.Y-480, I.F-6.118c.Y-1 to I.F-6.118c.Y-480).
Table 119c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-CF₃-pyridin-3-yl and the meaning for the combination of R⁶ and R⁷ for each individual compound corresponds in each case to one line of Table Y (compounds I.E-1.119c.Y-1 to I.E-1.119c.Y-480, I.E-2.119c.Y-1 to I.E-2.119c.Y-480, I.E-3.119c.Y-1 to I.E-3.119c.Y-480, I.E-4.119c.Y-1 to I.E-5.119c.Y-480, I.E-5.119c.Y-1 to I.E-3.119c.Y-480, I.E-6.119c.Y-1 to I.E-6.119c.Y-480; I.F-19c.Y-1 to I.F-1.119c.Y-480, I.F-2.119c.Y-1 to I.F-2.119c.Y-480, I.F-3.119c.Y-1 to I.F-3.119c.Y-480, I.F-4.119c.Y-1 to I.F-4.119c.Y-480, I.F-5.119c.Y-1 to I.F-5.119c.Y-480, I.F-6.119c.Y-1 to I.F-6.119c.Y-480).
Table 120c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-OCF₃-pyridin-3-yl and the meaning for the combination of R⁶ and R⁷ for each individual compound corresponds in each case to one line of Table Y (compounds I.E-1.120c.Y-1 to I.E-1.120c.Y-480, I.E-2.120c.Y-1 to I.E-2.120c.Y-480, I.E-3.120c.Y-1 to I.E-3.120c.Y-480, I.E-4.120c.Y-1 to I.E-5.120c.Y-480, I.E-5.120c.Y-1 to I.E-3.120c.Y-480, I.E-6.120c.Y-1 to I.E-6.120c.Y-480; I.F-19c.Y-1 to I.F-1.120c.Y-480, I.F-2.120c.Y-1 to I.F-2.120c.Y-480, I.F-3.120c.Y-1 to I.F-3.120c.Y-480, I.F-4.120c.Y-1 to I.F-4.120c.Y-480, I.F-5.120c.Y-1 to I.F-5.120c.Y-480, I.F-6.120c.Y-1 to I.F-6.120c.Y-480).
Table 121c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-OCF₃-pyridin-3-yl and the meaning for the combination of R⁶ and R⁷ for each individual compound corresponds in each case to one line of Table Y (compounds I.E-1.121c.Y-1 to I.E-1.121c.Y-480, I.E-2.121c.Y-1 to I.E-2.121c.Y-480, I.E-3.121c.Y-1 to I.E-3.121c.Y-480, I.E-4.121c.Y-1 to I.E-5.121c.Y-480, I.E-5.121c.Y-1 to I.E-3.121c.Y-480, I.E-6.121c.Y-1 to I.E-6.121c.Y-480; I.F-19c.Y-1 to I.F-1.121c.Y-480, I.F-2.121c.Y-1 to I.F-2.121c.Y-480, I.F-3.121c.Y-1 to I.F-3.121c.Y-480, I.F-4.121c.Y-1 to I.F-4.121c.Y-480, I.F-5.121c.Y-1 to I.F-5.121c.Y-480, I.F-6.121c.Y-1 to I.F-6.121c.Y-480).
Table 122c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-OCF₃-pyridin-3-yl and the meaning for the combination of R⁶ and R⁷ for each individual compound corresponds in each case to one line of Table Y (compounds I.E-1.122c.Y-1 to I.E-1.122c.Y-480, I.E-2.122c.Y-1 to I.E-2.122c.Y-480, I.E-3.122c.Y-1 to I.E-3.122c.Y-480, I.E-4.122c.Y-1 to I.E-5.122c.Y-480, I.E-5.122c.Y-1 to I.E-3.122c.Y-480, I.E-6.122c.Y-1 to I.E-6.122c.Y-480; I.F-19c.Y-1 to I.F-1.122c.Y-480, I.F-2.122c.Y-1 to I.F-2.122c.Y-480, I.F-3.122c.Y-1 to I.F-3.122c.Y-480, I.F-4.122c.Y-1 to I.F-4.122c.Y-480, I.F-5.122c.Y-1 to I.F-5.122c.Y-480, I.F-6.122c.Y-1 to I.F-6.122c.Y-480).
Table 123c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-OCF₃-pyridin-3-yl and the meaning for the combination of R⁶ and R⁷ for each individual compound corresponds in each case to one line of Table Y (compounds I.E-1.123c.Y-1 to I.E-1.123c.Y-480, I.E-2.123c.Y-1 to I.E-2.123c.Y-480, I.E-3.123c.Y-1 to I.E-3.123c.Y-480, I.E-4.123c.Y-1 to I.E-5.123c.Y-480, I.E-5.123c.Y-1 to I.E-3.123c.Y-480, I.E-6.123c.Y-1 to I.E-6.123c.Y-480; I.F-19c.Y-1 to I.F-1.123c.Y-480, I.F-2.123c.Y-1 to I.F-2.123c.Y-480, I.F-3.123c.Y-1 to I.F-3.123c.Y-480, I.F-4.123c.Y-1 to I.F-4.123c.Y-480, I.F-5.123c.Y-1 to I.F-5.123c.Y-480, I.F-6.123c.Y-1 to I.F-6.123c.Y-480).
Table 124c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-CN-pyridin-3-yl and the meaning for the combination of R⁶ and R⁷ for each individual compound corresponds in each case to one line of Table Y (compounds I.E-1.124c.Y-1 to I.E-1.124c.Y-480, I.E-2.124c.Y-1 to I.E-2.124c.Y-480, I.E-3.124c.Y-1 to I.E-3.124c.Y-480, I.E-4.124c.Y-1 to I.E-5.124c.Y-480, I.E-5.124c.Y-1 to I.E-3.124c.Y-480, I.E-6.124c.Y-1 to I.E-6.124c.Y-480; I.F-19c.Y-1 to I.F-1.124c.Y-480, I.F-2.124c.Y-1 to I.F-2.124c.Y-480, I.F-3.124c.Y-1 to I.F-3.124c.Y-480, I.F-4.124c.Y-1 to I.F-4.124c.Y-480, I.F-5.124c.Y-1 to I.F-5.124c.Y-480, I.F-6.124c.Y-1 to I.F-6.124c.Y-480).
Table 125c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 4-CN-pyridin-3-yl and the meaning for the combination of R⁶ and R⁷ for each individual compound corresponds in each case to one line of Table Y (compounds I.E-1.125c.Y-1 to I.E-1.125c.Y-480, I.E-2.125c.Y-1 to I.E-2.125c.Y-480, I.E-3.125c.Y-1 to I.E-3.125c.Y-480, I.E-4.125c.Y-1 to I.E-5.125c.Y-480, I.E-5.125c.Y-1 to I.E-3.125c.Y-480, I.E-6.125c.Y-1 to I.E-6.125c.Y-480; I.F-19c.Y-1 to I.F-1.125c.Y-480, I.F-2.125c.Y-1 to I.F-2.125c.Y-480, I.F-3.125c.Y-1 to I.F-3.125c.Y-480, I.F-4.125c.Y-1 to I.F-4.125c.Y-480, I.F-5.125c.Y-1 to I.F-5.125c.Y-480, I.F-6.125c.Y-1 to I.F-6.125c.Y-480).
Table 126c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 5-CN-pyridin-3-yl and the meaning for the combination of R⁶ and R⁷ for each individual compound corresponds in each case to one line of Table Y (compounds I.E-1.126c.Y-1 to I.E-1.126c.Y-480, I.E-2.126c.Y-1 to I.E-2.126c.Y-480, I.E-3.126c.Y-1 to I.E-3.126c.Y-480, I.E-4.126c.Y-1 to I.E-5.126c.Y-480, I.E-5.126c.Y-1 to I.E-3.126c.Y-480, I.E-6.126c.Y-1 to I.E-6.126c.Y-480; I.F-19c.Y-1 to I.F-1.126c.Y-480, I.F-2.126c.Y-1 to I.F-2.126c.Y-480, I.F-3.126c.Y-1 to I.F-3.126c.Y-480, I.F-4.126c.Y-1 to I.F-4.126c.Y-480, I.F-5.126c.Y-1 to I.F-5.126c.Y-480, I.F-6.126c.Y-1 to I.F-6.126c.Y-480).
Table 127c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 6-CN-pyridin-3-yl and the meaning for the combination of R⁶ and R⁷ for each individual compound corresponds in each case to one line of Table Y (compounds I.E-1.127c.Y-1 to I.E-1.127c.Y-480, I.E-2.127c.Y-1 to I.E-2.127c.Y-480, I.E-3.127c.Y-1 to I.E-3.127c.Y-480, I.E-4.127c.Y-1 to I.E-5.127c.Y-480, I.E-5.127c.Y-1 to I.E-3.127c.Y-480, I.E-6.127c.Y-1 to I.E-6.127c.Y-480; I.F-19c.Y-1 to I.F-1.127c.Y-480, I.F-2.127c.Y-1 to I.F-2.127c.Y-480, I.F-3.127c.Y-1 to I.F-3.127c.Y-480, I.F-4.127c.Y-1 to I.F-4.127c.Y-480, I.F-5.127c.Y-1 to I.F-5.127c.Y-480, I.F-6.127c.Y-1 to I.F-6.127c.Y-480).
Table 128c Compounds of the formula I.E-1, I.E-2, I.E-3, I.E-4, I.E-5, I.E-6; I.F-1, I.F-2, I.F-3, I.F-4, I.F-5, I.F-6 in which R⁸ is pyridine-4-yl and the meaning for the combination of R⁶ and R⁷ for each individual compound corresponds in each case to one line of Table Y (compounds I.E-1.128c.Y-1 to I.E-1.128c.Y-480, I.E-2.128c.Y-1 to I.E-2.128c.Y-480, I.E-3.128c.Y-1 to I.E-3.128c.Y-480, I.E-4.128c.Y-1 to I.E-5.128c.Y-480, I.E-5.128c.Y-1 to I.E-3.128c.Y-480, I.E-6.128c.Y-1 to I.E-6.128c.Y-480; I.F-19c.Y-1 to I.F-1.128c.Y-480, I.F-2.128c.Y-1 to I.F-2.128c.Y-480, I.F-3.128c.Y-1 to I.F-3.128c.Y-480, I.F-4.128c.Y-1 to I.F-4.128c.Y-480, I.F-5.128c.Y-1 to I.F-5.128c.Y-480, I.F-6.128c.Y-1 to I.F-6.128c.Y-480).
Table 129c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-F-pyridine-4-yl and the meaning for the combination of R⁶ and R⁷ for each individual compound corresponds in each case to one line of Table Y (compounds I.E-1.129c.Y-1 to I.E-1.129c.Y-480, I.E-2.129c.Y-1 to I.E-2.129c.Y-480, I.E-3.129c.Y-1 to I.E-3.129c.Y-480, I.E-4.129c.Y-1 to I.E-5.129c.Y-480, I.E-5.129c.Y-1 to I.E-3.129c.Y-480, I.E-6.129c.Y-1 to I.E-6.129c.Y-480; I.F-19c.Y-1 to I.F-1.129c.Y-480, I.F-2.129c.Y-1 to I.F-2.129c.Y-480, I.F-3.129c.Y-1 to I.F-3.129c.Y-480, I.F-4.129c.Y-1 to I.F-4.129c.Y-480, I.F-5.129c.Y-1 to I.F-5.129c.Y-480, I.F-6.129c.Y-1 to I.F-6.129c.Y-480).
Table 130c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-F-pyridine-4-yl and the meaning for the combination of R⁶ and R⁷ for each individual compound corresponds in each case to one line of Table Y (compounds I.E-1.130c.Y-1 to I.E-1.130c.Y-480, I.E-2.130c.Y-1 to I.E-2.130c.Y-480, I.E-3.130c.Y-1 to I.E-3.130c.Y-480, I.E-4.130c.Y-1 to I.E-5.130c.Y-480, I.E-5.130c.Y-1 to I.E-3.130c.Y-480, I.E-6.130c.Y-1 to I.E-6.130c.Y-480; I.F-19c.Y-1 to I.F-1.130c.Y-480, I.F-2.130c.Y-1 to I.F-2.130c.Y-480, I.F-3.130c.Y-1 to I.F-3.130c.Y-480, I.F-4.130c.Y-1 to I.F-4.130c.Y-480, I.F-5.130c.Y-1 to I.F-5.130c.Y-480, I.F-6.130c.Y-1 to I.F-6.130c.Y-480).
Table 131c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-Cl-pyridine-4-yl and the meaning for the combination of R⁶ and R⁷ for each individual compound corresponds in each case to one line of Table Y (compounds I.E-1.131c.Y-1 to I.E-1.131c.Y-480, I.E-2.131c.Y-1 to I.E-2.131c.Y-480, I.E-3.131c.Y-1 to I.E-3.131c.Y-480, I.E-4.131c.Y-1 to I.E-5.131c.Y-480, I.E-5.131c.Y-1 to I.E-3.131c.Y-480, I.E-6.131c.Y-1 to I.E-6.131c.Y-480; I.F-19c.Y-1 to I.F-1.131c.Y-480, I.F-2.131c.Y-1 to I.F-2.131c.Y-480, I.F-3.131c.Y-1 to I.F-3.131c.Y-480, I.F-4.131c.Y-1 to I.F-4.131c.Y-480, I.F-5.131c.Y-1 to I.F-5.131c.Y-480, I.F-6.131c.Y-1 to I.F-6.131c.Y-480).
Table 132c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-Cl-pyridine-4-yl and the meaning for the combination of R⁶ and R⁷ for each individual compound corresponds in each case to one line of Table Y (compounds I.E-1.132c.Y-1 to I.E-1.132c.Y-480, I.E-2.132c.Y-1 to I.E-2.132c.Y-480, I.E-3.132c.Y-1 to I.E-3.132c.Y-480, I.E-4.132c.Y-1 to I.E-5.132c.Y-480, I.E-5.132c.Y-1 to I.E-3.132c.Y-480, I.E-6.132c.Y-1 to I.E-6.132c.Y-480; I.F-19c.Y-1 to I.F-1.132c.Y-480, I.F-2.132c.Y-1 to I.F-2.132c.Y-480, I.F-3.132c.Y-1 to I.F-3.132c.Y-480, I.F-4.132c.Y-1 to I.F-4.132c.Y-480, I.F-5.132c.Y-1 to I.F-5.132c.Y-480, I.F-6.132c.Y-1 to I.F-6.132c.Y-480).
Table 133c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-CH₃-pyridine-4-yl and the meaning for the combination of R⁶ and R⁷ for each individual compound corresponds in each case to one line of Table Y (compounds I.E-1.133c.Y-1 to I.E-1.133c.Y-480, I.E-2.133c.Y-1 to I.E-2.133c.Y-480, I.E-3.133c.Y-1 to I.E-3.133c.Y-480, I.E-4.133c.Y-1 to I.E-5.133c.Y-480, I.E-5.133c.Y-1 to I.E-3.133c.Y-480, I.E-6.133c.Y-1 to I.E-6.133c.Y-480; I.F-19c.Y-1 to I.F-1.133c.Y-480, I.F-2.133c.Y-1 to I.F-2.133c.Y-480, I.F-3.133c.Y-1 to I.F-3.133c.Y-480, I.F-4.133c.Y-1 to I.F-4.133c.Y-480, I.F-5.133c.Y-1 to I.F-5.133c.Y-480, I.F-6.133c.Y-1 to I.F-6.133c.Y-480).
Table 134c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-CH₃-pyridine-4-yl₄ and the meaning for the combination of R⁶ and R⁷ for each individual compound corresponds in each case to one line of Table Y (compounds I.E-1.134c.Y-1 to I.E-1.134c.Y-480, I.E-2.134c.Y-1 to I.E-2.134c.Y-480, I.E-3.134c.Y-1 to I.E-3.134c.Y-480, I.E-4.134c.Y-1 to I.E-5.134c.Y-480, I.E-5.134c.Y-1 to I.E-3.134c.Y-480, I.E-6.134c.Y-1 to I.E-6.134c.Y-480; I.F-19c.Y-1 to I.F-1.134c.Y-480, I.F-2.134c.Y-1 to I.F-2.134c.Y-480, I.F-3.134c.Y-1 to I.F-3.134c.Y-480, I.F-4.134c.Y-1 to I.F-4.134c.Y-480, I.F-5.134c.Y-1 to I.F-5.134c.Y-480, I.F-6.134c.Y-1 to I.F-6.134c.Y-480).
Table 135c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-OCH₃-pyridine-4-yl and the meaning for the combination of R⁶ and R⁷ for each individual compound corresponds in each case to one line of Table Y (compounds I.E-1.135c.Y-1 to I.E-1.135c.Y-480, I.E-2.135c.Y-1 to I.E-2.135c.Y-480, I.E-3.135c.Y-1 to I.E-3.135c.Y-480, I.E-4.135c.Y-1 to I.E-5.135c.Y-480, I.E-5.135c.Y-1 to I.E-3.135c.Y-480, I.E-6.135c.Y-1 to I.E-6.135c.Y-480; I.F-19c.Y-1 to I.F-1.135c.Y-480, I.F-2.135c.Y-1 to I.F-2.135c.Y-480, I.F-3.135c.Y-1 to I.F-3.135c.Y-480, I.F-4.135c.Y-1 to I.F-4.135c.Y-480, I.F-5.135c.Y-1 to I.F-5.135c.Y-480, I.F-6.135c.Y-1 to I.F-6.135c.Y-480).
Table 136c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-OCH₃-pyridine-4-yl and the meaning for the combination of R⁶ and R⁷ for each individual compound corresponds in each case to one line of Table Y (compounds I.E-1.136c.Y-1 to I.E-1.136c.Y-480, I.E-2.136c.Y-1 to I.E-2.136c.Y-480, I.E-3.136c.Y-1 to I.E-3.136c.Y-480, I.E-4.136c.Y-1 to I.E-5.136c.Y-480, I.E-5.136c.Y-1 to I.E-3.136c.Y-480, I.E-6.136c.Y-1 to I.E-6.136c.Y-480; I.F-19c.Y-1 to I.F-1.136c.Y-480, I.F-2.136c.Y-1 to I.F-2.136c.Y-480, I.F-3.136c.Y-1 to I.F-3.136c.Y-480, I.F-4.136c.Y-1 to I.F-4.136c.Y-480, I.F-5.136c.Y-1 to I.F-5.136c.Y-480, I.F-6.136c.Y-1 to I.F-6.136c.Y-480).
Table 137c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-CHF₂-pyridine-4-yl and the meaning for the combination of R⁶ and R⁷ for each individual compound corresponds in each case to one line of Table Y (compounds I.E-1.137c.Y-1 to I.E-1.137c.Y-480, I.E-2.137c.Y-1 to I.E-2.137c.Y-480, I.E-3.137c.Y-1 to I.E-3.137c.Y-480, I.E-4.137c.Y-1 to I.E-5.137c.Y-480, I.E-5.137c.Y-1 to I.E-3.137c.Y-480, I.E-6.137c.Y-1 to I.E-6.137c.Y-480; I.F-19c.Y-1 to I.F-1.137c.Y-480, I.F-2.137c.Y-1 to I.F-2.137c.Y-480, I.F-3.137c.Y-1 to I.F-3.137c.Y-480, I.F-4.137c.Y-1 to I.F-4.137c.Y-480, I.F-5.137c.Y-1 to I.F-5.137c.Y-480, I.F-6.137c.Y-1 to I.F-6.137c.Y-480).
Table 138c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-CHF₂-pyridine-4-yl and the meaning for the combination of R⁶ and R⁷ for each individual compound corresponds in each case to one line of Table Y (compounds I.E-1.138c.Y-1 to I.E-1.138c.Y-480, I.E-2.138c.Y-1 to I.E-2.138c.Y-480, I.E-3.138c.Y-1 to I.E-3.138c.Y-480, I.E-4.138c.Y-1 to I.E-5.138c.Y-480, I.E-5.138c.Y-1 to I.E-3.138c.Y-480, I.E-6.138c.Y-1 to I.E-6.138c.Y-480; I.F-19c.Y-1 to I.F-1.138c.Y-480, I.F-2.138c.Y-1 to I.F-2.138c.Y-480, I.F-3.138c.Y-1 to I.F-3.138c.Y-480, I.F-4.138c.Y-1 to I.F-4.138c.Y-480, I.F-5.138c.Y-1 to I.F-5.138c.Y-480, I.F-6.138c.Y-1 to I.F-6.138c.Y-480).
Table 139c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-OCHF₂-pyridine-4-yl and the meaning for the combination of R⁶ and R⁷ for each individual compound corresponds in each case to one line of Table Y (compounds I.E-1.139c.Y-1 to I.E-1.139c.Y-480, I.E-2.139c.Y-1 to I.E-2.139c.Y-480, I.E-3.139c.Y-1 to I.E-3.139c.Y-480, I.E-4.139c.Y-1 to I.E-5.139c.Y-480, I.E-5.139c.Y-1 to I.E-3.139c.Y-480, I.E-6.139c.Y-1 to I.E-6.139c.Y-480; I.F-19c.Y-1 to I.F-1.139c.Y-480, I.F-2.139c.Y-1 to I.F-2.139c.Y-480, I.F-3.139c.Y-1 to I.F-3.139c.Y-480, I.F-4.139c.Y-1 to I.F-4.139c.Y-480, I.F-5.139c.Y-1 to I.F-5.139c.Y-480, I.F-6.139c.Y-1 to I.F-6.139c.Y-480).
Table 140c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-OCHF₂-pyridine-4-yl and the meaning for the combination of R⁶ and R⁷ for each individual compound corresponds in each case to one line of Table Y (compounds I.E-1.140c.Y-1 to I.E-1.140c.Y-480, I.E-2.140c.Y-1 to I.E-2.140c.Y-480, I.E-3.140c.Y-1 to I.E-3.140c.Y-480, I.E-4.140c.Y-1 to I.E-5.140c.Y-480, I.E-5.140c.Y-1 to I.E-3.140c.Y-480, I.E-6.140c.Y-1 to I.E-6.140c.Y-480; I.F-19c.Y-1 to I.F-1.140c.Y-480, I.F-2.140c.Y-1 to I.F-2.140c.Y-480, I.F-3.140c.Y-1 to I.F-3.140c.Y-480, I.F-4.140c.Y-1 to I.F-4.140c.Y-480, I.F-5.140c.Y-1 to I.F-5.140c.Y-480, I.F-6.140c.Y-1 to I.F-6.140c.Y-480).
Table 141c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-CF₃-pyridine-4-yl and the meaning for the combination of R⁶ and R⁷ for each individual compound corresponds in each case to one line of Table Y (compounds I.E-1.141c.Y-1 to I.E-1.141c.Y-480, I.E-2.141c.Y-1 to I.E-2.141c.Y-480, I.E-3.141c.Y-1 to I.E-3.141c.Y-480, I.E-4.141c.Y-1 to I.E-5.141c.Y-480, I.E-5.141c.Y-1 to I.E-3.141c.Y-480, I.E-6.141c.Y-1 to I.E-6.141c.Y-480; I.F-19c.Y-1 to I.F-1.141c.Y-480, I.F-2.141c.Y-1 to I.F-2.141c.Y-480, I.F-3.141c.Y-1 to I.F-3.141c.Y-480, I.F-4.141c.Y-1 to I.F-4.141c.Y-480, I.F-5.141c.Y-1 to I.F-5.141c.Y-480, I.F-6.141c.Y-1 to I.F-6.141c.Y-480).
Table 142c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-CF₃-pyridine-4-yl and the meaning for the combination of R⁶ and R⁷ for each individual compound corresponds in each case to one line of Table Y (compounds I.E-1.142c.Y-1 to I.E-1.142c.Y-480, I.E-2.142c.Y-1 to I.E-2.142c.Y-480, I.E-3.142c.Y-1 to I.E-3.142c.Y-480, I.E-4.142c.Y-1 to I.E-5.142c.Y-480, I.E-5.142c.Y-1 to I.E-3.142c.Y-480, I.E-6.142c.Y-1 to I.E-6.142c.Y-480; I.F-19c.Y-1 to I.F-1.142c.Y-480, I.F-2.142c.Y-1 to I.F-2.142c.Y-480, I.F-3.142c.Y-1 to I.F-3.142c.Y-480, I.F-4.142c.Y-1 to I.F-4.142c.Y-480, I.F-5.142c.Y-1 to I.F-5.142c.Y-480, I.F-6.142c.Y-1 to I.F-6.142c.Y-480).
Table 143c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-OCF₃-pyridine-4-yl and the meaning for the combination of R⁶ and R⁷ for each individual compound corresponds in each case to one line of Table Y (compounds I.E-1.143c.Y-1 to I.E-1.143c.Y-480, I.E-2.143c.Y-1 to I.E-2.143c.Y-480, I.E-3.143c.Y-1 to I.E-3.143c.Y-480, I.E-4.143c.Y-1 to I.E-5.143c.Y-480, I.E-5.143c.Y-1 to I.E-3.143c.Y-480, I.E-6.143c.Y-1 to I.E-6.143c.Y-480; I.F-19c.Y-1 to I.F-1.143c.Y-480, I.F-2.143c.Y-1 to I.F-2.143c.Y-480, I.F-3.143c.Y-1 to I.F-3.143c.Y-480, I.F-4.143c.Y-1 to I.F-4.143c.Y-480, I.F-5.143c.Y-1 to I.F-5.143c.Y-480, I.F-6.143c.Y-1 to I.F-6.143c.Y-480).
Table 144c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-OCF₃-pyridine-4-yl and the meaning for the combination of R⁶ and R⁷ for each individual compound corresponds in each case to one line of Table Y (compounds I.E-1.144c.Y-1 to I.E-1.144c.Y-480, I.E-2.144c.Y-1 to I.E-2.144c.Y-480, I.E-3.144c.Y-1 to I.E-3.144c.Y-480, I.E-4.144c.Y-1 to I.E-5.144c.Y-480, I.E-5.144c.Y-1 to I.E-3.144c.Y-480, I.E-6.144c.Y-1 to I.E-6.144c.Y-480; I.F-19c.Y-1 to I.F-1.144c.Y-480, I.F-2.144c.Y-1 to I.F-2.144c.Y-480, I.F-3.144c.Y-1 to I.F-3.144c.Y-480, I.F-4.144c.Y-1 to I.F-4.144c.Y-480, I.F-5.144c.Y-1 to I.F-5.144c.Y-480, I.F-6.144c.Y-1 to I.F-6.144c.Y-480).
Table 145c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 2-CN-pyridine-4-yl and the meaning for the combination of R⁶ and R⁷ for each individual compound corresponds in each case to one line of Table Y (compounds I.E-1.145c.Y-1 to I.E-1.145c.Y-480, I.E-2.145c.Y-1 to I.E-2.145c.Y-480, I.E-3.145c.Y-1 to I.E-3.145c.Y-480, I.E-4.145c.Y-1 to I.E-5.145c.Y-480, I.E-5.145c.Y-1 to I.E-3.145c.Y-480, I.E-6.145c.Y-1 to I.E-6.145c.Y-480; I.F-19c.Y-1 to I.F-1.145c.Y-480, I.F-2.145c.Y-1 to I.F-2.145c.Y-480, I.F-3.145c.Y-1 to I.F-3.145c.Y-480, I.F-4.145c.Y-1 to I.F-4.145c.Y-480, I.F-5.145c.Y-1 to I.F-5.145c.Y-480, I.F-6.145c.Y-1 to I.F-6.145c.Y-480).
Table 146c Compounds of the formula I.E-1, I.E-2, I.E-3, I.E-4, I.E-5, I.E-6; I.F-1, I.F-2, I.F-3, I.F-4, I.F-5, I.F-6 in which R⁸ is 3-CN-pyridine-4-yl and the meaning for the combination of R⁶ and R⁷ for each individual compound corresponds in each case to one line of Table Y (compounds I.E-1.146c.Y-1 to I.E-1.146c.Y-480, I.E-2.146c.Y-1 to I.E-2.146c.Y-480, I.E-3.146c.Y-1 to I.E-3.146c.Y-480, I.E-4.146c.Y-1 to I.E-5.146c.Y-480, I.E-5.146c.Y-1 to I.E-3.146c.Y-480, I.E-6.146c.Y-1 to I.E-6.146c.Y-480; I.F-19c.Y-1 to I.F-1.146c.Y-480, I.F-2.146c.Y-1 to I.F-2.146c.Y-480, I. F-3.146c.Y-1 to I. F-3.146c.Y-480, I. F-4.146c.Y-1 to I. F-4.146c.Y-480, I. F-5.146c.Y-1 to I. F-5.146c.Y-480, I. F-6.146c.Y-1 to I. F-6.146c.Y-480).

TABLE Y

| No. | R⁶ | R⁷ |
|---|---|---|
| Y-1 | CH₃ | H |
| Y-2 | C₂H₅ | H |
| Y-3 | n-C₃H₇ | H |
| Y-4 | CH(CH₃)₂ | H |
| Y-5 | n-C₄H₉ | H |
| Y-6 | CH(CH₃)—CH₂—CH₃ | H |
| Y-7 | CH₂—CH(CH₃)₂ | H |
| Y-8 | C(CH₃)₃ | H |
| Y-9 | n-C₅H₁₁ | H |
| Y-10 | CH(CH₃)—CH₂—CH₂—CH₃ | H |
| Y-11 | CH₂—CH(CH₃)—CH₂—CH₃ | H |
| Y-12 | (CH₂)₂—CH(CH₃)₂ | H |
| Y-13 | CH₂—C(CH₃)₃ | H |
| Y-14 | CH(CH₃)—CH(CH₃)—CH₃ | H |
| Y-15 | CH(C₂H₅)₂ | H |
| Y-16 | CH₂—CHF₂ | H |
| Y-17 | CH₂—CF₃ | H |
| Y-18 | (CH₂)₂—CF₃ | H |
| Y-19 | CH═CH₂ | H |
| Y-20 | CH═CHCl | H |
| Y-21 | C═CCl₂ | H |
| Y-22 | CCl═CH₂ | H |
| Y-23 | C(Cl)═CCl₂ | H |
| Y-24 | CH═CHF | H |
| Y-25 | CH═CF₂ | H |
| Y-26 | CF═CH₂ | H |
| Y-27 | CF═CF₂ | H |
| Y-28 | CH═CH—CH₃ | H |
| Y-29 | CH═C(CH₃)₂ | H |
| Y-30 | C(CH₃)═CH₂ | H |
| Y-31 | C(CH₃)═C(CH₃)₂ | H |
| Y-32 | C≡CH | H |
| Y-33 | C≡CCl | H |
| Y-34 | CC—CH₃ | H |
| Y-35 | C≡C—CF₃ | H |
| Y-36 | C≡C-cyclo-C₃H₅ | H |
| Y-37 | CH₂—CH═CH₂ | H |
| Y-38 | CH₂—CH═CHCl | H |
| Y-39 | CH₂—CH═CCl₂ | H |
| Y-40 | CH₂—CH(Cl)═CH₂ | H |
| Y-41 | CH₂—C(Cl)═CCl₂ | H |
| Y-42 | CH₂—CH═CHF | H |
| Y-43 | CH₂—CH═CF₂ | H |
| Y-44 | CH₂—CH(F)═CH₂ | H |
| Y-45 | CH₂—C(F)═CF₂ | H |
| Y-46 | CH₂—CH═CH—CH₃ | H |
| Y-47 | CH₂—CH═C(CH₃)₂ | H |
| Y-48 | CH₂—CH(CH₃)═CH₂ | H |
| Y-49 | CH₂—C(CH₃)═C(CH₃)₂ | H |
| Y-50 | CH₂—CH═(cyclo-C₃H₄) | H |
| Y-51 | CH₂≡CH | H |
| Y-52 | CH₂—C≡CCl | H |
| Y-53 | CH₂—C≡C—CH₃ | H |
| Y-54 | CH₂C≡C—CF₃ | H |
| Y-55 | CH₂C≡C-cyclo-C₃H₅ | H |
| Y-56 | cyclo-C₃H₅ | H |
| Y-57 | cyclo-C₄H₇ | H |
| Y-58 | cyclo-C₅H₉ | H |
| Y-59 | cyclo-C₆H₁₁ | H |
| Y-60 | 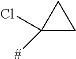 | H |
| Y-61 | 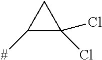 | H |
| Y-62 | 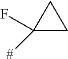 | H |
| Y-63 | 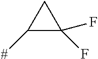 | H |
| Y-64 | 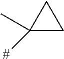 | H |
| Y-65 | 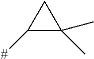 | H |
| Y-66 | 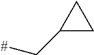 | H |
| Y-67 | 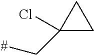 | H |
| Y-68 | 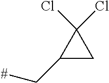 | H |
| Y-69 | 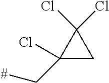 | H |
| Y-70 | 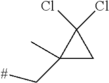 | H |
| Y-71 | 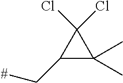 | H |
| Y-72 | 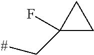 | H |
| Y-73 | 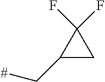 | H |
| Y-74 | 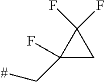 | H |
| Y-75 | 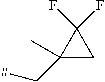 | H |
| Y-76 | 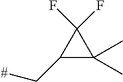 | H |
| Y-77 | 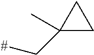 | H |
| Y-78 | 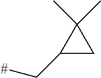 | H |
| Y-79 | 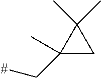 | H |
| Y-80 | 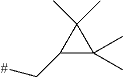 | H |
| Y-81 | CH₃ | CH₃ |
| Y-82 | C₂H₅ | CH₃ |
| Y-83 | n-C₃H₇ | CH₃ |
| Y-84 | CH(CH₃)₂ | CH₃ |
| Y-85 | n-C₄H₉ | CH₃ |
| Y-86 | CH(CH₃)—CH₂—CH₃ | CH₃ |
| Y-87 | CH₂—CH(CH₃)₂ | CH₃ |
| Y-88 | C(CH₃)₃ | CH₃ |
| Y-89 | n-C₅H₁₁ | CH₃ |
| Y-90 | CH(CH₃)—CH₂—CH₂—CH₃ | CH₃ |
| Y-91 | CH₂—CH(CH₃)—CH₂—CH₃ | CH₃ |
| Y-92 | (CH₂)₂—CH(CH₃)₂ | CH₃ |
| Y-93 | CH₂—C(CH₃)₃ | CH₃ |
| Y-94 | CH(CH₃)—CH(CH₃)—CH₃ | CH₃ |
| Y-95 | CH(C₂H₅)₂ | CH₃ |
| Y-96 | CH₂—CHF₂ | CH₃ |
| Y-97 | CH₂—CF₃ | CH₃ |
| Y-98 | (CH₂)₂—CF₃ | CH₃ |
| Y-99 | CH═CH₂ | CH₃ |
| Y-100 | CH═CHCl | CH₃ |
| Y-101 | C═CCl₂ | CH₃ |
| Y-102 | CCl═CH₂ | CH₃ |
| Y-103 | C(Cl)═CCl₂ | CH₃ |
| Y-104 | CH═CHF | CH₃ |
| Y-105 | CH═CF₂ | CH₃ |
| Y-106 | CF═CH₂ | CH₃ |
| Y-107 | CF═CF₂ | CH₃ |
| Y-108 | CH═CH—CH₃ | CH₃ |
| Y-109 | CH═C(CH₃)₂ | CH₃ |
| Y-110 | C(CH₃)═CH₂ | CH₃ |
| Y-111 | C(CH₃)═C(CH₃)₂ | CH₃ |
| Y-112 | C≡CH | CH₃ |
| Y-113 | C≡CCl | CH₃ |
| Y-114 | CC—CH₃ | CH₃ |
| Y-115 | C≡C—CF₃ | CH₃ |
| Y-116 | C≡C-cyclo-C₃H₅ | CH₃ |
| Y-117 | CH₂—CH═CH₂ | CH₃ |
| Y-118 | CH₂—CH═CHCl | CH₃ |
| Y-119 | CH₂—CH═CCl₂ | CH₃ |
| Y-120 | CH₂—CH(Cl)═CH₂ | CH₃ |
| Y-121 | CH₂—C(Cl)═CCl₂ | CH₃ |
| Y-122 | CH₂—CH═CHF | CH₃ |
| Y-123 | CH₂—CH═CF₂ | CH₃ |
| Y-124 | CH₂—CH(F)═CH₂ | CH₃ |
| Y-125 | CH₂—C(F)═CF₂ | CH₃ |
| Y-126 | CH₂—CH═CH—CH₃ | CH₃ |
| Y-127 | CH₂—CH═C(CH₃)₂ | CH₃ |
| Y-128 | CH₂—CH(CH₃)═CH₂ | CH₃ |
| Y-129 | CH₂—C(CH₃)═C(CH₃)₂ | CH₃ |
| Y-130 | CH₂—CH═(cyclo-C₃H₄) | CH₃ |
| Y-131 | CH₂≡CH | CH₃ |
| Y-132 | CH₂—C≡CCl | CH₃ |
| Y-133 | CH₂—C≡C—CH₃ | CH₃ |
| Y-134 | CH₂C≡C—CF₃ | CH₃ |
| Y-135 | CH₂C≡C-cyclo-C₃H₅ | CH₃ |
| Y-136 | cyclo-C₃H₅ | CH₃ |
| Y-137 | cyclo-C₄H₇ | CH₃ |
| Y-138 | cyclo-C₅H₉ | CH₃ |
| Y-139 | cyclo-C₆H₁₁ | CH₃ |
| Y-140 | 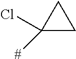 | CH₃ |
| Y-141 | 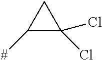 | CH₃ |
| Y-142 | 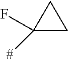 | CH₃ |
| Y-143 | 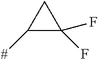 | CH₃ |
| Y-144 | 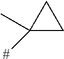 | CH₃ |
| Y-145 | 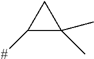 | CH₃ |
| Y-146 | 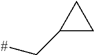 | CH₃ |
| Y-147 | 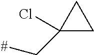 | CH₃ |
| Y-148 | 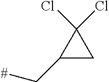 | CH₃ |
| Y-149 | 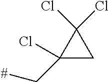 | CH₃ |
| Y-150 | 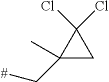 | CH₃ |
| Y-151 | 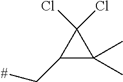 | CH₃ |
| Y-152 | 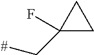 | CH₃ |
| Y-153 | 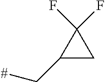 | CH₃ |
| Y-154 | 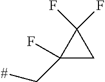 | CH₃ |
| Y-155 | 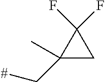 | CH₃ |
| Y-156 | 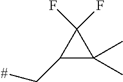 | CH₃ |
| Y-157 | 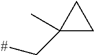 | CH₃ |
| Y-158 | 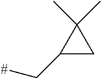 | CH₃ |
| Y-159 | 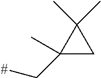 | CH₃ |
| Y-160 | 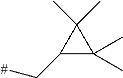 | CH₃ |
| Y-161 | CH₃ | C₂H₅ |
| Y-162 | C₂H₅ | C₂H₅ |
| Y-163 | n-C₃H₇ | C₂H₅ |
| Y-164 | CH(CH₃)₂ | C₂H₅ |
| Y-165 | n-C₄H₉ | C₂H₅ |
| Y-166 | CH(CH₃)—CH₂—CH₃ | C₂H₅ |
| Y-167 | CH₂—CH(CH₃)₂ | C₂H₅ |
| Y-168 | C(CH₃)₃ | C₂H₅ |
| Y-169 | n-C₅H₁₁ | C₂H₅ |
| Y-170 | CH(CH₃)—CH₂—CH₂—CH₃ | C₂H₅ |
| Y-171 | CH₂—CH(CH₃)—CH₂—CH₃ | C₂H₅ |
| Y-172 | (CH₂)₂—CH(CH₃)₂ | C₂H₅ |
| Y-173 | CH₂—C(CH₃)₃ | C₂H₅ |
| Y-174 | CH(CH₃)—CH(CH₃)—CH₃ | C₂H₅ |
| Y-175 | CH(C₂H₅)₂ | C₂H₅ |
| Y-176 | CH₂—CHF₂ | C₂H₅ |
| Y-177 | CH₂—CF₃ | C₂H₅ |
| Y-178 | (CH₂)₂—CF₃ | C₂H₅ |
| Y-179 | CH═CH₂ | C₂H₅ |
| Y-180 | CH═CHCl | C₂H₅ |
| Y-181 | C═CCl₂ | C₂H₅ |
| Y-182 | CCl═CH₂ | C₂H₅ |
| Y-183 | C(Cl)═CCl₂ | C₂H₅ |
| Y-184 | CH═CHF | C₂H₅ |
| Y-185 | CH═CF₂ | C₂H₅ |
| Y-186 | CF═CH₂ | C₂H₅ |
| Y-187 | CF═CF₂ | C₂H₅ |
| Y-188 | CH═CH—CH₃ | C₂H₅ |
| Y-189 | CH═C(CH₃)₂ | C₂H₅ |
| Y-190 | C(CH₃)═CH₂ | C₂H₅ |
| Y-191 | C(CH₃)═C(CH₃)₂ | C₂H₅ |
| Y-192 | C≡CH | C₂H₅ |
| Y-193 | C≡CCl | C₂H₅ |
| Y-194 | CC—CH₃ | C₂H₅ |
| Y-195 | C≡C—CF₃ | C₂H₅ |
| Y-196 | C≡C-cyclo-C₃H₅ | C₂H₅ |
| Y-197 | CH₂—CH═CH₂ | C₂H₅ |
| Y-198 | CH₂—CH═CHCl | C₂H₅ |
| Y-199 | CH₂—CH═CCl₂ | C₂H₅ |
| Y-200 | CH₂—CH(Cl)═CH₂ | C₂H₅ |
| Y-201 | CH₂—C(Cl)═CCl₂ | C₂H₅ |
| Y-202 | CH₂—CH═CHF | C₂H₅ |
| Y-203 | CH₂—CH═CF₂ | C₂H₅ |
| Y-204 | CH₂—CH(F)═CH₂ | C₂H₅ |
| Y-205 | CH₂—C(F)═CF₂ | C₂H₅ |
| Y-206 | CH₂—CH═CH—CH₃ | C₂H₅ |
| Y-207 | CH₂—CH═C(CH₃)₂ | C₂H₅ |
| Y-208 | CH₂—CH(CH₃)═CH₂ | C₂H₅ |
| Y-209 | CH₂—C(CH₃)═C(CH₃)₂ | C₂H₅ |
| Y-210 | CH₂—CH═(cyclo-C₃H₄) | C₂H₅ |
| Y-211 | CH₂≡CH | C₂H₅ |
| Y-212 | CH₂—C≡CCl | C₂H₅ |
| Y-213 | CH₂—C≡C—CH₃ | C₂H₅ |
| Y-214 | CH₂C≡C—CF₃ | C₂H₅ |
| Y-215 | CH₂C≡C-cyclo-C₃H₅ | C₂H₅ |
| Y-216 | cyclo-C₃H₅ | C₂H₅ |
| Y-217 | cyclo-C₄H₇ | C₂H₅ |
| Y-218 | cyclo-C₅H₉ | C₂H₅ |
| Y-219 | cyclo-C₆H₁₁ | C₂H₅ |
| Y-220 | 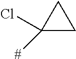 | C₂H₅ |
| Y-221 | 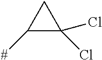 | C₂H₅ |
| Y-222 | 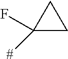 | C₂H₅ |
| Y-223 | 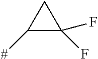 | C₂H₅ |
| Y-224 | 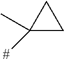 | C₂H₅ |
| Y-225 | 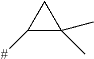 | C₂H₅ |
| Y-226 | 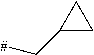 | C₂H₅ |
| Y-227 | 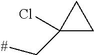 | C₂H₅ |
| Y-228 | 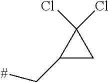 | C₂H₅ |
| Y-229 | 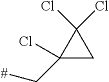 | C₂H₅ |
| Y-230 | 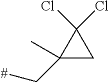 | C₂H₅ |
| Y-231 | 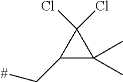 | C₂H₅ |
| Y-232 | 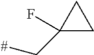 | C₂H₅ |
| Y-233 | 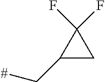 | C₂H₅ |
| Y-234 | 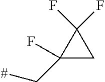 | C₂H₅ |
| Y-235 | 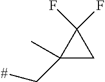 | C₂H₅ |
| Y-236 | 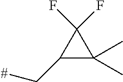 | C₂H₅ |
| Y-237 | 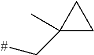 | C₂H₅ |
| Y-238 | 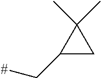 | C₂H₅ |
| Y-239 | 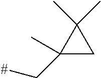 | C₂H₅ |
| Y-240 | 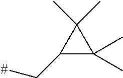 | C₂H₅ |
| Y-241 | CH₃ | F |
| Y-242 | C₂H₅ | F |
| Y-243 | n-C₃H₇ | F |
| Y-244 | CH(CH₃)₂ | F |
| Y-245 | n-C₄H₉ | F |
| Y-246 | CH(CH₃)—CH₂—CH₃ | F |
| Y-247 | CH₂—CH(CH₃)₂ | F |
| Y-248 | C(CH₃)₃ | F |
| Y-249 | n-C₅H₁₁ | F |
| Y-250 | CH(CH₃)—CH₂—CH₂—CH₃ | F |
| Y-251 | CH₂—CH(CH₃)—CH₂—CH₃ | F |
| Y-252 | (CH₂)₂—CH(CH₃)₂ | F |
| Y-253 | CH₂—C(CH₃)₃ | F |
| Y-254 | CH(CH₃)—CH(CH₃)—CH₃ | F |
| Y-255 | CH(C₂H₅)₂ | F |
| Y-256 | CH₂—CHF₂ | F |
| Y-257 | CH₂—CF₃ | F |
| Y-258 | (CH₂)₂—CF₃ | F |
| Y-259 | CH═CH₂ | F |
| Y-260 | CH═CHCl | F |
| Y-261 | C═CCl₂ | F |
| Y-262 | CCl═CH₂ | F |
| Y-263 | C(Cl)═CCl₂ | F |
| Y-264 | CH═CHF | F |
| Y-265 | CH═CF₂ | F |
| Y-266 | CF═CH₂ | F |
| Y-267 | CF═CF₂ | F |
| Y-268 | CH═CH—CH₃ | F |
| Y-269 | CH═C(CH₃)₂ | F |
| Y-270 | C(CH₃)═CH₂ | F |
| Y-271 | C(CH₃)═C(CH₃)₂ | F |
| Y-272 | C≡CH | F |
| Y-273 | C≡CCl | F |
| Y-274 | CC—CH₃ | F |
| Y-275 | C≡C—CF₃ | F |
| Y-276 | C≡C-cyclo-C₃H₅ | F |
| Y-277 | CH₂—CH═CH₂ | F |
| Y-278 | CH₂—CH═CHCl | F |
| Y-279 | CH₂—CH═CCl₂ | F |
| Y-280 | CH₂—CH(Cl)═CH₂ | F |
| Y-281 | CH₂—C(Cl)═CCl₂ | F |
| Y-282 | CH₂—CH═CHF | F |
| Y-283 | CH₂—CH═CF₂ | F |
| Y-284 | CH₂—CH(F)═CH₂ | F |
| Y-285 | CH₂—C(F)═CF₂ | F |
| Y-286 | CH₂—CH═CH—CH₃ | F |
| Y-287 | CH₂—CH═C(CH₃)₂ | F |
| Y-288 | CH₂—CH(CH₃)═CH₂ | F |
| Y-289 | CH₂—C(CH₃)═C(CH₃)₂ | F |
| Y-290 | CH₂—CH═(cyclo-C₃H₄) | F |
| Y-291 | CH₂≡CH | F |
| Y-292 | CH₂—C≡CCl | F |
| Y-293 | CH₂—C≡C—CH₃ | F |
| Y-294 | CH₂C≡C—CF₃ | F |
| Y-295 | CH₂C≡C-cyclo-C₃H₅ | F |
| Y-296 | cyclo-C₃H₅ | F |
| Y-297 | cyclo-C₄H₇ | F |
| Y-298 | cyclo-C₅H₉ | F |
| Y-299 | cyclo-C₆H₁₁ | F |
| Y-300 | 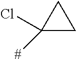 | F |
| Y-301 | 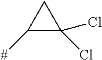 | F |
| Y-302 | 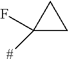 | F |
| Y-303 | 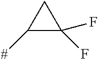 | F |
| Y-304 | 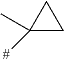 | F |
| Y-305 | 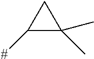 | F |
| Y-306 | 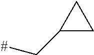 | F |
| Y-307 | 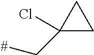 | F |
| Y-308 | 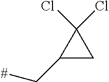 | F |
| Y-309 | 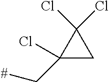 | F |
| Y-310 | 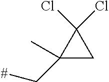 | F |
| Y-311 | 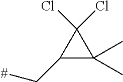 | F |
| Y-312 | 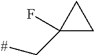 | F |
| Y-313 | 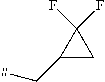 | F |
| Y-314 | 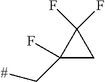 | F |
| Y-315 | 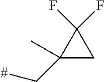 | F |
| Y-316 | 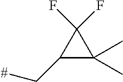 | F |
| Y-317 | 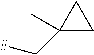 | F |
| Y-318 | 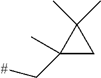 | F |
| Y-319 | 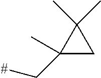 | F |
| Y-320 | 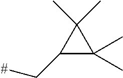 | F |
| Y-321 | CH₃ | Cl |
| Y-322 | C₂H₅ | Cl |
| Y-323 | n-C₃H₇ | Cl |
| Y-324 | CH(CH₃)₂ | Cl |
| Y-325 | n-C₄H₉ | Cl |
| Y-326 | CH(CH₃)—CH₂—CH₃ | Cl |
| Y-327 | CH₂—CH(CH₃)₂ | Cl |
| Y-328 | C(CH₃)₃ | Cl |
| Y-329 | n-C₅H₁₁ | Cl |
| Y-330 | CH(CH₃)—CH₂—CH₂—CH₃ | Cl |
| Y-331 | CH₂—CH(CH₃)—CH₂—CH₃ | Cl |
| Y-332 | (CH₂)₂—CH(CH₃)₂ | Cl |
| Y-333 | CH₂—C(CH₃)₃ | Cl |
| Y-334 | CH(CH₃)—CH(CH₃)—CH₃ | Cl |
| Y-335 | CH(C₂H₅)₂ | Cl |
| Y-336 | CH₂—CHF₂ | Cl |
| Y-337 | CH₂—CF₃ | Cl |
| Y-338 | (CH₂)₂—CF₃ | Cl |
| Y-339 | CH═CH₂ | Cl |
| Y-340 | CH═CHCl | Cl |
| Y-341 | C═CCl₂ | Cl |
| Y-342 | CCl═CH₂ | Cl |
| Y-343 | C(Cl)═CCl₂ | Cl |
| Y-344 | CH═CHF | Cl |
| Y-345 | CH═CF₂ | Cl |
| Y-346 | CF═CH₂ | Cl |
| Y-347 | CF═CF₂ | Cl |
| Y-348 | CH═CH—CH₃ | Cl |
| Y-349 | CH═C(CH₃)₂ | Cl |
| Y-350 | C(CH₃)═CH₂ | Cl |
| Y-351 | C(CH₃)═C(CH₃)₂ | Cl |
| Y-352 | C≡CH | Cl |
| Y-353 | C≡CCl | Cl |
| Y-354 | CC—CH₃ | Cl |
| Y-355 | C≡C—CF₃ | Cl |
| Y-356 | C≡C-cyclo-C₃H₅ | Cl |
| Y-357 | CH₂—CH═CH₂ | Cl |
| Y-358 | CH₂—CH═CHCl | Cl |
| Y-359 | CH₂—CH═CCl₂ | Cl |
| Y-360 | CH₂—CH(Cl)═CH₂ | Cl |
| Y-361 | CH₂—C(Cl)═CCl₂ | Cl |
| Y-362 | CH₂—CH═CHF | Cl |
| Y-363 | CH₂—CH═CF₂ | Cl |
| Y-364 | CH₂—CH(F)═CH₂ | Cl |
| Y-365 | CH₂—C(F)═CF₂ | Cl |
| Y-366 | CH₂—CH═CH—CH₃ | Cl |
| Y-367 | CH₂—CH═C(CH₃)₂ | Cl |
| Y-368 | CH₂—CH(CH₃)═CH₂ | Cl |
| Y-369 | CH₂—C(CH₃)═C(CH₃)₂ | Cl |
| Y-370 | CH₂—CH═(cyclo-C₃H₄) | Cl |
| Y-371 | CH₂≡CH | Cl |
| Y-372 | CH₂—C≡CCl | Cl |
| Y-373 | CH₂—C≡C—CH₃ | Cl |
| Y-374 | CH₂C≡C—CF₃ | Cl |
| Y-375 | CH₂C≡C-cyclo-C₃H₅ | Cl |
| Y-376 | cyclo-C₃H₅ | Cl |
| Y-377 | cyclo-C₄H₇ | Cl |
| Y-378 | cyclo-C₅H₉ | Cl |
| Y-379 | cyclo-C₆H₁₁ | Cl |
| Y-380 | 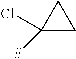 | Cl |
| Y-381 | 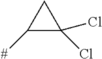 | Cl |
| Y-382 | 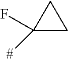 | Cl |
| Y-383 | 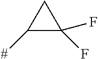 | Cl |
| Y-384 | 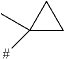 | Cl |
| Y-385 | 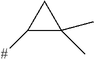 | Cl |
| Y-386 | 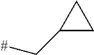 | Cl |
| Y-387 | 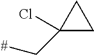 | Cl |
| Y-388 | 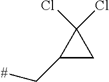 | Cl |
| Y-389 | 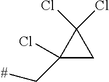 | Cl |
| Y-390 | 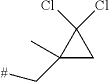 | Cl |
| Y-391 | 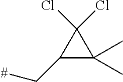 | Cl |
| Y-392 | 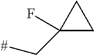 | Cl |
| Y-393 | 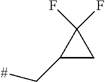 | Cl |
| Y-394 | 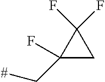 | Cl |
| Y-395 | 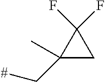 | Cl |
| Y-396 | 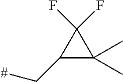 | Cl |
| Y-397 | 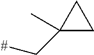 | Cl |
| Y-398 | 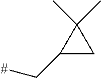 | Cl |
| Y-399 | 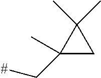 | Cl |
| Y-400 | 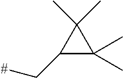 | Cl |
| Y-401 | CH₃ | CN |
| Y-402 | C₂H₅ | CN |
| Y-403 | n-C₃H₇ | CN |
| Y-404 | CH(CH₃)₂ | CN |
| Y-405 | n-C₄H₉ | CN |
| Y-406 | CH(CH₃)—CH₂—CH₃ | CN |
| Y-407 | CH₂—CH(CH₃)₂ | CN |
| Y-408 | C(CH₃)₃ | CN |
| Y-409 | n-C₅H₁₁ | CN |
| Y-410 | CH(CH₃)—CH₂—CH₂—CH₃ | CN |
| Y-411 | CH₂—CH(CH₃)—CH₂—CH₃ | CN |
| Y-412 | (CH₂)₂—CH(CH₃)₂ | CN |
| Y-413 | CH₂—C(CH₃)₃ | CN |
| Y-414 | CH(CH₃)—CH(CH₃)—CH₃ | CN |
| Y-415 | CH(C₂H₅)₂ | CN |
| Y-416 | CH₂—CHF₂ | CN |
| Y-417 | CH₂—CF₃ | CN |
| Y-418 | (CH₂)₂—CF₃ | CN |
| Y-419 | CH═CH₂ | CN |
| Y-420 | CH═CHCl | CN |
| Y-421 | C═CCl₂ | CN |
| Y-422 | CCl═CH₂ | CN |
| Y-423 | C(Cl)═CCl₂ | CN |
| Y-424 | CH═CHF | CN |
| Y-425 | CH═CF₂ | CN |
| Y-426 | CF═CH₂ | CN |
| Y-427 | CF═CF₂ | CN |
| Y-428 | CH═CH—CH₃ | CN |
| Y-429 | CH═C(CH₃)₂ | CN |
| Y-430 | C(CH₃)═CH₂ | CN |
| Y-431 | C(CH₃)═C(CH₃)₂ | CN |
| Y-432 | C≡CH | CN |
| Y-433 | C≡CCl | CN |
| Y-434 | CC—CH₃ | CN |
| Y-435 | C≡C—CF₃ | CN |
| Y-436 | C≡C-cyclo-C₃H₅ | CN |
| Y-437 | CH₂—CH═CH₂ | CN |
| Y-438 | CH₂—CH═CHCl | CN |
| Y-439 | CH₂—CH═CCl₂ | CN |
| Y-440 | CH₂—CH(Cl)═CH₂ | CN |
| Y-441 | CH₂—C(Cl)═CCl₂ | CN |
| Y-442 | CH₂—CH═CHF | CN |
| Y-443 | CH₂—CH═CF₂ | CN |
| Y-444 | CH₂—CH(F)═CH₂ | CN |
| Y-445 | CH₂—C(F)═CF₂ | CN |
| Y-446 | CH₂—CH═CH—CH₃ | CN |
| Y-447 | CH₂—CH═C(CH₃)₂ | CN |
| Y-448 | CH₂—CH(CH₃)═CH₂ | CN |
| Y-449 | CH₂—C(CH₃)═C(CH₃)₂ | CN |
| Y-450 | CH₂—CH═(cyclo-C₃H₄) | CN |
| Y-451 | CH₂≡CH | CN |
| Y-452 | CH₂—C≡CCl | CN |
| Y-453 | CH₂—C≡C—CH₃ | CN |
| Y-454 | CH₂C≡C—CF₃ | CN |
| Y-455 | CH₂C≡C-cyclo-C₃H₅ | CN |
| Y-456 | cyclo-C₃H₅ | CN |
| Y-457 | cyclo-C₄H₇ | CN |
| Y-458 | cyclo-C₅H₉ | CN |
| Y-459 | cyclo-C₆H₁₁ | CN |
| Y-460 | 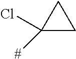 | CN |
| Y-461 | 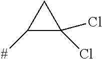 | CN |
| Y-462 | 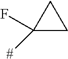 | CN |
| Y-463 | 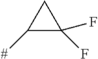 | CN |
| Y-464 | 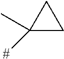 | CN |
| Y-465 | 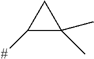 | CN |
| Y-466 | 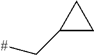 | CN |
| Y-467 | 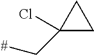 | CN |
| Y-468 | 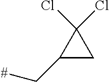 | CN |
| Y-469 | 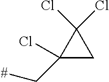 | CN |
| Y-470 | 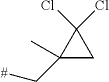 | CN |
| Y-471 | 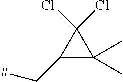 | CN |
| Y-472 | 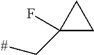 | CN |
| Y-473 | 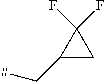 | CN |
| Y-474 | 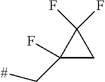 | CN |
| Y-475 | 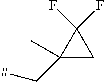 | CN |
| Y-476 | 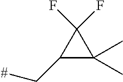 | CN |
| Y-477 | 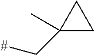 | CN |
| Y-478 | 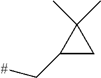 | CN |
| Y-479 | 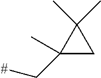 | CN |
| Y-480 | 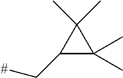 | CN |

Compounds of the present invention can be made as shown in the following schemes, in which, unless otherwise stated, the definition of each variable is as defined above for a compound of formula I. The compounds of the formula I can be prepared according to methods or in analogy to methods that are described in the prior art. The synthesis takes advantage of starting materials that are commercially available or may be prepared according to conventional procedures starting from readily available compounds.

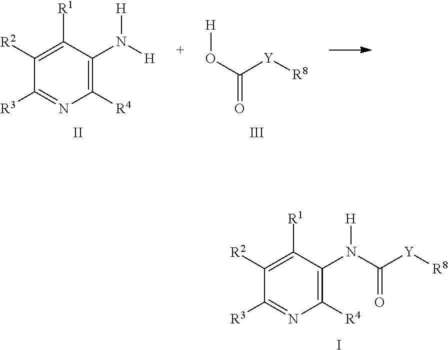

Alternatively compounds of formula I can be synthesized according to other reported methods by coupling derivatives of formula IV, in which X denotes for Cl, Br or I, with amides of formula V. Catalysts used for this reaction include Pd-catalysts such as PdCl₂, Pd(acetate)₂, Pd₂(dba)₃ or others in the presence of an optional ligand like XPhos, DPPF or P(Ph)₃ as described in JACS2002(124), 6043-6048, WO 2013164773, WO 20210075200, OrgLett5 (2003) 4611-4614 or copper-catalysts like Cul as described in RSC Advances Vol. 3(2013), 18787-18790, J Med-Chem54(2011) 6342-6363, WO2011092140.

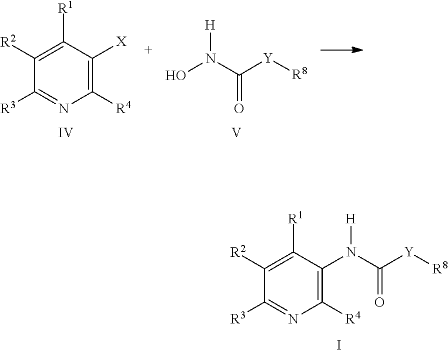

Additionally compounds I can be assessed by reacting esters of type VI with amines at elevated temperatures of 50-250° C., preferably between 120 and 180° C., or in the presence of catalysts like SiO₂ (Tetrahedron, 71(48), 9101-9111; 2015) or Al(CH3)3 (WO 2018-172133), or in the presence of bases like NaH (Indian Journal of Chemistry, Section B: Organic Chemistry Including Medicinal Chemistry, 52B(6), 776-786; 2013) or Ethyl-Mg—Br (Khimiya Geterotsiklich-eskikh Soedinenii, (11), 1515-17; 1988).

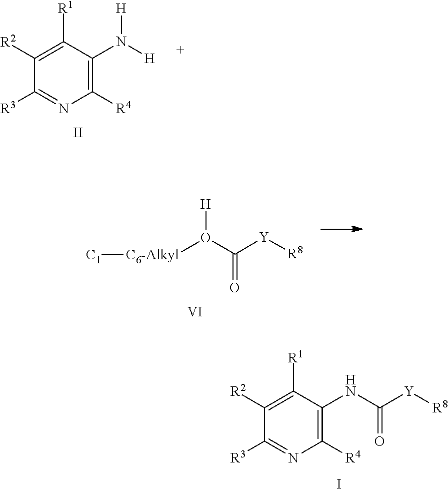

Alternatively compounds I can be synthesized by reacting amines of formula II with activated carboxylic derivatives VII, in which X¹ denotes for halogen (Cl, Br), unsubstituted or substituted phenoxy (for example C₆H₅—O, C₆F₅—O, para-NO₂—C₈H₄, para-Cl—C₆H₄, para-CH₃—C₆H₄, 2,4-Cl₂—C₆H₃), haloalkoxy, like Cl₃C—O or carboxylic groups like C₁-C₄-alkoxy-CO—O (for example CH₃—O—CO—O, C₂H₅—O—CO—O, (CH₃)₂C—CH₂—O—CO—O or halogenalkyl-CO—O (for example CF₃—C—O). Reactions conditions are typical for nucleophilic substitution in an inert solvent like toluene, ethyl acetate, acetone, methyl-t-butyl-ether or the like in the presence of a base like Na₂CO₃, K₂CO₃, NEt₃, NaOH, KOH, (CH₃)₂CH)₂N—C₂H₅) optionally in the presence of water and optionally in the presence of a phase transfer catalyst (i.e. (C₄H₉)₄N⁺Br⁻.(CH₃)₃N⁺(CH₂—C₆H₅)Br or the like) at temperatures between −20° C. to 150° C., preferred between 0° C. and 70° C.

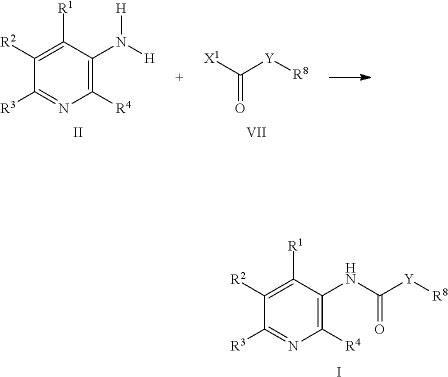

Compounds of formula I in which Y denotes for NR⁵ can be synthesized using compounds VIII wherein X² has the same meanings as X¹ under the conditions described for the reaction between and VII.

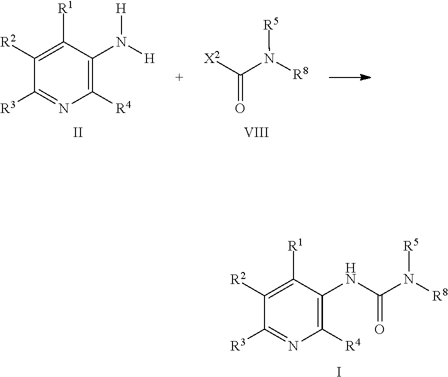

Thioamids of formula I (X═S) can be prepared from amides (X═O) by reaction with P₄S₁₀ (Synthesis 149 (1973)) or Lawesson's reagent (Bull. Soc. Chim. Belge 87, 223 (1978).

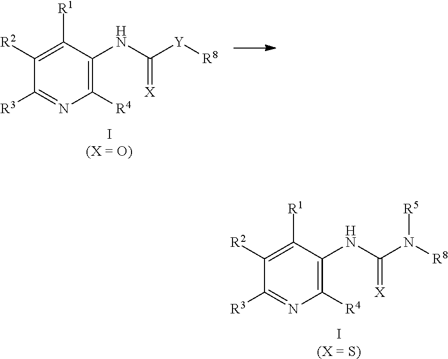

The compounds I and the compositions according to the invention are particularly important in the control of a multitude of phytopathogenic fungi on various cultivated plants, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e. g. conifers; and on the plant propagation material, such as seeds, and the crop material of these plants.

Preferably, compounds I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by mutagenesis or genetic engineering in order to provide a new trait to a plant or to modify an already present trait.

Mutagenesis includes techniques of random mutagenesis using X-rays or mutagenic chemicals, but also techniques of targeted mutagenesis, to create mutations at a specific locus of a plant genome. Targeted mutagenesis techniques frequently use oligonucleotides or proteins like CRISPR/Cas, zinc-finger nucleases, TALENs or meganucleases to achieve the targeting effect. Genetic engineering usually uses recombinant DNA techniques to create modifications in a plant genome which under natural circumstances cannot readily be obtained by cross breeding, mutagenesis or natural recombination. Typically, one or more genes are integrated into the genome of a plant to add a trait or improve a trait. These integrated genes are also referred to as transgenes in the art, while plant comprising such transgenes are referred to as transgenic plants. The process of plant transformation usually produces several transformation events, which differ in the genomic locus in which a transgene has been integrated. Plants comprising a specific transgene on a specific genomic locus are usually described as comprising a specific “event”, which is referred to by a specific event name. Traits which have been introduced in plants or have been modified include herbicide tolerance, insect resistance, increased yield and tolerance to abiotic conditions, like drought.

Herbicide tolerance has been created by using mutagenesis as well as using genetic engineering. Plants which have been rendered tolerant to acetolactate synthase (ALS) inhibitor herbicides by mutagenesis and breeding comprise plant varieties commercially available under the name Clearfield®.

Herbicide tolerance has been created via the use of transgenes to glyphosate, glufosinate, 2,4-D, dicamba, oxynil herbicides, like bromoxynil and ioxynil, sulfonylurea herbicides, ALS inhibitors and 4-hydroxyphenylpyruvate dioxygenase (HPPD) inhibitors, like isoxaflutole and mesotrione.

Transgenes which have been used to provide herbicide tolerance traits comprise: for tolerance to glyphosate: cp4 epsps, epsps grg23ace5, mepsps, 2mepsps, gat4601, gat4621, goxv247; for tolerance to glufosinate: pat and bar, for tolerance to 2,4-D: aad-1, aad-12; for tolerance to dicamba: dmo; for tolerance to oxynil herbicides: bxn; for tolerance to sulfonylurea herbicides: zm-hra, csr1-2, gm-hra, S4-HrA; for tolerance to ALS inhibitors: csr1-2; and for tolerance to HPPD inhibitors: hppdPF, W336, avhppd-03.

Transgenic corn events comprising herbicide tolerance genes include, but are not limited to, DAS40278, MON801, MON802, MON809, MON810, MON832, MON87411, MON87419, MON87427, MON88017, MON89034, NK603, GA21, MZHG0JG, HCEM485, VCO-Ø1981-5, 676, 678, 680, 33121, 4114, 59122, 98140, Bt10, Bt176, CBH-351, DBT418, DLL25, MS3, MS6, MZIR098, T25, TC1507 and TC6275.

Transgenic soybean events comprising herbicide tolerance genes include, but are not limited to, GTS 40-3-2, MON87705, MON87708, MON87712, MON87769, MON89788, A2704-12, A2704-21, A5547-127, A5547-35, DP356043, DAS44406-6, DAS68416-4, DAS-81419-2, GU262, SYHTØH2, W62, W98, FG72 and CV127.

Transgenic cotton events comprising herbicide tolerance genes include, but are not limited to, 19-51a, 31707, 42317, 81910, 281-24-236, 3006-210-23, BXN10211, BXN10215, BXN10222, BXN10224, MON1445, MON1698, MON88701, MON88913, GHB119, GHB614, LLCotton25, T303-3 and T304-40.

Transgenic canola events comprising herbicide tolerance genes are for example, but not excluding others, MON88302, HCR-1, HCN10, HCN28, HCN92, MS1, MS8, PHY14, PHY23, PHY35, PHY36, RF1, RF2 and RF3.

Insect resistance has mainly been created by transferring bacterial genes for insecticidal proteins to plants: Transgenes which have most frequently been used are toxin genes of Bacillus spp. and synthetic variants thereof, like cry1A, cry1Ab, cry1Ab-Ac, cry1Ac, cry1A.105, cry1F, cry1Fa2, cry2Ab2, cry2Ae, mcry3A, ecry3.1Ab, cry3Bb1, cry34Ab1, cry35Ab1, cry9C, vip3A(a), vip3Aa20. However, also genes of plant origin, such as genes coding for protease inhibitors, like CpTI and pinII, have been transferred to other plants. A further approach uses transgenes such as dvsnf7 to produce double-stranded RNA in plants.

Transgenic corn events comprising genes for insecticidal proteins or double stranded RNA include, but are not limited to, Bt10, Bt11, Bt176, MON801, MON802, MON809, MON810, MON863, MON87411, MON88017, MON89034, 33121, 4114, 5307, 59122, TC1507, TC6275, CBH-351, MIR162, DBT418 and MZIR098. Transgenic soybean events comprising genes for insecticidal proteins include, but are not limited to, MON87701, MON87751 and DAS-81419. Transgenic cotton events comprising genes for insecticidal proteins include, but are not limited to, SGK321, MON531, MON757, MON1076, MON15985, 31707, 31803, 31807, 31808, 42317, BNLA-601, Event1, COT67B, COT102, T303-3, T304-40, GFM Cry1A, GK12, MLS 9124, 281-24-236, 3006-210-23, GHB119 and SGK321.

Increased yield has been created by using the transgene athb17, being present for example in corn event MON87403, or by using the transgene bbx32, being present for example in the soybean event MON87712.

Cultivated plants comprising a modified oil content have been created by using the transgenes: gm-fad2-1, Pj.D6D, Nc.Fad3, fad2-1A and fatb1-A. Soybean events comprising at least one of these genes are: 260-05, MON87705 and MON87769.

Tolerance to abiotic conditions, such as drought, has been created by using the transgene cspB, comprised by the corn event MON87460 and by using the transgene Hahb-4, comprised by soybean event IND-00410-5.

Traits are frequently combined by combining genes in a transformation event or by combining different events during the breeding process resulting in a cultivated plant with stacked traits. Preferred combinations of traits are combinations of herbicide tolerance traits to different groups of herbicides, combinations of insect tolerance to different kind of insects, in particular tolerance to lepidopteran and coleopteran insects, combinations of herbicide tolerance with one or several types of insect resistance, combinations of herbicide tolerance with increased yield as well as combinations of herbicide tolerance and tolerance to abiotic conditions.

Plants comprising singular or stacked traits as well as the genes and events providing these traits are well known in the art. For example, detailed information as to the mutagenized or integrated genes and the respective events are available from websites of the organizations “International Service for the Acquisition of Agri-biotech Applications (ISAAA)” (http://www.isaaa.org/gmapprovaldatabase) and the “Center for Environmental Risk Assessment (CERA)” (http://cera-gmc.org/GMCropDatabase). Further information on specific events and methods to detect them can be found for canola events MS1, MS8, RF3, GT73, MON88302, KK179 in WO01/031042, WO01/041558, WO01/041558, WO02/036831, WO11/153186, WO13/003558, for cotton events MON1445, MON15985, MON531 (MON15985), LLCotton25, MON88913, COT102, 281-24-236, 3006-210-23, COT67B, GHB614, T304-40, GHB119, MON88701, 81910 in WO02/034946, WO02/100163, WO02/100163, WO03/013224, WO04/072235, WO04/039986, WO05/103266, WO05/103266, WO06/128573, WO07/017186, WO08/122406, WO08/151780, WO12/134808, WO13/112527; for corn events GA21, MON810, DLL25, TC1507, MON863, MIR604, LY038, MON88017, 3272, 59122, NK603, MIR162, MON89034, 98140, 32138, MON87460, 5307, 4114, MON87427, DAS40278, MON87411, 33121, MON87403, MON87419 in WO98/044140, U.S. Ser. No. 02/102,582, U.S. Ser. No. 03/126,634, WO04/099447, WO04/011601, WO05/103301, WO05/061720, WO05/059103, WO06/098952, WO06/039376, US2007/292854, WO07/142840, WO07/140256, WO08/112019, WO09/103049, WO09/111263, WO10/077816, WO11/084621, WO11/062904, WO11/022469, WO13/169923, WO14/116854, WO15/053998, WO15/142571; for potato events E12, F10, J3, J55, V11, X17, Y9 in WO14/178910, WO14/178913, WO14/178941, WO14/179276, WO16/183445, WO17/062831, WO17/062825; for rice events LLRICE06, LLRICE601, LLRICE62 in WO00/026345, WO00/026356, WO00/026345; and for soybean events H7-1, MON89788, A2704-12, A5547-127, DP305423, DP356043, MON87701, MON87769, CV127, MON87705, DAS68416-4, MON87708, MON87712, SYHTOH2, DAS81419, DAS81419 x DAS44406-6, MON87751 in WO04/074492, WO06/130436, WO06/108674, WO06/108675, WO08/054747, WO08/002872, WO09/064652, WO09/102873, WO10/080829, WO10/037016, WO11/066384, WO11/034704, WO12/051199, WO12/082548, WO13/016527, WO13/016516, WO14/201235.

The use of compounds I and compositions according to the invention, respectively, on cultivated plants may result in effects which are specific to a cultivated plant comprising a certain gene or event. These effects might involve changes in growth behavior or changed resistance to biotic or abiotic stress factors. Such effects may in particular comprise enhanced yield, enhanced resistance or tolerance to insects, nematodes, fungal, bacterial, mycoplasma, viral or viroid pathogens as well as early vigor, early or delayed ripening, cold or heat tolerance as well as changed amino acid or fatty acid spectrum or content.

The compounds I and compositions thereof, respectively, are particularly suitable for controlling the following plant diseases: Albugo spp. (white rust) on ornamentals, vegetables (e. g. A. candida) and sunflowers (e. g. A. tragopogonis); Alternaria spp. (Alternaria leaf spot) on vegetables (e.g. A. dauci or A. porri), oilseed rape (A. brassicicola or brassicae), sugar beets (A. tenuis), fruits (e.g. A. grandis), rice, soybeans, potatoes and tomatoes (e. g. A. solani, A. grandis or A. alternata), tomatoes (e. g. A. solani or A. alternata) and wheat (e.g. A. triticina); Aphanomyces spp. on sugar beets and vegetables; Ascochyta spp. on cereals and vegetables, e. g. A. tritici (anthracnose) on wheat and A. hordei on barley; Aureobasidium zeae (syn. Kapatiella zeae) on corn; Bipolaris and Drechslera spp. (teleomorph: Cochliobolus spp.), e. g. Southern leaf blight (D. maydis) or Northern leaf blight (B. zeicola) on corn, e. g. spot blotch (B. sorokiniana) on cereals and e. g. B. oryzae on rice and turfs; Blumeria (formerly Erysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages); B. squamosa or B. allii on onion family), oilseed rape, ornamentals (e.g. B eliptica), vines, forestry plants and wheat; Bremia lactucae (downy mildew) on lettuce; Ceratocystis (syn. Ophiostoma) spp. (rot or wilt) on broad-leaved trees and evergreens, e. g. C. ulmi (Dutch elm disease) on elms; Cercospora spp. (Cercospora leaf spots) on corn (e. g. Gray leaf spot: C. zeae-maydis), rice, sugar beets (e. g. C. beticola), sugar cane, vegetables, coffee, soybeans (e. g. C. sojina or C. kikuchii) and rice; Cladobotryum (syn. Dactylium) spp. (e.g. C. mycophilum (formerly Dactylium dendroides, teleomorph: Nectria albertinii, Nectria rosella syn. Hypomyces rosellus) on mushrooms; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypii), corn (e. g. C. graminicola: Anthracnose stalk rot), soft fruits, potatoes (e. g. C. coccodes: black dot), beans (e. g. C. lindemuthianum), soybeans (e. g. C. truncatum or C. gloeosporioides), vegetables (e.g. C. lagenarium or C. capsici), fruits (e.g. C. acutatum), coffee (e.g. C. coffeanum or C. kahawae) and C. gloeosporioides on various crops; Corticium spp., e. g. C. sasakii (sheath blight) on rice; Corynespora cassiicola (leaf spots) on soybeans, cotton and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (formerly Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e. g. E. pisi), such as cucurbits (e. g. E. cichoracearum), cabbages, oilseed rape (e. g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e. g. wheat or barley) and corn; Gibberella spp. on cereals (e. g. G. zeae) and rice (e. g. G. fujikuroi: Bakanae disease); Glomerella cingulata on vines, pome fruits and other plants and G. gossypii on cotton; Grain-staining complex on rice; Guignardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals, potatoes and rice; Hemileia spp., e. g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (syn. Monilia spp.: bloom and twig blight, brown rot) on stone fruits and other rosaceous plants; Mycosphaerella spp. on cereals, bananas, soft fruits and ground nuts, such as e. g. M. graminicola (anamorph: Zymoseptoria tritici formerly Septoria tritici: Septoria blotch) on wheat or M. fijiensis (syn. Pseudocercospora fijiensis: black Sigatoka disease) and M. musicola on bananas, M. arachidicola (syn. M. arachidis or Cercospora arachidis), M. berkeleyi on peanuts, M. pisi on peas and M. brassiciola on brassicas; Peronospora spp. (downy mildew) on cabbage (e. g. P. brassicae), oilseed rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (syn. Leptosphaeria biglobosa and L. maculans: root and stem rot) on oilseed rape and cabbage, P. betae (root rot, leaf spot and damping-off) on sugar beets and P. zeae-maydis (syn. Phyllostica zeae) on corn; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsici), soybeans (e. g. P. megasperma, syn. P. sojae), potatoes and tomatoes (e. g. P. infestans: late blight) and broad-leaved trees (e. g. P. ramorum: sudden oak death); Plasmodiophora brassicae (club root) on cabbage, oilseed rape, radish and other plants; Plasmopara spp., e. g. P. viticola (grapevine downy mildew) on vines and P. halstedii on sunflowers; Podosphaera spp. (powdery mildew) on rosaceous plants, hop, pome and soft fruits (e. g. P. leucotricha on apples) and curcurbits (P. xanthii); Polymyxa spp., e. g. on cereals, such as barley and wheat (P. graminis) and sugar beets (P. betae) and thereby transmitted viral diseases; Pseudocercosporella herpotrichoides (syn. Oculimacula yallundae, O. acuformis: eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or, rotbrenner, anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, P. kuehnii (orange rust) on sugar cane and P. asparagi on asparagus; Pyrenopeziza spp., e.g. P. brassicae on oilseed rape; Pyrenophora (anamorph: Drechslera) tritici-repentis (tan spot) on wheat or P. teres (net blotch) on barley; Pyricularia spp., e. g. P. oryzae (teleomorph: Magnaporthe grisea: rice blast) on rice and P. grisea on turf and cereals; Pythium spp. (damping-off) on turf, rice, corn, wheat, cotton, oilseed rape, sunflowers, soybeans, sugar beets, vegetables and various other plants (e. g. P. ultimum or P. aphanidermatum) and P. oligandrum on mushrooms; Ramularia spp., e. g. R. collo-cygni (Ramularia leaf spots, Physiological leaf spots) on barley, R. areola (teleomorph: Mycosphaerella areola) on cotton and R. beticola on sugar beets; Rhizoctonia spp. on cotton, rice, potatoes, turf, corn, oilseed rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis and R. commune (scald) on barley, rye and triticale; Sarocladium oryzae and S. attenuatum (sheath rot) on rice; Sclerotinia spp. (stem rot or white mold) on vegetables (S. minor and S. sclerotiorum) and field crops, such as oilseed rape, sunflowers (e. g. S. sclerotiorum) and soybeans, S. rolfsii (syn. Athelia rolfsii) on soybeans, peanut, vegetables, corn, cereals and ornamentals; Septoria spp. on various plants, e. g. S. glycines (brown spot) on soybeans, S. tritici (syn. Zymoseptoria tritici, Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setosphaeria spp. (leaf blight) on corn (e. g. S. turcicum, syn. Helminthosporium turcicum) and turf; Sphacelotheca spp. (smut) on corn, (e. g. S. reiliana, syn. Ustilago reiliana: head smut), sorghum und sugar cane; Sphaerotheca fuliginea (syn. Podosphaera xanthii: powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum, syn. Septoria nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici (syn. T. caries, wheat bunt) and T. controversa (dwarf bunt) on wheat; Trichoderma harzianum on mushrooms; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli), sugar beets (e. g. U. betae or U. beticola) and on pulses (e.g. U. vignae, U. pisi, U. viciae-fabae and U. fabae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. longisporum on oilseed rape, V. dahliae on strawberries, oilseed rape, potatoes and tomatoes, and V. fungicola on mushrooms; Zymoseptoria tritici on cereals.

In a preferred embodiment the compounds I, their mixtures with other active compounds as defined herein and compositions thereof, respectively, are particularly suitable for controlling the following plant diseases: Puccinia spp. (rusts) on various plants, for example, but not limited to P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, P. sorghi (common rust) on maize, P. polysora (southern rust) on maize; P. coronata e.g. on oats, P. sorghi und P. polysora on corn; Puccinia spp. on other crops, e.g. P. heliathi on sunflower, P. arachidis on peanuts; Uromyces spp. on pulses and other crops crops, e.g. Uromyces viciae-fabae, Uromyces vigniae, Uromyces pisi, U. ciceris-arietini, U. betae syn U. beticola; and Phakopsoraceae spp. on various plants, in particular Phakopsora pachyrhizi and P. meibomiae (soybean rust) on soybeans.

Additionally, to date, no cross-resistance has been observed between the compounds I and the current fungicidal solutions used to control phytopathogenic fungi including but not limited to Zymoseptoria tritici, Phakopsora pachyrhizi, Botrytis cinerea, Blumeria graminis, Pyrenophora tritici-repentis, Pyrenophora teres, Alternaria spp., Plasmopara viticola, preferably selected from Zymospeptoria tritici and Phakopsora pachyrhizi:

Fungicide-resistant strains of the above-mentioned phytopathgenic fungi have been reported, with strains resistant to one or more fungicides from various fungicidal mode of action classes being observed including but not limited to beta-tubulin assembly inhibitors, sterol demethylation-inhibitors (DMI), quinone-outside-inhibitors (QoI) and succinate dehydrogenase inhibitors (SDHI). Thus, compounds I are useful to control phytopathogenic fungi comprising at least one of the following mutations: E198A/G/K or F200Y in the beta-tubulin gene conferring resistance to beta-tubulin assembly inhibitors (Phytopathol (2008) 98: 397-404), 1365N/S, V368F, Q369H/P, N373S, T447S in the histidine kinase gene Os1 conferring resistance to MAP/histidin kinase inhibitors (dicarboximides; ibida); G143A, G137R or F129L in the mitochondrial cytochrome B Gene (Phytopathol (2003) 93: 891-900; Pest Manag Sci (2016) 72: 1211-1215) resulting in resistance to QoI; V136A, Y137F (homologous to Y144F/H e.g. in Parastagonospora nodorum or Y136F e.g. in Erysiphe necator), K147Q, A379G, 1381V, G461S or S509T in the Cyp51 gene resulting in resistance to DMI (Phytopathol (2016) 106: 1278-1284); P225T/L/F, N2251/T, R265P, T2681/A, H272R/Y/L, H277Y or N2301 in the succinate dehydrogenase ironsulfur subunit gene SdhB, K49E, R64K, N75S, G79R, T79N/1, W80S, P80H/L, N86S/A, G91R, H134R, S135R, H146R/LK, R151S/T/M, H152R, H153R, 1161S, V166M, T168R and G171D in the subunit C gene SdhC; and 150F, D124E, M114V, H134R, D145G in the subunit D gene SdhD conferring resistance to SDHI ((2002) 58: 876-88; Pest Manag Sci (2014) 70: 378-388; Environ Microbiol (2014) 16: 2253-66; Pest Manag Sci (2018) 74: 672-681; http://www.frac.info/working-group/sdhi-fungicides), and 186F in the subunit C gene SdhC (Journal of Plant Diseases and Protection 125, 21-26).

The compounds I and compositions thereof, respectively, are also suitable for controlling harmful fungi in the protection of stored products or harvest and in the protection of materials.

The term “protection of materials” is to be understood to denote the protection of technical and non-living materials, such as adhesives, glues, wood, paper and paperboard, textiles, leather, paint dispersions, plastics, cooling lubricants, fiber or fabrics, against the infestation and destruction by harmful microorganisms, such as fungi and bacteria. As to the protection of wood and other materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicillium spp., Trichoderma spp., Alternaria spp., Paecilomyces spp. and Zygomycetes such as Mucor spp., and in addition in the protection of stored products and harvest the following yeast fungi are worthy of note: Candida spp. and Saccharomyces cerevisae.

The method of treatment according to the invention can also be used in the field of protecting stored products or harvest against attack of fungi and microorganisms. According to the present invention, the term “stored products” is understood to denote natural substances of plant or animal origin and their processed forms, which have been taken from the natural life cycle and for which long-term protection is desired. Stored products of crop plant origin, such as plants or parts thereof, for example stalks, leafs, tubers, seeds, fruits or grains, can be protected in the freshly harvested state or in processed form, such as pre-dried, moistened, comminuted, ground, pressed or roasted, which process is also known as post-harvest treatment. Also falling under the definition of stored products is timber, whether in the form of crude timber, such as construction timber, electricity pylons and barriers, or in the form of finished articles, such as furniture or objects made from wood. Stored products of animal origin are hides, leather, furs, hairs and the like. The combinations according the present invention can prevent disadvantageous effects such as decay, discoloration or mold. Preferably “stored products” is understood to denote natural substances of plant origin and their processed forms, more preferably fruits and their processed forms, such as pomes, stone fruits, soft fruits and citrus fruits and their processed forms.

The compounds I and compositions thereof, respectively, may be used for improving the health of a plant. The invention also relates to a method for improving plant health by treating a plant, its propagation material and/or the locus where the plant is growing or is to grow with an effective amount of compounds I and compositions thereof, respectively.

The term “plant health” is to be understood to denote a condition of the plant and/or its products which is determined by several indicators alone or in combination with each other such as yield (e. g. increased biomass and/or increased content of valuable ingredients), plant vigor (e. g. improved plant growth and/or greener leaves (“greening effect”)), quality (e. g. improved content or composition of certain ingredients) and tolerance to abiotic and/or biotic stress. The above identified indicators for the health condition of a plant may be interdependent or may result from each other.

The compounds of formula I can be present in different crystal modifications whose biological activity may differ. They are likewise subject matter of the present invention.

The compounds I are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compounds I as such or a composition comprising at least one compound I prophylactically either at or before planting or transplanting.

The invention also relates to agrochemical compositions comprising an auxiliary and at least one compound I according to the invention.

An agrochemical composition comprises a fungicidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, their N-oxides and salts can be converted into customary types of agrochemical compositions, e. g. solutions, emulsions, suspensions, dusts, powders, pastes, granules, pressings, capsules, and mixtures thereof. Examples for composition types are suspensions (e. g. SC, OD, FS), emulsifiable concentrates (e. g. EC), emulsions (e. g. EW, EO, ES, ME), capsules (e. g. CS, ZC), pastes, pastilles, wettable powders or dusts (e. g. WP, SP, WS, DP, DS), pressings (e. g. BR, TB, DT), granules (e. g. WG, SG, GR, FG, GG, MG), insecticidal articles (e. g. LN), as well as gel formulations for the treatment of plant propagation materials such as seeds (e. g. GF). These and further compositions types are defined in the “Catalogue of pesticide formulation types and international coding system”, Technical Monograph No. 2, 6^{th} Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e. g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e. g. ethanol, propanol, butanol, benzyl alcohol, cyclohexanol; glycols; DMSO; ketones, e. g. cyclohexanone; esters, e. g. lactates, carbonates, fatty acid esters, gamma-butyrolactone; fatty acids; phosphonates; amines; amides, e. g. N-methyl pyrrolidone, fatty acid dimethyl amides; and mixtures thereof.

Suitable solid carriers or fillers are mineral earths, e. g. silicates, silica gels, talc, kaolins, limestone, lime, chalk, clays, dolomite, diatomaceous earth, bentonite, calcium sulfate, magnesium sulfate, magnesium oxide; polysaccharides, e. g. cellulose, starch; fertilizers, e. g. ammonium sulfate, ammonium phosphate, ammonium nitrate, ureas; products of vegetable origin, e. g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl naphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e. g. xanthan gum, carboxymethyl cellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e. g. in red, blue, or green) are pigments of low water solubility and water-soluble dyes. Examples are inorganic colorants (e. g. iron oxide, titan oxide, iron hexacyanoferrate) and organic colorants (e. g. alizarin-, azo- and phthalocyanine colorants).

Suitable tackifiers or binders are polyvinyl pyrrolidones, polyvinyl acetates, polyvinyl alcohols, polyacrylates, biological or synthetic waxes, and cellulose ethers.

Examples for composition types and their preparation are:

i) Water-soluble concentrates (SL, LS)

10-60 wt % of a compound I and 5-15 wt % wetting agent (e. g. alcohol alkoxylates) are dissolved in water and/or in a water-soluble solvent (e. g. alcohols) ad 100 wt %. The active substance dissolves upon dilution with water.

ii) Dispersible concentrates (DC)

5-25 wt % of a compound I and 1-10 wt % dispersant (e. g. polyvinyl pyrrolidone) are dissolved in organic solvent (e. g. cyclohexanone) ad 100 wt %. Dilution with water gives a dispersion.

iii) Emulsifiable concentrates (EC)

15-70 wt % of a compound I and 5-10 wt % emulsifiers (e. g. calcium dodecylbenzenesulfonate and castor oil ethoxylate) are dissolved in water-insoluble organic solvent (e. g. aromatic hydrocarbon) ad 100 wt %. Dilution with water gives an emulsion.

iv) Emulsions (EW, EO, ES)

5-40 wt % of a compound I and 1-10 wt % emulsifiers (e. g. calcium dodecylbenzenesulfonate and castor oil ethoxylate) are dissolved in 20-40 wt % water-insoluble organic solvent (e. g. aromatic hydrocarbon). This mixture is introduced into water ad 100 wt % by means of an emulsifying machine and made into a homogeneous emulsion. Dilution with water gives an emulsion.

v) Suspensions (SC, OD, FS)

In an agitated ball mill, 20-60 wt % of a compound I are comminuted with addition of 2-10 wt % dispersants and wetting agents (e. g. sodium lignosulfonate and alcohol ethoxylate), 0.1-2 wt % thickener (e. g. xanthan gum) and water ad 100 wt % to give a fine active substance suspension. Dilution with water gives a stable suspension of the active substance. For FS type composition up to 40 wt % binder (e. g. polyvinyl alcohol) is added.

vi) Water-dispersible granules and water-soluble granules (WG, SG)

50-80 wt % of a compound I are ground finely with addition of dispersants and wetting agents (e. g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

vii) Water-dispersible powders and water-soluble powders (WP, SP, WS)

50-80 wt % of a compound I are ground in a rotor-stator mill with addition of 1-5 wt % dispersants (e. g. sodium lignosulfonate), 1-3 wt % wetting agents (e. g. alcohol ethoxylate) and solid carrier (e. g. silica gel) ad 100 wt %. Dilution with water gives a stable dispersion or solution of the active substance.

viii) Gel (GW, GF)

In an agitated ball mill, 5-25 wt % of a compound I are comminuted with addition of 3-10 wt % dispersants (e. g. sodium lignosulfonate), 1-5 wt % thickener (e. g. carboxymethyl cellulose) and water ad 100 wt % to give a fine suspension of the active substance. Dilution with water gives a stable suspension of the active substance.

ix) Microemulsion (ME)

5-20 wt % of a compound I are added to 5-30 wt % organic solvent blend (e. g. fatty acid dimethyl amide and cyclohexanone), 10-25 wt % surfactant blend (e. g. alcohol ethoxylate and arylphenol ethoxylate), and water ad 100%. This mixture is stirred for 1 h to produce spontaneously a thermodynamically stable microemulsion.

x) Microcapsules (CS)

An oil phase comprising 5-50 wt % of a compound I, 0-40 wt % water insoluble organic solvent (e. g. aromatic hydrocarbon), 2-15 wt % acrylic monomers (e. g. methylmethacrylate, methacrylic acid and a di- or triacrylate) are dispersed into an aqueous solution of a protective colloid (e. g. polyvinyl alcohol). Radical polymerization results in the formation of poly(meth)acrylate microcapsules. Alternatively, an oil phase comprising 5-50 wt % of a compound I according to the invention, 0-40 wt % water insoluble organic solvent (e. g. aromatic hydrocarbon), and an isocyanate monomer (e. g. diphenylmethene-4,4′-diisocyanatae) are dispersed into an aqueous solution of a protective colloid (e. g. polyvinyl alcohol). The addition of a polyamine (e. g. hexamethylenediamine) results in the formation of polyurea microcapsules. The monomers amount to 1-10 wt %. The wt % relate to the total CS composition.

xi) Dustable powders (DP, DS)

1-10 wt % of a compound I are ground finely and mixed intimately with solid carrier (e. g. finely divided kaolin) ad 100 wt %.

xii) Granules (GR, FG)

0.5-30 wt % of a compound I is ground finely and associated with solid carrier (e. g. silicate) ad 100 wt %. Granulation is achieved by extrusion, spray-drying or fluidized bed.

xiii) Ultra-low volume liquids (UL)

1-50 wt % of a compound I are dissolved in organic solvent (e. g. aromatic hydrocarbon) ad 100 wt %.

The compositions types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, more preferably between 1 and 70%, and in particular between 10 and 60%, by weight of active substance. The active substances are employed in a purity of from 90% to 100%, preferably from 95% to 100% (according to NMR spectrum).

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed. The compositions in question give, after two-to-tenfold dilution, active substance concentrations of from 0.01 to 60% by weight, preferably from 0.1 to 40%, in the ready-to-use preparations. Application can be carried out before or during sowing. Methods for applying compound I and compositions thereof, respectively, onto plant propagation material, especially seeds, include dressing, coating, pelleting, dusting, and soaking as well as in-furrow application methods. Preferably, compound I or the compositions thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e. g. herbicides, insecticides, fungicides, growth regulators, safeners, biopesticides) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

A pesticide is generally a chemical or biological agent (such as pesticidal active ingredient, compound, composition, virus, bacterium, antimicrobial or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease. The term “pesticide” includes also plant growth regulators that alter the expected growth, flowering, or reproduction rate of plants; defoliants that cause leaves or other foliage to drop from a plant, usually to facilitate harvest; desiccants that promote drying of living tissues, such as unwanted plant tops; plant activators that activate plant physiology for defense of against certain pests; safeners that reduce unwanted herbicidal action of pesticides on crop plants; and plant growth promoters that affect plant physiology e.g. to increase plant growth, biomass, yield or any other quality parameter of the harvestable goods of a crop plant.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank or any other kind of vessel used for applications (e. g. seed treater drums, seed pelleting machinery, knapsack sprayer) and further auxiliaries may be added, if appropriate.

Consequently, one embodiment of the invention is a kit for preparing a usable pesticidal composition, the kit comprising a) a composition comprising component 1) as defined herein and at least one auxiliary; and b) a composition comprising component 2) as defined herein and at least one auxiliary; and optionally c) a composition comprising at least one auxiliary and optionally a further active component 3) as defined herein.

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained. In one embodiment of the invention compounds according to the Table 1a to table 43a, Table 1b to Table 43b and Table 1c to 146c can be preferably mixed with the pesticides II of the groups A to O as disclosed below.

The following list of pesticides II (e. g. pesticidally-active substances and biopesticides), in conjunction with which the compounds I can be used, is intended to illustrate the possible combinations but does not limit them:

### A) Respiration Inhibitors

Inhibitors of complex III at Qₒ site: azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxystrobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylideneaminooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21a), methyl-N-[2-[(1,4-dimethyl-5-phenylpyrazol-3-yl)oxylmethyl]phenyl]-N-methoxy-carbamate (A.1.22), metyltetrapole (A.1.25), (Z,2E)-5-[1-(2,4-dichlorophenyl)pyrazol-3-yl]-oxy-2-methoxyimino-N,3-dimethyl-pent-3-enamide (A.1.34), (Z,2E)-5-[1-(4-chlorophenyl)pyrazol-3-yl]oxy-2-methoxyimino-N,3-dimethylpent-3-enamide (A.1.35), pyriminostrobin (A.1.36), bifujunzhi (A.1.37), 2-(ortho-((2,5-dimethylphenyl-oxymethylen)phenyl)-3-methoxy-acrylic acid methylester (A.1.38);
inhibitors of complex III at Qi site: cyazofamid (A.2.1), amisulbrom (A.2.2), [(6S,7R,8R)-8-benzyl-3-[(3-hydroxy-4-methoxy-pyridine-2-carbonyl)amino]-6-methyl-4,9-dioxo-1,5-dioxonan-7-yl] 2-methylpropanoate (A.2.3), fenpicoxamid (A.2.4), florylpicoxamid (A.2.5);
inhibitors of complex II: benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.15), penthiopyrad (A.3.16), pydiflumetofen (A.3.17), pyraziflumid (A.3.18), sedaxane (A.3.19), tecloftalam (A.3.20), thifluzamide (A.3.21), inpyrfluxam (A.3.22), pyrapropoyne (A.3.23), fluindapyr (A.3.28), N-[2-[2-chloro-4-(trifluoro¬methyl)phenoxy]phenyl]-3-(difluoromethyl)-5-fluoro-1-methyl-pyrazole-4-carboxamide (A.3.29), methyl (E)-2-[2-[(5-cyano-2-methyl-phenoxy)methyl]phenyl]-3-methoxy-prop-2-enoate (A.3.30), isoflucypram (A.3.31), 2-(difluoromethyl)-N-(1,1,3-trimethyl-indan-4-yl)pyridine-3-carboxamide (A.3.32), 2-(difluoromethyl)-N-[(3R)-1,1,3-trimethylindan-4-yl]pyridine-3-carboxamide (A.3.33), 2-(difluoromethyl)-N-(3-ethyl-1,1-dimethyl-indan-4-yl)pyridine-3-carboxamide (A.3.34), 2-(difluoromethyl)-N-[(3R)-3-ethyl-1,1-dimethyl-indan-4-yl]pyridine-3-carboxamide (A.3.35), 2-(difluoromethyl)-N-(1,1-dimethyl-3-propyl-indan-4-yl)pyridine-3-carboxamide (A.3.36), 2-(difluoromethyl)-N-[(3R)-1,1-dimethyl-3-propyl-indan-4-yl]pyridine-3-carboxamide (A.3.37), 2-(difluoromethyl)-N-(3-isobutyl-1,1-dimethyl-indan-4-yl)pyridine-3-carboxamide (A.3.38), 2-(difluoromethyl)-N-[(3R)-3-isobutyl-1,1-dimethyl-indan-4-yl]pyridine-3-carboxamide (A.3.39);
other respiration inhibitors: diflumetorim (A.4.1); nitrophenyl derivates: binapacryl (A.4.2), dinobuton (A.4.3), dinocap (A.4.4), fluazinam (A.4.5), meptyldinocap (A.4.6), ferimzone (A.4.7); organometal compounds: fentin salts, e. g. fentin-acetate (A.4.8), fentin chloride (A.4.9) or fentin hydroxide (A.4.10); ametoctradin (A.4.11); silthiofam (A.4.12);

### B) Sterol Biosynthesis Inhibitors (SBI Fungicides)

C14 demethylase inhibitors: triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 2-(2,4-difluorophenyl)-1,1-difluoro-3-(tetrazol-1-yl)-1-[5-[4-(2,2,2-trifluoroethoxy)phenyl]-2-pyridyl]propan-2-ol (B.1.31), 2-(2,4-difluorophenyl)-1,1-difluoro-3-(tetrazol-1-yl)-1-[5-[4-(trifluoromethoxy)phenyl]-2-pyridyl]propan-2-ol (B.1.32), 4-[[6-[2-(2,4-difluorophenyl)-1,1-difluoro-2-hydroxy-3-(5-sulfanyl-1,2,4-triazol-1-yl)propyl]-3-pyridyl]oxy]benzonitrile (B.1.33), ipfentrifluconazole (B.1.37), mefentrifluconazole (B.1.38), 2-(chloromethyl)-2-methyl-5-(p-tolylmethyl)-1-(1,2,4-triazol-1-ylmethyl)cyclopentanol (B.1.43); imidazoles: imazalil (B.1.44), pefurazoate (B.1.45), prochloraz (B.1.46), triflumizol (B.1.47); pyrimidines, pyridines, piperazines: fenarimol (B.1.49), pyrifenox (B.1.50), triforine (B.1.51), [3-(4-chloro-2-fluoro-phenyl)-5-(2,4-difluorophenyl)isoxazol-4-yl]-(3-pyridyl)methanol (B.1.52);
Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);
Inhibitors of 3-keto reductase: fenhexamid (B.3.1);
Other Sterol biosynthesis inhibitors: chlorphenomizole (B.4.1);

### C) Nucleic Acid Synthesis Inhibitors

phenylamides or acyl amino acid fungicides: benalaxyl (C.1.1), benalaxyl-M (C.1.2), kiralaxyl (C.1.3), metalaxyl (C.1.4), metalaxyl-M (C.1.5), ofurace (C.1.6), oxadixyl (C.1.7);
other nucleic acid synthesis inhibitors: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7), 5-fluoro-2-(4-chlorophenylmethoxy)pyrimidin-4 amine (C.2.8);

### D) Inhibitors of Cell Division and Cytoskeleton

tubulin inhibitors: benomyl (D.1.1), carbendazim (D.1.2), fuberidazole (D1.3), thiabendazole (D.1.4), thiophanate-methyl (D.1.5), pyridachlometyl (D.1.6), N-ethyl-2-[(3-ethynyl-8-methyl-6-quinolyl)oxy]butanamide (D.1.8), N-ethyl-2-[(3-ethynyl-8-methyl-6-quinolyl)oxy]-2-methylsulfanyl-acetamide (D.1.9), 2-[(3-ethynyl-8-methyl-6-quinolyl)oxy]-N-(2-fluoroethyl)butanamide (D.1.10), 2-[(3-ethynyl-8-methyl-6-quinolyl)oxy]-N-(2-fluoroethyl)-2-methoxy-acetamide (D.1.11), 2-[(3-ethynyl-8-methyl-6-quinolyl)oxy]-N-propyl-butanamide (D.1.12), 2-[(3-ethynyl-8-methyl-6-quinolyl)oxy]-2-methoxy-N-propyl-acetamide (D.1.13), 2-[(3-ethynyl-8-methyl-6-quinolyl)oxy]-2-methylsulfanyl-N-propyl-acetamide (D.1.14), 2-[(3-ethynyl-8-methyl-6-quinolyl)oxy]-N-(2-fluoroethyl)-2-methylsulfanyl-acetamide (D.1.15), 4-(2-bromo-4-fluorophenyl)-N-(2-chloro-6-fluoro-phenyl)-2,5-dimethyl-pyrazol-3-amine (D.1.16);
other cell division inhibitors: diethofencarb (D.2.1), ethaboxam (D.2.2), pencycuron (D.2.3), fluopicolide (D.2.4), zoxamide (D.2.5), metrafenone (D.2.6), pyriofenone (D.2.7), phenamacril (D.2.8);

### E) Inhibitors of Amino Acid and Protein Synthesis

methionine synthesis inhibitors: cyprodinil (E.1.1), mepanipyrim (E.1.2), pyrimethanil (E.1.3);
protein synthesis inhibitors: blasticidin-S (E.2.1), kasugamycin (E.2.2), kasugamycin hydrochloride-hydrate (E.2.3), mildiomycin (E.2.4), streptomycin (E.2.5), oxytetracyclin (E.2.6);

### F) Signal Transduction Inhibitors

MAP/histidine kinase inhibitors: fluoroimid (F.1.1), iprodione (F.1.2), procymidone (F.1.3), vinclozolin (F.1.4), fludioxonil (F.1.5);
G protein inhibitors: quinoxyfen (F.2.1);

### G) Lipid and Membrane Synthesis Inhibitors

Phospholipid biosynthesis inhibitors: edifenphos (G.1.1), iprobenfos (G.1.2), pyrazophos (G.1.3), isoprothiolane (G.1.4);
lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7), zinc thiazole (G.2.8);
phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7);
compounds affecting cell membrane permeability and fatty acids: propamocarb (G.4.1);
inhibitors of oxysterol binding protein: oxathiapiprolin (G.5.1), fluoxapiprolin (G.5.3), 4-[1-[2-[3-(difluoromethyl)-5-methyl-pyrazol-1-yl]acetyl]-4-piperidyl]-N-tetralin-1-yl-pyridine-2-carboxamide (G.5.4), 4-[1-[2-[3,5-bis(difluoromethyl)pyrazol-1-yl]acetyl]-4-piperidyl]-N-tetralin-1-ylpyridine-2-carboxamide (G.5.5), 4-[1-[2-[3-(difluoromethyl)-5-(trifluoromethyl)pyrazol-1-yl]acetyl]-4-piperidyl]-N-tetralin-1-yl-pyridine-2-carboxamide (G.5.6), 4-[1-[2-[5-cyclopropyl-3-(difluoromethyl)pyrazol-1-yl]acetyl]-4-piperidyl]-N-tetralin-1-yl-pyridine-2-carboxamide (G.5.7), 4-[1-[2-[5-methyl-3-(trifluoromethyl)pyrazol-1-yl]acetyl]-4-piperidyl]-N-tetralin-1-ylpyridine-2-carboxamide (G.5.8), 4-[1-[2-[5-(difluoromethyl)-3-(trifluoromethyl)pyrazol-1-yl]acetyl]-4-piperidyl]-N-tetralin-1-yl-pyridine-2-carboxamide (G.5.9), 4-[1-[2-[3,5-bis(trifluoromethyl)pyrazol-1-yl]acetyl]-4-piperidyl]-N-tetralin-1-yl-pyridine-2-carboxamide (G.5.10), (4-[1-[2-[5-cyclopropyl-3-(trifluoromethyl)pyrazol-1-yl]acetyl]-4-piperidyl]-N-tetralin-1-yl-pyridine-2-carboxamide (G.5.11);
   H) Inhibitors with Multi Site Action
inorganic active substances: Bordeaux mixture (H.1.1), copper (H.1.2), copper acetate (H.1.3), copper hydroxide (H.1.4), copper oxychloride (H.1.5), basic copper sulfate (H.1.6), sulfur (H.1.7);
thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
organochlorine compounds: anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11);
guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6H)-tetraone (H.4.10);

### I) Cell Wall Synthesis Inhibitors

inhibitors of glucan synthesis: validamycin (1.1.1), polyoxin B (1.1.2);
melanin synthesis inhibitors: pyroquilon (1.2.1), tricyclazole (1.2.2), carpropamid (1.2.3), dicyclomet (1.2.4), fenoxanil (1.2.5);

### J) Plant Defence Inducers

acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), calcium phosphonate (J.1.11), potassium phosphonate (J.1.12), potassium or sodium bicarbonate (J.1.9), 4-cyclopropyl-N-(2,4-dimethoxyphenyl)thiadiazole-5-carboxamide (J.1.10);

### K) Unknown Mode of Action

bronopol (K.1.1), chinomethionat (K.1.2), cyflufenamid (K.1.3), cymoxanil (K.1.4), dazomet (K.1.5), debacarb (K.1.6), diclocymet (K.1.7), diclomezine (K.1.8), difenzoquat (K.1.9), difenzoquat-methylsulfate (K.1.10), diphenylamin (K.1.11), fenitropan (K.1.12), fenpyrazamine (K.1.13), flumetover (K.1.14), flusulfamide (K.1.15), flutianil (K.1.16), harpin (K.1.17), methasulfocarb (K.1.18), nitrapyrin (K.1.19), nitrothal-isopropyl (K.1.20), tolprocarb (K.1.21), oxin-copper (K.1.22), proquinazid (K.1.23), tebufloquin (K.1.24), tecloftalam (K.1.25), triazoxide (K.1.26), N′-(4-(4-chloro-3-trifluoromethyl-phenoxy)-2,5-dimethyl-phenyl)-N-ethyl-N-methyl formamidine (K.1.27), N′-(4-(4-fluoro-3-trifluoromethyl-phenoxy)-2,5-dimethyl-phenyl)-N-ethyl-N-methyl formamidine (K.1.28), N′-[4-[[3-[(4-chlorophenyl)methyl]-1,2,4-thiadiazol-5-yl]-oxy]-2,5-dimethyl-phenyl]-N-ethyl-N-methyl-formamidine (K.1.29), N′-(5-bromo-6-indan-2-yloxy-2-methyl-3-pyridyl)-N-ethyl-N-methyl-formamidine (K.1.30), N′-[5-bromo-6-[1-(3,5-difluorophenyl)ethoxy]-2-methyl-3-pyridyl]-N-ethyl-N-methyl-formamidine (K.1.31), N′-[5-bromo-6-(4-isopropylcyclohexoxy)-2-methyl-3-pyridyl]-N-ethyl-N-methyl-formamidine (K.1.32), N′-[5-bromo-2-methyl-6-(1-phenylethoxy)-3-pyridyl]-N-ethyl-N-methyl-formamidine (K.1.33), N′-(2-methyl-5-trifluoromethyl-4-(3-trimethylsilanyl-propoxy)-phenyl)-N-ethyl-N-methyl formamidine (K.1.34), N′-(5-difluoromethyl-2-methyl-4-(3-trimethylsilanyl-propoxy)-phenyl)-N-ethyl-N-methyl formamidine (K.1.35), 2-(4-chloro-phenyl)-N-[4-(3,4-dimethoxy-phenyl)-isoxazol-5-yl]-2-prop-2-ynyloxy-acetamide (K.1.36), 3-[5-(4-chloro-phenyl)-2,3-dimethyl-isoxazolidin-3-yl]-pyridine (pyrisoxazole) (K.1.37), 3-[5-(4-methylphenyl)-2,3-dimethyl-isoxazolidin-3 yl]-pyridine (K.1.38), 5-chloro-1-(4,6-dimethoxy-pyrimidin-2-yl)-2-methyl-1H-benzoimidazole (K.1.39), ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carbamate (K.1.42), but-3-ynyl N-[6-[[(Z)-[(1-methyltetrazol-5-yl)-phenyl-methylene]amino]oxymethyl]-2-pyridyl]carbamate (K.1.43), ipflufenoquin (K.1.44), quinofumelin (K.1.47), benziothiazolinone (K.1.48), bromothalonil (K.1.49), 2-(6-benzyl-2-pyridyl)quinazoline (K.1.50), 2-[6-(3-fluoro-4-methoxy-phenyl)-5-methyl-2-pyridyl]quinazoline (K.1.51), dichlobentiazox (K.1.52), N′-(2,5-dimethyl-4-phenoxy-phenyl)-N-ethyl-N-methyl-formamidine (K.1.53), pyrifenamine (K.1.54), fluopimomide (K.1.55), N′-[5-bromo-2-methyl-6-(1-methyl-2 propoxy-ethoxy)-3-pyridyl]-N-ethyl-N-methyl-formamidine (K.1.56);

### M) Growth Regulators

abscisic acid (M.1.1), amidochlor, ancymidol, 6-benzylaminopurine, brassinolide, butralin, chlormequat, chlormequat chloride, choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat, mepiquat chloride, naphthaleneacetic acid, N-6-benzyladenine, paclobutrazol, prohexadione, prohexadione-calcium, prohydrojasmon, thidiazuron, triapenthenol, tributyl phosphorotrithioate, 2,3,5-tri-iodobenzoic acid, trinexapac-ethyl, uniconazole;

N) Herbicides from Classes N.1 to N.15
N.1 Lipid biosynthesis inhibitors: alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, 4-(4′-chloro-4-cyclo-propyl-2′-fluoro[1,1′-biphenyl]-3-yl)-5-hydroxy-2,2,6,6-tetramethyl-2H-pyran-3(6H)-one (1312337-72-6); 4-(2′,4′-dichloro-4-cyclopropyl[1,1′-biphenyl]-3-yl)-5-hydroxy-2,2,6,6-tetramethyl-2H-pyran-3(6H)-one (1312337-45-3); 4-(4′-chloro-4-ethyl-2′-fluoro[1,1′-biphenyl]-3-yl)-5-hydroxy-2,2,6,6-tetramethyl-2H-pyran-3(6H)-one (1033757-93-5); 4-(2′,4′-dichloro-4-ethyl[1,1′-biphenyl]-3-yl)-2,2,6,6-tetramethyl-2H-pyran-3,5(4H,6H)-dione (1312340-84-3); 5-(acetyloxy)-4-(4′-chloro-4-cyclopropyl-2′-fluoro[1,1′-biphenyl]-3-yl)-3,6-dihydro-2,2,6,6-tetramethyl-2H-pyran-3-one (1312337-48-6); 5-(acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(acetyloxy)-4-(4′-chloro-4-ethyl-2′-fluoro[1,1′-biphenyl]-3-yl)-3,6-dihydro-2,2,6,6-tetramethyl-2H-pyran-3-one (1312340-82-1); 5-(acetyloxy)-4-(2′,4′-dichloro-4-ethyl[1,1′-biphenyl]-3-yl)-3,6-dihydro-2,2,6,6-tetramethyl-2H-pyran-3-one (1033760-55-2); 4-(4′-chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (1312337-51-1); 4-(2′,4′-dichloro 4-cyclopropyl-[1,1′-biphenyl]-3-yl)-5,6-dihydro-2,2,6,6-tetramethyl-5-oxo-2H-pyran-3-yl carbonic acid methyl ester; 4-(4′-chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (1312340-83-2); 4-(2′,4′-dichloro-4-ethyl¬[11′-biphenyl]-3-yl)-5,6-dihydro-2,2,6,6-tetramethyl-5-oxo-2H-pyran-3-yl carbonic acid methyl ester (1033760-58-5); benfuresate, butylate, cycloate, dalapon, dimepiperate, EPTC, esprocarb, ethofumesate, flupropanate, molinate, orbencarb, pebulate, prosulfocarb, TCA, thiobencarb, tiocarbazil, triallate, vemolate;
N.2 ALS inhibitors: amidosulfuron, azimsulfuron, bensulfuron, bensulfuron-methyl, chlorimuron, chlorimuron-ethyl, chlorsulfuron, cinosulfuron, cyclosulfamuron, ethametsulfuron, ethametsulfuron-methyl, ethoxysulfuron, flazasulfuron, flucetosulfuron, flupyrsulfuron, flupyrsulfuronmethyl-sodium, foramsulfuron, halosulfuron, halosulfuron-methyl, imazosulfuron, iodosulfuron, iodosulfuron-methyl-sodium, iofensulfuron, iofensulfuron-sodium, mesosulfuron, metazosulfuron, metsulfuron, metsulfuron-methyl, nicosulfuron, orthosulfamuron, oxasulfuron, primisulfuron, primisulfuron-methyl, propyrisulfuron, prosulfuron, pyrazosulfuron, pyrazosulfuron-ethyl, rimsulfuron, sulfometuron, sulfometuron-methyl, sulfosulfuron, thifensulfuron, thifensulfuron-methyl, triasulfuron, tribenuron, tribenuron-methyl, trifloxysulfuron, triflusulfuron, triflusulfuron-methyl, tritosulfuron, imazamethabenz, imazamethabenz-methyl, imazamox, imazapic, imazapyr, imazaquin, imazethapyr; cloransulam, cloransulam-methyl, diclosulam, flumetsulam, florasulam, metosulam, penoxsulam, pyrimisulfan, pyroxsulam; bispyribac, bispyribac-sodium, pyribenzoxim, pyriftalid, pyriminobac, pyriminobac-methyl, pyrithiobac, pyrithiobac-sodium, 4-[[[2-[(4,6-dimethoxy-2-pyrimidinyl)oxy]phenyl]methyl]amino]-benzoic acid-1-methyl-ethyl ester (420138-41-6), 4-[[[2-[(4,6-dimethoxy-2-pyrimidinyl)oxy]phenyl]-methyl]amino]-benzoic acid propyl ester (420138-40-5), N-(4-bromophenyl)-2-[(4,6-dimethoxy-2-pyrimidinyl)oxy]benzenemethanamine (420138-01-8); flucarbazone, flucarbazone-sodium, propoxycarbazone, propoxycarbazone-sodium, thiencarbazone, thiencarbazone-methyl; triafamone;
N.3 Photosynthesis inhibitors: amicarbazone; chlorotriazine; ametryn, atrazine, chloridazone, cyanazine, desmetryn, dimethametryn, hexazinone, metribuzin, prometon, prometryn, propazine, simazine, simetryn, terbumeton, terbuthylazin, terbutryn, trietazin; chlorobromuron, chlorotoluron, chloroxuron, dimefuron, diuron, fluometuron, isoproturon, isouron, linuron, metamitron, methabenzthiazuron, metobenzuron, metoxuron, monolinuron, neburon, siduron, tebuthiuron, thiadiazuron, desmedipham, karbutilat, phenmedipham, phenmediphamethyl, bromofenoxim, bromoxynil and its salts and esters, ioxynil and its salts and esters, bromacil, lenacil, terbacil, bentazon, bentazon-sodium, pyridate, pyridafol, pentanochlor, propanil; diquat, diquat-dibromide, paraquat, paraquat-dichloride, paraquat-dimetilsulfate, 1-(6-tert-butylpyrimidin-4-yl)-2-hydroxy-4-meth-oxy-3-methyl-2H-pyrrol-5-one (1654744-66-7), 1-(5-tert-butylisoxazol-3-yl)-2-hydroxy-4 methoxy-3-methyl-2H-pyrrol-5-one (1637455-12-9), 1-(5-tert-butylisoxazol-3-yl)-4-chloro-2 hydroxy-3-methyl-2H-pyrrol-5-one (1637453-94-1), 1-(5-tert-butyl-1-methyl-pyrazol-3-yl)-4 chloro-2-hydroxy-3-methyl-2H-pyrrol-5-one (1654057-29-0), 1-(5-tert-butyl-1-methyl-pyrazol-3-yl)-3-chloro-2-hydroxy-4-methyl-2H-pyrrol-5-one (1654747-80-4), 4-hydroxy-1 methoxy-5-methyl-3-[4-(trifluoromethyl)-2-pyridyl]imidazolidin-2-one; (2023785-78-4), 4 hydroxy-1,5-dimethyl-3-[4-(trifluoromethyl)-2-pyridyl]imidazolidin-2-one (2023785-79-5), 5 ethoxy-4-hydroxy-1-methyl-3-[4-(trifluoromethyl)-2-pyridyl]imidazolidin-2-one (1701416-69-4), 4-hydroxy-1-methyl-3-[4-(trifluoromethyl)-2-pyridyl]imidazolidin-2-one (1708087-22-2), 4 hydroxy-1,5-dimethyl-3-[1-methyl-5-(trifluoromethyl)pyrazol-3-yl]imidazolidin-2-one (2023785-80-8), 1-(5-tert-butylisoxazol-3-yl)-4-ethoxy-5-hydroxy-3-methyl-imidazolidin-2-one (1844836-64-1);
N.4 protoporphyrinogen-IX oxidase inhibitors: acifluorfen, acifluorfen-sodium, azafenidin, bencarbazone, benzfendizone, bifenox, butafenacil, carfentrazone, carfentrazone-ethyl, chlor-methoxyfen, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, tiafenacil, trifludimoxazin, ethyl [3-[2-chloro-4-fluoro-5-(1-methyl-6-trifluoromethyl-2,4-dioxo-1,2,3,4-tetrahydropyrmidin-3-yl)phenoxy]-2-pyridyloxy]acetate (353292-31-6), N-ethyl-3-(2,6-dichloro-4-trifluoromethylphenoxy)-5-methyl-1H-pyrazole-1-carboxamide (452098-92-9), N-tetrahydrofurfuryl-3-(2,6-dichloro-4-trifluoromethylphenoxy)-5-methyl-1H-pyrazole-1-carboxamide (915396-43-9), N-ethyl-3-(2-chloro-6-fluoro-4-trifluoromethyl-phenoxy)-5-methyl-1H-pyrazole-1-carboxamide (452099-05-7), N-tetrahydro-furfuryl-3-(2-chloro-6-fluoro-4-trifluoro¬methylphenoxy)-5-methyl-1H-pyrazole-1-carboxamide (452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (1300118-96-0), 1-methyl-6-trifluoro¬methyl-3-(2,2,7-tri-fluoro-3-oxo-4-prop-2-ynyl-3,4-dihydro-2H-benzo[1,4]oxazin-6-yl)-1H-pyrimidine-2,4-dione (1304113-05-0), methyl (E)-4-[2-chloro-5-[4-chloro-5-(difluoromethoxy)-1H-methylpyrazol-3-yl]-4-fluoro-phenoxy]-3-methoxy-but-2-enoate (948893-00-3), 3-[7-chloro-5-fluoro-2-(trifluoromethyl)-1H-benzimidazol-4-yl]-1-methyl-6-(trifluoromethyl)-1H-pyrimidine-2,4-dione (212754-02-4);
N.5 Bleacher herbicides: beflubutamid, diflufenican, fluridone, flurochloridone, flurtamone, norflurazon, picolinafen, 4-(3-trifluoromethyl-phenoxy)-2-(4-trifluoromethylphenyl)pyrimidine (180608-33-7); benzobicyclon, benzofenap, bicyclopyrone, clomazone, fenquintrione, isoxaflutole, mesotrione, pyrasulfotole, pyrazolynate, pyrazoxyfen, sulcotrione, tefuryltrione, tembotrione, tolpyralate, topramezone; aclonifen, amitrole, flumeturon;
N.6 EPSP synthase inhibitors: glyphosate, glyphosate-isopropylammonium, glyposate-potassium, glyphosate-trimesium (sulfosate);
N.7 Glutamine synthase inhibitors: bilanaphos (bialaphos), bilanaphos-sodium, glufosinate, glufosinate-P, glufosinate-ammonium;
N.8 DHP synthase inhibitors: asulam;
N.9 Mitosis inhibitors: benfluralin, butralin, dinitramine, ethalfluralin, fluchloralin, oryzalin, pendimethalin, prodiamine, trifluralin; amiprophos, amiprophos-methyl, butamiphos; chlorthal, chlorthal-dimethyl, dithiopyr, thiazopyr, propyzamide, tebutam; carbetamide, chlorpropham, flamprop, flamprop-isopropyl, flamprop-methyl, flamprop-M-isopropyl, flamprop-M-methyl, propham;
N.10 VLCFA inhibitors: acetochlor, alachlor, butachlor, dimethachlor, dimethenamid, dimethenamid-P, metazachlor, metolachlor, metolachlor-S, pethoxamid, pretilachlor, propachlor, propisochlor, thenylchlor, flufenacet, mefenacet, diphenamid, naproanilide, napropamide, napropamide-M, fentrazamide, anilofos, cafenstrole, fenoxasulfone, ipfencarbazone, piperophos, pyroxasulfone, isoxazoline compounds of the formulae II.1, II.2, II.3, II.4, II.5, II.6, II.7, II.8 and II.9

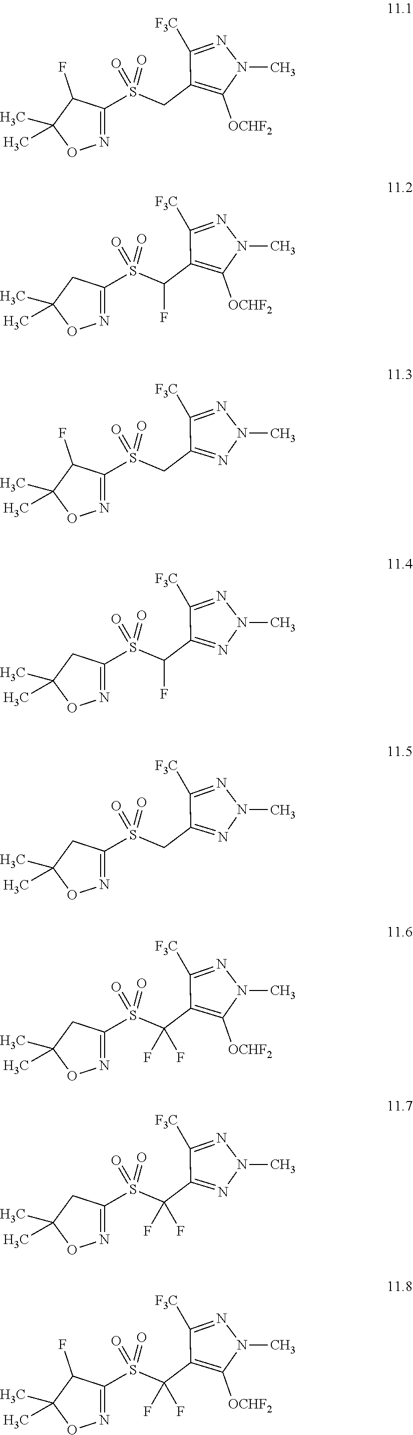

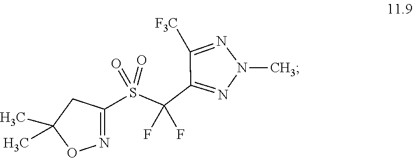 [SMILES: Cn1nc(C(F)(F)F)c(C(F)(F)S(=O)(=O)C2=NOC(C)(C)C2)n1]

N.11 Cellulose biosynthesis inhibitors: chlorthiamid, dichlobenil, flupoxam, indaziflam, isoxaben, triaziflam, 1-cyclohexyl-5-pentafluorphenyloxy-14-[1,2,4,6]thiatriazin-3-ylamine (175899-01-1);
N.12 Decoupler herbicides: dinoseb, dinoterb, DNOC and its salts;
N.13 Auxinic herbicides: 2,4-D and its salts and esters, clacyfos, 2,4-DB and its salts and esters, aminocyclopyrachlor and its salts and esters, aminopyralid and its salts such as aminopyralid-dimethylammonium, aminopyralid-tris(2-hydroxypropyl)ammonium and its esters, benazolin, benazolin-ethyl, chloramben and its salts and esters, clomeprop, clopyralid and its salts and esters, dicamba and its salts and esters, dichlorprop and its salts and esters, dichlorprop-P and its salts and esters, fluroxypyr, fluroxypyr-butometyl, fluroxypyr-meptyl, halauxifen and its salts and esters (943832-60-8); MCPA and its salts and esters, MCPAthioethyl, MCPB and its salts and esters, mecoprop and its salts and esters, mecoprop-P and its salts and esters, picloram and its salts and esters, quinclorac, quinmerac, TBA (2,3,6) and its salts and esters, triclopyr and its salts and esters, 4-amino-3-chloro-6-(4-chloro-2-fluoro-3-methoxyphenyl)-5-fluoropyridine-2-carboxylic acid, florpyrauxifen-benzyl, florpyrauxifen;
N.14 Auxin transport inhibitors: diflufenzopyr, diflufenzopyr-sodium, naptalam, naptalamsodium;
N.15 Other herbicides: bromobutide, chlorflurenol, chlorflurenol-methyl, cinmethylin, cumyluron, cyclopyrimorate (499223-49-3) and its salts and esters, dalapon, dazomet, difenzoquat, difenzoquat-metilsulfate, dimethipin, DSMA, dymron, endothal and its salts, etobenzanid, flurenol, flurenol-butyl, flurprimidol, fosamine, fosamine-ammonium, indanofan, maleic hydrazide, mefluidide, metam, methiozolin (403640-27-7), methyl azide, methyl bromide, methyl-dymron, methyl iodide, MSMA, oleic acid, oxaziclomefone, pelargonic acid, pyributicarb, quinoclamine, tridiphane;
   O) Insecticides from classes O.1 to O.29
O.1 Acetylcholine esterase (AChE) inhibitors: aldicarb (O.1.1), alanycarb (O.1.2), bendiocarb (O.1.3), benfuracarb (O.1.4), butocarboxim (O.1.5), butoxycarboxim (O.1.6), carbaryl (O.1.7), carbofuran (O.1.8), carbosulfan (O.1.9), ethiofencarb (O.1.10), fenobucarb (O.1.11), formetanate (O.1.12), furathiocarb (O.1.13), isoprocarb (O.1.14), methiocarb (O.1.15), methomyl (O.1.16), metolcarb (O.1.17), oxamyl (O.1.18), pirimicarb (O.1.19), propoxur (O.1.20), thiodicarb (O.1.21), thiofanox (O.1.22), trimethacarb (O.1.23), XMC (O.1.24), xylylcarb (O.1.25), triazamate (O.1.26), acephate (O.1.27), azamethiphos (O.1.28), azinphosethyl (O.1.29), azinphosmethyl (O.1.30), cadusafos (O.1.31), chlorethoxyfos (O.1.32), chlorfenvinphos (O.1.33), chlormephos (O.1.34), chlorpyrifos (O.1.35), chlorpyrifos-methyl (O.1.36), coumaphos (O.1.37), cyanophos (O.1.38), demeton-S-methyl (O.1.39), diazinon (O.1.40), dichlorvos/DDVP (O.1.41), dicrotophos (O.1.42), dimethoate (O.1.43), dimethylvinphos (O.1.44), disulfoton (O.1.45), EPN (O.1.46), ethion (O.1.47), ethoprophos (O.1.48), famphur (O.1.49), fenamiphos (O.1.50), fenitrothion (O.1.51), fenthion (O.1.52), fosthiazate (O.1.53), heptenophos (O.1.54), imicyafos (O.1.55), isofenphos (O.1.56), isopropyl O-(methoxyaminothio-phosphoryl) salicylate (O.1.57), isoxathion (O.1.58), malathion (O.1.59), mecarbam (O.1.60), methamidophos (O.1.61), methidathion (O.1.62), mevinphos (O.1.63), monocrotophos (O.1.64), naled (O.1.65), omethoate (O.1.66), oxydemeton-methyl (O.1.67), parathion (O.1.68), parathion-methyl (O.1.69), phenthoate (O.1.70), phorate (O.1.71), phosalone (O.1.72), phosmet (O.1.73), phosphamidon (O.1.74), phoxim (O.1.75), pirimiphos-methyl (O.1.76), profenofos (O.1.77), propetamphos (O.1.78), prothiofos (O.1.79), pyraclofos (O.1.80), pyridaphenthion (O.1.81), quinalphos (O.1.82), sulfotep (O.1.83), tebupirimfos (O.1.84), temephos (O.1.85), terbufos (O.1.86), tetrachlorvinphos (O.1.87), thiometon (O.1.88), triazophos (O.1.89), trichlorfon (O.1.90), vamidothion (O.1.91); 0.2 GABA-gated chloride channel antagonists: endosulfan (O.2.1), chlordane (O.2.2), ethiprole (O.2.3), fipronil (O.2.4), flufiprole (O.2.5), pyrafluprole (O.2.6), pyriprole (O.2.7); 0.3 Sodium channel modulators: acrinathrin (O.3.1), allethrin (O.3.2), d-cis-trans allethrin (O.3.3), d-trans allethrin (O.3.4), bifenthrin (O.3.5), kappa-bifenthrin (O.3.6), bioallethrin (O.3.7), bioallethrin S-cylclopentenyl (O.3.8), bioresmethrin (O.3.9), cycloprothrin (O.3.10), cyfluthrin (O.3.11), beta-cyfluthrin (O.3.12), cyhalothrin (O.3.13), lambda-cyhalothrin (O.3.14), gamma-cyhalothrin (O.3.15), cypermethrin (O.3.16), alpha-cypermethrin (O.3.17), beta-cypermethrin (O.3.18), theta-cypermethrin (O.3.19), zeta-cypermethrin (O.3.20), cyphenothrin (O.3.21), deltamethrin (O.3.22), empenthrin (O.3.23), esfenvalerate (O.3.24), etofenprox (O.3.25), fenpropathrin (O.3.26), fenvalerate (O.3.27), flucythrinate (O.3.28), flumethrin (O.3.29), tau-fluvalinate (O.3.30), halfenprox (O.3.31), heptafluthrin (O.3.32), imiprothrin (O.3.33), meperfluthrin (O.3.34), metofluthrin (O.3.35), momfluorothrin (O.3.36), epsilon-momfluorothrin (O.3.37), permethrin (O.3.38), phenothrin (O.3.39), prallethrin (O.3.40), profluthrin (O.3.41), pyrethrin (pyrethrum) (O.3.42), resmethrin (O.3.43), silafluofen (O.3.44), tefluthrin (O.3.45), kappa-tefluthrin (O.3.46), tetramethylfluthrin (O.3.47), tetramethrin (O.3.48), tralomethrin (O.3.49), transfluthrin (O.3.50), DDT (O.3.51), methoxychlor (O.3.52); 0.4 Nicotinic acetylcholine receptor agonists (nAChR): acetamiprid (O.4.1), clothianidin (O.4.2), cycloxaprid (O.4.3), dinotefuran (O.4.4), imidacloprid (O.4.5), nitenpyram (O.4.6), thiacloprid (O.4.7), thiamethoxam (O.4.8), 4,5-dihydro-N-nitro-1-(2-oxiranylmethyl)-1H-imidazol-2-amine (O.4.9), (2E)-1-[(6-chloropyridin-3-yl)methyl]-N′-nitro-2-pentylidenehydrazinecarboximidamide (O.4.10), 1-[(6-chloropyridin-3-yl)methyl]-7-methyl-8-nitro-5-propoxy-1,2,3,5,6,7-hexahydroimidazo[1,2-a]pyridine (O.4.11), nicotine (O.4.12), sulfoxaflor (O.4.13), flupyradifurone (O.4.14), triflumezopyrim (O.4.15), (3R)-3-(2-chlorothiazol-5-yl)-8-methyl-5-oxo-6-phenyl-2,3-dihydrothiazolo[3,2-a]pyrimidin-8-ium-7-olate (O.4.16), (3S)-3-(6-chloro-3-pyridyl)-8-methyl-5-oxo-6-phenyl-2,3-dihydrothiazolo[3,2-a]pyrimidin-8-ium-7-olate (O.4.17), (3S)-8-methyl-5-oxo-6-phenyl-3-pyrimidin-5-yl-2,3-dihydrothiazolo[3,2-a]pyrimidin-8-ium-7-olate (O.4.18), (3R)-3-(2-chlorothiazol-5-yl)-8-methyl-5-oxo-6-[3-(trifluoromethyl)phenyl]-2,3-dihydrothiazolo[3,2-a]pyrimidin-8-ium-7-olate (O.4.19), (3R)-3-(2-chlorothiazol-5-yl)-6-(3,5-dichlorophenyl)-8-methyl-5-oxo-2,3-dihydrothiazolo[3,2-a]pyrimidin-8-ium-7-olate (O.4.20), (3R)-3-(2-chlorothiazol-5-yl)-8-ethyl-5-oxo-6-phenyl-2,3-dihydrothiazolo[3,2-a]pyrimidin-8-ium-7-olate (O.4.21);
O.5 Nicotinic acetylcholine receptor allosteric activators: spinosad (O.5.1), spinetoram (O.5.2);
O.6 Chloride channel activators: abamectin (O.6.1), emamectin benzoate (O.6.2), ivermectin (O.6.3), lepimectin (O.6.4), milbemectin (O.6.5);
O.7 Juvenile hormone mimics: hydroprene (O.7.1), kinoprene (O.7.2), methoprene (O.7.3), fenoxycarb (O.7.4), pyriproxyfen (O.7.5);
O.8 miscellaneous non-specific (multi-site) inhibitors: methyl bromide (O.8.1) and other alkyl halides, chloropicrin (O.8.2), sulfuryl fluoride (O.8.3), borax (O.8.4), tartar emetic (O.8.5);
O.9 Chordotonal organ TRPV channel modulators: pymetrozine (O.9.1), pyrifluquinazon (O.9.2), flonicamid (O.9.3);
O.10 Mite growth inhibitors: clofentezine (O.10.1), hexythiazox (O.10.2), diflovidazin (O.10.3), etoxazole (O.10.4);
O.11 Microbial disruptors of insect midgut membranes: Bacillus thuringiensis, Bacillus sphaericus and the insecticidal proteins they produce: Bacillus thuringiensis subsp. Israelensis (O.11.1), Bacillus sphaericus (O.11.2), Bacillus thuringiensis subsp. aizawai (O.11.3), Bacillus thuringiensis subsp. kurstaki (O.11.4), Bacillus thuringiensis subsp. tenebrionis (O.11.5), the Bt crop proteins: Cry1Ab (O.11.6), Cry1Ac (O.11.7), Cry1Fa (O.11.8), Cry2Ab (O.11.9), mCry3A (O.11.10), Cry3Ab (O.11.11), Cry3Bb (O.11.12), Cry34/35Ab1 (O.11.13);
O.12 Inhibitors of mitochondrial ATP synthase: diafenthiuron (O.12.1), azocyclotin (O.12.2), cyhexatin (O.12.3), fenbutatin oxide (O.12.4), propargite (O.12.5), tetradifon (O.12.6);
O.13 Uncouplers of oxidative phosphorylation via disruption of the proton gradient: chlorfenapyr (O.13.1), DNOC (O.13.2), sulfluramid (O.13.3);
O.14 Nicotinic acetylcholine receptor (nAChR) channel blockers: bensultap (O.14.1), cartap hydrochloride (O.14.2), thiocyclam (O.14.3), thiosultap sodium (O.14.4);
O.15 Inhibitors of the chitin biosynthesis type 0: bistrifluron (O.15.1), chlorfluazuron (O.15.2), diflubenzuron (O.15.3), flucycloxuron (O.15.4), flufenoxuron (O.15.5), hexaflumuron (O.15.6), lufenuron (O.15.7), novaluron (O.15.8), noviflumuron (O.15.9), teflubenzuron (O.15.10), triflumuron (O.15.11);
O.16 Inhibitors of the chitin biosynthesis type 1: buprofezin (O.16.1);
O.17 Moulting disruptors: cyromazine (O.17.1);
O.18 Ecdyson receptor agonists: methoxyfenozide (O.18.1), tebufenozide (O.18.2), halofenozide (O.18.3), fufenozide (O.18.4), chromafenozide (O.18.5);
O.19 Octopamin receptor agonists: amitraz (O.19.1);
O.20 Mitochondrial complex Ill electron transport inhibitors: hydramethylnon (O.20.1), acequinocyl (O.20.2), fluacrypyrim (O.20.3), bifenazate (O.20.4);
O.21 Mitochondrial complex I electron transport inhibitors: fenazaquin (O.21.1), fenpyroximate (O.21.2), pyrimidifen (O.21.3), pyridaben (O.21.4), tebufenpyrad (O.21.5), tolfenpyrad (O.21.6), rotenone (O.21.7);
O.22 Voltage-dependent sodium channel blockers: indoxacarb (O.22.1), metaflumizonev (O.22.2), 2-[2-(4-cyanophenyl)-1-[3-(trifluoromethyl)phenyl]ethylidene]-N-[4-(difluoromethoxy)phenyl]-hydrazinecarboxamide (O.22.3), N-(3-chloro-2-methylphenyl)-2-[(4-chloro-phenyl)-[4-[methyl(methylsulfonyl)amino]phenyl]methylene]-hydrazinecarboxamide (O.22.4);
O.23 Inhibitors of the of acetyl CoA carboxylase: spirodiclofen (O.23.1), spiromesifen (O.23.2), spirotetramat (O.23.3), spiropidion (O.23.4);
O.24 Mitochondrial complex IV electron transport inhibitors: aluminium phosphide (O.24.1), calcium phosphide (O.24.2), phosphine (O.24.3), zinc phosphide (O.24.4), cyanide (O.24.5);
O.25 Mitochondrial complex II electron transport inhibitors: cyenopyrafen (O.25.1), cyflumetofen (O.25.2);
O.26 Ryanodine receptor-modulators: flubendiamide (O.26.1), chlorantraniliprole (O.26.2), cyantraniliprole (O.26.3), cyclaniliprole (O.26.4), tetraniliprole (O.26.5), (R)-3-chloro-N-{2-methyl-4-[1,2,2,2-tetrafluoro-1-(trifluoromethyl)ethyl]phenyl}-N²-(1-methyl-2-methylsulfonylethyl)phthalamide (O.26.6), (S)-3-chloro-N-{2-methyl-4-[1,2,2,2-tetrafluoro-1-(trifluoromethyl)ethyl]phenyl}-N²-(1-methyl-2-methylsulfonylethyl)phthalamide (O.26.7), methyl-2-[3,5-dibromo-2-({[3-bromo-1-(3-chloropyrdin-2-yl)-1H-pyrazol-5-yl]carbonyl}amino)benzoyl]-1,2-dimethylhydrazinecarboxylate (O.26.8), N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide (O.26.9), N-[4-chloro-2-[(diethyl-lambda-4-sulfanylidene)carbamoyl]-6-methyl-phenyl]-2-(3-chloro-2-pyidyl)-5-(trifluoromethyl)pyrazole-3-carboxamide (O.26.10), N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide (O.26.11), N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide (O.26.12), N-[4,6-dibromo-2-[(diethyl-lambda-4-sulfanylidene)carbamoyl]-phenyl]-2-(3-chloro-2-pyidyl)-5-(trifluoromethyl)pyrazole-3-carboxamide (O.26.13), N-[2-(5-amino-1,3,4-thiadiazol-2-yl)-4-chloro-6-methylphenyl]-3-bromo-1-(3-chloro-2-pyridinyl)-1H-pyrazole-5-carboxamide (O.26.14), 3-chloro-1-(3-chloro-2-pyridinyl)-N-[2,4-dichloro-6-[[(1-cyano-1-methylethyl)amino]carbonyl]phenyl]-1H-pyrazole-5-carboxamide (O.26.15), tetrachlorantraniliprole (O.26.16), N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide (O.26.17), cyhalodiamide (O.26.18);
O.27: Chordotonal organ Modulators—undefined target site: flonicamid (O.27.1);
O.28. insecticidal active compounds of unknown or uncertain mode of action: afidopyropen (O.28.1), afoxolaner (O.28.2), azadirachtin (O.28.3), amidoflumet (O.28.4), benzoximate (O.28.5), broflanilide (O.28.6), bromopropylate (O.28.7), chinomethionat (O.28.8), cryolite (O.28.9), dicloromezotiaz (O.28.10), dicofol (O.28.11), flufenerim (O.28.12), flometoquin (O.28.13), fluensulfone (O.28.14), fluhexafon (O.28.15), fluopyram (O.28.16), fluralaner (O.28.17), metoxadiazone (O.28.18), piperonyl butoxide (O.28.19), pyflubumide (O.28.20), pyridalyl (O.28.21), tioxazafen (O.28.22), 11-(4-chloro-2,6-dimethylphenyl)-12-hydroxy-1,4-dioxa-9-azadispiro[4.2.4.2]-tetradec-11-en-10-one, 3-(4′-fluoro-2,4-dimethylbiphenyl-3-yl)-4-hydroxy-8-oxa-1-azaspiro[4.5]dec-3-en-2-one, 1-[2-fluoro-4-methyl-5-[(2,2,2-trifluoroethyl)sulfinyl]phenyl]-3-(trifluoromethyl)-1H-1,2,4-triazole-5-amine (O.28.23), Bacillus firmus I-1582 (O.28.24), flupyrimin (O.28.25), fluazaindolizine (O.28.26), 4-[5-(3,5-dichlorophenyl)-5-(trifluoromethyl)-4H-isoxazol-3-yl]-2-methyl-N-(1-oxothietan-3-yl)benzamide (O.28.27), fluxametamide (O.28.28), 5-[3-[2,6-dichloro-4-(3,3-dichloroallyloxy)phenoxy]propoxy]-1H-pyrazole (O.28.1), 4-cyano-N-[2-cyano-5-[[2,6-dibromo-4-[1,2,2,3,3,3-hexafluoro-1-(trifluoromethyl)propyl]phenyl]carbamoyl]phenyl]-2-methyl-benzamide (O.28.29), 4-cyano-3-[(4-cyano-2-methyl-benzoyl)amino]-N-[2,6-dichloro-4-[1,2,2,3,3,3-hexafluoro-1-(trifluoromethyl)propyl]phenyl]-2-fluoro-benzamide (O.28.30), N-[5-[[2-chloro-6-cyano-4-[1,2,2,3,3,3-hexafluoro-1-(trifluoromethyl)propyl]phenyl]carbamoyl]-2-cyano-phenyl]-4-cyano-2-methyl-benzamide (O.28.31), N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide (O.28.32), N-[5-[[2-bromo-6-chloro-4-[1,2,2,3,3,3-hexafluoro-1-(trifluoromethyl)propyl]phenyl]carbamoyl]-2-cyano-phenyl]-4-cyano-2-methyl-benzamide (O.28.33), 4-cyano-N-[2-cyano-5-[[2,6-dichloro-4-[1,2,2,3,3,3-hexafluoro-1-(trifluoromethyl)propyl]phenyl]carbamoyl]phenyl]-2-methyl-benzamide (O.28.34), 4-cyano-N-[2-cyano-5-[[2,6-dichloro-4-[1,2,2,2-tetrafluoro-1-(trifluoromethyl)ethyl]phenyl]carbamoyl]phenyl]-2-methyl-benzamide (O.28.35), N-[5-[[2-bromo-6-chloro-4-[1,2,2,2-tetrafluoro-1-(trifluoromethyl)ethyl]phenyl]carbamoyl]-2-cyano-phenyl]-4-cyano-2-methyl-benzamide (O.28.36); 2-(1,3-dioxan-2-yl)-6-[2-(3-pyridinyl)-5-thiazolyl]-pyridine (O.28.37), 2-[6-[2-(5-fluoro-3-pyridinyl)-5-thiazolyl]-2-pyridinyl]-pyrimidine (O.28.38), 2-[6-[2-(3-pyridinyl)-5-thiazolyl]-2-pyridinyl]-pyrimidine (O.28.39), N-methylsulfonyl-6-[2-(3-pyridyl)thiazol-5-yl]pyridine-2-carboxamide (O.28.40), N-methylsulfonyl-6-[2-(3-pyridyl)thiazol-5-yl]pyridine-2-carboxamide (O.28.41), 1-[(6-chloro-3-pyridinyl)methyl]-1,2,3,5,6,7-hexahydro-5-methoxy-7-methyl-8-nitro-imidazo[1,2-a]pyridine (O.28.42), 1-[(6-chloropyridin-3-yl)methyl]-7-methyl-8-nitro-1,2,3,5,6,7-hexahydroimidazo[1,2-a]pyridin-5-ol (O.28.43), 1-isopropyl-N,5-dimethyl-N-pyridazin-4-yl-pyrazole-4-carboxamide (O.28.44), 1-(1,2-dimethylpropyl)-N-ethyl-5-methyl-N-pyridazin-4-yl-pyrazole-4-carboxamide (O.28.45), N,5-dimethyl-N-pyridazin-4-yl-1-(2,2,2-trifluoro-1-methyl-ethyl)pyrazole-4-carboxamide (O.28.46), 1-[1-(1-cyanocyclopropyl)ethyl]-N-ethyl-5-methyl-N-pyridazin-4-yl-pyrazole-4-carboxamide (O.28.47), N-ethyl-1-(2-fluoro-1-methyl-propyl)-5-meth-yl-N-pyridazin-4-yl-pyrazole-4-carboxamide (O.28.48), 1-(1,2-dimethylpropyl)-N,5-dimethyl-N-pyridazin-4-yl-pyrazole-4-carboxamide (O.28.49), 1-[1-(1-cyanocyclopropyl)ethyl]-N,5-dimethyl-N-pyridazin-4-yl-pyrazole-4-carboxamide (O.28.50), N-methyl-1-(2-fluoro-1-methyl-propyl]-5-methyl-N-pyridazin-4-yl-pyrazole-4-carboxamide (O.28.51), 1-(4,4-difluorocyclohexyl)-N-ethyl-5-methyl-N-pyridazin-4-yl-pyrazole-4-carboxamide (O.28.52), 1-(4,4-difluorocyclohexyl)-N,5-dimethyl-N-pyridazin-4-yl-pyrazole-4-carboxamide (O.28.53), N-(1-methylethyl)-2-(3-pyridinyl)-2H-indazole-4-carboxamide (O.28.54), N-cyclopropyl-2-(3-pyridinyl)-2H-indazole-4-carboxamide (O.28.55), N-cyclohexyl-2-(3-pyridinyl)-2H-indazole-4-carboxamide (O.28.56), 2-(3-pyridinyl)-N-(2,2,2-trifluoroethyl)-2H-indazole-4-carboxamide (O.28.57), 2-(3-pyridinyl)-N-[(tetrahydro-2-furanyl)methyl]-2H-indazole-5-carboxamide (O.28.58), methyl 2-[[2-(3-pyridinyl)-2H-indazol-5-yl]carbonyl]hydrazinecarboxylate (O.28.59), N-[(2,2-difluorocyclopropyl)methyl]-2-(3-pyridinyl)-2H-indazole-5-carboxamide (O.28.60), N-(2,2-difluoropropyl)-2-(3-pyridinyl)-2H-indazole-5-carboxamide (O.28.61), 2-(3-pyridinyl)-N-(2-pyrimidinylmethyl)-2H-indazole-5-carboxamide (O.28.62), N-[(5-methyl-2-pyrazinyl)methyl]-2-(3-pyridinyl)-2H-indazole-5-carboxamide (O.28.63), tyclopyrazoflor (O.28.64), sarolaner (O.28.65), lotilaner (O.28.66), N-[4-chloro-3-[[(phenylmethyl)amino]carbonyl]phenyl]-1-methyl-3-(1,1,2,2,2-pentafluoroethyl)-4-(trifluoromethyl)-1H-pyrazole-5-carboxamide (O.28.67), M.UN.22a 2-(3-ethylsulfonyl-2-pyridyl)-3-methyl-6-(trifluoromethyl)imidazo[4,5-b]pyridine (O.28.68), 2-[3-ethylsulfonyl-5-(trifluoromethyl)-2-pyridyl]-3-methyl-6-(trifluoromethyl)imidazo[4,5-b]pyridine (O.28.69), iscocycloseram (O.28.70), N-[4-chloro-3-(cyclopropylcarbamoyl)phenyl]-2-methyl-5-(1,1,2,2,2-pentafluoroethyl)-4-(trifluoromethyl)pyrazole-3-carboxamide (O.28.72), N-[4-chloro-3-[(1-cyanocyclopropyl)carbamoyl]phenyl]-2-methyl-5-(1,1,2,2,2-pentafluoroethyl)-4-(trifluoromethyl)pyrazole-3-carboxamide (O.28.73), acynonapyr (O.28.74), benzpyrimoxan (O.28.75), chloro-N-(1-cyanocyclopropyl)-5-[1-[2-methyl-5-(1,1,2,2,2-pentafluoroethyl)-4-(trifluoromethyl)pyrazol-3-yl]pyrazol-4-yl]benzamide (O.28.76), oxazosulfyl (O.28.77), [(2S,3R,4R,5S,6S)-3,5-dimethoxy-6-methyl-4-propoxy-tetrahydropyran-2-yl]-N-[4-[1-[4-(trifluoromethoxy)phenyl]-1,2,4-triazol-3-yl]phenyl]carbamate (O.28.78), [(2S,3R,4R,5S,6S)-3,4,5-trimethoxy-6-methyl-tetrahydropyran-2-yl] N-[4-[1-[4-(trifluoromethoxy)phenyl]-1,2,4-triazol-3-yl]phenyl]carbamate (O.28.79), [(2S,3R,4R,5S,6S)-3,5-dimethoxy-6-methyl-4-propoxy-tetrahydropyran-2-yl]-N-[4-[1-[4-(1,1,2,2,2-pentafluoroethoxy)phenyl]-1,2,4-triazol-3-yl]phenyl]carbamate (O.28.80), [(2S,3R,4R,5S,6S)-3,4,5-trimethoxy-6-methyl-tetrahydropyran-2-yl]-N-[4-[1-[4-(1,1,2,2,2-pentafluoroethoxy)phenyl]-1,2,4-triazol-3-yl]phenyl]carbamate (O.28.81), (2Z)-3-(2-isopropylphenyl)-2-[(E)-[4-[1-[4-(1,1,2,2,2-pentafluoroethoxy)phenyl]-1,2,4-triazol-3-yl]phenyl]methylenehydrazono]thiazolidin-4-one (O.28.82), 2-(6-chloro-3-ethylsulfonyl-imidazo[1,2-a]pyridin-2-yl)-3-methyl-6-(trifluoromethyl)imidazo[4,5-b]pyridine (O.28.83), 2-(6-bromo-3-ethylsulfonyl-imidazo[1,2-a]pyridin-2-yl)-3-methyl-6-(trifluoromethyl)imidazo[4,5-b]pyridine (O.28.84), 2-(3-ethylsulfonyl-6-iodo-imidazo[1,2-a]pyridin-2-yl)-3-methyl-6-(trifluoromethyl)imidazo[4,5-b]pyridine (O.28.85), 2-[3-ethylsulfonyl-6-(trifluoromethyl)imidazo[1,2-a]pyridin-2-yl]-3-methyl-6-(trifluoromethyl)imidazo[4,5-b]pyridine (O.28.86), 2-(7-chloro-3-ethylsulfonyl-imidazo[1,2-a]pyridin-2-yl)-3-methyl-6-(trifluoromethyl)imidazo[4,5-b]pyridine (O.28.87), 2-(3-ethylsulfonyl-7-iodo-imidazo[1,2-a]pyridin-2-yl)-3-methyl-6-(trifluoromethyl)imidazo[4,5-b]pyridine (O.28.88), 3-ethylsulfonyl-6-iodo-2-[3-methyl-6-(trifluoromethyl)imidazo[4,5-b]pyridin-2-yl]imidazo[1,2-a]pyridine-8-carbonitrile (O.28.89), 2-[3-ethylsulfonyl-8-fluoro-6-(trifluoromethyl)imidazo[1,2-a]pyridin-2-yl]-3-methyl-6-(trifluoromethyl)imidazo[4,5-b]pyridine (O.28.90), 2-[3-ethylsulfonyl-7-(trifluoromethyl)imidazo[1,2-a]pyridin-2-yl]-3-methyl-6-(trifluoromethylsulfinyl)imidazo[4,5-b]pyridine (O.28.91), 2-[3-ethylsulfonyl-7-(trifluoromethyl)imidazo[1,2-a]pyridin-2-yl]-3-methyl-6-(trifluoromethyl)imidazo[4,5-c]pyridine (O.28.92), 2-(6-bromo-3-ethylsulfonyl-imidazo[1,2-a]pyridin-2-yl)-6-(trifluoromethyl)pyrazolo[4,3-c]pyridine (O.28.93).

The active substances referred to as component 2, their preparation and their activity e. g. against harmful fungi is known (cf.: http://www.alanwood.net/pesticides/); these substances are commercially available. The compounds described by IUPAC nomenclature, their preparation and their pesticidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. Nos. 3,296,272; 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 10/139271, WO 11/028657, WO 12/168188, WO 07/006670, WO 11/77514; WO 13/047749, WO 10/069882, WO 13/047441, WO 03/16303, WO 09/90181, WO 13/007767, WO 13/010862, WO 13/127704, WO 13/024009, WO 13/24010, WO 13/047441, WO 13/162072, WO 13/092224, WO 11/135833, CN 1907024, CN 1456054, CN 103387541, CN 1309897, WO 12/84812, CN 1907024, WO 09094442, WO 14/60177, WO 13/116251, WO 08/013622, WO 15/65922, WO 94/01546, EP 2865265, WO 07/129454, WO 12/165511, WO 11/081174, WO 13/47441, JP2015089883, JP2015120675, WO2015119246, WO2011135827, WO2012084812).

The present invention furthermore relates to agrochemical compositions comprising a mixture of at least one compound I (component 1) and at least one further active substance useful for plant protection, e. g. selected from the groups A) to 0) (component 2), in particular one further fungicide, e. g. one or more fungicide from the groups A) to K), as described above, and if desired one suitable solvent or solid carrier. Those mixtures are of particular interest, since many of them at the same application rate show higher efficiencies against harmful fungi. Furthermore, combating harmful fungi with a mixture of compounds I and at least one fungicide from groups A) to K), as described above, is more efficient than combating those fungi with individual compounds I or individual fungicides from groups A) to K).

By applying compounds I together with at least one active substance from groups A) to O) a synergistic effect can be obtained, i.e. more than simple addition of the individual effects is obtained (synergistic mixtures).

This can be obtained by applying the compounds I and at least one further active substance simultaneously, either jointly (e. g. as tank-mix) or separately, or in succession, wherein the time interval between the individual applications is selected to ensure that the active substance applied first still occurs at the site of action in a sufficient amount at the time of application of the further active substance(s). The order of application is not essential for working of the present invention.

When applying compounds I and a pesticide II sequentially the time between both applications may vary e. g. between 2 hours to 7 days. Also, a broader range is possible ranging from 0.25 hour to 30 days, preferably from 0.5 hour to 14 days, particularly from 1 hour to 7 days or from 1.5 hours to 5 days, even more preferred from 2 hours to 1 day.

In the binary mixtures and compositions according to the invention the weight ratio of the component 1) and the component 2) generally depends from the properties of the active components used, usually it is in the range of from 1:10,000 to 10,000:1, often it is in the range of from 1:100 to 100:1, regularly in the range of from 1:50 to 50:1, preferably in the range of from 1:20 to 20:1, more preferably in the range of from 1:10 to 10:1, even more preferably in the range of from 1:4 to 4:1 and in particular in the range of from 1:2 to 2:1.

According to further embodiments of the binary mixtures and compositions, the weight ratio of the component 1) and the component 2) usually is in the range of from 1000:1 to 1:1, often in the range of from 100:1 to 1:1, regularly in the range of from 50:1 to 1:1, preferably in the range of from 20:1 to 1:1, more preferably in the range of from 10:1 to 1:1, even more preferably in the range of from 4:1 to 1:1 and in particular in the range of from 2:1 to 1:1.

According to a further embodiments of the binary mixtures and compositions, the weight ratio of the component 1) and the component 2) usually is in the range of from 1:1 to 1:1000, often in the range of from 1:1 to 1:100, regularly in the range of from 1:1 to 1:50, preferably in the range of from 1:1 to 1:20, more preferably in the range of from 1:1 to 1:10, even more preferably in the range of from 1:1 to 1:4 and in particular in the range of from 1:1 to 1:2.

In the ternary mixtures, i.e. compositions according to the invention comprising the component 1) and component 2) and a compound Ill (component 3), the weight ratio of component 1) and component 2) depends from the properties of the active substances used, usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1, and the weight ratio of component 1) and component 3) usually it is in the range of from 1:100 to 100:1, regularly in the range of from 1:50 to 50:1, preferably in the range of from 1:20 to 20:1, more preferably in the range of from 1:10 to 10:1 and in particular in the range of from 1:4 to 4:1.

Any further active components are, if desired, added in a ratio of from 20:1 to 1:20 to the component 1).

These ratios are also suitable for inventive mixtures applied by seed treatment.

Accordingly, the present invention furthermore relates to mixtures comprising one compound of the formula I (component 1, a group represented by the expression “(1)”) and one pesticide II (component 2), wherein pesticide II is an active ingredient selected from the groups A) to O) defined above.

Further embodiments B-1 to B-705 listed in Table B below relate to mixtures comprising as active components one of the in the present specification individualized compounds of the formula I, which is selected from the group of compounds I.A.A-1 to I.A.A-1364, I.B.A-1 to I.B.A-1364, I.C.A-1 to I.C.A-1364, I.D.A-1 to I.D.A-1364, I.E.A-1 to I.E.A-1364, I.F.A-1 to I.F.A-1364, I.G.A-1 to I.G.A-1364 and I.H.A-1 to I.H.A-1364 as defined in tables 1 to 8 (component 1, a group represented by the expression “(I)”), and one pesticide II selected from the groups A) to O) as defined herein (component 2, for example, (A.1.1) or azoxystrobin, in embodiment B-1).

The mixtures of active substances can be prepared as compositions comprising besides the active ingredients at least one inert ingredient (auxiliary) by usual means, e. g. by the means given for the compositions of compounds I.

Concerning usual ingredients of such compositions reference is made to the explanations given for the compositions containing compounds I.

The mixtures of active substances according to the present invention are suitable as fungicides, as are the compounds of formula I. They are distinguished by an outstanding effective-ness against a broad spectrum of phytopathogenic fungi, especially from the classes of the Ascomycetes, Basidiomycetes, Deuteromycetes and Peronosporomycetes (syn. Oomycetes). In addition, it is referred to the explanations regarding the fungicidal activity of the compounds and the compositions containing compounds I, respectively.

### I. Synthesis Examples

The compounds of the formula I can be prepared according to the methods outlined below.

### II. Synthesis Examples

I.1) (Ex-8): (2E)-N-(5,6-dimethyl-3-pyridyl)-2-isobutoxyimino-2-phenyl-acetamide Step 1: Preparation of N-(5,6-dimethyl-3-pyridyl)-2-oxo-2-phenyl-acetamide 2-oxo-2-phenyl-acetyl chloride (1.1 g, 1.0 eq) in dichloromethane (10 mL) was added dropwise to the cooled (10° C.) solution of 5,6-dimethylpyridin-3-amine (O.8 g, 1 eq) and triethylamine (O.66 g, 1 eq) in dichloromethane (15 ml) and stirring was continued for 0.5 f at 23° C. The mixture was freed from solvent under reduced pressure. Flash chromatography on silica gel using methyl tert-butylether/n-heptane provided the titled product afforded the titled product as a white solid, which was used directly in the next step without further purification.

¹H NMR (400 MHz, Chloroform-d) δ 9.11 (s, 1H), 8.50 (d, J=2.5 Hz, 1H), 8.42-8.27 (m, 2H), 8.03 (d, J=2.6 Hz, 1H), 7.70-7.56 (m, 1H), 7.57-7.43 (m, 2H), 2.49 (s, 3H), 2.31 (s, 3H).

### Step 2: Preparation of (2E)-N-(5,6-dimethyl-3-pyridyl)-2-isobutoxyimino-2-phenyl-acetamide (Ex-8)

N-(5,6-dimethyl-3-pyridyl)-2-oxo-2-phenyl-acetamide (220 mg, 1 eq) in EtOH (5 mL) was added to the solution O-isobutylhydroxylamine hydrochloride (109 mg, 1 eql) and triethylamine in ethanol (15 ml) and the reaction mixture was stirred for 8 h at 80° C. and for 12 h at 23° C. The reaction mixture was quenched with H2O (30 ml) and extracted with dichloromethane (2×30 mL). The combined extracts were dried over sodium sulfate and freed from solvent under reduced pressure. Flash chromatography on silica gel using ethyl acetate/n-heptane provided the titled product as a white solid. MP: 169° C.

¹H NMR (400 MHz, Chloroform-d) δ: 8.40 (s, 1H), 8.25 (d, J=2.5 Hz, 1H), 8.08 (d, J=2.5 Hz, 1H), 7.71-7.57 (m, 2H), 7.42-7.30 (m, 3H), 4.04 (d, J=6.8 Hz, 2H), 2.44 (s, 3H), 2.29 (s, 3H), 2.08 (dp, J=13.4, 6.8 Hz, 1H), 0.95 (d, J=6.7 Hz, 6H).

The compounds listed in Table I were prepared in an analogous manner.

| | | | | | I |
|---|---|---|---|---|---|
| 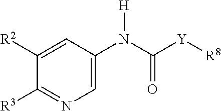 | | | | | |
| Ex-Nr | Melting Point | RT | 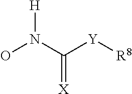 | R² | R³ |
| B-1 | | 0.79 | 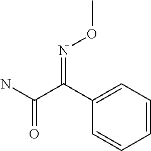 | CH₃ | CH₃ |
| B-2 | | 0.75 | 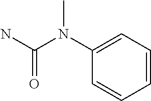 | CH₃ | CH₃ |
| B-3 | | 0.93 | 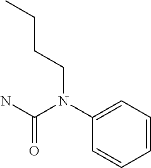 | CH₃ | CH₃ |
| B-4 | | 0.86 | 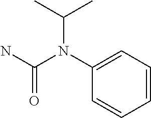 | CH₃ | CH₃ |
| B-5 | | 0.92 | 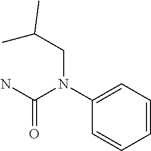 | CH₃ | CH₃ |
| B-6 | | 0.91 | 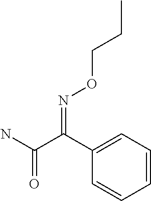 | CH₃ | CH₃ |
| B-7 | | 0.91 | 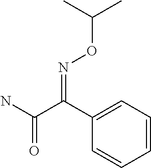 | CH₃ | CH₃ |
| B-8 | | 0.97 | 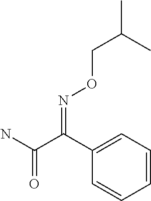 | CH₃ | CH₃ |
| B-9 | | 0.97 | 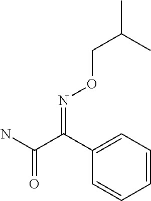 | CH₃ | CH₃ |
| B-10 | | 0.86 | 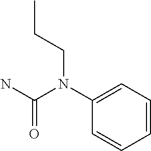 | CH₃ | CH₃ |
| B-11 | | 0.98 | 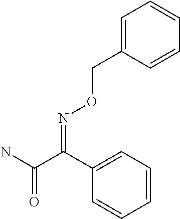 | CH₃ | CH₃ |
| B-12 | | 1.25 | 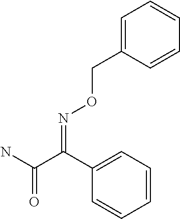 [SMILES: NC(=O)/C(=N/OCc1ccccc1)c1ccccc1] | CH₃ | CHF₂ |
| B-13 | | 1.08 | 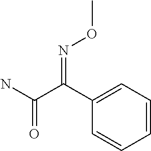 | CH₃ | CHF₂ |
| B-14 | | 1.20 | 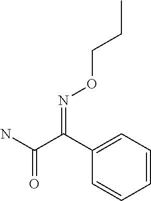 | CH₃ | CHF₂ |
| B-15 | | 1.14 | 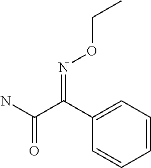 | CH₃ | CHF₂ |
| B-16 | | 1.20 | 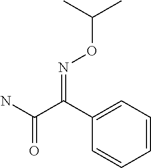 | CH₃ | CHF₂ |
| B-17 | | 0.85 | 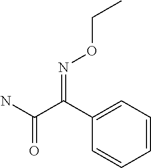 | CH₃ | CH₃ |
| B-18 | | 0.90 | 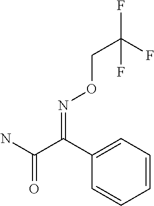 [SMILES: NC(=O)/C(=N/OCC(F)(F)F)c1ccccc1] | CH₃ | CHF₂ |
| B-19 | | 1.25 | 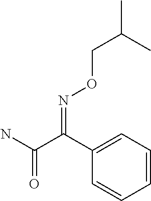 [SMILES: CC(C)CO/N=C(/C(N)=O)c1ccccc1] | CH₃ | CHF₂ |
| B-20 | | 1.17 | 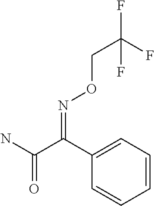 [SMILES: NC(=O)/C(=N/OCC(F)(F)F)c1ccccc1] | CH₃ | CHF₂ |
| B-21 | | 0.85 | 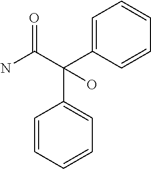 | CH₃ | CH₃ |
| B-22 | | 0.90 | 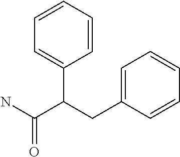 [SMILES: NC(=O)C(Cc1ccccc1)c1ccccc1] | CH₃ | CHF₂ |
| B-23 | | 0.87 | 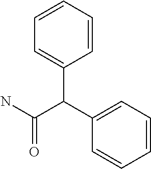 [SMILES: NC(=O)C(c1ccccc1)c1ccccc1] | CH₃ | CHF₂ |
| B-24 | | 1.13 | 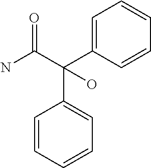 [SMILES: NC(=O)C(O)(c1ccccc1)c1ccccc1] | CH₃ | CHF₂ |
| B-25 | | 1.20 | 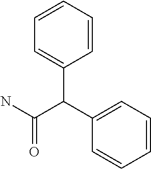 [SMILES: NC(=O)C(c1ccccc1)c1ccccc1] | CH₃ | CHF₂ |
| B-26 | | 1.23 | 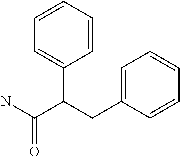 [SMILES: NC(=O)C(Cc1ccccc1)c1ccccc1] | CH₃ | CHF₂ |
| B-27 | | 0.95 | 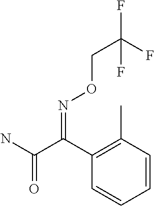 | CH₃ | CH₃ |
| B-28 | | 1.16 | 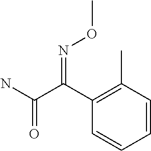 | CH₃ | CHF₂ |
| B-29 | | 1.22 | 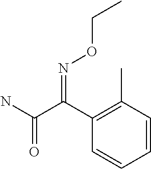 | CH₃ | CHF₂ |
| B-30 | | 1.22 | 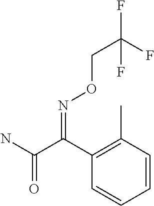 [SMILES: Cc1ccccc1/C(=N\OCC(F)(F)F)C(N)=O] | CH₃ | CHF₂ |
| B-31 | | 0.88 | 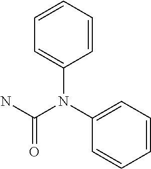 | CH₃ | CH₃ |
| B-32 | | 1.53 | 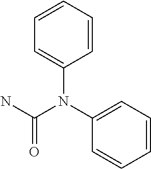 [SMILES: NC(=O)N(c1ccccc1)c1ccccc1] | CH₃ | CHF₂ |
| B-33 | | 0.95 | 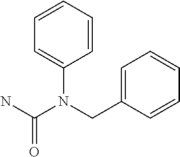 | CH₃ | CH₃ |
| B-34 | | 1.20 | 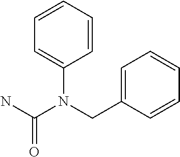 [SMILES: NC(=O)N(Cc1ccccc1)c1ccccc1] | CH₃ | CHF₂ |
| B-35 | | 1.26 | 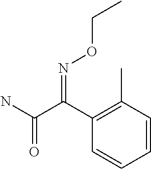 | CH₃ | CHF₂ |
| B-36 | | 1.31 | 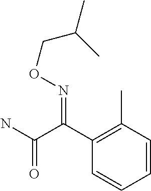 [SMILES: Cc1ccccc1/C(=N/OCC(C)C)C(N)=O] | CH₃ | CHF₂ |
| B-37 | | 0.95 | 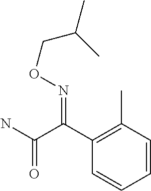 | CH₃ | CH₃ |
| B-38 | | 0.99 | 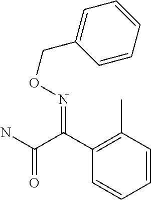 | CH₃ | CH₃ |
| B-39 | | 0.89 | 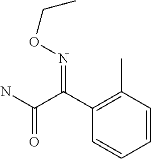 | CH₃ | CH₃ |
| B-40 | | 0.95 | 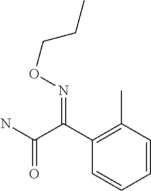 | CH₃ | CH₃ |
| B-41 | | 1.27 | 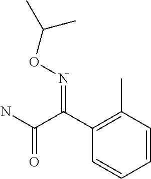 [SMILES: Cc1ccccc1/C(=N/OC(C)C)C(N)=O] | CH₃ | CHF₂ |
| B-42 | | 1.30 | 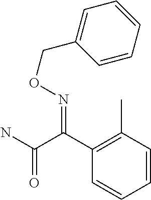 [SMILES: Cc1ccccc1/C(=N/OCc1ccccc1)C(N)=O] | CH₃ | CHF₂ |
| B-43 | | 0.83 | 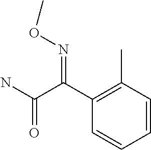 | CH₃ | CH₃ |
| B-44 | | 0.99 | 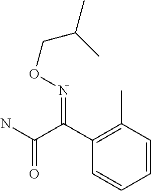 | CH₃ | CH₃ |
| B-45 | | 0.92 | 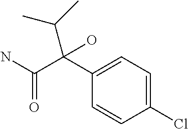 | CH₃ | CH₃ |
| B-46 | | 1.22 | 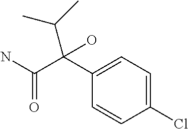 | CH₃ | CHF₂ |
| B-47 | | 0.847 | 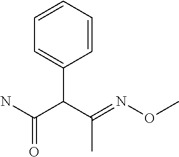 | CH₃ | CH₃ |
| B-48 | | 0.957 | 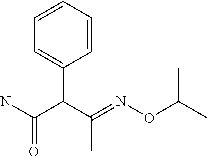 | CH₃ | CH₃ |
| B-49 | | 1.014 | 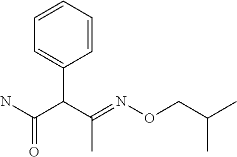 | CH₃ | CH₃ |
| B-50 | | 0.908 | 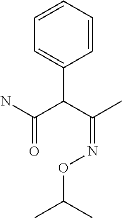 | CH₃ | CH₃ |
| B-51 | | 0.959 | 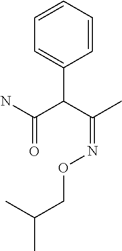 | CH₃ | CH₃ |
| B-52 | | 0.921 | 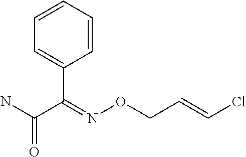 | CH₃ | CH₃ |
| B-53 | 146 | 380 | 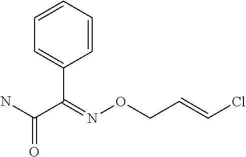 [SMILES: NC(=O)/C(=N/OC/C=C/Cl)c1ccccc1] | CH₃ | CHF₂ |
| B-54 | 141 | 1.249 | 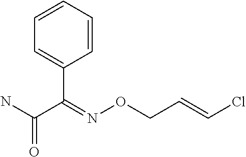 [SMILES: NC(=O)/C(=N/OC/C=C/Cl)c1ccccc1] | 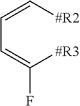 | |
| B-55 | 165 | 1 | 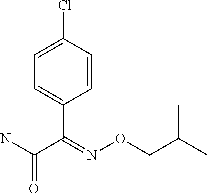 | CH₃ | CH₃ |
| B-56 | 132 | 1.373 | 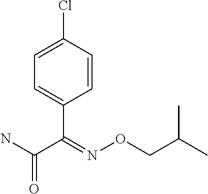 [SMILES: CC(C)CO/N=C(/C(N)=O)c1ccc(Cl)cc1] | CH₃ | CHF₂ |
| B-57 | | 1.365 | 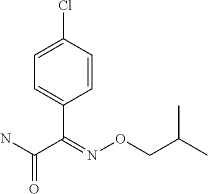 [SMILES: CC(C)CO/N=C(/C(N)=O)c1ccc(Cl)cc1] | 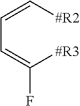 | |
| B-58 | 176 | 0.97 | 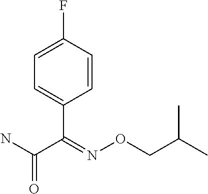 | CH₃ | CH₃ |
| B-59 | 137 | 1.302 | 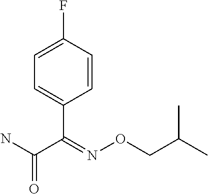 [SMILES: CC(C)CO/N=C(/C(N)=O)c1ccc(F)cc1] | 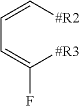 | |
| B-60 | 165 | 1.009 | 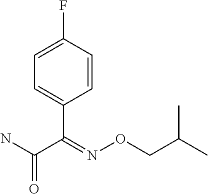 | CH₃ | CH₃ |
| B-61 | | 0.921 | 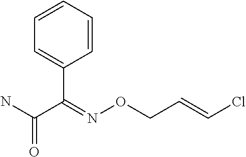 | CH₃ | CH₃ |
| B-62 | | 1.284 | 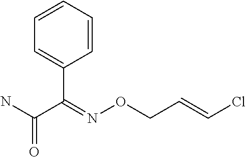 [SMILES: NC(=O)/C(=N/OC/C=C/Cl)c1ccccc1] | CH₃ | CHF₂ |
| B-63 | | 1.282 | 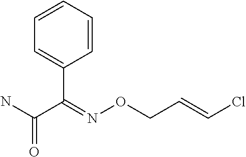 [SMILES: NC(=O)/C(=N/OC/C=C/Cl)c1ccccc1] | 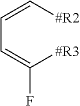 | |
| B-64 | | 0.777 | 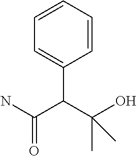 | CH₃ | CH₃ |
| B-65 | | 0.959 | 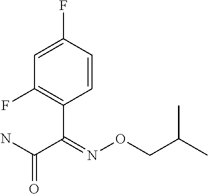 | CH₃ | CH₃ |
| B-66 | | 1.305 | 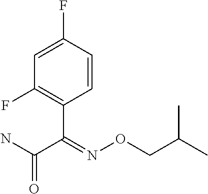 [SMILES: CC(C)CO/N=C(/C(N)=O)c1ccc(F)cc1F] | CH₃ | CHF₂ |
| B-67 | | 1.3 | 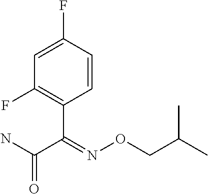 [SMILES: CC(C)CO/N=C(/C(N)=O)c1ccc(F)cc1F] | 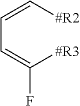 | |
| B-68 | 123 | 1.306 | 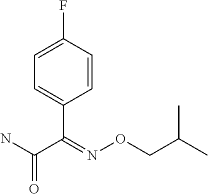 [SMILES: CC(C)CO/N=C(/C(N)=O)c1ccc(F)cc1] | CH₂ | CHF₃ |
| B-69 | 173 | 1.068 | 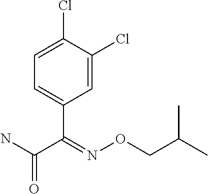 | CH₃ | CH₃ |
| B-70 | 146 | 1.437 | 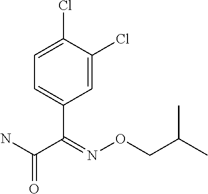 [SMILES: CC(C)CO/N=C(/C(N)=O)c1ccc(Cl)c(Cl)c1] | CH₃ | CHF₂ |
| B-71 | | 1.437 | 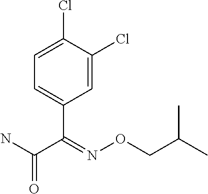 [SMILES: CC(C)CO/N=C(/C(N)=O)c1ccc(Cl)c(Cl)c1] | 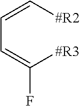 | |
| B-72 | 110 | 0.915 | 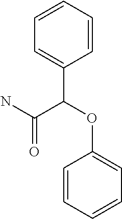 | CH₃ | CH₃ |
| B-73 | | 1.234 | 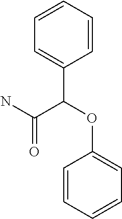 [SMILES: NC(=O)C(Oc1ccccc1)c1ccccc1] | CH₃ | CHF₂ |
| B-74 | 153 | 1.223 | 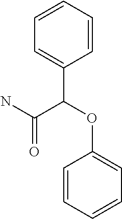 [SMILES: NC(=O)C(Oc1ccccc1)c1ccccc1] | 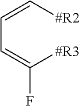 | |
| B-75 | 157 | 0.962 | 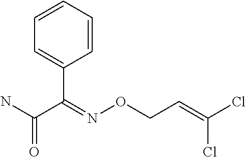 | CH₃ | CH₃ |
| B-76 | | 1.286 | 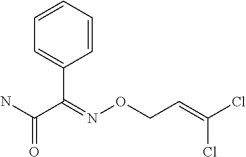 [SMILES: NC(=O)/C(=N/OCC=C(Cl)Cl)c1ccccc1] | CH₃ | CHF₂ |
| B-77 | 59 | 0.947 | 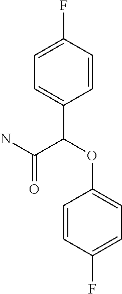 | CH₃ | CH₃ |
| B-78 | 129 | 1.25 | 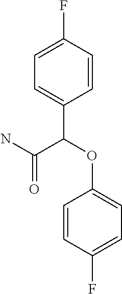 [SMILES: NC(=O)C(Oc1ccc(F)cc1)c1ccc(F)cc1] | CH₃ | CHF₂ |
| B-79 | 80 | 1.238 | 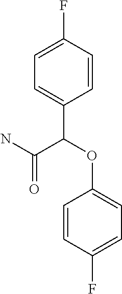 [SMILES: NC(=O)C(Oc1ccc(F)cc1)c1ccc(F)cc1] | 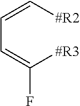 | |
| B-80 | | 1.303 | 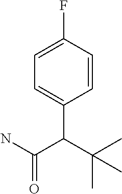 | CH₃ | CHF₂ |
| B-81 | 116 | 0.86 | 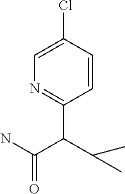 | CH₃ | CH₃ |
| B-82 | 90 | 1.153 | 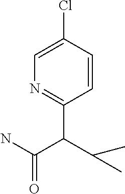 | CH₃ | CHF₂ |
| B-83 | | 1.286 | 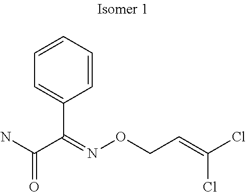 [SMILES: NC(=O)/C(=N/OCC=C(Cl)Cl)c1ccccc1] | 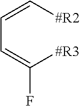 | |
| B-84 | | 1.324 | 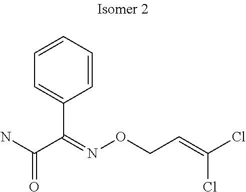 [SMILES: NC(=O)/C(=N/OCC=C(Cl)Cl)c1ccccc1] | 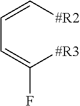 | |
| B-85 | | 0.891 | 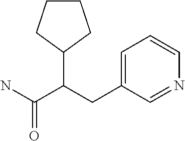 [SMILES: NC(=O)C(Cc1cccnc1)C1CCCC1] | 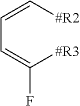 | |
| B-86 | | 0.97 | 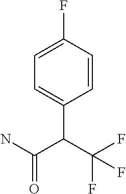 | CH₃ | CH₃ |
| B-87 | 198 | 1,292 | 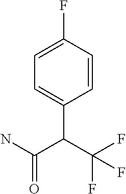 [SMILES: NC(=O)C(c1ccc(F)cc1)C(F)(F)F] | 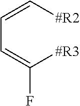 | |
| B-88 | 163 | 0.937 | 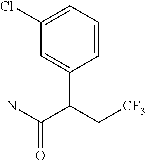 | CH₃ | CH₃ |
| B-89 | 129 | 1.24 | 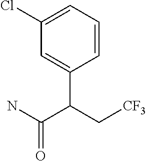 [SMILES: NC(=O)C(CC(F)(F)F)c1cccc(Cl)c1] | CH₃ | CHF₂ |
| B-90 | 184 | 1.236 | 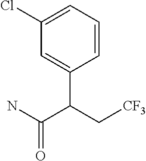 [SMILES: NC(=O)C(CC(F)(F)F)c1cccc(Cl)c1] | 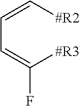 | |
| B-91 | 173 | 0.92 | 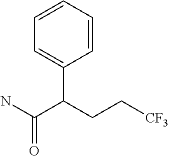 | CH₃ | CH₃ |
| B-92 | 71 | 0.954 | 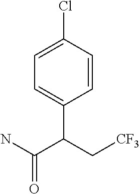 | CH₃ | CH₃ |
| B-93 | 140 | 0.252 | 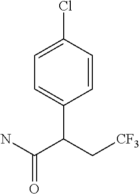 [SMILES: NC(=O)C(CC(F)(F)F)c1ccc(Cl)cc1] | CH₃ | CHF₂ |
| B-94 | 203 | 1.247 | 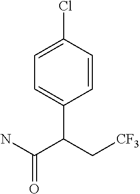 [SMILES: NC(=O)C(CC(F)(F)F)c1ccc(Cl)cc1] | 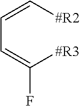 | |
| B-95 | | 1.234 | 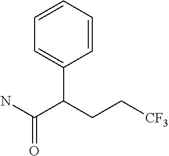 [SMILES: NC(=O)C(CCC(F)(F)F)c1ccccc1] | CH₃ | CHF₂ |
| B-96 | 178 | 1.225 | 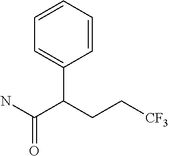 [SMILES: NC(=O)C(CCC(F)(F)F)c1ccccc1] | 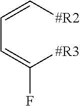 | |
| B-97 | 164 | 0.874 | 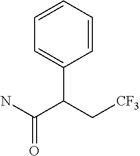 | CH₃ | CH₃ |
| B-98 | 121 | 1.179 | 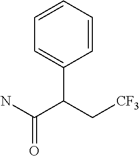 [SMILES: NC(=O)C(CC(F)(F)F)c1ccccc1] | CH₃ | CHF₂ |
| B-99 | 186 | 1.174 | 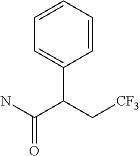 [SMILES: NC(=O)C(CC(F)(F)F)c1ccccc1] | 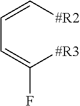 | |
| B-100 | | 0.834 | 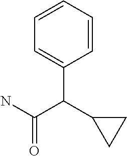 | CH₃ | CH₃ |
| B-101 | 138 | 1.148 | 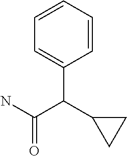 [SMILES: NC(=O)C(c1ccccc1)C1CC1] | CH₃ | CHF₂ |
| B-102 | 133 | 1.133 | 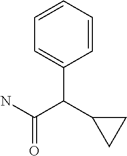 [SMILES: NC(=O)C(c1ccccc1)C1CC1] | 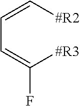 | |
| B-103 | | 0.8555 | 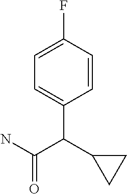 | CH₃ | CH₃ |
| B-104 | 125 | 1.164 | 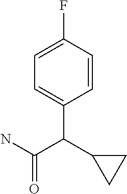 [SMILES: NC(=O)C(c1ccc(F)cc1)C1CC1] | CH₃ | CHF₂ |
| B-105 | | 1.153 | 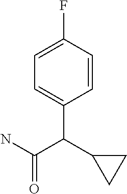 [SMILES: NC(=O)C(c1ccc(F)cc1)C1CC1] | 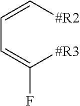 | |
| B-106 | | 0.913 | 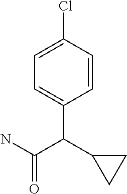 | CH₃ | CH₃ |
| B-107 | 151 | 1.228 | 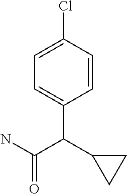 [SMILES: NC(=O)C(c1ccc(Cl)cc1)C1CC1] | CH₃ | CHF₂ |
| B-108 | | 1.218 | 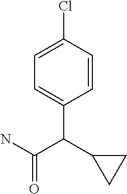 [SMILES: NC(=O)C(c1ccc(Cl)cc1)C1CC1] | 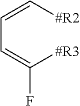 | |
| B-109 | | 0.963 | 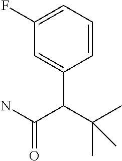 | CH₃ | CH₃ |
| B-110 | | 1.294 | 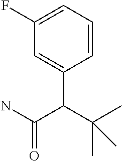 | CH₃ | CHF₂ |
| B-111 | | 1.315 | 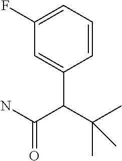 | 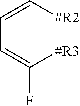 | |
| B-112 | 185 | 1.107 | 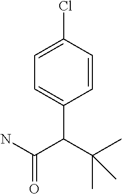 | CH₃ | CH₃ |
| B-113 | | 1.378 | 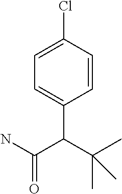 | CH₃ | CHF₂ |
| B-114 | 184 | 1.377 | 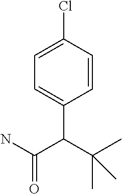 | 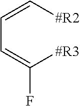 | |
| B-115 | 171 | 0.899 | 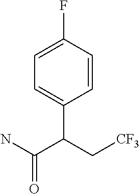 | CH₃ | CH₃ |
| B-116 | 135 | 1.185 | 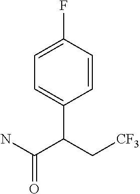 [SMILES: NC(=O)C(CC(F)(F)F)c1ccc(F)cc1] | CH₃ | CHF₂ |
| B-117 | 188 | 1.178 | 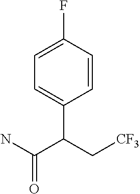 [SMILES: NC(=O)C(CC(F)(F)F)c1ccc(F)cc1] | 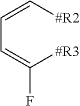 | |
| B-118 | 170 | 0.899 | 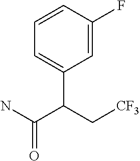 | CH₃ | CH₃ |
| B-119 | 146 | 1.188 | 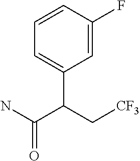 [SMILES: NC(=O)C(CC(F)(F)F)c1cccc(F)c1] | CH₃ | CHF₂ |
| B-120 | 190 | 1.183 | 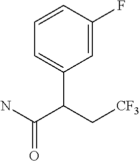 [SMILES: NC(=O)C(CC(F)(F)F)c1cccc(F)c1] | 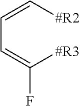 | |
| B-121 | | 1.009 | 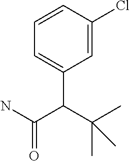 | CH₃ | CH₃ |
| B-122 | | 1.375 | 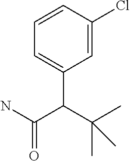 | CH₃ | CHF₂ |
| B-123 | | 1.371 | 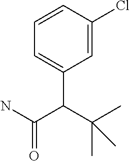 | 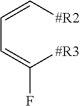 | |
| B-124 | | 0.947 | 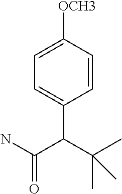 | CH₃ | CH₃ |
| B-125 | | 1.288 | 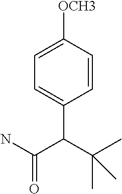 [SMILES: COc1ccc(C(C(N)=O)C(C)(C)C)cc1] | CH₃ | CHF₂ |
| B-126 | 128 | 0.885 | 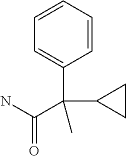 | CH₃ | CH₃ |
| B-127 | | 1.216 | 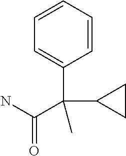 | CH₃ | CHF₂ |
| B-128 | | 0.954 | 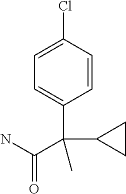 | CH₃ | CH₃ |
| B-129 | 150 | 1.288 | 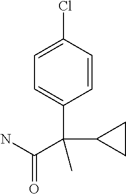 | CH₃ | CHF₂ |
| B-130 | | 1.273 | 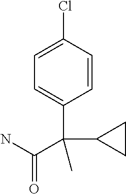 | 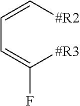 | |
| B-131 | | 1.28 | 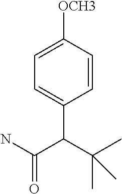 [SMILES: COc1ccc(C(C(N)=O)C(C)(C)C)cc1] | 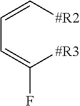 | |
| B-132 | | 1.008 | 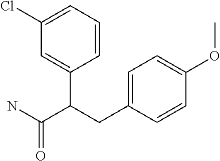 | CH₃ | CH₃ |
| B-133 | | 1.102 | 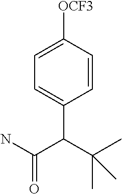 | CH₃ | CH₃ |
| B-134 | | 1.41 | 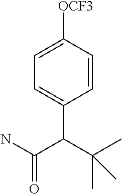 [SMILES: CC(C)(C)C(C(N)=O)c1ccc(OC(F)(F)F)cc1] | CH₃ | CHF₂ |
| B-135 | | 1.409 | 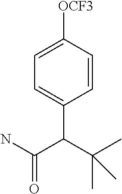 [SMILES: CC(C)(C)C(C(N)=O)c1ccc(OC(F)(F)F)cc1] | 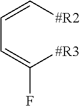 | |
| B-136 | | 0.925 | 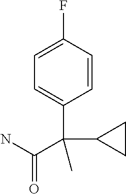 | CH₃ | CH₃ |
| B-137 | | 1.206 | 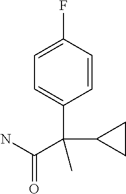 | CH₃ | CHF₂ |
| B-138 | | 1.065 | 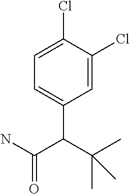 | CH₃ | CH₃ |
| B-139 | | 1.404 | 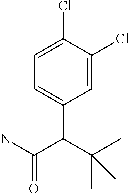 [SMILES: CC(C)(C)C(C(N)=O)c1ccc(Cl)c(Cl)c1] | CH₃ | CHF₂ |
| B-140 | | 1.408 | 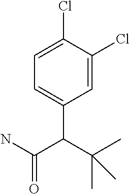 [SMILES: CC(C)(C)C(C(N)=O)c1ccc(Cl)c(Cl)c1] | 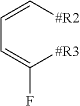 | |
| B-141 | | 1.006 | 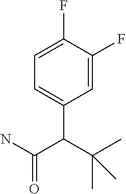 | CH₃ | CH₃ |
| B-142 | | 1.311 | 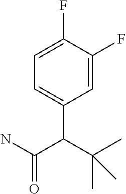 [SMILES: CC(C)(C)C(C(N)=O)c1ccc(F)c(F)c1] | CH₃ | CHF₂ |
| B-143 | | 1.145 | 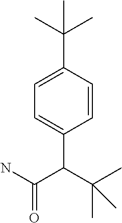 | CH₃ | CH₃ |
| B-144 | | 1.445 | 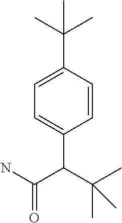 [SMILES: CC(C)(C)c1ccc(C(C(N)=O)C(C)(C)C)cc1] | CH₃ | CHF₂ |
| B-145 | | 1.447 | 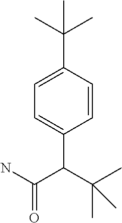 [SMILES: CC(C)(C)c1ccc(C(C(N)=O)C(C)(C)C)cc1] | 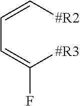 | |
| B-146 | | 0.9 | 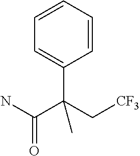 | CH₃ | CH₃ |
| B-147 | 96 | 0.865 | 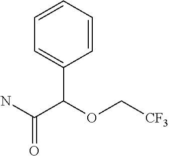 | CH₃ | CH₃ |
| B-148 | | 0.863 | 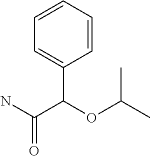 | CH₃ | CH₃ |
| B-149 | | 0.936 | 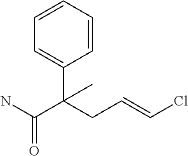 | CH₃ | CH₃ |
| B-150 | | 0.891 | 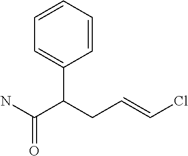 | CH₃ | CH₃ |
| B-151 | 116 | 0.955 | 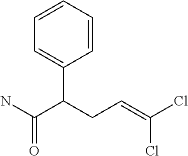 | CH₃ | CH₃ |
| B-152 | | 1.227 | 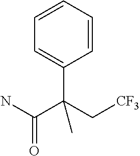 [SMILES: CC(CC(F)(F)F)(C(N)=O)c1ccccc1] | CH₃ | CHF₂ |
| B-153 | | 1.171 | 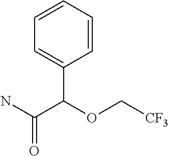 [SMILES: NC(=O)C(OCC(F)(F)F)c1ccccc1] | CH₃ | CHF₂ |
| B-154 | | 1.206 | 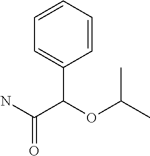 | CH₃ | CHF₂ |
| B-155 | | 1.27 | 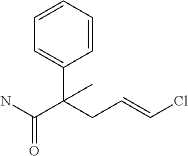 | CH₃ | CHF₂ |
| B-156 | | 1.217 | 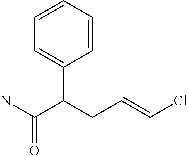 [SMILES: NC(=O)C(C/C=C/Cl)c1ccccc1] | CH₃ | CHF₂ |
| B-157 | | 1.293 | 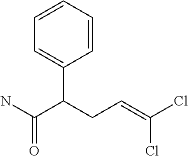 [SMILES: NC(=O)C(CC=C(Cl)Cl)c1ccccc1] | CH₃ | CHF₂ |
| B-158 | 89 | 1.214 | 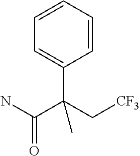 [SMILES: CC(CC(F)(F)F)(C(N)=O)c1ccccc1] | 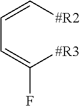 | |
| B-159 | 151 | 1.14 | 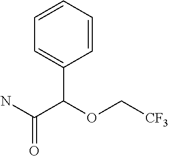 [SMILES: NC(=O)C(OCC(F)(F)F)c1ccccc1] | 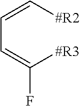 | |
| B-160 | 160 | 1.169 | 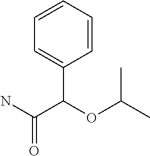 | 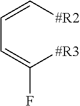 | |
| B-161 | 67 | 1.255 | 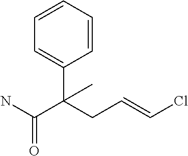 | 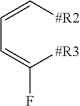 | |
| B-162 | 68 | 1.207 | 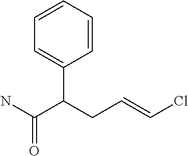 [SMILES: NC(=O)C(C/C=C/Cl)c1ccccc1] | 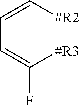 | |
| B-163 | 69 | 1.285 | 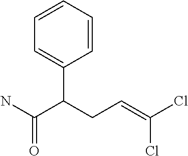 [SMILES: NC(=O)C(CC=C(Cl)Cl)c1ccccc1] | 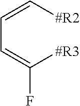 | |
| B-164 | | 1.001 | 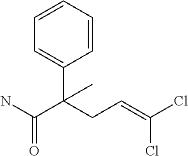 | CH₃ | CH₃ |
| B-165 | | 1.346 | 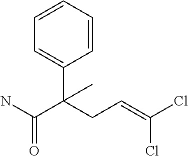 [SMILES: CC(CC=C(Cl)Cl)(C(N)=O)c1ccccc1] | CH₃ | CHF₂ |
| B-166 | 80 | 1.336 | 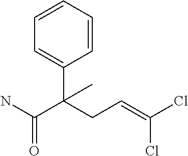 [SMILES: CC(CC=C(Cl)Cl)(C(N)=O)c1ccccc1] | 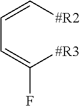 | |
| B-167 | | 0.923 | 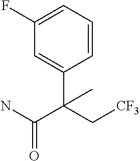 | CH₃ | CH₃ |
| B-168 | 101 | 1.238 | 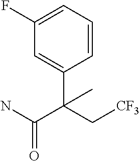 [SMILES: CC(CC(F)(F)F)(C(N)=O)c1cccc(F)c1] | CH₃ | CHF₂ |
| B-169 | 89 | 1.225 | 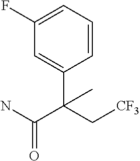 [SMILES: CC(CC(F)(F)F)(C(N)=O)c1cccc(F)c1] | 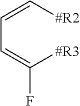 | |
| B-170 | 114 | 0.925 | 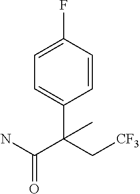 | CH₃ | CH₃ |
| B-171 | 67 | 0.977 | 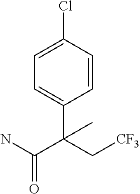 | CH₃ | CH₃ |
| B-172 | 61 | 0.964 | 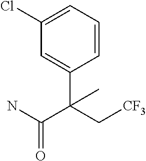 | CH₃ | CH₃ |
| B-173 | 95 | 1.225 | 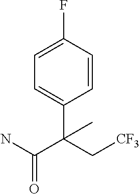 [SMILES: CC(CC(F)(F)F)(C(N)=O)c1ccc(F)cc1] | 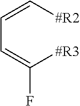 | |
| B-174 | 108 | 1.28 | 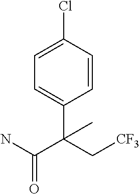 [SMILES: CC(CC(F)(F)F)(C(N)=O)c1ccc(Cl)cc1] | 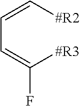 | |
| B-175 | 86 | 1.279 | 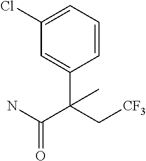 [SMILES: CC(CC(F)(F)F)(C(N)=O)c1cccc(Cl)c1] | 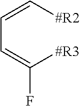 | |
| B-176 | 113 | 1.237 | 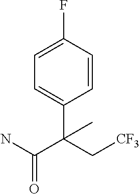 [SMILES: CC(CC(F)(F)F)(C(N)=O)c1ccc(F)cc1] | CH₃ | CHF₂ |
| B-177 | 86 | 1.287 | 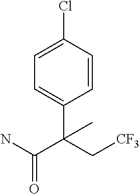 [SMILES: CC(CC(F)(F)F)(C(N)=O)c1ccc(Cl)cc1] | CH₃ | CHF₂ |
| B-178 | 56 | 1.281 | 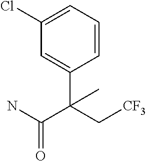 [SMILES: CC(CC(F)(F)F)(C(N)=O)c1cccc(Cl)c1] | CH₃ | CHF₂ |
| B-179 | 53 | 0.95 | 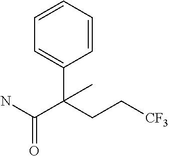 | CH₃ | CH₃ |
| B-180 | 50 | 1.27 | 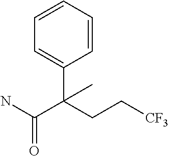 [SMILES: CC(CCC(F)(F)F)(C(N)=O)c1ccccc1] | CH₃ | CHF₂ |
| B-181 | 82 | 1.257 | 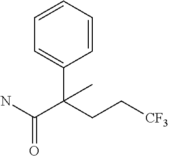 [SMILES: CC(CCC(F)(F)F)(C(N)=O)c1ccccc1] | 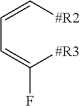 | |
| B-182 | 64 | 0.84 | 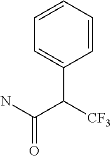 | CH₃ | CH₃ |
| B-183 | 51 | 0.853 | 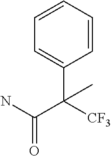 | CH₃ | CH₃ |

### Green House

The compound was dissolved in a mixture of acetone and/or dimethylsulfoxide and the wetting agent/emulsifier Wettol, which is based on ethoxylated alkylphenoles, in a ratio (volume) solvent-emulsifier of 99 to 1 to give a total volume of 5 ml. Subsequently, water was added to total volume of 100 ml.

This stock solution was then diluted with the described solvent-emulsifier-water mixture to the final concentration given in the table below.

### Example 1—Preventative Fungicidal Control of Botrytis cinerea on Leaves of Green Pepper

Young seedlings of green pepper were grown in pots to the 4 to 5 leaf stage. These plants were sprayed to run-off with previously described spray solution, containing the concentration of active ingredient or mixture mentioned in the table below. The next day the plants were inoculated with an aqueous DOB solution (or a DOB solution containing 10 percent glycerine), containing the spore suspension of Botrytis cinerea. Then the plants were immediately transferred to a humid chamber. After 5 days at 22 to 241C and a saturated relative humidity, the extent of fungal attack on the leaves was visually assessed as % diseased leaf area.

In this test, the samples which had been treated with 250 ppm of the active substance from examples Ex-3, Ex-4, Ex-22, Ex-26, Ex-80, Ex-86, Ex-87, Ex-95, Ex-103, Ex-108, ex-109, Ex-110, Ex-111, Ex-112, Ex-113, Ex-114, Ex-120, Ex-121, Ex-122, Ex-123, Ex-124, Ex-125, Ex-130, Ex-131, Ex-133, Ex. 134, Ex-135, Ex-136, Ex-137, Ex-141, ex-143, Ex-144, Ex-148, Ex-150, Ex-151, Ex-152, Ex-153, ex-154, Ex-155, Ex-156, Ex-157, ex-158, Ex-161, ex-162, ex-163, Ex-164, Ex-166, ex-191, Ex-193, Ex-194 respectively, showed up to at most 20% growth of the pathogen whereas the untreated plants were 100% infected.

1. A compound of formula I 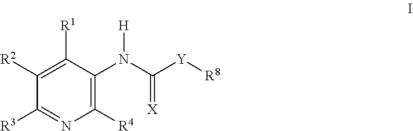
wherein
X O or S;
R¹ is in each case independently selected from hydrogen, halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl, C₃-C₆-cycloalkyl, wherein the acyclic and cyclic moieties of R¹ are unsubstituted or substituted by one to six groups R^{1a} which independently of one another are selected from:
halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl; and wherein the groups R^{1a} are unsubstituted or substituted by one to six halogen or CN;
R² is in each case independently selected from halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl, C₃-C₆-cycloalkyl, wherein the acyclic and cyclic moieties of R² are unsubstituted or substituted by one to six groups R^{2a} which independently of one another are selected from:
halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl; and wherein the groups R^{2a} are unsubstituted or substituted by one to six halogen or CN;
R³ is in each case independently selected from halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl, C₃-C₆-cycloalkyl, wherein the acyclic and cyclic moieties of R³ are unsubstituted or substituted by one to six groups R^{3a} which independently of one another are selected from:
halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl; and wherein the groups R^{3a} are unsubstituted or substituted by one to six halogen or CN;
R² and R³ together with the carbon atoms they are attached to can form a substituted ring of the formula II: 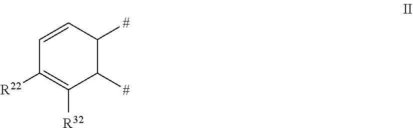
wherein
# means the connection to the pyridine ring of the formula I:
R²² is selected from the group consisting of H, halogen, C₁-C₆-alkyl, C₁-C₆-halogenalkyl.
R³² is selected from the group consisting of halogen, C₁-C₆-alkyl, C₁-C₆-halogenalkyl; and
R⁴ is in each case independently selected from hydrogen, halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl, C₃-C₆-cycloalkyl, wherein the acyclic and cyclic moieties of R⁴ are unsubstituted or substituted by one to six groups R^{4a} which independently of one another are selected from:
halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl; and wherein the groups R^{4a} are unsubstituted or substituted by one to six halogen or CN;
Y is NR⁵, CR⁶R⁷;
R⁵ is in each case independently selected from CN, C₁-C₆-alkyl, C₂-C₆-alkenyl, C₂-C₆-alkynyl, O—C₁-C₆-alkyl, O—C₂-C₆-alkenyl, O—C₂-C₆-alkynyl, C₃-C₆-cycloalkyl, CH₂—C₃-C₆-cycloalkyl, O—C₃-C₆-cycloalkyl, C₃-C₆-cycloalkenyl, O—C₃-C₆-cycloalkenyl, C(R″)═N—OR′, phenyl, —CH₂-phenyl, a five- or six-membered heteroaryl, or CH₂-heteroaryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
wherein the acyclic and cyclic moieties of R⁵ are unsubstituted or substituted by one to six groups R^{5a} which independently of one another are selected from:
halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
the acyclic and cyclic moieties of R^{5a} are unsubstituted or substituted by one to six groups R^{5b} halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C(R″)═N—OR′;
wherein the groups R^{5b} are unsubstituted or substituted by one to six halogen or CN;
R′ is in each case independently selected from C₁-C₆-alkyl, C₂-C₆-alkenyl, C₂-C₆-alkynyl, wherein the acyclic moieties of R′ are unsubstituted or substituted by one to six groups R^{R′} independently of one another are selected from:
halogen, CN, O—C₁-C₆-alkyl, C₃-C₆-cycloalkyl, O—C₃-C₆-cycloalkyl, phenyl, phenoxy;
wherein the acyclic and cyclic moieties of R^{R′} are unsubstituted or substituted by one to six groups selected from halogen, CN, C₁-C₆-alkyl, C₁-C₆-halogenalkyl, C₃-C₆-cycloalkyl, O—C₁-C₆-alkyl, O—C₁-C₆-halogenalkyl;
R″ is in each case independently selected from hydrogen, C₁-C₆-alkyl, C₂-C₆-alkenyl, C₂-C₆-alkynyl, C₃-C₆-cycloalkyl, C₃-C₆-cycloalkenyl, C(R″)═N—OR′, phenyl, a five- or six-membered heteroaryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
the acyclic and cyclic moieties of R″ are unsubstituted or substituted by one to six groups halogen, CN, C₁-C₆-alkyl, C₁-C₆-halogenalkyl, C₃-C₆-cycloalkyl, O—C₁-C₆—O-alkyl, O—C₁-C₆-halogenalkyl;
R⁶ is in each case independently selected from halogen, CN, C₁-C₈-alkyl, C₂-C₆-alkenyl, C₂-C₆-alkynyl, C₁-C₆-halogenalkyl, C₂-C₆-halogenalkenyl, C₂-C₆-halogenalkynyl, O—C₁-C₈-alkyl, O—C₂-C₈-alkenyl, O—C₂-C₈-alkynyl, C₃-C₆-cycloalkyl, O—C₃-C₆-cycloalkyl, CH₂—C₃-C₆-cycloalkyl, C₃-C₆-cycloalkenyl, O—C₃-C₆-cycloalkenyl, CH₂—C₃-C₆-cycloalkenyl, C(R″)═N—OR′, phenyl, phenoxy, —CH₂-phenyl, a five- or six-membered heteroaryl, heteroaryloxy or CH₂-heteroaryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
wherein the acyclic and cyclic moieties of R⁶ are unsubstituted or substituted by one to six groups R^{6a} which independently of one another are selected from:
halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C₃-C₆-cycloalkenyl, O—C₃-C₆-cycloalkenyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
the acyclic and cyclic moieties of R^{6a} are unsubstituted or substituted by one to six groups R^{6b} halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C(R″)═N—OR′;
wherein the groups R^{6b} are unsubstituted or substituted by one to six halogen or CN;
R⁷ is in each case independently selected from hydrogen, OH, halogen, CN, C₁-C₈-alkyl, C₂-C₆-alkenyl, C₂-C₆-alkynyl, C₁-C₈-halogenalkyl, C₂-C₆-halogenalkenyl, C₂-C₈-halogenalkynyl, O—C₁-C₈-alkyl, O—C₂-C₈-alkenyl, O—C₂-C₈-alkynyl, C₃-C₆-cycloalkyl, O—C₃-C₆-cycloalkyl, CH₂—C₃-C₆-cycloalkyl, C₃-C₆-cycloalkenyl, O—C₃-C₆-cycloalkenyl, CH₂—C₃-C₆-cycloalkenyl, C(R″)═N—OR′, phenyl, phenoxy, —CH₂-phenyl, a five- or six-membered heteroaryl, heteroaryloxy or CH₂-heteroaryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
wherein the acyclic and cyclic moieties of R⁷ are unsubstituted or substituted by one to six groups R^{7a} which independently of one another are selected from:
halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C₃-C₆-cycloalkenyl, O—C₃-C₆-cycloalkenyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
the acyclic and cyclic moieties of R^{7a} are unsubstituted or substituted by one to six groups R^{7b} halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C(R″)═N—OR′;
wherein the groups R^{7b} are unsubstituted or substituted by one to six halogen or CN;
or
R⁶ and R⁷ form together with the C atom to which they are bound a group ═N—OR′;
R⁸ is in each case independently selected from C₃-C₁₀-cycloalkyl, C₃-C₁₀-cycloalkenyl, phenyl or a five- or six-membered heteroaryl, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
wherein the acyclic and cyclic moieties of R⁸ are unsubstituted or substituted by one to six groups R^{8a} which independently of one another are selected from:
halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C₃-C₆-cycloalkenyl, O—C₃-C₆-cycloalkenyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
the acyclic and cyclic moieties of R^{8a} are unsubstituted or substituted by one to six groups R^{8b} halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C₃-C₆-cycloalkenyl, O—C₃-C₆-cycloalkenyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
the groups R^{8b} are unsubstituted or substituted by one to six halogen or CN;
with the proviso that:
if Y is CR⁶R⁷, R¹ and R⁴ are H and
R⁶ is unsubstituted C₁-C₄-alkyl and
R⁷ is H, OH, CH₃, C₂H₅,
R⁸ is not an unsubstituted phenyl;
if Y is CR⁶R⁷, R¹ and R⁴ are H and
R⁶ is F and
R⁷ is F,
R⁸ is not an unsubstituted phenyl;
if Y is CR⁶R⁷, R¹ and R⁴ are H and
R⁶ is CH₃ and
R⁷ is CH₃,
R⁸ is not cyclopropyl;
if Y is CR⁶R⁷, R¹ and R⁴ are H and
R⁶ is CH₂-phenyl and
R⁷ is H,
R⁸ is not cyclopropyl;
if Y is CR⁶R⁷, R¹ is H and R⁴ is CH₃ and
R⁶ is F or CH₃ and
R⁷ is F or CH₃,
R⁸ is not phenyl;
and following compounds are excluded

| | | | | I.A-1 |
|---|---|---|---|---|
| 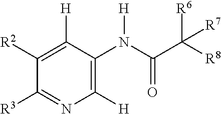 | | | | |
| R² | R³ | R⁶ | R⁷ | R⁸ |
| —CH₃ | —CH₃ | cyclopentyl | H | phenyl |
| —CH₃ | —CH₃ | CF₃ | —OCH₃ | phenyl |
| —CH₃ | —CH₃ | O—C(CH₃)₃ | H | phenyl |

and the N-oxides and the agriculturally acceptable salts thereof for use as fungicides.

2. The compound of claim 1, wherein R¹ is H.

3. The compound of claim 1, wherein R² is selected from C₁-C₆-alkyl, C₁-C₆-halogenalkyl, O—C₁-C₆-alkyl, O—C₁-C₆-halogenalkyl.

4. The compound of claim 1, wherein R³ is selected from C₁-C₆-alkyl, C₁-C₆-halogenalkyl, and O—C₁-C₆-alkyl.

5. The compound of claim 1, wherein R⁴ is H.

6. The compound of claim 1, wherein R⁵ is selected from C₁-C₆-alkyl, C₁-C₆-halogenalkyl, C₂-C₆-alkenyl, C₂-C₆-halogenalkenyl, C₃-C₆-cycloalkyl, CH₂—C₃-C₆-cycloalkyl, C(R″)═N—OR, phenyl, and benzyl; wherein the acyclic and cyclic moieties of R⁵ are unsubstituted or substituted by one to six groups R^{5a} which independently of one another are selected from:
halogen, C₁-C₆-alkyl, C₁-C₆-halogenalkyl, and O—C₁-C₆-alkyl.

7. The compound of claim 1, wherein R⁶ is selected from CN, C₁-C₆-alkyl, C₁-C₆-halogenalkyl, C₂-C₆-alkenyl, C₂-C₆-halogenalkenyl, C₂-C₆-alkinyl, C₂-C₆-halogenalkynyl, C₃-C₆-cycloalkyl, CH₂—C₃-C₆-cycloalkyl, phenyl, benzyl, thienyl-2, and pyridinyl-2, wherein the acyclic and cyclic moieties of R⁶ are unsubstituted or substituted by one to six groups R^{6a} which independently of one another are selected from:
halogen, C₁-C₆-alkyl, C₁-C₆-halogenalkyl, and O—C₁-C₆-alkyl.

8. The compound of claim 1, wherein R⁷ is selected from H, OH, halogen, CN, and C₁-C₆-alkyl.

9. The compound of claim 1, wherein R⁶ and R⁷ form together with the C atom to which they are bound a group ═N—OR′; wherein R′ is C₁-C₆-alkyl, C₁-C₆-halogenalkyl, C₂-C₆-alkenyl, C₂-C₆-halogenalkenyl, C₃-C₆-cycloalkyl, CH₂—C₃-C₆-cycloalkyl, phenyl, or benzyl, wherein the acyclic and cyclic moieties of R′ are unsubstituted or substituted by one to six groups which independently of one another are selected from: halogen, C₁-C₆-alkyl, C₁-C₆-halogenalkyl, and O—C₁-C₆-alkyl.

10. The compound of claim 1, wherein
R¹ is H, CH₃;
R² and R³ together with the carbon atoms they are attached to can form a substituted ring of the formula II: 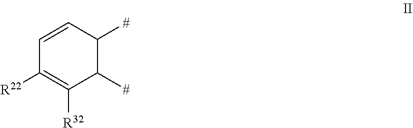
R²² is selected from the group consisting of H, halogen, C₁-C₂-alkyl, gad
C₁-C₂-halogenalkyl.
R³² is selected from the group consisting of halogen, C₁-C₂-alkyl, and C₁-C₂-halogenalkyl;
R⁴ H, CH₃;
Y CR⁶R⁷.

11. The compound of formula I as defined in claim 1,
wherein X, Y, R¹, R³, R⁴, R⁵ and R⁸ are as defined above and
R² is in each case independently selected from CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, O—C₂-C₆-alkynyl, and C₃-C₆-cycloalkyl, wherein the acyclic and cyclic moieties of R² are unsubstituted or substituted by one to six groups R^{2a} which independently of one another are selected from:
halogen, CN, C₁-C₆-alkyl, C₁-C₆-halogenalkyl, C₂-C₆-alkenyl, C₂-C₆-halogenalkenyl, C₂-C₆-alkynyl, C₂-C₆-halogenalkynyl, O—C₁-C₆-alkyl, O—C₂-C₆-alkenyl, and O—C₂-C₆-alkynyl;
and wherein the groups R^{2a} are unsubstituted or substituted by one to six halogen or CN;
R⁶ is in each case independently selected from halogen, CN, C₃-C₆-alkyl, C₂-C₈-alkenyl, C₂-C₈-alkynyl, C₁-C₈-halogenalkyl, C₂-C₈-halogenalkenyl, C₂-C₈-halogenalkynyl, O—C₁-C₆-alkyl, O—C₂-C₈-alkenyl, O—C₂-C₈-alkynyl, C₃-C₆-cycloalkyl, O—C₃-C₆-cycloalkyl, CH₂—C₃-C₆-cycloalkyl, C₃-C₆-cycloalkenyl, O—C₃-C₆-cycloalkenyl, CH₂—C₃-C₆-cycloalkenyl, C(R″)═N—OR′, phenyl, phenoxy, —CH₂-phenyl, a five- or six-membered heteroaryl, heteroaryloxy or CH₂-heteroaryl; wherein the heteroaryl contains 1, 2 or 3 heteroatoms selected from N, O and S; and wherein in each case one or two CH₂ groups of the carbo- or heterocycle may be replaced by a group independently selected from C(═O) and C(═S);
wherein the acyclic and cyclic moieties of R⁶ are unsubstituted or substituted by one to six groups R^{6a} which independently of one another are selected from:
halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, C₃-C₆-cycloalkenyl, O—C₃-C₆-cycloalkenyl, C(R″)═N—OR′, phenyl, phenoxy, a five- or six-membered heteroaryl or heteroaryloxy, wherein the heteroaryl contains 1, 2 or 3 heteroatoms selected from N, O and S; and wherein in each case one or two CH₂ groups of the carbo- or heterocycle may be replaced by a group independently selected from C(═O) and C(═S); and wherein
the acyclic and cyclic moieties of R^{6a} are unsubstituted or substituted by one to six groups R^{6b} halogen, CN, C₁-C₆-alkyl, C₁-C₆-halogenalkyl, C₂-C₆-alkenyl, C₂-C₆-halogenalkenyl, C₂-C₆-alkynyl, O—C₁-C₆-alkyl, O—C₂-C₆-alkenyl, O—C₂-C₆-alkynyl, C₃-C₆-cycloalkyl, O—C₃-C₆-cycloalkyl, and C(R″)═N—OR′;
wherein the groups R^{6b} are unsubstituted or substituted by one to six halogen or CN.

12. The compound of claim 11, wherein R⁶ is selected from C₁-C₆-alkyl, C₂-C₆-halogenalkyl, C₂-C₆-alkenyl, C₂-C₆-halogenalkenyl, C₃-C₆-cycloalkyl, and CH₂—C₃-C₆-cycloalkyl, wherein the cycloalkyl groups are unsubstituted or substituted by one to six halogen or C₁-C₆-alkyl.

13. The compound of claim 1, wherein R⁶ is selected from n-propyl, i-propyl, n-butyl, i-butyl, tert-butyl, n-pentyl, CH(CH₃)—(CH₂)₂—CH₃, (CH₂)₂—CH(CH₃)₂, or CH₂—C(CH₃)₃, CH₂—CF₃, (CH₂)₂—CF₃, CH₂—CH═CHCl, CH₂—CH═CCl₂, CH₂—CCl═CCl₂, cyclopropyl, 1-Cl-cyclopropyl, and CH₂-(1-Cl-cyclopropyl).

14. A composition comprising a compound of formula I, as defined in claim 1, an N-oxide or an agriculturally acceptable salt thereof.

15. A method for combating phytopathogenic fungi, comprising treating the fungi or the materials, plants, the soil or seeds to be protected against fungal attack with an effective amount of at least one compound of formula I, as defined in claim 1.
